(19) United States
(12) Patent Application Publication
(10) Pub. No.: US 2009/0298872 A1
(43) Pub. Date: Dec. 3, 2009

(54) COMPOUNDS AND COMPOSITIONS AS MODULATORS OF STEROIDAL RECEPTORS AND CALCIUM CHANNEL ACTIVITIES

(75) Inventors: Pierre-Yves Michellys, San Marcos, CA (US); Wei Pei, San Diego, CA (US); John Wityak, Carlsbad, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 11/720,907

(22) PCT Filed: Dec. 13, 2005
(86) PCT No.: PCT/US05/45449

§ 371 Date: Jun. 2, 2009

Related U.S. Application Data
(60) Provisional application No. 60/635,760, filed on Dec. 13, 2004.
(60) Provisional application No. 60/652,248, filed on Feb. 11, 2005.

(51) Int. Cl. A61K 31/4418; C07D 211/90; C07D 401/02; C07D 409/04; C07D 215/48; C07D 405/04; A61K 31/444; A61K 31/4436; A61K 31/47; A61K 31/443

(52) U.S. Cl. 514/311; 546/288; 546/258; 546/280.4; 546/165; 546/283.4; 514/344; 514/334; 514/336

(57) ABSTRACT

The invention provides compounds, pharmaceutical compositions comprising such compounds and methods of using such compounds to treat or prevent diseases or disorder associated with the activation of steroid hormone nuclear receptors.

### CROSS-REFERENCE TO RELATED APPLICATIONS

This application claims the benefit of priority to U.S. Provisional Patent Application No. 60/635,760, filed 13 Dec. 2004 and U.S. Provisional Patent Application No. 60/652,248, filed 11 Feb. 2005. The full disclosures of these applications are incorporated herein by reference in their entirety and for all purposes.

### BACKGROUND OF THE INVENTION

1. Field of the Invention

The invention provides compounds, pharmaceutical compositions comprising such compounds and methods of using such compounds to treat or prevent diseases or disorders associated with the activation of steroid hormone nuclear receptors and calcium channel blockade activity.

2. Background

Steroid hormone receptors represent a subset of the nuclear hormone receptor superfamily. So named according to the cognate ligand which complexes with the receptor in its native state, the steroid hormone nuclear receptors include the glucocorticoid receptor (GR), the androgen receptor (AR), the mineralocorticoid receptor (MR), the estrogen receptor (ER), and the progesterone receptor (PR). MR is expressed in epithelial tissues, heart, kidneys, brain, vascular tissues and bone. Aldosterone is the endogenous ligand of MR and is primarily synthesized in the adrenal glands, heart, brain and blood vessels. Several detrimental effects are attributable to aldosterone, for example: sodium/water retention, renal fibrosis, vascular inflammation, vascular fibrosis, endothelial dysfunction, coronary inflammation, decrease in coronary blood flow, ventricular arrhythmias, myocardial fibrosis, ventricular hypertrophy and direct damage to cardiovascular systems, primarily the heart, vasculature and kidneys. Aldosterone action on all target organs is through activation of the MR receptor. GR is expressed in almost all tissues and organ systems and is crucial for the integrity of the function of the central nervous system and the maintenance of cardiovascular, metabolic, and immune homeostasis.

Calcium channel antagonists have long been used as drugs to treat various cardiovascular diseases like coronary vasodilation, angina, arrythmias, congestive heart failure, cardiomyopathy, atheriosclerosis and high blood pressure.

The novel compounds of the invention modulate the activity of the steroid hormone nuclear receptors and calcium channels and are, therefore, expected to be useful in the treatment of diseases in which aberrant activity of steroidal nuclear hormone receptors and/or calcium channels contribute to the pathology and/or symptomatology of the disease.

### SUMMARY OF THE INVENTION

In one aspect, the present invention provides compounds of Formula I:

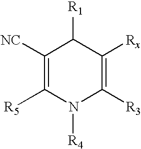

in which:

R₁ is selected from C₆₋₁₀aryl and C₅₋₁₀heteroaryl; wherein any aryl or heteroaryl of R₁ is optionally substituted by 1 to 3 radicals independently selected from halo, C₁₋₆alkyl, C₁₋₆alkoxy, phenyl, halo-substituted-C₁₋₆alkyl and halo-substituted-C₁₋₆alkoxy;

Rₓ is selected from cyano and —C(O)R₂; wherein R₂ is selected from —NR₆R₇ and —OR₇; wherein R₆ is selected from hydrogen, C₁₋₆alkyl and 1-hydroxy-vinyl; and R₇ is selected from C₁₋₆alkyl, halo-substituted-C₁₋₆alkyl, C₃₋₁₂cycloalkyl, C₆₋₁₀aryl and C₅₋₁₀heteroaryl; wherein any cycloalkyl, aryl or heteroaryl of R₇ is optionally substituted by 1 to 3 radicals independently selected from halo, nitro, C₁₋₆alkyl, C₁₋₆alkoxy, phenyl, phenoxy, halo-substituted-C₁₋₆alkyl and halo-substituted-C₁₋₆alkoxy; or R₆ and R₇ together with the nitrogen to which they are both attached form C₅₋₁₀heteroaryl or C₃₋₈heterocycloalkyl;

R₃ is selected from C₁₋₆alkyl, C₃₋₁₂cycloalkyl-C₀₋₄alkyl, C₆₋₁₀aryl-C₀₋₄alkyl and C₅₋₁₀heteroaryl-C₀₋₄alkyl; wherein any alkyl of R₃ can optionally have a methylene replaced with a divalent radical independently selected from —O—, —OC(O)—, —NR₆— and —S(O)₀₋₂—; wherein any alkyl of R₃ can optionally be substituted by 1 to 3 radicals independently selected from halo-substituted-C₁₋₆alkyl; wherein any cycloalkyl, aryl or heteroaryl of R₃ can optionally be substituted with 1 to 2 radicals independently selected from halo, C₁₋₆alkyl and C₁₋₆alkoxy; or R₂ and R₃ together with the atoms to which R₂ and R₃ are attached form C₃₋₁₂cycloalkyl optionally substituted with 1 to 2 radicals independently selected from halo, nitro, C₁₋₆alkyl, C₁₋₆alkoxy, phenyl, halo-substituted-C₁₋₆alkyl and halo-substituted-C₁₋₆alkoxy;

R₄ is selected from hydrogen, C₁₋₆alkyl, halo-substituted-C₁₋₆alkyl and —C(O)R₈; wherein R₈ is selected from hydrogen and C₁₋₆alkyl;

R₅ is selected from C₁₋₆alkyl, —SXC(O)OR₉, —SXOC(O)R₉, —SXR₉, —SXC(O)R₉, —SXNR₉R₉ and —XR₉; wherein X is a bond or C₁₋₆alkylene; R₉ is independently selected from hydroxy, C₁₋₆alkyl, halo-substituted-C₁₋₆alkyl, C₆₋₁₀aryl and C₅₋₁₀heteroaryl; wherein any aryl or heteroaryl of R₉ is optionally substituted with 1 to 3 radicals independently selected from halo, hydroxy, nitro, amino, cyano, C₁₋₆alkyl, C₁₋₆alkoxy, halo-substituted-C₁₋₆alkyl, halo-substituted-C₁₋₆alkoxy, —C(O)OR₁₀, —OR₁₀ and —C(O)R₁₀; wherein R₁₀ is selected from methyl and phenyl; and the N-oxide derivatives, prodrug derivatives, protected derivatives, individual isomers and mixture of isomers thereof; and the pharmaceutically acceptable salts and solvates (e.g. hydrates) of such compounds.

In a second aspect, the present invention provides a pharmaceutical composition which contains a compound of Formula I or a N-oxide derivative, individual isomers and mixture of isomers thereof; or a pharmaceutically acceptable salt thereof, in admixture with one or more suitable excipients.

In a third aspect, the present invention provides a method of treating a disease in an animal in which modulation of steroid nuclear hormone receptor activities and/or calcium channel activities can prevent, inhibit or ameliorate the pathology and/or symptomatology of the diseases, which method comprises administering to the animal a therapeutically effective amount of a compound of Formula I or a N-oxide derivative, individual isomers and mixture of isomers thereof, or a pharmaceutically acceptable salt thereof.

In a fourth aspect, the present invention provides the use of a compound of Formula I in the manufacture of a medicament for treating a disease in an animal in which steroid nuclear hormone receptor activity and/or calcium channel activity contributes to the pathology and/or symptomatology of the disease.

In a fifth aspect, the present invention provides a process for preparing compounds of Formula I and the N-oxide derivatives, prodrug derivatives, protected derivatives, individual isomers and mixture of isomers thereof, and the pharmaceutically acceptable salts thereof.

### DETAILED DESCRIPTION OF THE INVENTION

### Definitions

“Alkyl” as a group and as a structural element of other groups, for example halo-substituted-alkyl and alkoxy, can be either straight-chained or branched. C₁₋₆alkoxy includes, methoxy, ethoxy, and the like. Halo-substituted alkyl includes trifluoromethyl, pentafluoroethyl, and the like.

“Aryl” means a monocyclic or fused bicyclic aromatic ring assembly containing six to ten ring carbon atoms. For example, aryl can be phenyl or naphthyl, preferably phenyl. “Arylene” means a divalent radical derived from an aryl group. “Heteroaryl” is as defined for aryl where one or more of the ring members are a heteroatom. For example heteroaryl includes pyridyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, furanyl, oxazolyl, isoxazolyl, triazolyl, tetrazolyl, pyrazolyl, thienyl, etc. “C₆₋₁₀arylC₀₋₄alkyl” means an aryl as described above connected via a alkylene grouping. For example, C₆₋₁₀arylC₀₋₄alkyl includes phenethyl, benzyl, etc.

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc. “Heterocycloalkyl” means cycloalkyl, as defined in this application, provided that one or more of the ring carbons indicated, are replaced by a moiety selected from —O—, —N═, —NR—, —C(O)—, —S—, —S(O)— or —S(O)₂—, wherein R is hydrogen, C₁₋₄alkyl or a nitrogen protecting group. For example, C₃₋₈heterocycloalkyl as used in this application to describe compounds of the invention includes morpholino, pyrrolidinyl, piperazinyl, piperidinyl, piperidinylone, 1,4-dioxa-8-aza-spiro[4.5]dec-8-yl, etc.

“Halogen” (or halo) preferably represents chloro or fluoro, but can also be bromo or iodo.

“Treat”, “treating” and “treatment” refer to a method of alleviating or abating a disease and/or its attendant symptoms.

### DESCRIPTION OF THE PREFERRED EMBODIMENTS

The present invention provides compounds, compositions and methods for the treatment of diseases, in which modulation of aberrant steroid nuclear hormone receptor activity can prevent, inhibit or ameliorate the pathology and/or symptomatology of the diseases, which method comprises administering to the animal a therapeutically effective amount of a compound of Formula I.

In one embodiment of the invention, with respect to compounds of Formula I, R₁ is selected from phenyl, pyridinyl, thienyl and quinolinyl; wherein any aryl or heteroaryl of R₁ is optionally substituted with 1 to 3 radicals independently selected from chloro, bromo, fluoro, trifluoromethyl, methyl, ethyl, methoxy, allyloxy and phenyl; Rₓ is selected from cyano and —C(O)R₂; wherein R₂ is selected from —NR₆R₇ and —OR₇; wherein R₆ is selected from hydrogen and C₁₋₆alkyl; and R₇ is selected from methyl, ethyl, isopropyl, trifluoro-butyl, trifluoropropyl, cyclopropylmethyl, 2,2-dimethyl-propyl, 3,3-dimethyl-butyl, phenyl and pyridinyl; wherein any aryl or heteroaryl of R₇ is optionally substituted by 1 to 3 radicals independently selected from halo, methoxy, ethoxy and phenoxy; R₃ is selected from methyl, ethyl, propyl, cyclopropyl, butyl, isobutyl, phenyl, furanyl, optionally substituted with halo; wherein any alkyl of R₃ can optionally have a methylene replaced with —O—; wherein any cycloalkyl, aryl or heteroaryl of R₃ can optionally be substituted with 1 to 2 radicals independently selected from halo and methoxy; or R₂ and R₃ together with the atoms to which R₂ and R₃ are attached form cyclohexanone optionally substituted with 1 to 2 radicals independently selected from methyl, ethyl, propyl, isopropyl and phenyl; R₄ is hydrogen; and R₅ is selected from C₁₋₆alkyl, —SXC(O)OR₉, —SXOC(O)R₉, —SXR₉, —SXC(O)R₉, —SXNR₉R₉ and —XR₉; wherein X is a bond or C₁₋₆alkylene; R₉ is independently selected from hydroxy, C₁₋₆alkyl, halo-substituted-C₁₋₆alkyl, C₆₋₁₀aryl and C₅₋₁₀heteroaryl; wherein any aryl or heteroaryl of R₉ is optionally substituted with 1 to 3 radicals independently selected from halo, hydroxy, nitro, amino, cyano, C₁₋₆alkyl, C₁₋₆alkoxy, halo-substituted-C₁₋₆alkyl, halo-substituted-C₁₋₆alkoxy, —C(O)OR₁₀, OR₁₀ and —C(O)R₁₀; wherein R₁₀ is selected from methyl and phenyl.

In another embodiment, R₅ is selected from methyl, isobutyl, phenethyl, benzyl, phenyl, furanyl, —SCH₂C(O)OC₂H₅, —S(CH₂)₁₋₃CF₃, —S(CH₂)₀₋₃CH₃, —SCH₂C(O)R₉, —SCH₃, —SC₂H₅, —S(CH₂)₁₋₃F, —S(CH₂)₁₋₃OH, —S(CH₂)₁₋₃OC(O)N(C₂H₅)₂ and —S(CH₂)₁₋₃OH; wherein R₉ is phenyl; wherein any aryl of R₅ or R₉ is optionally substituted with benzaldehyde or 1 to 3 radicals independently selected from halo, cyano, methyl, hydroxy, nitro and —COOCH₃.

In another embodiment, are compounds of Formula Ia:

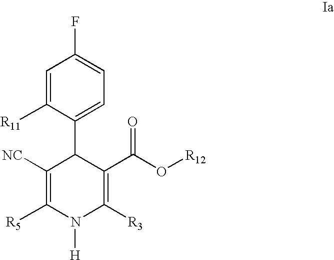

in which: R₃ is selected from methyl, ethyl, propyl, methoxy-methyl, methoxy-ethyl, methoxy-propyl, methyl-carbonyl-oxy-propyl, hydroxy-propyl, phenethyl, trifluoromethyl-butyl, cyclopropyl, cyclopropyl-methyl, cyclopropyl-ethyl and difluoromethyl; R₅ is selected from methyl, propyl, benzyl optionally substituted with fluoro, bromo, chloro or methoxy, phenethyl optionally substituted with methoxy, phenyl optionally substituted with chloro, isobutyl, furanyl, methoxy-methyl and trifluoromethyl-ethyl; R₁₁ is selected from chloro, bromo, fluoro, trifluoromethyl and methoxy; and R₁₂ is selected from cyclopropyl-methyl, isopropyl, methyl, ethyl, propyl, butyl, isobutyl, trifluoromethyl-propyl, trifluoromethyl-ethyl, t-butyl, t-butyl-methyl, t-butyl-ethyl, isopropyl-ethyl, 1,1-dimethyl-propyl, cyclobutyl-methyl and allyl.

Preferred compounds of Formula Ia are selected from: 5-cyclopropylmethyl-2-methyl-3-cyano-4-(2-chloro-4-fluorophenyl)-6-ethyl-1,4-dihydro-pyridine-5-carboxylate; 5-isopropyl-2-methyl-3-cyano-4-(2-chloro-4-fluorophenyl)-6-(methoxymethyl)-1,4-dihydro-pyridine-5-carboxylate; 5-isopropyl-2-methyl-3-cyano-4-(2,4-difluorophenyl)-6-(methoxymethyl)-1,4-dihydro-pyridine-5-carboxylate; 5-isopropyl-2-methyl-3-cyano-4-(2-trifluoromethyl-4-fluorophenyl)-6-(methoxymethyl)-1,4-dihydro-pyridine-5-carboxylate; 5-methyl-2-methyl-3-cyano-4-(2-chloro-4-fluorophenyl)-6-(methoxymethyl)-1,4-dihydro-pyridine-5-carboxylate; 5-isopropyl-2-methyl-3-cyano-4-(2-bromo-4-fluorophenyl)-6-(methoxymethyl)-1,4-dihydro-pyridine-5-carboxylate; 5-methyl-2-methyl-3-cyano-4-(2-bromo-4-fluorophenyl)-6-(methoxymethyl)-1,4-dihydro-pyridine-5-carboxylate; 5-ethyl-2-methyl-3-cyano-4-(2-trifluoromethyl-4-fluorophenyl)-6-propyl-1,4-dihydro-pyridine-5-carboxylate; 5-methyl-2-methyl-3-cyano-4-(2-trifluoromethyl-4-fluorophenyl)-6-(methoxyethyl)-1,4-dihydro-pyridine-5-carboxylate; 5-isopropyl-2-methyl-3-cyano-4-(4-fluorophenyl)-6-(methoxymethyl)-1,4-dihydro-pyridine-5-carboxylate; 5-ethyl-2-methyl-3-cyano-4-(2-chloro-4-fluorophenyl)-6-(2-methoxymethyl)-1,4-dihydro-pyridine-5-carboxylate; 5-butyl-2-methyl-3-cyano-4-(2-chloro-4-fluorophenyl)-6-(2-methoxymethyl)-1,4-dihydro-pyridine-5-carboxylate; 5-(3-methylpropyl)-2-methyl-3-cyano-4-(2-chloro-4-fluorophenyl)-6-(2-methoxymethyl)-1,4-dihydro-pyridine-5-carboxylate; 5-methyl-2-methyl-3-cyano-4-(2-chloro-4-fluorophenyl)-6-(2-methoxyethyl)-1,4-dihydro-pyridine-5-carboxylate; 5-methyl-2-methyl-3-cyano-4-(2-bromo-4-fluorophenyl)-6-(2-methoxyethyl)-1,4-dihydro-pyridine-5-carboxylate; 5-methyl-2-phenylmethyl-3-cyano-4-(2-chloro-4-fluorophenyl)-6-methyl-1,4-dihydro-pyridine-5-carboxylate; 5-methyl-2-(2-phenyl)ethyl-3-cyano-4-(2-chloro-4-fluorophenyl)-6-methyl-1,4-dihydro-pyridine-5-carboxylate; 5-(3,3,3-trifluorobutyl)-2-methyl-3-cyano-4-(2-chloro-4-fluorophenyl)-1,4-dihydro-pyridine-5-carboxylate; 5-(3,3,3-trifluoroproryl)-2-methyl-3-cyano-4-(2-chloro-4-fluorophenyl)-6-methoxymethyl-1,4-dihydro-pyridine-5-carboxylate; 5-iso-propyl-2-methyl-3-cyano-4-(2-trifluoromethyl-4-fluorophenyl)-6-propyl-1,4-dihydro-pyridine-5-carboxylate; 5-tert-butyl-2-methyl-3-cyano-4-(2-trifluoromethyl-4-fluorophenyl)-6-propyl-1,4-dihydro-pyridine-5-carboxylate; 5-methyl-2,6-dimethyl-3-cyano-4-(2-trifluoromethyl-4-fluorophenyl)-1,4-dihydro-pyridine-5-carboxylate; 5-(2-methylpropyl)-2-methyl-3-cyano-4-(2-trifluoromethyl-4-fluorophenyl)-6-methyl-1,4-dihydro-pyridine-5-carboxylate; 5-ethyl-2-methyl-3-cyano-4-(2-trifluoromethyl-4-fluorophenyl)-6-(2-methoxymethyl)-1,4-dihydro-pyridine-5-carboxylate; 5-ethyl-2-methyl-3-cyano-4-(2-bromo-4-fluorophenyl)-6-(2-methoxymethyl)-1,4-dihydro-pyridine-5-carboxylate; 5-butyl-2-methyl-3-cyano-4-(2-trifluoromethyl-4-fluorophenyl)-6-(2-methoxymethyl)-1,4-dihydro-pyridine-5-carboxylate; 5-butyl-2-methyl-3-cyano-4-(2-bromo-4-fluorophenyl)-6-(2-methoxymethyl)-1,4-dihydro-pyridine-5-carboxylate; 5-(2-methylpropyl)-2-methyl-3-cyano-4-(2-trifluoromethyl-4-fluorophenyl)-6-(2-methoxymethyl)-1,4-dihydro-pyridine-5-carboxylate; 5-(2-methylpropyl)-2-methyl-3-cyano-4-(2-bromo-4-fluorophenyl)-6-(2-methoxymethyl)-1,4-dihydro-pyridine-5-carboxylate; 5-methyl-2-phenyl-3-cyano-4-(2-chloro-4-fluorophenyl)-6-methyl-1,4-dihydro-pyridine-5-carboxylate; 5-tert-amyl-2-methyl-3-cyano-4-(2-chloro-4-fluorophenyl)-6-propyl-1,4-dihydro-pyridine-5-carboxylate; 5-tert-amyl-2-methyl-3-cyano-4-(2-bromo-4-fluorophenyl)-6-propyl-1,4-dihydro-pyridine-5-carboxylate; 5-tert-amyl-2-methyl-3-cyano-4-(2-trifluoromethyl-4-fluorophenyl)-6-propyl-1,4-dihydro-pyridine-5-carboxylate; 5-tert-amyl-2-methyl-3-cyano-4-(2-methoxy-4-fluorophenyl)-6-propyl-1,4-dihydro-pyridine-5-carboxylate; 5-(3,3-dimethylbutyl)-2-methyl-3-cyano-4-(2-chloro-4-fluorophenyl)-6-propyl-1,4-dihydro-pyridine-5-carboxylate; 5-(3,3-dimethylbutyl)-2-methyl-3-cyano-4-(2-bromo-4-fluorophenyl)-6-propyl-1,4-dihydro-pyridine-5-carboxylate; 5-(3,3-dimethylbutyl)-2-methyl-3-cyano-4-(2-trifluoromethyl-4-fluorophenyl)-6-propyl-1,4-dihydro-pyridine-5-carboxylate; 5-(3,3-dimethylbutyl)-2-methyl-3-cyano-4-(2-methoxy-4-fluorophenyl)-6-propyl-1,4-dihydro-pyridine-5-carboxylate; 5-ethyl-2-methyl-3-cyano-4-(2-methoxy-4-fluorophenyl)-6-propyl-1,4-dihydro-pyridine-5-carboxylate; 5-butyl-2-methyl-3-cyano-4-(2-bromo-4-fluorophenyl)-6-(2-methoxymethyl)-1,4-dihydro-pyridine-5-carboxylate; 5-isopropyl-2-methyl-3-cyano-4-(2-trifluoromethyl-4-fluorophenyl)-6-propyl-1,4-dihydro-pyridine-5-carboxylate; 5-propyl-2-methyl-3-cyano-4-(2-chloro-4-fluorophenyl)-6-propyl-1,4-dihydro-pyridine-5-carboxylate; 5-propyl-2-methyl-3-cyano-4-(2-bromo-4-fluorophenyl)-6-propyl-1,4-dihydro-pyridine-5-carboxylate; 5-propyl-2-methyl-3-cyano-4-(2-trifluoromethyl-4-fluorophenyl)-6-propyl-1,4-dihydro-pyridine-5-carboxylate; 5-propyl-2-methyl-3-cyano-4-(2-methoxy-4-fluorophenyl)-6-propyl-1,4-dihydro-pyridine-5-carboxylate; 5-propyl-2-methyl-3-cyano-4-(2-chloro-4-fluorophenyl)-6-(2-methoxypropyl)-1,4-dihydro-pyridine-5-carboxylate; 5-propyl-2-methyl-3-cyano-4-(2-bromo-4-fluorophenyl)-6-(2-methoxypropyl)-1,4-dihydro-pyridine-5-carboxylate; 5-propyl-2-methyl-3-cyano-4-(2-trifluoromethyl-4-fluorophenyl)-6-(2-methoxypropyl)-1,4-dihydro-pyridine-5-carboxylate; 5-propyl-2-methyl-3-cyano-4-(2-methoxy-4-fluorophenyl)-6-(2-methoxypropyl)-1,4-dihydro-pyridine-5-carboxylate; 5-(3,3-dimethylbutyl)-2-methyl-3-cyano-4-(2-chloro-4-fluorophenyl)-6-methyl-1,4-dihydro-pyridine-5-carboxylate; 5-(3,3-dimethylbutyl)-2-methyl-3-cyano-4-(2-bromo-4-fluorophenyl)-6-methyl-1,4-dihydro-pyridine-5-carboxylate; 5-(3,3-dimethylbutyl)-2-methyl-3-cyano-4-(2-trifluoromethyl-4-fluorophenyl)-6-methyl-1,4-dihydro-pyridine-5-carboxylate; 5-(3,3-dimethylbutyl)-2-methyl-3-cyano-4-(2-methoxy-4-fluorophenyl)-6-methyl-1,4-dihydro-pyridine-5-carboxylate; 5-methyl-2-tert-butyl-3-cyano-4-(2-chloro-4-fluorophenyl)-6-methyl-1,4-dihydro-pyridine-5-carboxylate; 5-methyl-2-(4-chlorophenyl)-3-cyano-4-(2-chloro-4-fluorophenyl)-6-methyl-1,4-dihydro-pyridine-5-carboxylate; 5-methyl-2-(2-furanyl)-3-cyano-4-(2-chloro-4-fluorophenyl)-6-methyl-1,4-dihydro-pyridine-5-carboxylate; 5-tert-butyl-2-methyl-3-cyano-4-(2-trifluoromethyl-4-fluorophenyl)-6-(2-methoxymethyl)-1,4-dihydro-pyridine-5-carboxylate; 5-iso-propyl-2-methyl-3-cyano-4-(2-methoxy-4-fluorophenyl)-6-methyl-1,4-dihydro-pyridine-5-carboxylate; 5-methyl-2-propyl-3-cyano-4-(2-chloro-4-fluorophenyl)-6-methoxymethyl-1,4-dihydro-pyridine-5-carboxylate; 5-methyl-2-propyl-3-cyano-4-(2-trifluoromethyl-4-fluorophenyl)-6-methoxymethyl-1,4-dihydro-pyridine-5-carboxylate; 5-methyl-2-(4-fluorophenyl)methyl-3-cyano-4-(2-trifluoromethyl-4-fluorophenyl)-6-methyl-1,4-dihydro-pyridine-5-carboxylate; 5-methyl-2-(4-fluorophenyl)methyl-3-cyano-4-(2-trifluoromethyl-4-fluorophenyl)-6-methyl-1,4-dihydro-pyridine-5-carboxylate; 2-(2-phenyl)ethyl-3,5-dicyano-4-(2-chloro-4-fluorophenyl)-6-methyl-1,4-dihydro-pyridine; 2,6-dimethyl-3-cyano-4-(2,4-dichloro)-5-(2-methoxyphenyl)carbamoyl-1,4-dihydropyridine; 5-methyl-2-methyl-3-cyano-4-(2-chloro-4-fluorophenyl)-6-(3-methoxypropyl)-1,4-dihydro-pyridine-5-carboxylate; 5-tert-butyl-2-methyl-3-cyano-4-(2-chloro-4-fluorophenyl)-6-(3-methoxypropyl)-1,4-dihydro-pyridine-5-carboxylate; 5-methyl-2-methyl-3-cyano-4-(2-trifluoromethyl-4-fluorophenyl)-6-(3-methoxypropyl)-1,4-dihydro-pyridine-5-carboxylate; 5-methyl-2-methyl-3-cyano-4-(2-chloro-4-fluorophenyl)-6-(3-acetoxypropyl)-1,4-dihydro-pyridine-5-carboxylate; 5-methyl-2-methyl-3-cyano-4-(2-trifluoromethyl-4-fluorophenyl)-6-(3-acetoxypropyl)-1,4-dihydro-pyridine-5-carboxylate; 5-methyl-2-methyl-3-cyano-4-(2-chloro-4-fluorophenyl)-6-(3-hydroxypropyl)-1,4-dihydro-pyridine-5-carboxylate; 5-methyl-2-methyl-3-cyano-4-(2-trifluoromethyl-4-fluorophenyl)-6-(3-hydroxypropyl)-1,4-dihydro-pyridine-5-carboxylate; 5-methyl-2-methyl-3-cyano-4-(2-trifluoromethyl-4-fluorophenyl)-6-(3-hydroxypropyl)-1,4-dihydro-pyridine-5-carboxylate; 5-methyl-2-methyl-3-cyano-4-(2-chloro-4-fluorophenyl)-6-(2-phenylethyl)-1,4-dihydro-pyridine-5-carboxylate; 5-methyl-2-methyl-3-cyano-4-(2-trifluoromethyl-4-fluorophenyl)-6-(2-phenylethyl)-1,4-dihydro-pyridine-5-carboxylate; 5-methyl-2-methyl-3-cyano-4-(2-chloro-4-fluorophenyl)-6-(5,5,5-trifluoropentyl)-1,4-dihydro-pyridine-5-carboxylate; 5-methyl-2-methyl-3-cyano-4-(2-trifluoromethyl-4-fluorophenyl)-(5,5,5-trifluoropentyl)-1,4-dihydro-pyridine-5-carboxylate; 5-methyl-2,6-dimethyl-3-cyano-4-(2-chloro-4-fluorophenyl)-1,4-dihydro-pyridine-5-carboxylate; 5-methyl-2-methyl-3-cyano-4-(2-trifluoromethyl-4-fluorophenyl)-6-cyclopropyl-1,4-dihydro-pyridine-5-carboxylate; 5-methyl-2-methyl-3-cyano-4-(2-chloro-4-fluorophenyl)-6-cyclopropyl-1,4-dihydro-pyridine-5-carboxylate; 5-methyl-2-methyl-3-cyano-4-(2-chloro-4-fluorophenyl)-6-2-cyclopropylethyl-1,4-dihydro-pyridine-5-carboxylate; 5-cyclopropylmethyl-2-methyl-3-cyano-4-(2-chloro-4-fluorophenyl)-6-(2-methoxyethyl)-1,4-dihydro-pyridine-5-carboxylate; 5-methyl-2-phenylmethyl-3-cyano-4-(2-trifluoromethyl-4-fluorophenyl)-6-methyl-1,4-dihydro-pyridine-5-carboxylate; 5-methyl-2-(2-furyl)-3-cyano-4-(2-chloro-4-fluorophenyl)-6-(2-methoxymethyl)-1,4-dihydro-pyridine-5-carboxylate; 5-methyl-2-(2-phenylethyl)-3-cyano-4-(2-chloro-4-fluorophenyl)-6-(2-methoxymethyl)-1,4-dihydro-pyridine-5-carboxylate; 5-methyl-2-(4-chlorophenyl)-3-cyano-4-(2-chloro-4-fluorophenyl)-6-(2-methoxymethyl)-1,4-dihydro-pyridine-5-carboxylate; 5-methyl-2-(2-furyl)-3-cyano-4-(2-trifluoromethyl-4-fluorophenyl)-6-(2-methoxymethyl)-1,4-dihydro-pyridine-5-carboxylate; 5-methyl-2-(2-phenylethyl)-3-cyano-4-(2-trifluoromethyl-4-fluorophenyl)-6-(2-methoxymethyl)-1,4-dihydro-pyridine-5-carboxylate; 5-(3,3,3-trifluoropropyl)-2-methyl-3-cyano-4-(2-trifluoromethyl-4-fluorophenyl)-6-(2-methoxymethyl)-1,4-dihydro-pyridine-5-carboxylate; 5-ethyl-2-(2-furyl)-3-cyano-4-(2-chloro-4-fluorophenyl)-6-propyl1,4-dihydro-pyridine-5-carboxylate; 5-ethyl-2-(2-phenylethyl)-3-cyano-4-(2-chloro-4-fluorophenyl)-6-propyl1,4-dihydro-pyridine-5-carboxylate; 5-(4,4,4-trifluorobutyl)-2-methyl-3-cyano-4-(2-methoxy-4-fluorophenyl)-6-propyl1,4-dihydro-pyridine-5-carboxylate; 5-(4,4,4-trifluorobutyl)-2-methyl-3-cyano-4-(2-trifluoromethyl-4-fluorophenyl)-6-propyl1,4-dihydro-pyridine-5-carboxylate; 5-tert-butyl-2-methyl-3-cyano-4-(2-chloro-4-fluorophenyl)-6-(2-methoxymethyl1,4-dihydro-pyridine-5-carboxylate; 5-(3,3-dimethylbutyl)-2-methyl-3-cyano-4-(2-chloro-4-fluorophenyl)-6-(2-methoxymethyl1,4-dihydro-pyridine-5-carboxylate; 5-(3,3-dimethylbutyl)-2-methyl-3-cyano-4-(2-trifluoromethyl-4-fluorophenyl)-6-(2-methoxymethyl1,4-dihydro-pyridine-5-carboxylate; 5-methyl-2-[2-(4-methoxyphenyl)ethyl]-3-cyano-4-(2-chloro-4-fluorophenyl)-6-methyl-1,4-dihydro-pyridine-5-carboxylate; 5-(2,2-dimethylpropyl)-2-methyl-3-cyano-4-(2-chloro-4-fluorophenyl)-6-methyl-1,4-dihydro-pyridine-5-carboxylate; 5-(2,2-dimethylpropyl)-2-methyl-3-cyano-4-(2-trifluoromethyl-4-fluorophenyl)-6-(2-methoxymethyl)-1,4-dihydro-pyridine-5-carboxylate; 5-(1,1-dimethylpropyl)-2-methyl-3-cyano-4-(2-chloro-4-fluorophenyl)-6-)2-methoxymethyl)-1,4-dihydro-pyridine-5-carboxylate; 5-(1,1-dimethylpropyl)-2methyl-3-cyano-4-(2-trifluoromethyl-4-fluorophenyl)-6-(2-methoxymethyl)-1,4-dihydro-pyridine-5-carboxylate; 5-methyl-2-(2-methoxymethyl)-3-cyano-4-(2-trifluoromethyl-4-fluorophenyl)-6-(2-methoxymethyl)-1,4-dihydro-pyridine-5-carboxylate; 5-methyl-2-(3,3,3-trifluoropropyl)-3-cyano-4-(2-chloro-4-fluorophenyl)-6-methyl-1,4-dihydro-pyridine-5-carboxylate; 5-methyl-2-(3-methoxyphenylmethyl)-3-cyano-4-(2-chloro-4-fluorophenyl)-6-methyl-1,4-dihydro-pyridine-5-carboxylate; 5-methyl-2-(4-methoxyphenylmethyl)-3-cyano-4-(2-chloro-4-fluorophenyl)-6-methyl-1,4-dihydro-pyridine-5-carboxylate; 5-methyl-2-(3-methoxyphenylmethyl)-3-cyano-4-(2-chloro-4-fluorophenyl)-6-(2-methoxymethyl)-1,4-dihydro-pyridine-5-carboxylate; 5-methyl-2-(4-methoxyphenylmethyl)-3-cyano-4-(2-chloro-4-fluorophenyl)-6-(2-methoxymethyl)-1,4-dihydro-pyridine-5-carboxylate; 5-methyl-2-(2-fluorophenylmethyl)-3-cyano-4-(2-chloro-4-fluorophenyl)-6-methyl-1,4-dihydro-pyridine-5-carboxylate; 5-methyl-2-(3-fluorophenylmethyl)-3-cyano-4-(2-chloro-4-fluorophenyl)-6-methyl-1,4-dihydro-pyridine-5-carboxylate; 5-methyl-2-(4-chlorophenylmethyl)-3-cyano-4-(2-chloro-4-fluorophenyl)-6-methyl-1,4-dihydro-pyridine-5-carboxylate; 5-methyl-2-(4-bromophenylmethyl)-3-cyano-4-(2-chloro-4-fluorophenyl)-6-methyl-1,4-dihydro-pyridine-5-carboxylate; 5-methyl-2-(2-fluorophenylmethyl)-3-cyano-4-(2-trifluoromethyl-4-fluorophenyl)-6-methyl-1,4-dihydro-pyridine-5-carboxylate; 5-methyl-2-(3-fluorophenylmethyl)-3-cyano-4-(2-trifluoromethyl-4-fluorophenyl)-6-methyl-1,4-dihydro-pyridine-5-carboxylate; 5-methyl-2-(4-chlorophenylmethyl)-3-cyano-4-(2-trifluoromethyl-4-fluorophenyl)-6-methyl-1,4-dihydro-pyridine-5-carboxylate; 5-methyl-2-(4-bromophenylmethyl)-3-cyano-4-(2-trifluoromethyl-4-fluorophenyl)-6-methyl-1,4-dihydro-pyridine-5-carboxylate; 5-cyclopropylmethyl-2-methyl-3-cyano-4-(2-chloro-4-fluorophenyl)-6-(2-cyclopropyl)ethyl-1,4-dihydro-pyridine-5-carboxylate; 5-ethyl-2-methyl-3-cyano-4-(2-chloro-4-fluorophenyl)-6-(2-cyclopropyl)ethyl-1,4-dihydro-pyridine-5-carboxylate; 5-ethyl-2-methyl-3-cyano-4-(2-trifluoromethyl-4-fluorophenyl)-6-ethyl-1,4-dihydro-pyridine-5-carboxylate; 5-cyclopropylmethyl-2-methyl-3-cyano-4-(2-trifluoromethyl-4-fluorophenyl)-6-(2-methoxyethyl)-1,4-dihydro-pyridine-5-carboxylate; 5-cyclobutylmethyl-2-methyl-3-cyano-4-(2-trifluoromethyl-4-fluorophenyl)-6-(2-methoxyethyl)-1,4-dihydro-pyridine-5-carboxylate; 5-ethyl-2-methyl-3-cyano-4-(2-chloro-4-fluorophenyl)-6-difluoromethyl-1,4-dihydro-pyridine-5-carboxylate; 5-ethyl-2-methyl-3-cyano-4-(2-trifluoromethyl-4-fluorophenyl)-6-difluoromethyl-1,4-dihydro-pyridine-5-carboxylate; 5-cyclopropylmethyl-2-methyl-3-cyano-4-(2-trifluoromethyl-4-fluorophenyl)-6-propyl-1,4-dihydro-pyridine-5-carboxylate; 5-allyl-2-(2-fluorophenyl)methyl-3-cyano-4-(2-trifluoromethyl-4-fluorophenyl)-6-methyl-1,4-dihydro-pyridine-5-carboxylate; 5-(2,2-dimethylpropyl)-2-methyl-3-cyano-4-(2-trifluoromethyl-4-fluorophenyl)-6-ethyl-1,4-dihydro-pyridine-5-carboxylate; 5-cyclopropylmethyl-2-methyl-3-cyano-4-(2-trifluoromethyl-4-fluorophenyl)-6-ethyl-1,4-dihydro-pyridine-5-carboxylate; 5-cyclopropylmethyl-2-methyl-3-cyano-4-(2-trifluoromethyl-4-fluorophenyl)-6-propyl-1,4-dihydro-pyridine-5-carboxylate; 5-(2-methyl)propyl-2-methyl-3-cyano-4-(2-trifluoromethyl-4-fluorophenyl)-6-ethyl-1,4-dihydro-pyridine-5-carboxylate; and 5-(2,2-dimethyl)propyl-2-methyl-3-cyano-4-(2-chloro-4-fluorophenyl)-6-ethyl-1,4-dihydro-pyridine-5-carboxylate.

In another embodiment are compounds of Formula Ib:

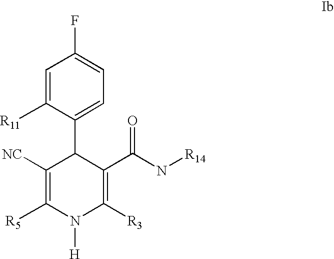

in which: R₃ is selected from methyl, ethyl, propyl, methoxy-methyl, methoxy-ethyl, methoxy-propyl, methyl-carbonyl-oxy-propyl, hydroxy-propyl, phenethyl, trifluoromethyl-butyl, cyclopropyl, cyclopropyl-methyl, cyclopropyl-ethyl and difluoromethyl; R₅ is selected from methyl, propyl, benzyl optionally substituted with fluoro, bromo, chloro or methoxy, methyl-thio, ethyl-thio, propyl-thio, butyl-thio, trifluoromethyl-propyl-thio, phenethyl optionally substituted with methoxy, phenyl optionally substituted with chloro, isobutyl, furanyl, methoxy-methyl and trifluoromethyl-ethyl; R₁₁ is selected from chloro, bromo, fluoro, trifluoromethyl and methoxy; and R₁₂ is selected from cyclopropyl-methyl, isopropyl, methyl, ethyl, propyl, butyl, isobutyl, trifluoromethyl-propyl, trifluoromethyl-ethyl, t-butyl, t-butyl-methyl, t-butyl-ethyl, isopropyl-ethyl, 1,1-dimethyl-propyl, cyclobutyl-methyl and allyl.

Preferred compounds of Formula Ib are selected from: 2-ethylthio-3-cyano-4-(2,4-difluorophenyl)-5-(2-methoxyphenyl)carbamoyl-6-methyl-1,4-dihydro-pyridine; 2-butylthio-3-cyano-4-(2,4-difluorophenyl)-5-(2-methoxyphenyl)carbamoyl-6-methyl-1,4-dihydro-pyridine; 2-(4,4,4-trifluorobutyl)thio-3-cyano-4-(2,4-difluorophenyl)-5-(2-methoxyphenyl)carbamoyl-6-methyl-1,4-dihydro-pyridine; and 2-(2-phenylmethyl)-3-cyano-4-(2-chloro-4-fluorophenyl)-5-(2-chloro-4-fluorophenyl)carbamoyl-6-methyl-1,4-dihydro-pyridine.

Further preferred compounds of the invention are selected from: N-methyl-4-morpholinium-6-methyl-4-(2-fluoro-4-bromophenyl)-5-(2-methoxyphenyl)carbamoyl-3-cyano-1,4-dihydro-pyridine-2-thiolate 1; 2-(4-methylbenzyl)thio-3-cyano-4-(2-chlorophenyl)-5-(2-methoxyphenyl)carbamoyl-6-methyl-1,4-dihydro-pyridine; 2,6-dimethyl-3-cyano-4-(2-chlorophenyl)-5-(2-methoxyphenyl)carbamoyl-1,4-dihydro-pyridine; 2-(4,4,4-trifluorobutyl)thio-3-cyano-4-(2-chlorophenyl)-5-(2-methoxyphenyl)carbamoyl-6-methyl-1,4-dihydro-pyridine; 2-(2-methylbenzyl)thio-3-cyano-4-(2-chlorophenyl)-5-(2-methoxyphenyl)carbamoyl-6-methyl-1,4-dihydro-pyridine; 2-(3,5-dimethylbenzylbenzyl)thio-3-cyano-4-(2-chlorophenyl)-5-(2-methoxyphenyl)carbamoyl-6-methyl-1,4-dihydro-pyridine; 2-(3-nitrobenzylbenzyl)thio-3-cyano-4-(2-chlorophenyl)-5-(2-methoxyphenyl)carbamoyl-6-methyl-1,4-dihydro-pyridine; 2-methylthio-3-cyano-4-(2,4-dichlorophenyl)-5-(2-methoxyphenyl)carbamoyl-6-methyl-1,4-dihydro-pyridine; 2-ethylthio-3-cyano-4-(2,4-dichlorophenyl)-5-(2-methoxyphenyl)carbamoyl-6-methyl-1,4-dihydro-pyridine; 2-butylthio-3-cyano-4-(2,4-dichlorophenyl)-5-(2-methoxyphenyl)carbamoyl-6-methyl-1,4-dihydro-pyridine; 2-(3-fluoropropyl)thio-3-cyano-4-(2,4-dichlorophenyl)-5-(2-methoxyphenyl)carbamoyl-6-methyl-1,4-dihydro-pyridine; 2-(4,4,4-trifluorofluorobutyl)thio-3-cyano-4-(2,4-dichlorophenyl)-5-(2-methoxyphenyl)carbamoyl-6-methyl-1,4-dihydro-pyridine; 2-benzylthio-3-cyano-4-(2,4-dichlorophenyl)-5-(2-methoxyphenyl)carbamoyl-6-methyl-1,4-dihydro-pyridine; 2-methylthio-3-cyano-4-(2-bromophenyl)-5-(2-methoxyphenyl)carbamoyl-6-methyl-1,4-dihydro-pyridine; 2-butylthio-3-cyano-4-(2-bromophenyl)-5-(2-methoxyphenyl)carbamoyl-6-methyl-1,4-dihydro-pyridine; 2-(4,4,4-trifluorobutylthio-3-cyano-4-(2-bromophenyl)-5-(2-methoxyphenyl)carbamoyl-6-methyl-1,4-dihydro-pyridine; 2-(3-nitrobenzyl)thio-3-cyano-4-(2,4-dichlorophenyl)-5-(2-methoxyphenyl)carbamoyl-6-methyl-1,4-dihydro-pyridine; 2-(3-nitrobenzylthio-3-cyano-4-(2-bromophenyl)-5-(2-methoxyphenyl)carbamoyl-6-methyl-1,4-dihydro-pyridine; 2-(4-carboxymethylbenzylthio-3-cyano-4-(2-bromophenyl)-5-(2-methoxyphenyl)carbamoyl-6-methyl-1,4-dihydro-pyridine; 2-(2-cyanobenzylbenzyl)thio-3-cyano-4-(2-chlorophenyl)-5-(2-methoxyphenyl)carbamoyl-6-methyl-1,4-dihydro-pyridine; 2-(3-cyanobenzylbenzyl)thio-3-cyano-4-(2-chlorophenyl)-5-(2-methoxyphenyl)carbamoyl-6-methyl-1,4-dihydro-pyridine; 2-(3-hydroxymethyl)thio-3-cyano-4-(2-chlorophenyl)-5-(2-methoxyphenyl)carbamoyl-6-methyl-1,4-dihydro-pyridine; 2-(2-cyanobenzylthio-3-cyano-4-(2-bromophenyl)-5-(2-methoxyphenyl)carbamoyl-6-methyl-1,4-dihydro-pyridine; 2-(4-cyanobenzylbenzyl)thio-3-cyano-4-(2-bromophenyl)-5-(2-methoxyphenyl)carbamoyl-6-methyl-1,4-dihydro-pyridine; 2-butylthio-3-cyano-4-(2-trifluoromethylphenyl)-5-(2-methoxyphenyl)carbamoyl-6-methyl-1,4-dihydro-pyridine; 2-(2-hydroxyethyl)thio-3-cyano-4-(2-chlorophenyl)-5-(2-methoxyphenyl)carbamoyl-6-methyl-1,4-dihydro-pyridine; 2-(acetoxyethyl)thio-3-cyano-4-(2-chlorophenyl)-5-(2-methoxyphenyl)carbamoyl-6-methyl-1,4-dihydro-pyridine; 2-(hydroxyethyl)thio-3-cyano-4-(2-bromophenyl)-5-(2-methoxyphenyl)carbamoyl-6-methyl-1,4-dihydro-pyridine; 2-(N,N-diethylaminoethyl)thio-3-cyano-4-(2-chlorophenyl)-5-(2-methoxyphenyl)carbamoyl-6-methyl-1,4-dihydro-pyridine; 2-benzylthio-3-cyano-4-(2-trifluoromethylphenyl)-5-(2-methoxyphenyl)carbamoyl-6-methyl-1,4-dihydro-pyridine; 5-ethyl-2-(hydroxyethyl)thio-3-cyano-4-(2,4-dichlorophenyl)-6-propyl-1,4-dihydro-pyridine-5-carboxylate; 5-ethyl-2-(hydroxypropyl)thio-3-cyano-4-(2,4-dichlorophenyl)-6-propyl-1,4-dihydro-pyridine-5-carboxylate; 2-(4-methylbenzyl)thio-3-cyano-4-(2-bromophenyl)-5-(2-methoxyphenyl)carbamoyl-6-methyl-1,4-dihydro-pyridine; 2-methylthio-3-cyano-4-(2-fluoro-4-bromophenyl)-5-(2-methoxyphenyl)carbamoyl-6-methyl-1,4-dihydro-pyridine; 2-methylthio-3-cyano-4-[3-(2-chloropyridine)]-5-(2-methoxyphenyl)carbamoyl-6-methyl-1,4-dihydro-pyridine; 2-methylthio-3-cyano-4-(2-methoxyphenyl)-5-(2-methoxyphenyl)carbamoyl-6-methyl-1,4-dihydro-pyridine; 2-ethylthio-3-cyano-4-(2-methoxyphenyl)-5-(2-methoxyphenyl)carbamoyl-6-methyl-1,4-dihydro-pyridine; 2-butylthio-3-cyano-4-(2-methoxyphenyl)-5-(2-methoxyphenyl)carbamoyl-6-methyl-1,4-dihydro-pyridine; 2-ethylthio-3-cyano-4-[3-(2-chloropyridine)]-5-(2-methoxyphenyl)carbamoyl-6-methyl-1,4-dihydro-pyridine; 2-(4,4,4-trifluorobutyl)thio-3-cyano-4-[3-(2-chloropyridine)]-5-(2-methoxyphenyl)carbamoyl-6-methyl-1,4-dihydro-pyridine; 2-butylthio-3-cyano-4-[3-(2-chloropyridine)]-5-(2-methoxyphenyl)carbamoyl-6-methyl-1,4-dihydro-pyridine; 2-benzylthio-3-cyano-4-[2-(5-bromothiophene)]-carbamoyl-6-methyl-1,4-dihydro-pyridine; 2-methylthio-3-cyano-4-(2-fluoro-4-chlorophenyl)-carbamoyl-6-methyl-1,4-dihydro-pyridine; 2-ethylthio-3-cyano-4-(2-fluoro-4-chlorophenyl)-carbamoyl-6-methyl-1,4-dihydro-pyridine, 2-propylthio-3-cyano-4-(2-fluoro-4-chlorophenyl)-carbamoyl-6-methyl-1,4-dihydro-pyridine; 2-butylthio-3-cyano-4-(2-fluoro-4-chlorophenyl)-carbamoyl-6-methyl-1,4-dihydro-pyridine; 2-(3-nitro-4-methylbenzyl)thio-3 cyano-4-(2-trifluoromethylphenyl)-carbamoyl-6-methyl-1,4-dihydro-pyridine; 2,6-dimethyl-3-cyano-4-(2,4-dichlorophenyl)-5-(2-methoxyphenyl)carbamoyl-1,4-dihydro-pyridine; 2,6-dimethyl-3-cyano-4-(4-bromophenyl)-5-(2-methoxyphenyl)carbamoyl-1,4-dihydro-pyridine; 2,6-dimethyl-3-cyano-4-(2-allyloxyphenyl)-5-(2-methoxyphenyl)carbamoyl-1,4-dihydro-pyridine; 2,6-dimethyl-3-cyano-4-(2-methoxyphenyl)-5-(2-methoxyphenyl)carbamoyl-1,4-dihydro-pyridine; 2,6-dimethyl-3-cyano-4-[3-(2-methoxypyridine)]-5-(2-methoxyphenyl)carbamoyl-1,4-dihydro-pyridine; 2,6-dimethyl-3-cyano-4-(2,4-dichlorophenyl)-5-phenylcarbamoyl-1,4-dihydro-pyridine; 5-methyl-2-methyl-3-cyano-4-(2,4-dichlorophenyl)-6-cyclopropyl-1,4-dihydro-pyridine-5-carboxylate; 5-methyl-2-methyl-3-cyano-4-(2,4-dichlorophenyl)-6-(methoxymethyl)-1,4-dihydro-pyridine-5-carboxylate; 2,6-dimethyl-3-cyano-4-(2,4-dichlorophenyl)-5-N-(2-methoxyphenyl)-N-(1-hydroxyvynyl)carbamoyl-1,4-dihydro-pyridine; 2-methyl-3-cyano-4-(2,4-dichlorophenyl)-5,6-cyclo-3-methyl-hexyl-1,4-dihydro-pyridine; 2-methyl-3-cyano-4-(2,4-dichlorophenyl)-5,6-cyclo-3-isopropyl-hexyl-1,4-dihydro-pyridine; 2-methyl-3-cyano-4-(2,4-dichlorophenyl)-5,6-cyclo-3-phenyl-hexyl-1,4-dihydro-pyridine; 2,6-dimethyl-3-cyano-4-(4-phenylphenyl)-5-(2-methoxyphenyl)-carbamoyl-1,4-dihydro-pyridine; 2,6-dimethyl-3-cyano-4-(2-bromo-4-methylphenyl)-5-(2-methoxyphenyl)carbamoyl-1,4-dihydro-pyridine; 5-isopropyl-2,6-dimethyl-3-cyano-4-(2,4-dichlorophenyl)-1,4-dihydro-pyridine-5-carboxylate; 5-methyl-2-methyl-3-cyano-4-(2,4-dichlorophenyl)-6-isopropyl-1,4-dihydro-pyridine-5-carboxylate; 5-methyl-2-methyl-3-cyano-4-(2,4-dichlorophenyl)-6-propyl-1,4-dihydro-pyridine-5-carboxylate; 5-ethyl-2-methyl-3-cyano-(2,4-dichlorophenyl)-6-isopropyl-1,4-dihydro-pyridine-5-carboxylate; 5-ethyl-2-methyl-3-cyano-4-(2,4-dichlorophenyl)-6-ethyl-1,4-dihydro-pyridine-5-carboxylate; 5-ethyl-2-methyl-3-cyano-4-(2,4-dichlorophenyl)-6-propyl-1,4-dihydro-pyridine-5-carboxylate; 5-ethyl-2-methyl-3-cyano-4-(2,4-dichlorophenyl)-6-(2-fluorophenyl)-1,4-dihydro-pyridine-5-carboxylate; 5-ethyl-2-methyl-3-cyano-4-(2,4-dichlorophenyl)-6-phenyl-1,4-dihydro-pyridine-5-carboxylate; 5-ethyl-2-methyl-3-cyano-4-(2,4-dichlorophenyl)-6-(4-methoxyphenyl)-1,4-dihydro-pyridine-5-carboxylate; 5-ethyl-2-methyl-3-cyano-4-(2,4-dichlorophenyl)-6-(3-furyl)-1,4-dihydro-pyridine-5-carboxylate; 5-ethyl-2-methyl-3-cyano-4-(2,4-dichlorophenyl)-6-(2-furyl)-1,4-dihydro-pyridine-5-carboxylate; 5-isopropyl-2-methyl-3-cyano-4-(2-fluoro-4-chlorophenyl)-6-(methoxymethyl)-1,4-dihydro-pyridine-5-carboxylate; 5-isopropyl-2-methyl-3-cyano-4-(2-fluoro-4-trifluoromethylphenyl)-6-(methoxymethyl)-1,4-dihydro-pyridine-5-carboxylate; 5-isopropyl-2-methyl-3-cyano-4-(2,4-bistrifluoromethylphenyl)-6-(methoxymethyl)-1,4-dihydro-pyridine-5-carboxylate; 5-isopropyl-2-methyl-3-cyano-4-(2-chloro-5-trifluoromethylphenyl)-6-(methoxymethyl)-1,4-dihydro-pyridine-5-carboxylate; 5-isopropyl-2-methyl-3-cyano-4-(3-trifluoromethyl-4-chlorophenyl)-6-(methoxymethyl)-1,4-dihydro-pyridine-5-carboxylate; 5-isopropyl-2-methyl-3-cyano-4-(2-fluoro-4-bromophenyl)-6-(methoxymethyl)-1,4-dihydro-pyridine-5-carboxylate; 5-isopropyl-2-methyl-3-cyano-4-(2-bromo-4-fluorophenyl)-6-(methoxymethyl)-1,4-dihydro-pyridine-5-carboxylate; 5-methyl-2-methyl-3-cyano-4-(2-fluoro-4-bromophenyl)-6-(methoxymethyl)-1,4-dihydro-pyridine-5-carboxylate; 5-methyl-2-methyl-3-cyano-4-(2-bromo-4-methylphenyl)-6-(methoxymethyl)-1,4-dihydro-pyridine-5-carboxylate; 5-methyl-2-methyl-3-cyano-4-(2-fluoro-4-chlorophenyl)-6-(methoxymethyl)-1,4-dihydro-pyridine-5-carboxylate; 2,6-dimethyl-3-cyano-4-(2-fluoro-4-bromophenyl)-5-(2-methoxyphenyl)carbamoyl-1,4-dihydro-pyridine; 2,6-dimethyl-3-cyano-4-(2-fluoro-4-chlorophenyl)-5-(2-methoxyphenyl)carbamoyl-1,4-dihydro-pyridine; 2,6-dimethyl-3-cyano-4-(2-fluoro-4-trifluoromethylphenyl)-5-(2-methoxyphenyl)carbamoyl-1,4-dihydro-pyridine; 5-isopropyl-2-methyl-3-cyano-4-(2,6-dichlorophenyl)-6-(methoxymethyl)-1,4-dihydro-pyridine-5-carboxylate; 5-methyl-2-methyl-3-cyano-4-(2,6-dichlorophenyl)-6-(methoxymethyl)-1,4-dihydro-pyridine-5-carboxylate; 2,6-dimethyl-3-cyano-4-(2-fluoro-6-chlorophenyl)-5-(2-methoxyphenyl)carbamoyl-1,4-dihydro-pyridine; 2,6-dimethyl-3-cyano-4-(2,6-difluorophenyl)-5-(2-methoxyphenyl)carbamoyl-1,4-dihydro-pyridine; 2,6-dimethyl-3-cyano-4-(4-fluoro-5-trifluoromethylphenyl)-5-(2-methoxyphenyl)carbamoyl-1,4-dihydro-pyridine; 2-methyl-3-cyano-4-(2-trifluoromethyl-4-fluorophenyl)-5,6-(3,3-dimethyl)-cyclohexan-2-one-1,4-dihydro-pyridine; 5-methyl-2-methyl-3-cyano-4-[4-(2-bromopyridine)]-6-(methoxymethyl)-1,4-dihydro-pyridine-5-carboxylate; 5-methyl-2-methyl-3-cyano-4-[3-(2-methoxypyridine)]-6-propyl-1,4-dihydro-pyridine-5-carboxylate; 5-methyl-2-methyl-3-cyano-4-[3-(2,5-dichlorothiophene)]-6-propyl-1,4-dihydro-pyridine-5-carboxylate; 5-methyl-2-methyl-3-cyano-4-[3-(2,5-dichlorothiophene)]-6-cyclopropyl-1,4-dihydro-pyridine-5-carboxylate; 5-isopropyl-2-methyl-3-cyano-4-[3-(2,5-dichlorothiophene)]-6-methoxymethyl)-1,4-dihydro-pyridine-5-carboxylate; 5-methyl-2-methyl-3-cyano-4-(3,4-difluorophenyl)-6-propyl-1,4-dihydro-pyridine-5-carboxylate; 5-isopropyl-2-methyl-3-cyano-4-(4-quinoline)-6propyl-1,4-dihydro-pyridine-5-carboxylate; 5-methyl-2-methyl-3-cyano-4-[3-(2,5-dimethylthiophene)]-6-propyl-1,4-dihydro-pyridine-5-carboxylate; 5-methyl-2-methyl-3-cyano-4-[3-(2,5-dimethylthiophene)]-6-cyclopropyl-1,4-dihydro-pyridine-5-carboxylate; 2,6-dimethyl-3-cyano-4-(2-ethoxyphenyl)-5-(2,4-dichlorophenyl)carbamoyl-1,4-dihydro-pyridine; 5-ethyl-2-thiomethyl-3-cyano-4-(2-chloro-3-pyridine)-6-propyl-1,4-dihydro-pyridine-5-carboxylate; 5-ethyl)-2-thiomethyl-3-cyano-4-(2-methoxy-3-pyridine)-6-propyl-1,4-dihydro-pyridine-5-carboxylate; 5-ethyl-2-methyl-3-cyano-4-(2-methyl-3-pyridine)-6-propyl-1,4-dihydro-pyridine-5-carboxylate; 5-iso-propyl-2-methyl-3-cyano-4-(2-methyl-3-pyridine)-6-propyl-1,4-dihydro-pyridine-5-carboxylate; 5-methyl-2-methyl-3-cyano-4-(2-chlorophenyl)-6-(2-methoxymethyl)-1,4-dihydro-pyridine-5-carboxylate; 5-methyl-2-methyl-3-cyano-4-(2-cbromophenyl)-6-(2-methoxymethyl) 1,4-dihydro-pyridine-5-carboxylate; 5-methyl-2-methyl-3-cyano-4-(2-methylphenyl)-6-(2-methoxymethyl)-1,4-dihydro-pyridine-5-carboxylate; 5-iso-propyl-2-methyl-3-cyano-4-(2-chlorophenyl)-6-(2-methoxymethyl)-1,4-dihydro-pyridine-5-carboxylate; 5-iso-propyl-2-methyl-3-cyano-4-(2-bromophenyl)-6-(2-methoxymethyl)-1,4-dihydro-pyridine-5-carboxylate; 5-iso-propyl-2-methyl-3-cyano-4-(2-methylphenyl)-6-(2-methoxymethyl)-1,4-dihydro-pyridine-5-carboxylate; 5-iso-propyl-2-methyl-3-cyano-4-(2-ethylphenyl)-6-(2-methoxymethyl)-1,4-dihydro-pyridine-5-carboxylate; 5-methyl-2-methyl-3-cyano-4-(2,4-dichlorophenyl)-6-(2-phenylethyl)-1,4-dihydro-pyridine-5-carboxylate; 5-methyl-2-methyl-3-cyano-4-(2-methoxy-3,4-difluorophenyl)-6-(2-methoxyethyl)-1,4-dihydro-pyridine-5-carboxylate; 5-methyl-2-methyl-3-cyano-4-(2-methoxy-3,4-difluorophenyl)-6-(2-methoxymethyl)-1,4-dihydro-pyridine-5-carboxylate; 5-methyl-2-methyl-3-cyano-4-(2-chloro-3,4-difluorophenyl)-6-(2-methoxyethyl)-1,4-dihydro-pyridine-5-carboxylate; 2,6-dimethyl-3-cyano-4-(2-fluoro-4-chloro)-5-(2-methoxyphenyl)carbamoyl-1,4-dihydropyridine; 5-methyl-2-methyl-3-cyano-4-(2,4-dichlorophenyl)-6-(5,5,5-trifluoropentyl)-1,4-dihydro-pyridine-5-carboxylate; 5-methyl-2-methyl-3-cyano-4-(2-fluoro-4-chlorophenyl)-6-(5,5,5-trifluoropentyl)-1,4-dihydro-pyridine-5-carboxylate; 5-methyl-2-methyl-3-cyano-4-(2-methoxy-3,4-difluorophenyl)-6-cyclopropyl-1,4-dihydro-pyridine-5-carboxylate; 5-methyl-2-methyl-3-cyano-4-(2-bromo-4-pyridyl)-6-cyclopropyl-1,4-dihydro-pyridine-5-carboxylate; 5-methyl-2-methyl-3-cyano-4-(2-bromo-4-pyridyl)-6-2-methoxyethyl-1,4-dihydro-pyridine-5-carboxylate; 5-methyl-2-methyl-3-cyano-4-(2-trifluoromethyl-4-fluorophenyl)-6-2-cyclopropylethyl-1,4-dihydro-pyridine-5-carboxylate; 5-methyl-2-methyl-3-cyano-4-(2-methoxy-3,4-difluorophenyl)-6-2-cyclopropylethyl-1,4-dihydro-pyridine-5-carboxylate; 5-methyl-2-methyl-3-cyano-4-(2-bromo-4-pyridyl)-6-(2-cyclopropylethyl)-1,4-dihydro-pyridine-5-carboxylate; 5-cyclopropylmethyl-2-methyl-3-cyano-4-(2-trifluoromethyl-4-fluorophenyl)-6-(2-methoxyethyl)-1,4-dihydro-pyridine-5-carboxylate; and 5-cyclopropylmethyl-2-methyl-3-cyano-4-(2-methoxy-3,4-difluorophenyl)-6-(2-methoxyethyl)-1,4-dihydro-pyridine-5-carboxylate.

In a further embodiment, the present invention provides compounds, compositions and methods for the treatment of diseases, in which modulation of aberrant steroid nuclear hormone receptor activity and calcium channel activity, preferable L-type calcium channels, can prevent, inhibit or ameliorate the pathology and/or symptomatology of the diseases, which method comprises administering to the animal a therapeutically effective amount of a compound of Formula I in which: R₁ is selected from phenyl and pyridinyl; wherein any phenyl or pyridinyl of R₁ is optionally substituted with 1 to 3 radicals independently selected from chloro, bromo, fluoro, trifluoromethyl, methyl, ethyl and C₁₋₆alkoxy, Rₓ is selected from C(O)OC₁₋₁₀alkyl and halo-substituted-C(O)OC₁₋₁₀alkyl; R₃ is selected from C₁₋₆alkyl optionally substituted with 1-5 halo radicals; wherein any alkyl of R₃ can optionally have a methylene replaced with —O—; R₄ is hydrogen; and R₅ is selected from C₁₋₆alkyl and —XR₉; wherein X is a bond or C₁₋₆alkylene; R₉ is independently selected from hydroxy, C₁₋₆alkyl, halo-substituted-C₁₋₆alkyl, C₆₋₁₀aryl and C₅₋₁₀heteroaryl; wherein any aryl or heteroaryl of R₉ is optionally substituted with 1 to 3 radicals independently selected from halo, hydroxy, nitro, amino, cyano, C₁₋₆alkyl, C₁₋₆alkoxy, halo-substituted-C₁₋₆alkyl, halo-substituted-C₁₋₆alkoxy, —C(O)OR₁₀, —OR₁₀ and —C(O)R₁₀; and wherein R₁₀ is selected from methyl and phenyl.

In a further embodiment, R₅ is selected from C₁₋₆alkyl, halo-C₁₋₆alkyl and —XR₉; wherein X is a bond or C₁₋₆alkylene; R₉ is independently selected from hydroxy, C₁₋₆alkyl, halo-substituted-C₁₋₆alkyl, C₆₋₁₀aryl and C₅₋₁₀heteroaryl; wherein any aryl or heteroaryl of R₉ is optionally substituted with 1 to 3 radicals independently selected from halo, hydroxy, nitro, amino, cyano, C₁₋₆alkyl, C₁₋₆alkoxy, halo-substituted-C₁₋₆alkyl, halo-substituted-C₁₋₆alkoxy, C(O)OR₁₀, —OR₁₀ and —C(O)R₁₀; wherein R₁₀ is selected from methyl and phenyl.

Preferred compounds of Formula I are selected from the examples and tables, 1, 2 and 3, infra.

### Pharmacology and Utility

Compounds of the invention modulate the activity of steroidal nuclear hormone receptors and, as such, are useful for treating diseases or disorders in which aberrant steroidal nuclear hormone receptor activity contributes to the pathology and/or symptomatology of the disease. The invention further provides compounds for use in the preparation of medicaments for the treatment of diseases or disorders in which steroidal nuclear hormone receptor activity contributes to the pathology and/or symptomatology of the disease.

Mineralocorticoids and glucocorticoids exert profound influences on a multitude of physiological functions by virtue of their diverse roles in growth, development, and maintenance of homeostasis. Their actions are mediated by the MR and GR.

In visceral tissues, such as the kidney and the gut, MR regulates sodium retention, potassium excretion, and water balance in response to aldosterone. Elevations in aldosterone levels, or excess stimulation of mineralocorticoid receptors, are linked to several pathological disorders or pathological disease states including, Conn's Syndrome, primary and secondary hyperaldosteronism, increased sodium retention, increased magnesium and potassium excretion (diuresis), increased water retention, hypertension (isolated systolic and combined systolic/diastolic), arrhythmias, myocardial fibrosis, myocardial infarction, Barter's Syndrome, congestive heart failure (CHF), and disorders associated with excess catecholamine levels. In addition, MR expression in the brain appears to play a role in the control of neuronal excitability, in the negative feedback regulation of the hypothalamic-pituitary-adrenal axis, and in the cognitive aspects of behavioral performance. Further, aldosterone antagonists are useful in the treatment of subjects suffering from one or more cognitive dysfunctions including, but not limited to psychoses, cognitive disorders (such as memory disturbances), mood disorders (such as depression and bipolar disorder), anxiety disorders, and personality disorders. In particular, mineralocorticoid receptors, and modulation of MR activity, are involved in anxiety and major depression. Finally, expression of MR may be related to differentiation of breast carcinomas. Thus MR modulators may also have utility in treating cancer, particularly of the breast.

GR is expressed in almost all tissues and organ systems and is crucial for the integrity of the function of the central nervous system and the maintenance of cardiovascular, metabolic, and immune homeostasis. Glucocorticoids (e.g. cortisol, corticosterone, and cortisone), and the glucocorticoid receptor, have been implicated in the etiology of a variety of pathological disorders or pathologic disease states. For example, cortisol hypo-secretion is implicated in the pathogenesis of diseases resulting in muscle weakness, increased melanin pigmentation of the skin, weight loss, hypotension, and hypoglycemia. On the other hand, excessive or prolonged secretion of glucocorticoids has been correlated to Cushing's Syndrome and can also result in obesity, hypertension, glucose intolerance, hyperglycemia, diabetes mellitus, osteoporosis, polyuria, and polydipsia.

Further, GR selective agents could modulate GR activity and, thus, be useful in the treatment of inflammation, tissue rejection, auto-immunity, malignancies such as leukemias and lymphomas, Cushing's syndrome, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, modulation of the Th1/Th2 cytokine balance, chronic kidney disease, stroke and spinal cord injury, hypocalcaemia, hyperglycemia, acute adrenal insufficiency, chronic primary adrenal insufficiency, secondary adrenal insufficiency, congenital adrenal hyperplasia, cerebral edema, thrombocytopenia, and Little's syndrome. It has been reported that GR modulators are especially useful in disease states involving systemic inflammation such as inflammatory bowel disease, systemic lupus erythematosus, polyartitis nodosa, Wegener's granulomatosis, giant cell arthritis, rheumatoid arthritis, osteoarthritis, hay fever, allergic rhinitis, urticaria, angioneurotic edema, chronic obstructive pulmonary disease, asthma, tendonitis, bursitis, Crohn's disease, ulcerative colitis, autoimmune chronic active hepatitis, organ transplantation, hepatitis, and cirrhosis; and that GR modulating compounds have been used as immunostimulants, repressors, and as wound healing and tissue repair agents. In addition, GR modulators have also found use in a variety of topical diseases such as inflammatory scalp alopecia, panniculitis, psoriasis, discoid lupus erythematosus, inflamed cysts, atopic dermatitis, pyoderma gangrenosum, pemphigus vulgaris, bullous pemphigoid, systemic lupus erythematosus, dermatomyositis, eosinophilic fasciitis, relapsing polychondritis, inflammatory vasculitis, sarcoidosis, Sweet's disease, type 1 reactive leprosy, capillary hemangiomas, contact dermatitis, atopic dermatitis, lichen planus, exfoliative dermatitis, erythema nodosum, acne, hirsutism, toxic epidermal necrolysis, erythema multiform, and cutaneous T-cell lymphoma. Finally, GR Modulators may also have utility in treating respiratory disorders, such as emphysema, and neuroinflammatory disorders, such as multiple sclerosis and Alzheimer's disease.

Calcium channels are membrane-spanning, multi-subunit proteins that allow calcium entry from the external milieu and concurrent depolarization of the cell's membrane potential. Traditionally, calcium channels have been classified based on their functional characteristics such as low voltage or high voltage activated and their kinetics (L, T, N, P, Q). Calcium channel antagonists have long been used as drugs to treat various diseases, particularly cardiovascular diseases, such as coronary vasodilation, angina, arrhythmias, congestive heart failure, cardiomyopathy, atherosclerosis, high blood pressure and the like. Modulation of calcium channel activity combined to the modulation of the nuclear hormone receptors (especially MR) in the same molecular entity offers an attractive novel way to treat cardiovascular diseases associated with the modulation of both these functional entities.

Accordingly, the present invention provides a method for treating any of the diseases or disorders described above in a subject in need of such treatment, which method comprises administering to said subject a therapeutically effective amount (See, “Administration and Pharmaceutical Compositions”, infra) of a compound of Formula I or a pharmaceutically acceptable salt thereof. For any of the above uses, the required dosage will vary depending on the mode of administration, the particular condition to be treated and the effect desired.

### Administration and Pharmaceutical Compositions

In general, compounds of the invention will be administered in therapeutically effective amounts via any of the usual and acceptable modes known in the art, either singly or in combination with one or more therapeutic agents. A therapeutically effective amount can vary widely depending on the severity of the disease, the age and relative health of the subject, the potency of the compound used and other factors. In general, satisfactory results are indicated to be obtained systemically at daily dosages of from about 0.03 to 2.5 mg/kg of body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets, also c) binders, e.g., magnesium aluminum silicate, starch paste, gelatin, tragacanth, methylcellulose, sodium carboxymethylcellulose and or polyvinylpyrollidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or Suspensions, and suppositories can be prepared from fatty emulsions or suspensions. The compositions can be sterilized and/or contain adjuvants, such as preserving, stabilizing, wetting or emulsifying agents, solution promoters, salts for regulating the osmotic pressure and/or buffers. In addition, they can also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations can also be used. Suitable formulations for topical application, e.g., to the skin and eyes, are preferably aqueous solutions, ointments, creams or gels well-known in the art. Such can contain solubilizers, stabilizers, tonicity enhancing agents, buffers and preservatives.

Compounds of the invention can be administered in therapeutically effective amounts in combination with one or more therapeutic agents (pharmaceutical combinations). For example, synergistic effects can occur with other calcium channel blockers and/or other substances used in the treatment of hypokalemia, hypertension, congestive heart failure, renal failure, in particular chronic renal failure, restenosis, atherosclerosis, syndrome X, obesity, nephropathy, post-myocardial infarction, coronary heart disease, increased formation of collagen, fibrosis and remodeling following hypertension and endothelial dysfunction. Examples of such compounds include anti-obesity agents, such as orlistat, anti-hypertensive agents, inotropic agents and hypolipidemic agents e.g., loop diuretics, such as ethacrynic acid, furosemide and torsemide; angiotensin converting enzyme (ACE) inhibitors, such as benazepril, captopril, enalapril, fosinopril, lisinopril, moexipril, perinodopril, quinapril, ramipril and trandolepril; inhibitors of the Na—K-ATPase membrane pump, such as digoxin; neutralendopeptidase (NEP) inhibitors; ACE/NEP inhibitors, such as omapatrilat, sampatrilat, and fasidotril; angiotensin II antagonists, such as candesartan, eprosartan, irbesartan, losartan, telmisartan and valsartan, in particularvalsartan; β-adrenergic receptor blockers, such as acebutolol, betaxolol, bisoprolol, metoprolol, nadolol, propanolol, sotalol and timolol; inotropic agents, such as digoxin, dobutamine and milrinone; calcium channel blockers, such as amlodipine, bepridil, diltiazem, felodipine, nicardipine, nimodipine, nifedipine, nisoldipine and verapamil; and 3-hydroxy-3-methyl-glutaryl coenzyme A reductase (HMG-CoA) inhibitors, such as lovastatin, pitavastatin, simvastatin, pravastatin, cerivastatin, mevastatin, velostatin, fluvastatin, dalvastatin, atorvastatin, rosuvastatin and rivastatin. Where the compounds of the invention are administered in conjunction with other therapies, dosages of the co-administered compounds will of course vary depending on the type of co-drug employed, on the specific drug employed, on the condition being treated and so forth.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### Processes for Making Compounds of the Invention

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

Compounds of Formula I, in which R₄ is hydrogen and R₅ is any of the definitions of R₅ in the Summary of the Invention in which a sulfur atom is attached to the dihydro pyridine ring of Formula I (-SR₉ is shown in reaction schemes A and B) can be synthesized according to reaction schemes A or B:

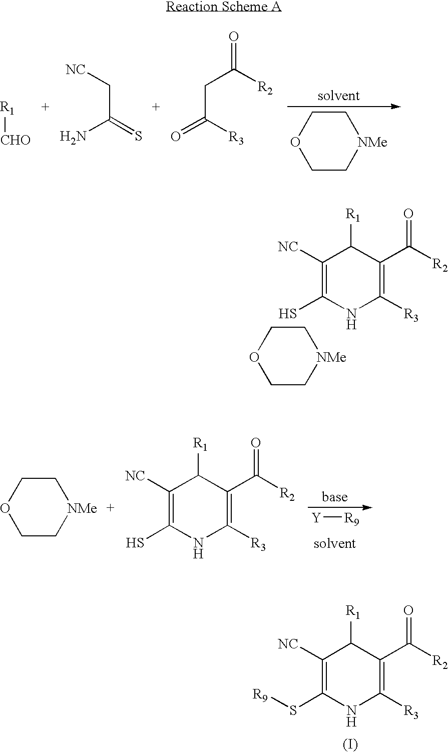

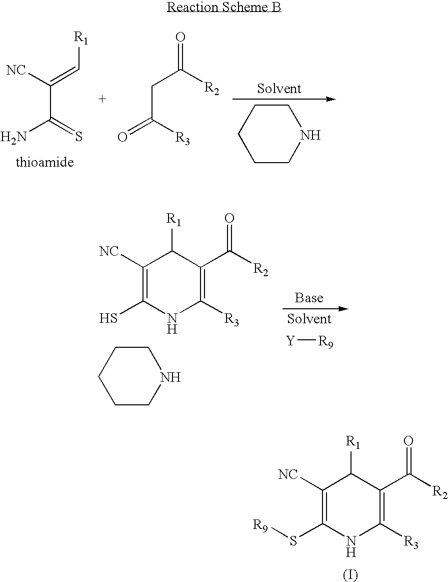

in which R₁, R₂, R₃, and R₉ are as described in the Summary of the Invention. In each case, an intermediate is formed by reaction of an aldehyde, a dicarbonyl derivative a base (piperidine or N-methyl morpholine for example) and a thioamide in an alcoholic solvent (e.g., ethanol, or the like). The reaction proceeds for up to about 16 hours in a temperature range from about 5° C. to about 50° C. This intermediate can also be synthesized from the reaction of a more elaborated thioamide and a dicarbonyl compound under the same conditions (scheme B). Alkylation of this intermediate with various alkyl or benzyl halides in the presence of a base (e.g., cesium fluoride, or the like) in a solvent (e.g., ethanol, or the like) in a temperature range from about 5° C. to about 50° C. affords the desired compounds of the Invention.

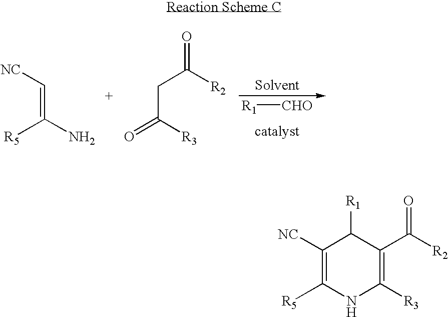

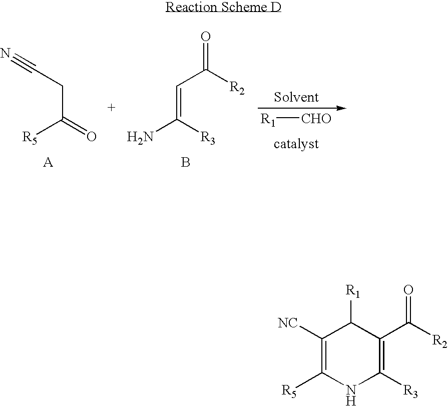

Compounds prepared from scheme C and D are prepared by mixing a 1,3-dicarbonyl compound with a amino-cyano crotonate derivative and an aldehyde in an alcoholic solvent (e.g., isopropanol, or the like) and optionally in the presence of a base catalyst (e.g., piperidine, or the like). The reactions proceed in a temperature range from about room temperature to about 100° C. for up to about 16 hours. The intermediates A and B are prepared according to scheme E (G. Zhu, and al, J. Org. Chem. 1999, 64, 6907; A. Bhandari and al, Synthesis, 1999, 11, 1951; F. F. Fleming and al, J. Org. Chem., 1997, 62, 3036; D. N. Ridge, and al, J. Med. Chem., 1979 22, 1385).

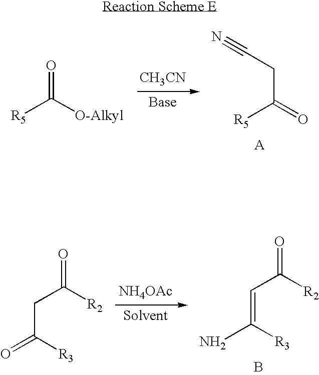

Specific examples of synthesis of compounds of the invention are detailed in Examples 1 to 3, infra.

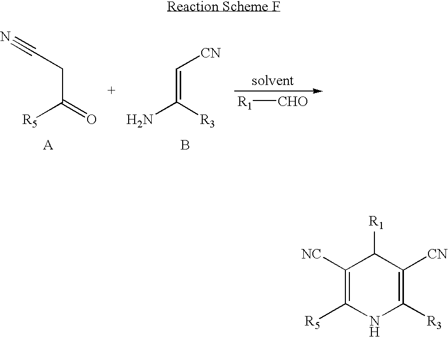

### Additional Processes for Making Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base. Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxane, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

In summary, the compounds of Formula I can be made by a process, which involves:

(a) that of reaction schemes A, B, C and D; and

(b) optionally converting a compound of the invention into a pharmaceutically acceptable salt;

(c) optionally converting a salt form of a compound of the invention to a non-salt form;

(d) optionally converting an unoxidized form of a compound of the invention into a pharmaceutically acceptable N-oxide;

(e) optionally converting an N-oxide form of a compound of the invention to its unoxidized form;

(f) optionally resolving an individual isomer of a compound of the invention from a mixture of isomers;

(g) optionally converting a non-derivatized compound of the invention into a pharmaceutically acceptable prodrug derivative; and

(h) optionally converting a prodrug derivative of a compound of the invention to its non-derivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present invention, and that other well known methods can similarly be used.

### EXAMPLES

The present invention is further exemplified, but not limited, by the following reference examples (intermediates) and examples that illustrate the preparation of compounds of Formula I according to the invention.

### Example 1

### N-methyl-4-morpholinium-6-methyl-4-(2-fluoro-4-bromophenyl)-5-(2-methoxyphenyl)carbamoyl-3-cyano-1,4-dihydro-pyridine-2-thiolate 1 (Scheme A)

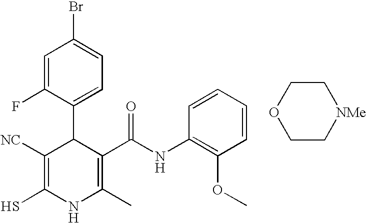

A round bottomed flask equipped with a magnetic stir bar is charged with 4.06 g (20 mmol) of 2-fluoro-4-bromo benzaldehyde, 2.0 g (20 mmol) of 2-cyanothioacetamide and 4.14 g (20 mmol) of o-acetocetaniside. 50 ml of ethanol is then added followed by 3 ml (30 mmol) of N-methylmorpholine. The resulting reddish solution is stirred at room temperature until a yellow precipitate forms (typically 2 hours). The precipitate is filtrated and washed twice with ethanol, ether and hexanes to give N-methyl-4-morpholinium-6-methyl-4-(2-fluoro-4-bromophenyl)-5-(2-methoxyphenyl)carbamoyl-3-cyano-1,4-dihydro-pyridine-2-thiolate 1 as a pale orange powder: ¹H NMR (DMSO-d6, 400 MHz): 8.089 (broads, 1H), 8.035 (d, J=7.6 Hz, 1H), 7.802 (broads, 1H), 7.319 (d, J=9.6 Hz, 1H), 7.712 (t, J=8.0 Hz, 1H), 6.93 (m, 2H), 6.82 (m, 1H), 4.64 (s, 1H), 3.75 (m, 1H), 3.71 (s, 3H), 3.32 (m, 1H), 3.09 (m, 2H), 2.51 (s, 3H), 2.22 (s, 3H). MS (ES⁺) 475, m/z (M+1)⁺ C₂₆H₂₈BrFN₄O₃S minus C₅H₁₁NO requires 475.

By repeating the procedures described in the above example, using appropriate starting materials, compounds of Formula I, as identified in Table I infra, are obtained.

### Example 2

### 2-(4-methylbenzyl)thio-3-cyano-4-(2-chlorophenyl)-5-(2-methoxyphenyl)carbamoyl-6-methyl-1,4-dihydro-pyridine

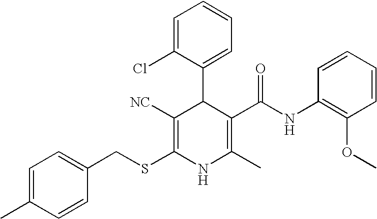

In a scintillation vial containing 55 mg (0.1 mmol) of N-methyl-4-morpholinium-6-methyl-4-(2,4-dichlorophenyl)-5-(2-methoxyphenyl)carbamoyl-3-cyano-1,4-dihydro-pyridine-2-thiolate diluted in 2 ml of EtOH is added 20 mg (0.108 mmol) of 4-methylbenzyl bromide and 23 mg (0.15 mmol) of CsF. The solution is stirred at room temperature overnight and EtOAc is added. The remaining solution is filtrated over a short silica plug and the solvents evaporated under reduced pressure. The remaining pale yellow oil is recrystallized from EtOAc/hexanes to give example 2 as a pale yellow solid: ¹H NMR (CDCl₃, 400 MHz): 8.23 (d, J=7.6 Hz, 1H), 7.53 (broad s, 1H), 7.42 (dd, J=5.6, 3.6 Hz, 1H), 7.24 (dd, J=6.4, 4.0 Hz, 2H), 7.20 (m, 1H), 7.14 (d, J=8.0 Hz, 2H), 7.08 (d, J=8.0 Hz, 2H), 6.97 (t, J=7.6 Hz, 1H), 6.88 (t, J=7.6 Hz, 1H), 6.75 (d, J=8.0 Hz, 1H), 5.93 (broad s, 1H), 5.22 (s, 1H), 4.08 (d, J=13.6 Hz, 2H), 3.98 (d, J=13.6 Hz, 2H), 3.66 (s, 3H), 2.34 (s, 3H), 2.14 (s, 3H),). MS (ES⁺) 516, m/z (M+1)⁺ C₂₉H₂₆ClN₃O₂S requires 516.

By repeating the procedures described in the above example, using appropriate starting materials, compounds of Formula I, as identified in Table 2, are obtained.

### Example 3

### 2,6-dimethyl-3-cyano-4-(2-chlorophenyl)-5-(2-methoxyphenyl)carbamoyl-1,4-dihydro-pyridine

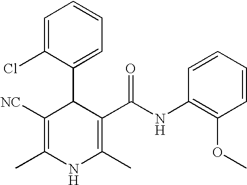

This compound is prepared from 1.46 g (10 mmol) of 2-chlorobenzaldehyde, 1.0 g (10 mmol) of 3-aminocyanocrotonate and 2.07 g (101 mmol) of o-acetoacetaniside in refluxing iPrOH for 24 hours. After cooling at room temperature and evaporation of the solvents under reduced pressure, the crude pasty oil is recrystallized twice from MeOH to give 2,6-dimethyl-3-cyano-4-(2,4-dichlorophenyl)-5-(2-methoxyphenyl)carbamoyl-1,4-dihydro-pyridine as a white crystal: ¹H NMR (DMSO-d6, 400 MHz): 8.96 (broad s, 1H), 8.29 (broad s, 1H), 7.76 (dd, J=8.0, 1.6 Hz, 1H), 7.43 (m, 2H), 7.38 (t, J=8.0 Hz, 1H), 7.27 (td, J=8.0, 1.6 Hz, 1H), 6.98 (td, J=8.0, 1.2 Hz, 1H), 6.94 (dd, J=8.0, 1.2 Hz, 1H), 6.81 (td, J=8.0, 1.2 Hz, 1H), 5.17 (s, 1H), 3.70 (s, 3H), 2.15 (s, 3H), 2.00 (s, 3H). MS (ES⁺) 394, m/z (M+1)⁺ C₂₂H₂₀ClN₃O₂ requires 394.

By repeating the procedures described in the above example, using appropriate starting materials, compounds of Formula I, as identified in Table 3, are obtained.

TABLE I

| | | Physical Data |
|---|---|---|
| Example | | ¹H NMR 400 MHz (DMSO-d6) and/or |
| Number | Structure | MS (m/z) |
| 4 | 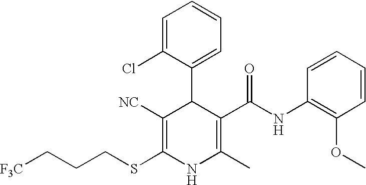 2-(4,4,4-trifluorobutyl)thio-3-cyano-4-(2- chlorophenyl)-5-(2-methoxyphenyl)carbamoyl-6- methyl-1,4-dihydro-pyridine | ¹H NMR (DMSO-d6, 400 MHz): 9.33 (broad s, 1H), 8.47 (broad s, 1H), 7.71 (dd, J = 8.0, 1.6 Hz, 1H), 7.43 (dd, J = 8.0, 1.2 Hz, 1H), 7.40 (dd, J = 8.0, 1.6 Hz, 1H), 7.37 (td, J = 8.0, 1.2 Hz, 1H), 7.29 (td, J = 8.0, 1.2 Hz, 1H), 7.02 (dd, J = 7.6, 1.2 Hz, 1H), 6.96 (dd, J = 7.6, 1.2 Hz, 1H), 6.83 (td, J = 7.6, 1.2 Hz, 1H), 5.22 (s, 1H), 3.71 (s, 3H), 3.13 (m, 1H), 2.98 (m, 1H), 2.39 (m, 2H), 2.17 (s, 3H), 1.73 (m, 2H). MS (ES⁺) 522, m/z (M + 1)⁺ C₂₅H₂₃ClF₃N₃O₂S requires 522. |
| 5 | 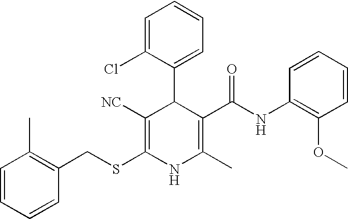 2-(2-methylbenzyl)thio-3-cyano-4-(2- chlorophenyl)-5-(2-methoxyphenyl)carbamoyl-6- methyl-1,4-dihydro-pyridine | ¹H NMR (DMSO-d6, 400 MHz): 9.21 (broad s, 1H), 8.29 (broad s, 1H), 7.64 (dd, J = 8.0, 1.6 Hz, 1H), 7.34 (dd, J = 8.0, 1.6 Hz, 1H), 7.24 (td, J = 8.0, 1.6 Hz, 1H), 7.20 (td, J = 8.0, 1.6 Hz, 1H), 7.14 (m, 2H), 7.01 (m, 2H), 6.92 (td, J = 7.2, 1.6 Hz, 1H), 6.88 (dd, J = 7.2, 1.6 Hz, 1H), 6.75 (dd, J = 7.2, 1.6 Hz, 1H), 5.03 (s, 1H), 4.19 (d, J = 13.2 Hz, 1H), 4.12 (d, J = 13.2 Hz, 1H), 3.62 (s, 3H), 2.20 (s, 3H), 2.10 (s, 3H). MS (ES⁺) 517, m/z (M + 1)⁺ C₂₉H₂₆ClN₃O₂S requires 517. |
| 6 | 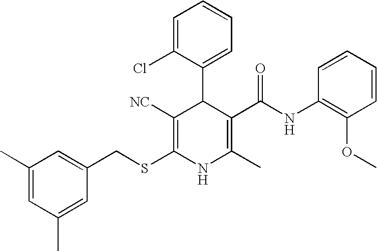 2-(3,5-dimethylbenzylbenzyl)thio-3-cyano-4-(2- chlorophenyl)-5-(2-methoxyphenyl)carbamoyl-6- methyl-1,4-dihydro-pyridine | ¹H NMR (CDCl₃, 400 MHz): 8.24 (dd, J = 8.0, 1.6 Hz, 1H), 7.54 (broad s, 1H), 7.44 (m, 1H), 7.26 (m, 3H), 6.98 (td, J = 7.6, 1.6 Hz, 1H), 6.88 (td, J = 7.6, 1.6 Hz, 1H), 6.77 (dd, J = 7.6, 1.6 Hz, 1H), 5.73 (broad s, 1H), 5.24 (s, 1H), 4.01 (s, 2H), 3.67 (s, 3H), 2.32 (s, 6H), 2.10 (s, 3H). MS (ES⁺) 531, m/z (M + 1)⁺ C₃₀H₂₈ClN₃O₂S requires 531. |
| 7 | 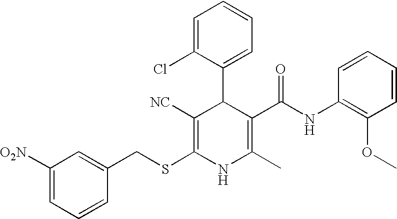 2-(3-nitrobenzylbenzyl)thio-3-cyano-4-(2- chlorophenyl)-5-(2-methoxyphenyl)carbamoyl-6- methyl-1,4-dihydro-pyridine | ¹H NMR (DMSO-d6, 400 MHz): 9.35 (broad s, 1H), 8.17 (broad s, 1H), 8.15 (d, J = 8.0, Hz, 1H), 7.69 (td, J = 8.0, 1.2 Hz, 2H), 7.61 (t, J = 7.6 Hz, 1H), 7.38 (m, 1H), 7.26 (m, 2H), 7.10 (m, 1H), 7.01 (td, J = 7.6, 1.2 Hz, 1H), 6.94 (dd, J = 7.6, 1.2 Hz, 1H), 6.80 (td, J = 7.6, 1.2 Hz, 1H), 5.07 (s, 1H), 4.44 (d, J = 13.5 Hz, 1H), 4.38 (d, J = 13.5 Hz, 1H), 3.69 (s, 3H), 2.17 (s, 3H). MS (ES⁺) 548, m/z (M + 1)⁺ C₂₈H₂₃ClN₄O₄S requires 548. |
| 8 | 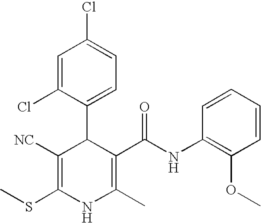 2-methylthio-3-cyano-4-(2,4-dichlorophenyl)-5- (2-methoxyphenyl)carbamoyl-6-methyl-1,4- dihydro-pyridine | ¹H NMR (DMSO-d6, 400 MHz): 9.16 (broad s, 1H), 8.62 (broad s, 1H), 7.68 (dd, J = 8.0, 1.6 Hz, 1H), 7.61 (d, J = 2.0 Hz, 1H), 7.48 (dd, J = 8.4, 2.0 Hz, 1H), 7.40 (d, J = 8.4 Hz, 1H), 7.03 (td, J = 8.0, 1.6 Hz, 1H), 6.98 (dd, J = 8.0, 1.6 Hz, 1H), 6.84 (td, J = 8.0, 1.6 Hz, 1H), 5.17 (s, 1H), 3.73 (s, 3H), 3.34 (s, 3H), 2.15 (s, 3H). MS (ES⁺) 461, m/z (M + 1)⁺ C₂₂H₁₉Cl₂N₃O₂S requires 461. |
| 9 | 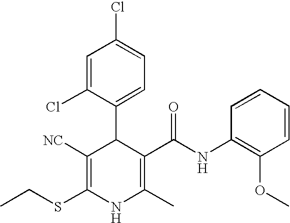 2-ethylthio-3-cyano-4-(2,4-dichlorophenyl)-5-(2- methoxyphenyl)carbamoyl-6-methyl-1,4-dihydro- pyridine | ¹H NMR (DMSO-d6, 400 MHz): 9.24 (broad s, 1H), 8.62 (broad s, 1H), 7.65 (dd, J = 8.0, 1.6 Hz, 1H), 7.60 (d, J = 2.0 Hz, 1H), 7.48 (dd, J = 8.4, 2.0 Hz, 1H), 7.42 (d, J = 8.4 Hz, 1H), 7.05 (td, J = 8.0, 1.6 Hz, 1H), 6.97 (dd, J = 8.0, 1.6 Hz, 1H), 6.84 (td, J = 8.0, 1.6 Hz, 1H), 5.21 (s, 1H), 3.73 (s, 3H), 3.05 (m, 1H), 2.96 (m, 1H), 2.15 (s, 3H), 1.20 (t, J = 7.3 Hz, 3H). MS (ES⁺) 475, m/z (M + 1)⁺ C₂₃H₂₁Cl₂N₃O₂S requires 475. |
| 10 | 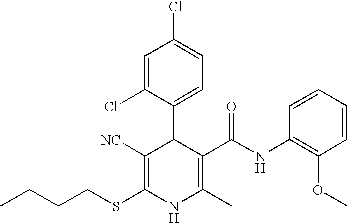 2-butylthio-3-cyano-4-(2,4-dichlorophenyl)-5-(2- methoxyphenyl)carbamoyl-6-methyl-1,4-dihydro- pyridine | ¹H NMR (DMSO-d6, 400 MHz): 9.27 (broad s, 1H), 8.60 (broad s, 1H), 7.67 (dd, J = 8.0, 1.6 Hz, 1H), 7.61 (d, J = 2.0 Hz, 1H), 7.48 (dd, J = 8.4, 2.0 Hz, 1H), 7.40 (d, J = 8.4 Hz, 1H), 7.04 (td, J = 8.0, 1.6 Hz, 1H), 6.98 (dd, J = 8.0, 1.6 Hz, 1H), 6.83 (td, J = 8.0, 1.6 Hz, 1H), 5.19 (s, 1H), 3.72 (s, 3H), 3.06 (m, 1H), 2.93 (m, 1H), 2.15 (s, 3H), 1.49 (m, 1H), 1.37 (m, 1H), 0.85 (t, J = 7.2 Hz, 3H). MS (ES⁺) 503, m/z (M + 1)⁺ C₂₅H₂₅Cl₂N₃O₂S requires 503. |
| 11 | 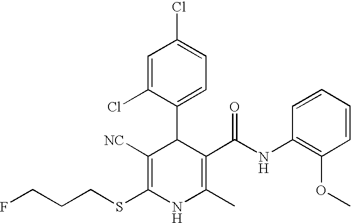 2-(3-fluoropropyl)thio-3-cyano-4-(2,4- dichlorophenyl)-5-(2-methoxyphenyl)carbamoyl- 6-methyl-1,4-dihydro-pyridine | ¹H NMR (DMSO-d6, 400 MHz): 9.32 (broad s, 1H), 8.63 (broad s, 1H), 7.66 (dd, J = 8.0, 1.6 Hz, 1H), 7.61 (d, J = 2.0 Hz, 1H), 7.48 (dd, 8.4, 2.0 Hz, 1H), 7.42 (d, J = 8.4 Hz, 1H), 7.05 (td, J = 8.0, 1.6 Hz, 1H), 6.97 (dd, J = 8.0, 1.6 Hz, 1H), 6.84 (td, J = 8.0, 1.6 Hz, 1H), 5.22 (s, 1H), 4.61 (m, 1H), 4.49 (m, 1H), 3.73 (s, 3H), 3.14 (m, 1H), 3.00 (m, 1H), 2.14 (s, 3H), 1.91 (m, 2H). MS (ES⁺) 507, m/z (M + 1)⁺ C₂₄H₂₂Cl₂FN₃O₂S requires 507. |
| 12 | 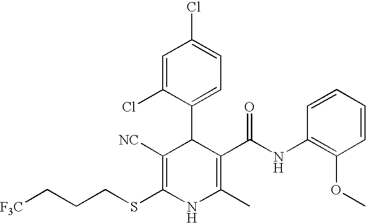 2-(4,4,4-trifluorofluorobutyl)thio-3-cyano-4-(2,4- dichlorophenyl)-5-(2-methoxyphenyl)carbamoyl- 6-methyl-1,4-dihydro-pyridine | ¹H NMR (DMSO-d6, 400 MHz): 9.34 (broad s, 1H), 8.61 (broad s, 1H), 7.66 (dd, J = 8.0, 1.2 Hz, 1H), 7.61 (d, J = 2.0 Hz, 1H), 7.46 (dd, J = 8.0, 2.0 Hz, 1H), 7.41 (d, J = 8.4 Hz, 1H), 7.05 (td, J = 8.0, 1.2 Hz, 1H), 6.97 (dd, J = 8.0, 1.2 Hz, 1H), 6.82 (td, J = 8.0, 1.2 Hz, 1H), 5.22 (s, 1H), 3.73 (s, 3H), 3.13 (m, 1H), 2.99 (m, 1H), 2.40 (m, 2H), 2.15 (s, 3H), 1.74 (m, 2H). MS (ES⁺) 557, m/z (M + 1)⁺ C₂₅H₂₂Cl₂F₃N₃O₂S requires 557. |
| 13 | 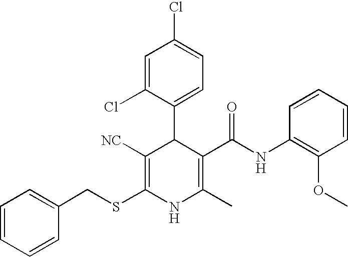 2-benzylthio-3-cyano-4-(2,4-dichlorophenyl)-5- (2-methoxyphenyl)carbamoyl-6-methyl-1,4- dihydro-pyridine | ¹H NMR (DMSO-d6, 400 MHz): 9.23 (broad s, 1H), 8.46 (broad s, 1H), 7.56 (dd, J = 8.0, 1.2 Hz, 1H), 7.49 (d, J = 2.0 Hz, 1H), 7.31 (dd, J = 8.0, 2.0 Hz, 1H), 7.23 (m, 5H), 7,09 (d, J = 8.4 Hz, 1H), 6.98 (td, J = 8.0, 1.2 Hz, 1H), 6.89 (dd, J = 8.0, 1.2 Hz, 1H), 6.76 (td, J = 8.0, 1.2 Hz, 1H), 5.03 (s, 1H), 4.22 (d, J = 13.2 Hz, 1H), 4.18 (d, J = 13.2 Hz, 1H), 3.65 (s, 3H), 2.08 (s, 3H). MS (ES⁺) 537, m/z (M + 1)⁺ C₂₈H₂₃Cl₂F₃N₃O₂S requires 537. |
| 14 | 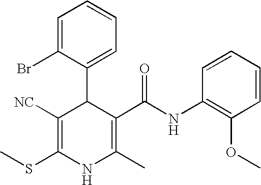 2-methylthio-3-cyano-4-(2-bromophenyl)-5-(2- methoxyphenyl)carbamoyl-6-methyl-1,4-dihydro- pyridine | ¹H NMR (DMSO-d6, 400 MHz): 9.14 (broad s, 1H), 8.44 (broad s, 1H),, 7.72 (dd, J = 8.0, 1.6 Hz, 1H), 7.61 (dd, J = 8.0, 1.2 Hz, 1H), 7.42 (m, 2H), 7.21 (m, 1H), 7.01 (td, J = 8.0, 1.2 Hz, 1H), 6.96 (dd, J = 8.0, 1.2 Hz, 1H), 6.84 (td, J = 8.0, 1.2 Hz, 1H), 5.14 (s, 1H), 3.70 (s, 3H), 3.34 (s, 3H), 2.16 (s, 3H). MS (ES⁺) 471, m/z (M + 1)⁺ C₂₂H₂₀BrN₃O₂S requires 471. |
| 15 | 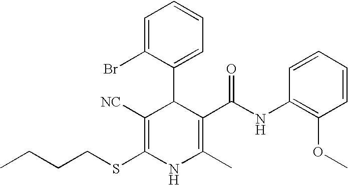 2-butylthio-3-cyano-4-(2-bromophenyl)-5-(2- methoxyphenyl)carbamoyl-6-methyl-1,4-dihydro- pyridine | ¹H NMR (CDCl₃, 400 MHz): 8.24 (dd, J = 7.6, 1.2 Hz, 1H), 7.62 (d, J = 8.0 Hz, 1H), 7.56 (broad s, 1R), 7.39 (dd, J = 8.0, 2.0 Hz, 1H), 7.34 (t, J = 7.2 Hz, 1H), 7.17 (td, J = 7.2, 1.6 Hz, 1H), 6.97 (td, J = 7.6, 1.6 Hz, 1H), 6.88 (td, J = 7.6, 1.6 Hz, 1H), 6.77 (dd, J = 7.6, 1.6 Hz, 1H), 6.27 (broad s, 1H), 5.26 (s, 1H), 3.66 (s, 3H), 2.95 (m, 1H), 2.79 (m, 1H), 2.34 (s, 3H), 1.54 (m, 2H), 1.34 (m, 2H), 0.86 (t, J = 7.2 Hz, 3H). MS (ES⁺) 513 and 515, m/z (M + 1)⁺ C₂₅H₂₆BrN₃O₂S requires 513. |
| 16 | 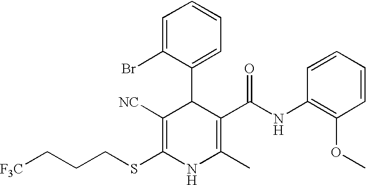 2-(4,4,4-trifluorobutylthio-3-cyano-4-(2- bromophenyl)-5-(2-methoxyphenyl)carbamoyl-6- methyl-1,4-dihydro-pyridine | ¹H NMR (DMSO-d6, 400 MHz): 9.32 (broad s, 1H), 8.45 (broad s,1H), , 7.71 (dd, J = 8.0, 1.6 Hz, 1H), 7.60 (d, J = 8.0 Hz, 1H), 7.42 (m, 2H), 7.23 (m, 1H), 7.03 (td, J = 8.0, 1.2 Hz, 1H), 6.95 (dd, J = 8.0, 1.2 Hz, 1H), 6.83 (td, J = 8.0, 1.2 Hz, 1H), 5.19 (s, 1H), 3.70 (s, 3H), 3.13 (dt, J = 13.6, 6.8 Hz, 1H), 3.01 (dt, J = 13.6, 6.8 Hz, 1H), 2.39 (m, 2H), 2.15 (s, 3H), 1.74 (m, 2H). MS (ES⁺) 567, m/z (M + 1)⁺ C₂₅H₂₃BrF₃N₃O₂S requires 567. |
| 17 | 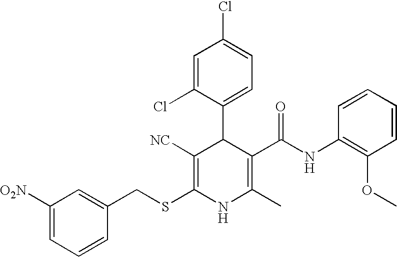 2-(3-nitrobenzyl)thio-3-cyano-4-(2,4- dichlorophenyl)-5-(2-methoxyphenyl)carbamoyl- 6-methyl-1,4-dihydro-pyridine | ¹H NMR (DMSO-d6, 400 MHz): 8.21 (dd, J = 8.0, 1.6 Hz, 1H), 8.15 (t, J = 2.0 Hz, 1H), 8.10 (dd, J = 8.0, 1.2 Hz, 1H), 7.55 (d, J = 8.0 Hz, 1H), 7.46 (s, broad s, 1H), 7.42 (d, J = 2.0 Hz, 1H), 7.36 (t, J = 8.0 Hz, 1H), 7.13 (dd, J = 8.4, 2.0 Hz, 1H), 6.97 (td, J = 8.0, 1.2 Hz, 1H), 6.96)d, J = 8.0 Hz, 1H), 6.88 (dd, J = 8.0, 1.2 Hz, 1H), 6.77 (td, J = 8.0, 1.2 Hz, 1H), 5.92 (broad s, 1H), 5.14 (s, 1H), 4.36 (d, J = 14.0 Hz, 1H), 4.07 (d, J = 14.0 Hz, 1H), 3.69 (s, 3H), 2.26 (s, 3H). MS (ES⁺) 582, m/z (M + 1)⁺ C₂₈H₂₂Cl₂N₄O₄S requires 582. |
| 18 | 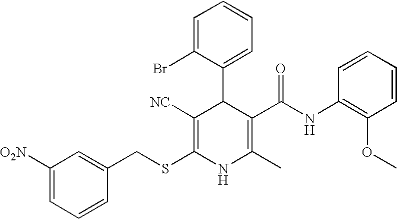 2-(3-nitrobenzylthio-3-cyano-4-(2-bromophenyl)- 5-(2-methoxyphenyl)carbamoyl-6-methyl-1,4- dihydro-pyridine | ¹H NMR (Acetone-d6, 400 MHz): 8.48 (broad s, 1H), 8.24 (broad s, 1H),, 8.18 (dd, J = 8.0, 1.6 Hz, 1H), 8.16 (dd, J = 8.0, 1.2 Hz, 1H), 7.78 (s, 1H), 7.60 (d, J = 7.6 Hz, 1H), 7.29 (td, J = 7.6, 1.2 Hz, 1H), 7.26 (dd, J = 8.0, 2.0 Hz, 1H), 7.19 (td, J = 7.6, 2.0 Hz, 1H), 6.96 (td, J = 7.6, 1.2 Hz, 1H), 6.92 (dd, J = 7.6, 1.2 Hz, 1H), 6.84 (td, J = 7.6, 1.2 Hz, 1H), 5.17 (s, 1H), 4.47 (d, J = 13.8 Hz, 1H), 4.35 (d, J = 13.8 Hz, 1H), 3.74 (s, 3H), 1.95 (s, 3H). MS (ES⁺) 592, m/z (M + 1)⁺ C₂₈H₂₃BrN₄O₄S requires 592 |
| 19 | 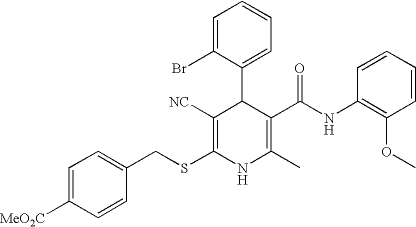 2-(4-carboxymethylbenzylthio-3-cyano-4-(2- bromophenyl)-5-(2-methoxyphenyl)carbamoyl-6- methyl-1,4-dihydro-pyridine | ¹H NMR (DMSO-d6, 400 MHz): 9.30 (broad s, 1H), 8.36 (broad s, 1H),, 7.89 (d, J = 8.0 Hz, 2H), 7.67 (dd, J = 8.0, 1.2 Hz, 1H), 7.54 (dd, J = 8.0, 1.2 Hz, 1H), 7.41 (d, J = 8.0 Hz, 2H), 7.28 (td, J = 7.6, 1.2 Hz, 1H), 7.16 (td, J = 8.0, 2.0 Hz, 1H), 7.12 (dd, J = 7.6, 1.6 Hz, 1H), 7.02 (td, J = 8.0, 1.2 Hz, 1H), 6.94 (dd, J = 8.0, 1.2 Hz, 1H), 6.84 (td, J = 8.0, 1.2 Hz, 1H), 5.06 (s, 1H), 4.34 (s, 2H), 3.87 (s, 3H), 3.69 (s, 3H), 2.16 (s, 3H). MS (ES⁺) 605, m/z (M + 1)⁺ C₃₀H₂₆BrN₃O₄S requires 605. |
| 20 | 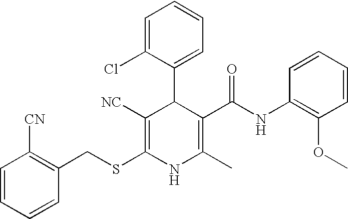 2-(2-cyanobenzylbenzyl)thio-3-cyano-4-(2- chlorophenyl)-5-(2-methoxyphenyl)carbamoyl-6- methyl-1,4-dihydro-pyridine | ¹H NMR (DMSO-d6, 400 MHz): 9.43 (broad s, 1H), 8.38 (broad s, 1H), 7.84 (dd, J = 7.6, 0.8 Hz, 1H), 7.69 (dd, J = 7.6, 1.2 Hz, 1H), 7.61 (td, J = 8.0, 1.2 Hz, 1H), 7.49 (d, J = 8.0 Hz, 1H), 7.45 (d, J = 8.0 Hz, 1H), 7.37 (d, J = 8.0 Hz, 2H), 7.39 (dd, J = 7.6, 0.8 Hz, 1H), 7.35 (td, J = 7.6, 0.8 Hz, 1H), 7.27 (td, J = 7.6, 2.0 Hz, 1H), 7.21 (td, J = 7.6, 2.0 Hz, 1H), 7.00 (td, J = 7.6, 1.2 Hz, 1H), 6.96 (dd, J = 7.6, 1.2 Hz, 1H), 6.82 (td, J = 7.6, 1.2 Hz, 1H), 5.09 (s, 1H), 4.41 (s, 2H), 3.70 (s, 3H), 2.18 (s, 3H). MS (ES⁺) 528, m/z (M + 1)⁺ C₂₉H₂₃ClN₄O₂S requires 528. |
| 21 | 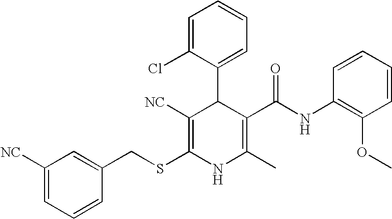 2-(3-cyanobenzylbenzyl)thio-3-cyano-4-(2- chlorophenyl)-5-(2-methoxyphenyl)carbamoyl-6- methyl-1,4-dihydro-pyridine | ¹H NMR (CDCl₃, 400 MHz): 8.15 (dd, J = 8.0, 1.6 Hz, 1H), 7.43 (m, 2H), 7.39 (dd, J = 8.0, 1.6 Hz, 1H), 7.35 (d, J = 8.0 Hz, 1H), 7.19 (m 5H), 7,02 (dd, J = 7.6, 2.0 Hz, 1H), 6.89 (td, J = 8.0, 2.0 Hz, 1H), 6.80 (td, J = 8.0, 2.0 Hz, 1H), 6.67 (dd, J = 8.0, 2.0 Hz, 1H), 5.82 (broad s, 1H), 5.13 (s, 1H), 4.16 (d, J = 14.0 Hz, 1H), 3.90 (d, J = 14.0 Hz, 1H), 3.57 (s, 3H), 2.20 (s, 3H). MS (ES⁺) 528, m/z (M + 1)⁺ C₂₉H₂₃ClN₄O₂S requires 528. |
| 22 | 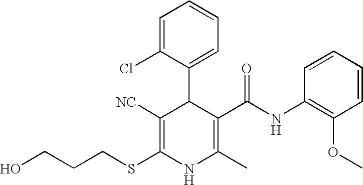 2-(3-hydroxymethyl)thio-3-cyano-4-(2- chlorophenyl)-5-(2-methoxyphenyl)carbamoyl-6- methyl-1,4-dihydro-pyridine | ¹H NMR (CDCl₃, 400 MHz): 9.04 (broad s, 1H), 8.25 (broad s, 1H), 7.51 (dd, J = 8.0, 1.6 Hz, 1H), 7.23 (d, J = 8.0 Hz, 1H), 7.17 (m, 2H), 7.08 (m, 1H), 6.82 (td, J = 8.0, 1.2 Hz, 1H), 6.75 (dd, J = 8.0, 1.2 Hz, 1H), 6.61 (td, J = 8.0, 1.2 Hz, 1H), 4.98 (s, 1H), 4.42 (t, J = 5.2 Hz, 1H), 3.97 (s, 3H), 3.29 (m, 2H), 2.99 (m, 1H), 2.48 (m, 1H), 1.96 (s, 3H), 1,47 (m, 2H). MS (ES⁺) 471 m/z (M + 1)⁺ C₂₄H₂₄ClN₃O₃S requires 471. |
| 23 | 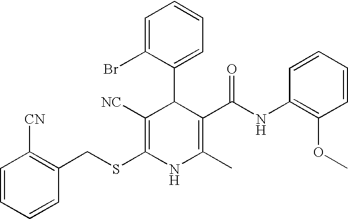 2-(2-cyanobenzylthio-3-cyano-4-(2- bromophenyl)-5-(2-methoxyphenyl)carbamoyl-6- methyl-1,4-dihydro-pyridine | ¹H NMR (DMSO-d6, 400 MHz): 9.30 (broad s, 1H), 8.36 (broad s, 1H),, 7.89 (d, J = 8.0 Hz, 2H), 7.67 (dd, J = 8.0, 1.2 Hz, 1H), 7.54 (dd, J = 8.0, 1.2 Hz, 1H), 7.41 (d, J = 8.0 Hz, 2H), 7.28 (td, J = 7.6, 1.2 Hz, 1H), 7.16 (td, J = 8.0, 2.0 Hz, 1H), 7.12 (dd, J = 7.6, 1.6 Hz, 1H), 7.02 (td, J = 8.0, 1.2 Hz, 1H), 6.94 (dd, J = 8.0, 1.2 Hz, 1H), 6.84 (td, J = 8.0, 1.2 Hz, 1H), 5.06 (s, 1H), 4.34 (s, 2H), 3.87 (s, 3H), 3.69 (s, 3H), 2.16 (s, 3H). MS (ES⁺) 571 m/z (M + 1)⁺ C₂₉H₂₃BrN₄O₂S requires 571. |
| 24 | 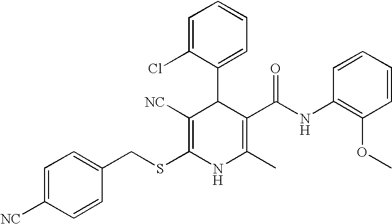 2-(4-cyanobenzylbenzyl)thio-3-cyano-4-(2- bromophenyl)-5-(2-methoxyphenyl)carbamoyl-6- methyl-1,4-dihydro-pyridine | ¹H NMR (CDCl₃, 400 MHz): 8.21 (dd, J = 8.0, 1.6 Hz, 1H), 7.64 (dd, J = 8.0, 0.8 Hz, 1H), 7.52 (m, 2H), 7.49 (d, J = 10.0 Hz, 1H), 7.44 (d, J = 10.0 Hz, 1H), 7.30 (t, J = 8.0 Hz, 2H), 7.19 (td, J = 8.0, 1.6 Hz, 1H), 7.10 (dd, J = 7.6, 2.0 Hz, 1H), 6.97 (td, J = 8.0, 1.2 Hz, 1H), 6.86 (td, J = 8.0, 1.2 Hz, 1H), 6.77 (dd, J = 8.0, 1.2 Hz, 1H), 5.93 (broad s, 1H), 5,21 (s, 1H), 4.24 (d, J = 12.4 Hz, 1H), 3.98 (d, J = 12.4 Hz, 1H), 3.65 (s, 3H), 2.26 (s, 3H). MS (ES⁺) 528 m/z (M + 1)⁺ C₂₉H₂₃ClN₄O₂S requires 528. |
| 25 | 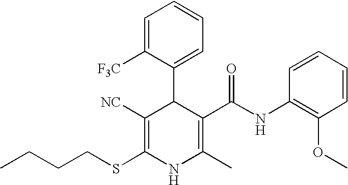 2-butylthio-3-cyano-4-(2-trifluoromethylphenyl)- 5-(2-methoxyphenyl)carbamoyl-6-methyl-1,4- dihydro-pyridine | ¹H NMR (CDCl₃, 400 MHz): 9.24 (broad s, 1H), 8.85 (broad s, 1H), 7.82 t, J = 7.6 Hz, 1H), 7.77 (d, J = 7.6 Hz, 1H), 7.24 (d, J = 8.0 Hz, 1H), 7.59 (d, J = 8.0 Hz, 1H), 7.54 (t, J = 7.6 Hz, 1H), 7.14 (td, J = 7.6, 1.2 Hz, 1H), 7.04 (dd, J = 7.6, 1.2 Hz, 1H), 6.89 (td, J = 7.6, 1.2 Hz, 1H), 5.00 (s, 1H), 3.78 (s, 3H), 3.13 (m, 1H), 3.02 (m, 1H), 2.11 (s, 3H), 1.59 (m, 2H), 1.47 (m, 2H), 0.94 (t, J = 7.3 Hz, 3H). MS (ES⁺) 502 m/z (M + 1)⁺ C₂₆H₂₆F₃N₃O₂S requires 502. |
| 26 | 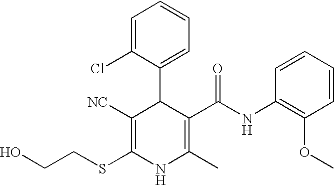 2-(2-hydroxyethyl)thio-3-cyano-4-(2- chlorophenyl)-5-(2-methoxyphenyl)carbamoyl-6- methyl-1,4-dihydro-pyridine | ¹H NMR (CDCl₃, 400 MHz): 9.42 (broad s, 1H), 8.44 (broad s, 1H), 7.74 (dd, J = 8.0, 1.6 Hz, 1H), 7.44 (d, J = 8.0 Hz, 1H), 7.37 (m, 2H), 7.29 (m, 1H), 7.02 (td, J = 8.0, 1.2 Hz, 1H), 6.96 (dd, J = 8.0, 1.2 Hz, 1H), 6.84 (td, J = 8.0, 1.2 Hz, 1H), 5.14 (broad s, 1H), 5.18 (s, 1H), 3.71 (s, 3H), 3.64 (m, 1H), 3.58 (m, 1H), 3.11 (m, 1H), 3.04 (m, 1H), 2.16 (s, 3H). MS (ES⁺) 457 m/z (M + 1)⁺ C₂₃H₂₂ClN₃O₃S requires 457. |
| 27 | 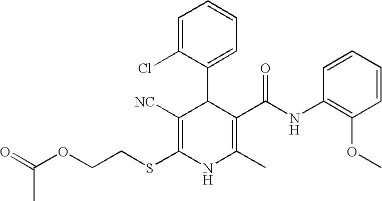 2-(acetoxyethyl)thio-3-cyano-4-(2-chlorophenyl)- 5-(2-methoxyphenyl)carbamoyl-6-methyl-1,4- dihydro-pyridine | ¹H NMR (CDCl₃, 400 MHz): 9.30 (broad s, 1H), 8.44 (broad s, 1H), 7.73 (dd, J = 8.0, 1.2 Hz, 1H), 7.44 (d, J = 8.0 Hz, 1H), 7.40 (m, 2H), 7.28 (m, 1H), 7.03 (td, J = 8.0, 1.6 Hz, 1H), 6.96 (dd, J = 8.0, 1.6 Hz, 1H), 6.86 (td, J = 8.0, 1.6 Hz, 1H), 5.20 (s, 1H), 4.16 (m, 2H), 3.71 (s, 3H), 3.56 (m, 1H), 3.16 (m, 1H), 3.11 (m, 1H), 2.17 (s, 3H), 2.02 (s, 3H). MS (ES⁺) 499 m/z (M + 1)⁺ C₂₅H₂₄ClN₃O₄S requires 499. |
| 28 | 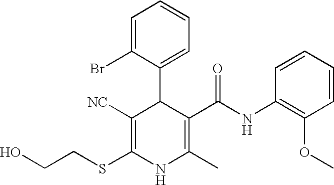 2-(hydroxyethyl)thio-3-cyano-4-(2-bromophenyl)- 5-(2-methoxyphenyl)carbamoyl-6-methyl-1,4- dihydro-pyridine | ¹H NMR (CDCl₃, 400 MHz): 9.41 (broad s, 1H), 8.40 (broad s, 1H), 7.74 (dd, J = 8.0, 1.6 Hz, 1H), 7.60 (d, J = 7.6 Hz, 1H), 7.42 (m, 2H), 7.21 (m, 1H), 7.01 (td, J = 8.0, 1.6 Hz, 1H), 6.95 (dd, J = 8.0, 1.6 Hz, 1H), 6.88 (td, J = 8.0, 1.6 Hz, 1H), 5.53 (t, J = 4.9 Hz, 1H), 5.15 (s, 1H), 3.71 (s, 3H), 3.65 (m, 1H), 3.59 (m, 1H), 3.12 (m, 1H), 3.05 (m, 1H), 1.99 (s, 3H). MS (ES⁺) 501 and 503 m/z (M + 1)⁺ C₂₃H₂₂BrN₃O₃S requires 501. |
| 29 | 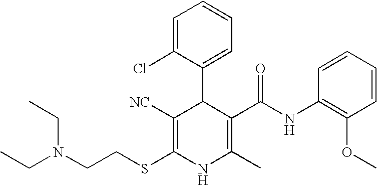 2-(N,N-diethylaminoethyl)thio-3-cyano-4-(2- chlorophenyl)-5-(2-methoxyphenyl)carbamoyl-6- methyl-1,4-dihydro-pyridine | ¹H NMR (CD₂Cl₂, 400 MHz): 11.02 (broad s, 1H), 8.00 (dd, J = 8.0, 1.6 Hz, 1H), 7.35 (broad s, 1H), 7.25 (dd, J = 7.6, 1.2 Hz, 1H), 7.20 (dd, J = 7.6, 1.6 Hz, 1H), 7.12 (td, J = 7.2, 1.2 Hz, 1H), 7.06 (td, J = 7.6, 2.0 Hz, 1H), 6.75 (td, J = 8.0, 1.6 Hz, 1H), 6.67 (td, J = 8.0, 1.6 Hz, 1H), 6.59 (dd, J = 8.0, 1.6 Hz, 1H), 4.98 (s, 1H), 3.48 (s, 3H), 2.73 (m, 2H), 2.55 (m, 2H), 2.11 (s, 3H), 0.94 (t, J = 7.2 Hz, 3H). MS (ES⁺) 512 m/z (M + 1)⁺ C₂₇H₃₁ClN₄O₂S requires 512. |
| 30 | 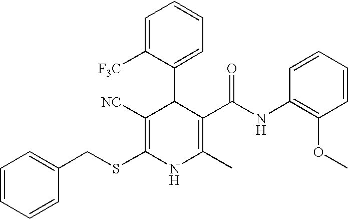 2-benzylthio-3-cyano-4-(2- trifluoromethylphenyl)-5-(2- methoxyphenyl)carbamoyl-6-methyl-1,4-dihydro- pyridine | ¹H NMR (CDCl₃, 400 MHz): 9.17 (broad s, 1H), 8.65 (broad s, 1H), 7.64 (t, J = 7.6 Hz, 1H), 7.58 (d, J = 7.6 Hz, 1H), 7.47 (d, J = 7.6 Hz, 1H), 7.39 (d, J = 7.6 Hz, 1H), 7.30 (m, 5H), 6.99 (td, J = 8.0, 1.6 Hz, 1H), 6.95 (dd, J = 8.0, 1.6 Hz, 1H), 6.79 (td, J = 8.0, 1.6 Hz, 1H), 4.83 (s, 1H), 4.30 (d, J = 15.1 Hz, 1H), 4.24 (d, J = 15.1 Hz, 1H), 3.68 (s, 3H), 2.01 (s, 3H). MS (ES⁺) 536 m/z (M + 1)⁺ C₂₉H₂₄F₃N₃O₂S requires 536. |
| 31 | 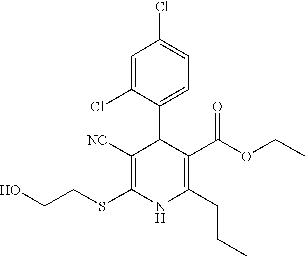 5-ethyl-2-(hydroxyethyl)thio-3-cyano-4-(2,4- dichlorophenyl)-6-propyl-1,4-dihydro-pyridine-5- carboxylate | ¹H NMR (CDCl₃, 400 MHz): 8.75 (broad s, 1H), 7.27 (s, 1H), 7.11 (s, 2H), 5.18 (s, 1H), 3.96 (m, 3H), 3.73 (m, 1H), 2.91 (m, 2H), 2.64 (t, J = 7.6 Hz, 2H), 1.60 (m, 2H), 1.05 (t, J = 7.2 Hz, 3H), 0.94 (t, J = 7.2 Hz, 3H). MS (ES⁺) 442 m/z (M + 1)⁺ C₂₀H₂₂Cl₂N₂O₃S requires 442. |
| 32 | 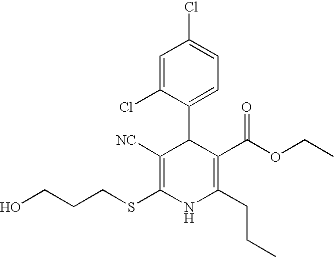 5-ethyl-2-(hydroxypropyl)thio-3-cyano-4-(2,4- dichlorophenyl)-6-propyl-1,4-dihydro-pyridine-5- carboxylate | ¹H NMR (CDCl₃, 400 MHz): 7.36 (broad s, 1H), 7.27 (s, 1H), 7.18 (s, 2H), 5.26 (s, 1H), 3.96 (m, 2H), 3.87 (dt, J = 10.4, 5.2 Hz, 1H), 3.11 (dt, J = 12.0, 6.0 Hz, 1H), 2.99 (dt, J = 12.0, 6.0 Hz, 1H), 2.73 (m, 2H), 21.46 (broad s, 1H), 1.91 (m, 1H), 1.82 (m, 2H), 1.65 (m, 2H), 1.25 (m, 1H), 1.12 (t, J = 7.2 Hz, 3H), 1.02 (t, J = 7.6 Hz, 3H). MS (ES⁺) 456 m/z (M + 1)⁺ C₂₁H₂₄Cl₂N₂O₃S requires 456. |
| 33 | 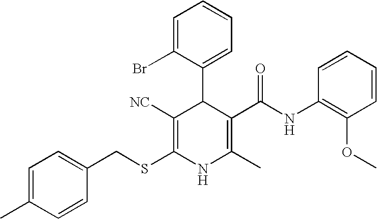 2-(4-methylbenzyl)thio-3-cyano-4-(2- bromophenyl)-5-(2-methoxyphenyl)carbamoyl-6- methyl-1,4-dihydro-pyridine | ¹H NMR (DMSO-d6, 400 MHz): 9.27 (broad s, 1H), 8.36 (broad s, 1H), 7.25 (d, J = 7.6 Hz, 1H), 7.59 (d, J = 8.0 Hz, 1H), 7.39 (t, J = 7.6 Hz, 1H), 7.22 (d, J = 7.6 Hz, 1H), 7.20 (d, J = 8.0 Hz, 1H), 7.15 (d, J = 8.0 Hz, 1H), 7.05 (td, J = 8.0, 1.2 Hz, 1H), 6.98 (dd, J = 8.0, 1.2 Hz, 1H), 6.85 (td, J = 8.0, 1.2 Hz, 1H), 5.11 (s, 1H), 4.29 (d, J = 13.6 Hz, 1H), 4.23 (d, J = 13.6 Hz, 1H), 3.80 (s, 3H), 2.32 (s, 3H), 2.18 (s, 3H). MS (ES⁺) 561 m/z (M + 1)⁺ C₂₉H₂₆BrN₃O₂S requires 561. |
| 34 | 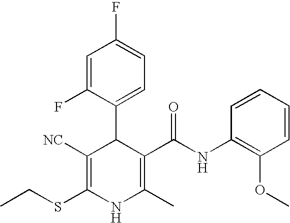 2-ethylthio-3-cyano-4-(2,4-difluorophenyl)-5-(2- methoxyphenyl)carbamoyl-6-methyl-1,4-dihydro- pyridine | ¹H NMR (DMSO-d6, 400 MHz): 9.24 (broad s, 1H), 8.60 (broad s, 1H), 7.68 (dd, J = 8.0, 1.6 Hz, 1H), 7.35 (m, 1H), 7.25 (td, J = 10.4, 2.4 Hz, 1H), 7.11 (td, J = 8.4, 2.0 Hz, 1H), 7.04 (td, J = 8.4, 1.2 Hz, 1H), 6.97 (dd, J = 8.4, 1.2 Hz, 1H), 6.85 (td, J = 8.4, 1.2 Hz, 1H), 4.99 (s, 1H), 3.73 (s, 3H), 3.04 (m, 1H), 2.96 (m, 1H), 2.18 (s, 3H), 1.20 (t, J = 7.6 Hz, 3H). MS (ES⁺) 442 m/z (M + 1)⁺ C₂₃H₂₁F₂N₃O₂S requires 442. |
| 35 | 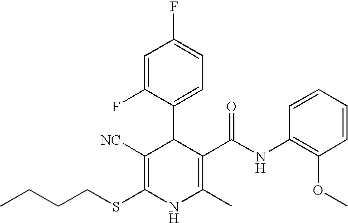 2-butylthio-3-cyano-4-(2,4-difluorophenyl)-5-(2- methoxyphenyl)carbamoyl-6-methyl-1,4-dihydro- pyridine | ¹H NMR (DMSO-d6, 400 MHz): 9.26 (broad s, 1H), 8.59 (broad s, 1H), 7,69 (dd, J = 8.0, 1.2 Hz, 1H), 7.34 (m, 1H), 7.26 (td, J = 10.4, 2.4 Hz, 1H), 7.12 (td, J = 8.4, 2.0 Hz, 1H), 7.05 (td, J = 8.4, 1.2 Hz, 1H), 6.98 (dd, J = 8.4, 1.2 Hz, 1H), 6.84 (td, J = 8.4, 1.2 Hz, 1H), 4.98 (s, 1H), 3.73 (s, 3H), 3.06 (m, 1H), 2.92 (m, 1H), 2.18 (s, 3H), 1.49 (m, 2H), 1.36 (m, 2H), 0.85 (t, J = 7.6 Hz, 3H). MS (ES⁺) 470 m/z (M + 1)⁺ C₂₅H₂₅F₂N₃O₂S requires 470. |
| 36 | 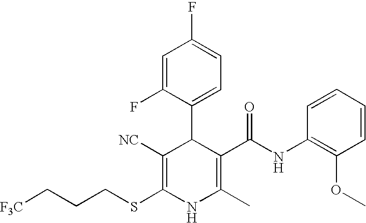 2-(4,4,4-trifluorobutyl)thio-3-cyano-4-(2,4- difluorophenyl)-5-(2-methoxyphenyl)carbamoyl- 6-methyl-1,4-dihydro-pyridine | ¹H NMR (DMSO-d6, 400 MHz): 9.34 (broad s, 1H), 8.60 (broad s, 1H), 7.68 (dd, J = 7.6 1.2 Hz, 1H), 7.35 (m, 1H), 7.25 (td, J = 10.4, 2.4 Hz, 1H), 7.09 (td, J = 8.4, 2.0 Hz, 1H), 7.04 (td, J = 8.0, 1.2 Hz, 1H), 6.97 (dd, J = 8.0, 1.2 Hz, 1H), 6.86 (td, J = 8.0, 1.2 Hz, 1H), 5.01 (s, 1H), 3.32 (s, 3H), 3.13 (m, 1H), 2.99 (m, 1H), 2.39 (m, 2H), 2.17 (s, 3H), 1.72 (m, 2H). MS (ES⁺) 524 m/z (M + 1)⁺ C₂₅H₂₂F₅N₃O₂S requires 524. |
| 37 | 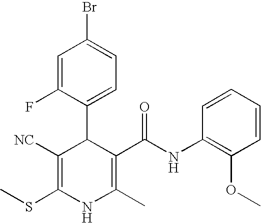 2-methylthio-3-cyano-4-(2-fluoro-4- bromophenyl)-5-(2-methoxyphenyl)carbamoyl-6- methyl-1,4-dihydro-pyridine | ¹H NMR (DMSO-d6, 400 MHz): 9.17 (broad s, 1H), 8.65 (broad s, 1H), 7.66 (dd, J = 8.0 1.2 Hz, 1H), 7.56 (dd, J = 10.0, 2.0 Hz, 1H), 7.46 (dd, J = 8.4, 1.6 Hz, 1H), 7.26 (t, J = 8.0 Hz, 1H), 7.04 (td, J = 8.0, 1.6 Hz, 1H), 6.98 (dd, J = 8.0, 1.6 Hz, 1H), 6.84 (td, J = 8.0, 1.6 Hz, 1H), 4.96 (s, 1H), 3.73 (s, 3H), 3.32 (s, 3H), 2.17 (s, 3H). MS (ES⁺) 489 m/z (M + 1)⁺ C₂₂H₁₉BrFN₃O₂S requires 489. |
| 38 | 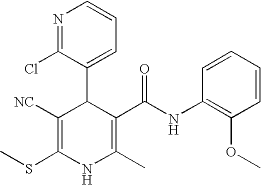 2-methylthio-3-cyano-4-[3-(2-chloropyridine)]-5- (2-methoxyphenyl)carbamoyl-6-methyl-1,4- dihydro-pyridine | ¹H NMR (DMSO-d6, 400 MHz): 9.19 (broad s, 1H), 8.72 (broad s, 1H), 8.32 (dd, J = 4.8, 2.0 Hz, 1H), 7.81 (dd, J = 7.6, 2.0 Hz, 1H), 7.63 (dd, J = 8.0, 1.2 Hz, 1H), 7.49 (dd, J = 7.6, 4.4 Hz, 1H), 7.04 (td, J = 8.0, 1.6 Hz, 1H), 6.90 (dd, J = 8.0, 1.6 Hz, 1H), 6.84 (td, J = 8.0, 1.6 Hz, 1H), 5.14 (s, 1H), 3.73 (s, 3H), 3.32 (s, 3H), 2.16 (s, 3H). MS (ES⁺) 427, m/z (M + 1)⁺ C₂₁H₁₉ClN₄O₂S requires 427. |
| 39 | 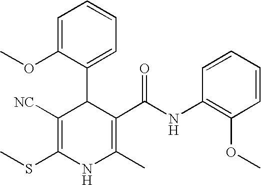 2-methylthio-3-cyano-4-(2-methoxyphenyl)-5-(2- methoxyphenyl)carbamoyl-6-methyl-1,4-dihydro- pyridine | ¹H NMR (DMSO-d6, 400 MHz): 9.10 (broad s, 1H), 8.15 (broad s, 1H), 7.90 (dd, J = 8.0, 1.2 Hz, 1H), 7.27 (td, J = 8.0, 1.6 Hz, 1H), 7.16 (dd, J = 7.6, 1.6 Hz, 1H), 7.05 (d, J = 8.4 Hz, 1H), 6.97 (m, 2H), 6.85 (dd, J = 8.0, 1.2 Hz, 1H), 4.94 (s, 1H), 3.77 (s, 3H), 3.66 (s, 3H), 3.31 (s, 3H), 2.22 (s, 3H). MS (ES⁺) 422, m/z (M + 1)⁺ C₂₃H₂₃N₃O₃S requires 422. |
| 40 | 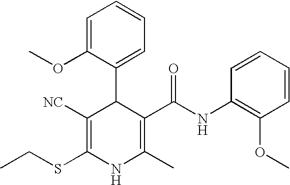 2-ethylthio-3-cyano-4-(2-methoxyphenyl)-5-(2- methoxyphenyl)carbamoyl-6-methyl-1,4-dihydro- pyridine | ¹H NMR (DMSO-d6, 400 MHz): 9.18 (broad s, 1H), 8.13 (broad s, 1H), 7.90 (dd, J = 8.0, 1.2 Hz, 1H), 7.26 (td, J = 8.0, 1.6 Hz, 1H), 7.18 (dd, J = 8.0, 1.6 Hz, 1H), 7.06 (d, J = 7.6 Hz, 1H), 6.93 (m, 2H), 6.86 (dd, J = 8.0, 1.6 Hz, 1H), 4.97 (s, 1H), 3.77 (s, 3H), 3.66 (s, 3H), 3.04 (m, 1H), 2.89 (m, 1H), 2.27 (s, 3H), 1.17 (t, J = 7.2 Hz, 3H), MS (ES⁺) 436, m/z (M + 1)⁺ C₂₃H₂₃N₃O₃S requires 436. |
| 41 | 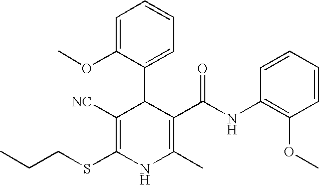 2-butylthio-3-cyano-4-(2-methoxyphenyl)-5-(2- methoxyphenyl)carbamoyl-6-methyl-1,4-dihydro- pyridine | ¹H NMR (DMSO-d6, 400 MHz): 9.19 (broad s, 1H), 8.13 (broad s, 1H), 7.89 (dd, J = 8.0, 1.2 Hz, 1H), 7.27 (td, J = 8.0, 1.2 Hz, 1H), 7.17 (dd, J = 7.6, 1.2 Hz, 1H), 7.06 (d, J = 8.0 Hz, 1H), 6.98 (m, 2H), 6.84 (dd, J = 8.0, 1.6 Hz, 1H), 4.96 (s, 1H), 3.77 (s, 3H), 3.66 (s, 3H), 3.05 (m, 1H), 2.83 (m, 1H), 2.22 (s, 3H), 1.50 (m, 2H), 0.94 (t, J = 7.2 Hz, 3H), MS (ES⁺) 450, m/z (M + 1)⁺ C₂₃H₂₃N₃O₃S requires 450. |
| 42 | 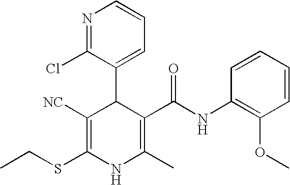 2-ethylthio-3-cyano-4-[3-(2-chloropyridine)]-5-(2- methoxyphenyl)carbamoyl-6-methyl-1,4-dihydro- pyridine | ¹H NMR (DMSO-d6, 400 MHz): 9.27 (broad s, 1H), 8.71 (broad s, 1H), 8.31 (dd, J = 4.7, 1.8 Hz, 1H), 7.80 (dd, J = 7.6, 2.0 Hz, 1H), 7.65 (dd, J = 8.0, 1.2 Hz, 1H), 7.42 (dd, J = 7.6, 4.4 Hz, 1H), 7.03 (td, J = 8.0, 1.6 Hz, 1H), 6.92 (dd, J = 8.0, 1.6 Hz, 1H), 6.82 (td, J = 8.0, 1.6 Hz, 1H), 5.18 (s, 1H), 3.73 (s, 3H), 3.07 (m, 1H), 2.96 (m, 1H), 2.16 (s, 3H), 1.21 (t, J = 7.2 Hz, 3H). MS (ES⁺) 441, m/z (M + 1)⁺ C₂₁H₁₉ClN₄O₂S requires 442. |
| 43 | 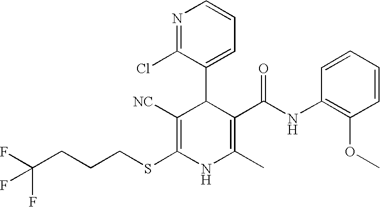 2-(4,4,4-trifluorobutyl)thio-3-cyano-4-[3-(2- chloropyridine)]-5-(2-methoxyphenyl)carbamoyl- 6-methyl-1,4-dihydro-pyridine | ¹H NMR (DMSO-d6, 400 MHz): 9.36 (broad s, 1H), 8.67 (broad s, 1H), 8.33 (dd, J = 4.8, 2.0 Hz, 1H), 7.82 (dd, J = 7.6, 1.6 Hz, 1H), 7.62 (dd, J = 8.0, 1.2 Hz, 1H), 7.47 (dd, J = 7.6, 4.8 Hz, 1H), 7.05 (td, J = 7.6, 1.6 Hz, 1H), 6.91 (dd, J = 7.6, 1.6 Hz, 1H), 6.82 (td, J = 7.6, 1.6 Hz, 1H), 5.20 (s, 1H), 3.73 (s, 3H), 3.13 (m, 1H), 3.01 (m, 1H), 2.39 (m, 2H), 2.16 (s, 3H), 1.75 (m, 2H). MS (ES⁺) 522, m/z (M + 1)⁺ C₂₁H₁₉ClN₄O₂S requires 523. |
| 44 | 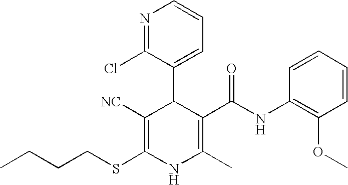 2-butylthio-3-cyano-4-[3-(2-chloropyridine)]-5-(2- methoxyphenyl)carbamoyl-6-methyl-1,4-dihydro- pyridine | ¹H NMR (DMSO-d6, 400 MHz): 9.48 (broad s, 1H), 8.88 (broad s, 1H), 8.52 (dd, J = 4.8, 2.0 Hz, 1H), 8.00 (dd, J = 7.6, 1.6 Hz, 1H), 7.82 (dd, J = 8.0, 1.2 Hz, 1H), 7.67 (dd, J = 7.6, 4.4 Hz, 1H), 7.23 (td, J = 7.6, 1.6 Hz, 1H), 7.16 (dd, J = 7.6, 1.6 Hz, 1H), 7.03 (td, J = 7.6, 1.6 Hz, 1H), 5.34 (s, 1H), 3.51 (s, 3H), 3.26 (m, 1H), 3.11 (m, 1H), 2.36 (m, 3H), 1.67 (m, 2H), 1.55 (m, 2H), 1.04 (t, J = 7.6 Hz, 3H). MS (ES⁺) 469, m/z (M + 1)⁺ C₂₁H₁₉ClN₄O₂S requires 469. |
| 45 | 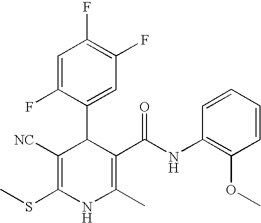 2-methylthio-3-cyano-4-(2,4,5trifluorophenyl)-5- (2-methoxyphenyl)carbamoyl-6-methyl-1,4- dihydro-pyridine | ¹H NMR (DMSO-d6, 400 MHz): 8.97 (broad s, 1H), 8.59 (broad s, 1H), 7.45 (dd, J = 8.0, 1.2 Hz, 1H), 7.37 (m, 1H), 7.15 (m, 1H), 6.87 (td, J = 7.6, 1.6 Hz, 1H), 6.79 (dd, J = 7.6, 1.6 Hz, 1H), 7.66 (td, J = 7.6, 1.6 Hz, 1H), 5.56 (s, 1H), 3.55 (s, 3H), 2.36 (s, 3H), 1.98 (s, 3H). MS (ES⁺) 445, m/z (M + 1)⁺ C₂₂H₁₈F₃N₃O₂S requires 446. |
| 46 | 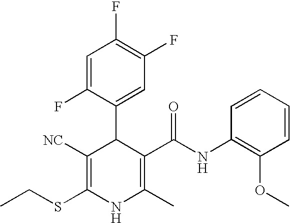 2-ethylthio-3-cyano-4-(2,4,5trifluorophenyl)-5-(2- methoxyphenyl)carbamoyl-6-methyl-1,4-dihydro- pyridine | ¹H NMR (DMSO-d6, 400 MHz): 9.25 (broad s, 1H), 8.78 (broad s, 1H), 7.64 (dd, J = 8.0, 1.2 Hz, 1H), 7.57 (m, 1H), 7.38 (m, 1H), 7.07 (td, J = 7.6, 1.6 Hz, 1H), 6.98 (dd, J = 7.6, 1.6 Hz, 1H), 6.83 (td, J = 7.6, 1.6 Hz, 1H), 5.01 (s, 1H), 3.32 (s, 3H), 3.04 (m, 1H), 2.96 (m, 1H), 2.18 (s, 3H), 1.21 (t, J = 7.2 Hz, 3H). MS (ES⁺) 459, m/z (M + 1)⁺ C₂₃H₂₀F₃N₃O₂S requires 459. |
| 47 | 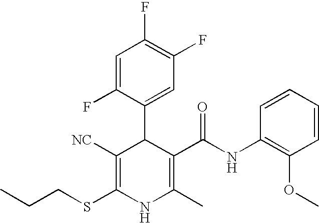 2-propylthio-3-cyano-4-(2,4,5trifluorophenyl)-5- (2-methoxyphenyl)carbamoyl-6-methyl-1,4- dihydro-pyridine | ¹H NMR (DMSO-d6, 400 MHz): 9.37 (broad s, 1H), 8.87 (broad s, 1H), 7.75 (dd, J = 8.0, 1.6 Hz, 1H), 7.67 (m, 1H), 7.46 (m, 1H), 7.17 (td, J = 7.6, 1.6 Hz, 1H), 7.10 (dd, J = 7.6, 1.6 Hz, 1H), 6.97 (td, J = 7.6, 1.6 Hz, 1H), 5.11 (s, 1H), 3.86 (s, 3H), 3.17 (m, 1H), 3.00 (m, 1H), 2.29 (s, 3H), 1.67 (m, 2H), 1.07 (t, J = 7.2 Hz, 3H). MS (ES⁺) 473, m/z (M + 1)⁺ C₂₄H₂₂F₃N₃O₂S requires 473. |
| 48 | 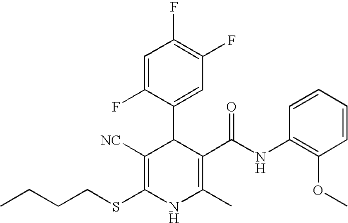 2-butylthio-3-cyano-4-(2,4,5trifluorophenyl)-5-(2- methoxyphenyl)carbamoyl-6-methyl-1,4-dihydro- pyridine | ¹H NMR (DMSO-d6, 400 MHz): 9.47 (broad s, 1H), 8.96 (broad s, 1H), 7.84 (dd, J = 8.0, 1.6 Hz, 1H), 7.77 (m, 1H), 7.56 (m, 1H), 7.26 (td, J = 7.6, 1.2 Hz, 1H), 7.18 (dd, J = 7.6, 1.2 Hz, 1H), 7.05 (td, J = 7.6, 1.2 Hz, 1H), 5.19 (s, 1H), 3.94 (s, 3H), 3.26 (m, 1H), 3.12 (m, 1H), 2.38 (s, 3H), 1.67 (m, 2H), 1.57 (m, 2H), 1.05 (t, J = 7.2 Hz, 3H). MS (ES⁺) 487, m/z (M + 1)⁺ C₂₅H₂₄F₃N₃O₂S requires 487. |
| 49 | 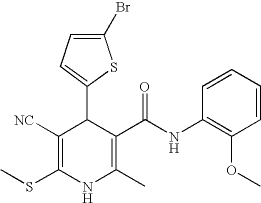 2-methylthio-3-cyano-4-[2-(5-bromothiophene)]- carbamoyl-6-methyl-1,4-dihydro-pyridine | ¹H NMR (DMSO-d6, 400 MHz): 9.62 (broad s, 1H), 8.89 (broad s, 1H), 7.97 (dd, J = 8.0, 2.4 Hz, 1H), 7.80 (d, J = 1.2 Hz, 1H), 7.28 (td, J = 7.6, 1.2 Hz, 1H), 7.22 (dd, J = 7.6, 1.2 Hz, 1H), 7.17 (d, J = 0.8 Hz, 1H), 7.09 (td, J = 7.6, 1.2 Hz, 1H), 5.21 (s, 1H), 3.99 (s, 3H), 2.74 (m, 1H), 2.41 (s, 3H). MS (ES⁺) 476, m/z (M + 1)⁺ C₂₀H₁₃BrN₃O₂S₂ requires 476 |
| 50 | 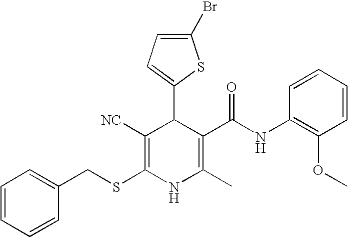 2-benzylthio-3-cyano-4-[2-(5-bromothiophene)]- carbamoyl-6-methyl-1,4-dihydro-pyridine | ¹H NMR (DMSO-d6, 400 MHz): 9.68 (broad s, 1H), 8.73 (broad s, 1H), 7.89 (dd, J = 8.0, 1.6 Hz, 1H), 7.72 (d, J = 1.2 Hz, 1H), 7.45 (m, 5H), 7.21 (dd, J = 7.6, 1.2 Hz, 1H), 7.16 (d, J = 0.8 Hz, 1H), 7.05 (td, J = 7.6, 1.2 Hz, 1H), 7.00 (d, J = 1.2 Hz, 1H), 5.07 (s, 1H), 4.49 (d, J = 13.2 Hz, 1H), 4.39 (d, J = 13.2 Hz, 1H), 3.90 (s, 3H), 2.35 (s, 3H). MS (ES⁺) 552, m/z (M + 1)⁺ C₂₆H₂₂BrN₃O₂S₂ requires 552 |
| 51 | 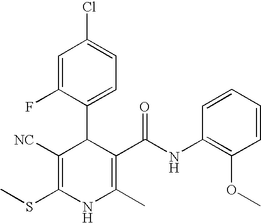 2-methylthio-3-cyano-4-(2-fluoro-4- chlorophenyl)-carbamoyl-6-methyl-1,4-dihydro- pyridine | ¹H NMR (DMSO-d6, 400 MHz): 9.18 (broad s, 1H), 8.67 (broad s, 1H), 7.66 (dd, J = 7.6, 1.6 Hz, 1H), 7.45 (d, J = 8.0 Hz, 1H), 7.32 (m, 2H), 7.05 (dt, J = 7.6, 1.6 H, 1H), 6.98 (dd, J = 7.6, 1.6 Hz, 1H), 6.85 (td, J = 7.6, 1.6 Hz, 4.97 (s, 1H), 3.73 (s, 3H), 2.51 (s, 3H), 2.17 (s, 3H). MS (ES⁺) 443, m/z (M + 1)⁺ C₂₂H₁₉ClFN₃O₂S requires 443 |
| 52 | 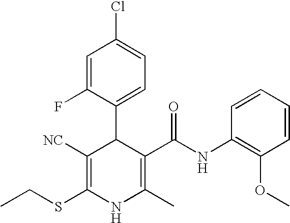 2-ethylthio-3-cyano-4-(2-fluoro-4-chlorophenyl)- carbamoyl-6-methyl-1,4-dihydro-pyridine | ¹H NMR (DMSO-d6, 400 MHz): 9.23 (broad s, 1H), 8.62 (broad s, 1H), 7.67 (dd, J = 7.6, 1.6 Hz, 1H), 7.46 (d, J = 8.0 Hz, 1H), 7.33 (m, 2H), 7.03 (dt, J = 7.6, 1.2 H, 1H), 6.97 (dd, J = 7.6, 1.2 Hz, 1H), 6.84 (td, J = 7.6, 1.2 Hz, 5.00 (s, 1H), 3.73 (s, 3H), 3.06 (m, 1H), 2.95 (m, 1H), 2.17 (s, 3H), 1.20 (t, J = 7.2 Hz, 3H). MS (ES⁺) 458, m/z (M + 1)⁺ C₂₃H₂₁ClFN₃O₂S requires 458 |
| 53 | 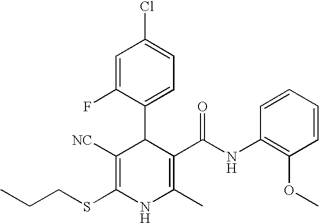 2-propylthio-3-cyano-4-(2-fluoro-4- chlorophenyl)-carbamoyl-6-methyl-1,4-dihydro- pyridine | ¹H NMR (DMSO-d6, 400 MHz): 9.45 (broad s, 1H), 8.84 (broad s, 1H), 7.84 (dd, J = 7.6, 1.6 Hz, 1H), 7.63 (d, J = 8.0 Hz, 1H), 7.51 (m, 2H), 7.23 (dt, J = 7.6, 1.6 H, 1H), 7.15 (dd, J = 7.6, 1.6 Hz, 1H), 7.02 (td, J = 7.6, 1.6 Hz, 5.17 (s, 1H), 3.91 (s, 3H), 3.25 (m, 1H), 3.05 (m, 1H), 2.35 (s, 3H), 1.73 (m, 2H), 1.14 (t, J = 7.6 Hz, 3H). MS (ES⁺) 472, m/z (M + 1)⁺ C₂₄H₂₃ClFN₃O₂S requires 472 |
| 54 | 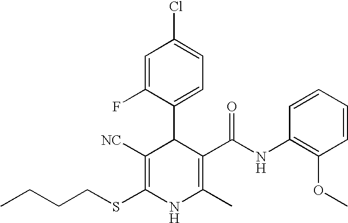 2-butylthio-3-cyano-4-(2-fluoro-4-chlorophenyl)- carbamoyl-6-methyl-1,4-dihydro-pyridine | ¹H NMR (DMSO-d6, 400 MHz): 9.44 (broad s, 1H), 8.80 (broad s, 1H), 7.83 (dd, J = 8.0, 1.6 Hz, 1H), 7.61 (d, J = 8.0 Hz, 1H), 7.48 (m, 2H), 7.20 (dt, J = 7.6, 1.2 H, 1H), 7.13 (dd, J = 7.6, 1.2 Hz, 1H), 7.01 (td, J = 7.6, 1.2 Hz, 5.15 (s, 1H), 3.88 (s, 3H), 3.23 (m, 1H), 3.08 (m, 1H), 2.33 (s, 3H), 1.64 (m, 2H), 1.51 (m, 2H), 1,00 (t, J = 7.2 Hz, 3H). MS (ES⁺) 486, m/z (M + 1)⁺ C₂₅H₂₅ClFN₃O₂S requires 486 |
| 55 | 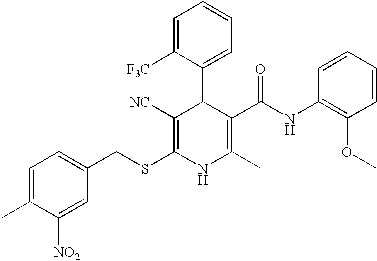 2-(3-nitro-4-methylbenzyl)thio-3-cyano-4-(2- trifluoromethylphenyl)-carbamoyl-6-methyl-1,4- dihydro-pyridine | ¹H NMR (CD₂Cl₂, 400 MHz): 8.01 (dd, J = 8.0, 1.6 Hz, 1H), 7.83 (dd, J = 1.6 Hz, 1H), 7.56 (d, J = 8.0 Hz, 1H), 7.42 (t, J = 7.6 Hz, 1H), 7.37 (dd, J = 8.0, 2.0 Hz, 1H), 7.25 (d, J = 8.0 Hz, 1H), 7.29 (broad s, 1H), 7.16 (d, J = 8.0 Hz, 1H), 6.90 (td, J = 8.0, 1.6 Hz, 1H), 6.81 (dd, J =8.0, 1.6 Hz, 1H), 6.72 (td, J = 8.0, 1.6 Hz, 1H), 5.86 (broad s, 1H), 4.85 (s, 1H), 4.17 (d, J = 13.6 Hz, 1H), 4.00 (d, J = 13.6 Hz, 1H), 3.58 (s, 3H), 2.51 (s, 3H) 1.98 (s, 3H). MS (ES⁺) 594, m/z (M + 1)⁺ C₃₀H₂₅F₃N₄O₄S requires 594 |

TABLE 2

| | | Physical Data |
|---|---|---|
| Compound | | ¹H NMR 400 MHz (DMSO-d6) and/or MS |
| Number | Structure | (mz/) |
| 56 | 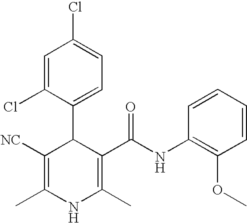 2,6-dimethyl-3-cyano-4-(2,4- dichlorophenyl)-5-(2- methoxyphenyl)carbamoyl-1,4- dihydro-pyridine | ¹H NMR (DMSO-d6, 400 MHz): 9.12 (broad s, 1H), 8.58 (broad s, 1H), 7.83 (dd, J = 8.0, 1.2 Hz, 1H), 7.74 (s, 1H), 7.61 (m, 2H), 7.17 (td, J = 8.0, 1.6 Hz, 1H), 7.11 (dd, J = 8.0, 1.6 Hz, 1H), 6.97 (td, J = 8.0, 1.6 Hz, 1H), 5.17 (s, 1H), 3.70 (s, 3H), 2.15 (s, 3H), 2.00 (s, 3H). MS (ES⁺) 429, m/z (M + 1) 428 C₂₂H₁₉Cl₂N₃O₂ requires 429. |
| 57 | 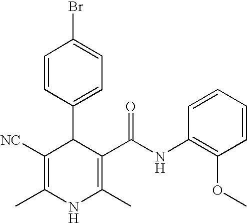 2,6-dimethyl-3-cyano-4-(4- bromophenyl)-5-(2- methoxyphenyl)carbamoyl-1,4- dihydro-pyridine | ¹H NMR (Acetone-d6, 400 MHz): 8.27 (dd, J = 8.0, 1.6 Hz, 1H), 8.13 (broad s, 1H), 7.88 (broad s, 1H), 7.60 (d, J = 8.4 Hz, 2H), 7.39 (d, J = 8.4 Hz, 2H), 6.97 (td, J = 8.0, 1.2 Hz, 1H), 6.92 (dd, J = 8.0, 1.2 Hz,1H), 6.83 (td, J = 8.0, 1.2 Hz, 1H), 4.63 (s, 1H), 3.77 (s, 3H), 2.08 (s, 3H), 2.06 (s, 3H). MS (ES⁺) 439, m/z (M + 1)⁺ C₂₂H₂₀BrN₃₋O₂ requires 439 |
| 58 | 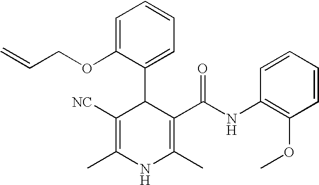 2,6-dimethyl-3-cyano-4-(2- allyloxyphenyl)-5-(2- methoxyphenyl)carbamotl-1,4- dihydro-pyridine | ¹H NMR (DMSO-d6, 400 MHz): 8.67 (broad s, 1H), 7.78 (broad s, 1H), 7.68 (dd, J = 7.6, 1.2 Hz, 1H), 7.00 (m, 2H), 7.80 (d, J = 8.0 Hz, 1H), 6.73 (td, J = 8.0, 1.2 Hz, 1H), 6.69 (dd, J = 8.0, 1.2 Hz, 1H), 6.58 (td, J = 8.0, 1.2 Hz, 1H), 5.80 (m, 1H), 5.16 (dd, J = 17.6, 2.4 Hz, 1H), 4.97 (dd, J = 17.6, 2.4 HZ, 1H), 4.76 (s, 1H), 4.35 (m, 2H), 3.41 (s, 3H), 1.96 (s, 3H), 1.75 (s, 3H). MS (ES⁺) 416, m/z (M + 1)⁺ C₂₅H₂₅N₃O₃ requires 416 |
| 59 | 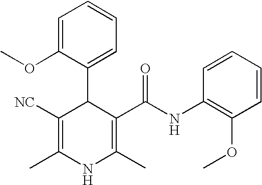 2,6-dimethyl-3-cyano-4-(2- methoxyphenyl)-5-(2- methoxyphenyl)carbamoyl-1,4- dihydro-pyridine | ¹H NMR (DMSO-d6, 400 MHz): 8.99 (broad s, 1H), 8.11 (broad s, 1H), 8.01 (dd, J = 8.0, 1.2 Hz, 1H), 7.32 (td, J = 8.0, 1.6 Hz, 1H), 7.29 (dd, J = 7.6, 1.6 Hz, 1H), 7.13 (d, J = 8.0 Hz, 1H), 7.045 (td, J = 8.0, 1.2 Hz, 1H), 7.02 (dd, J = 8.0, 1.2 Hz, 1H), 6.90 (td, J = 8.0, 1.2 Hz, 1H), 5.04 (s, 1H), 3.84 (s, 3H), 3.73 (s, 3H), 2.28 (s, 3H), 2.06 (s, 3H). MS (ES⁺) 390, m/z (M + 1)⁺ C₂₃H₂₃N₃O₃ requires 390 |
| 60 | 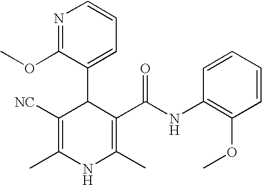 2,6-dimethyl-3-cyano-4-[3-(2- methoxypyridine)]-5-(2- methoxyphenyl)carbamoyl-1,4- dihydro-pyridine | ¹H NMR (DMSO-d6, 400 MHz): 8.29 (dd, J = 8.0, 1.6 Hz, 1H), 8.21 (dd, J = 5.2, 2.0 Hz, 1H), 7.65 (broad s, 1H), 7.61 (dd, J = 7.2, 2.0 Hz, 1H), 7.35 (broad s, 1H), 7.04 (td, J = 7.6, 2.0 Hz, 1H), 7.00 (m, 2H), 6.85 (td, J = 7.6, 2.0 Hz, 1H), 6.09 (m, 1H), 5.06 (s, 1H), 4.08 (s, 3H), 3.74 (s, 3H), 2.45 (s, 3H), 2.16 (s, 3H). MS (ES⁺) 391, m/z (M + H)⁺ C₂₂H₂₂N₄O₃ requires 391 |
| 61 | 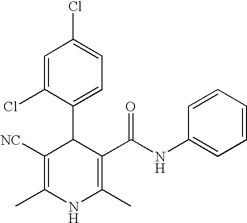 2,6-dimethyl-3-cyano-4-(2,4- dichlorophenyl)-5-phenylcarbamoyl- 1,4-dihydro-pyridine | ¹H NMR (DMSO-d6, 400 MHz): 7.59 (d, J = 2.0 Hz, 1H), 7.42 (broad s, 1H), 7.37 (m, 2H), 7.35 (dd, J = 8.8, 2.0 Hz, 1H), 7.30 (m, 2H), 7.09 (m, 2H), 5.92 (broad s, 1H), 5.24 (s, 1H), 2.30 (s, 3H), 2.12 (s, 3H). MS (ES⁺) 399, m/z (M + 1)⁺C₂₁H₁₇Cl₂N₃O requires 399 |
| 62 | 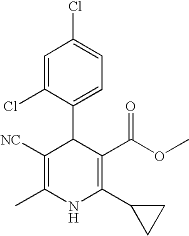 5-methyl-2-methyl-3-cyano-4-(2,4- dichlorophenyl)-6-cyclopropyl-1,4- dihydro-pyridine-5-carboxylate | ¹H NMR (DMSO-d6, 400 MHz): 8.23 (broad s, 1H), 7.64 (d, J = 2.0 Hz, 1H), 7.52 (dd, J = 8.0, 2.0 Hz, 1H), 7.33 (d, J = 8.0 Hz, 1H), 5.30 (s, 1H), 3.55 (s, 3H), 2.96 (m, 1H), 2.09 (s, 3H), 109 (m, 1H), 0.97 (m, 3H). MS (ES⁺) 364, m/z (M + 1)⁺ C₁₈H₁₆Cl₂N₂O₂ requires 364 |
| 63 | 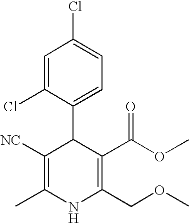 5-methyl-2-methyl-3-cyano-4-(2,4- dichlorophenyl)-6-(methoxymethyl)- 1,4-dihydro-pyridin-5-carboxylate | ¹H NMR (DMSO-d6, 400 MHz): 9.12 (broad s, 1H), 7.56 (d, J = 2.0 Hz, 1H), 7.44 (dd, J = 8.4, 2.0 Hz, 1H), 7.30 (d, J = 8.4 Hz, 1H), 5.08 (s, 1H), 4.56 (s, 2H), 3.46 (s, 3H), 3.34 (s, 3H), 2.03 (s, 3H). MS (ES⁺) 368, m/z (M + 1)⁺ C₁₇H₁₆Cl₂N₂O₃ requires 368 |
| 64 | 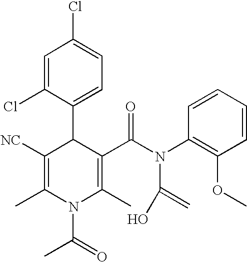 2,6-dimethyl-3-cyano-4-(2,4- dichlorophenyl)-5-N-(2- methoxyphenyl)-N-(1- hydroxyvynyl)carbamoyl-1,4-dihydro- pyridine | ¹H NMR (CDCl₃, 400 MHz): 8.23 (d, J = 8.0 Hz, 1H), 7.47 (broad s, 1H), 7.41 (s, 1H), 7.27 (m, 2H), 7.01 (td, J = 8.0, 1.2 Hz, 1H), 6.90 (td, J = 8.0, 1.2 Hz, 1H), 6.79 (dd, J = 8.0, 1.2 Hz, 1H), 5.51 (d, J = 2.0 Hz, 1H), 5.11 (d, J = 2.0 Hz, 2.0 Hz, 1H), 5.08 (broad s, 1H), 3.71 (s, 3H), 2.23 (s,9H). MS (ES⁺) 513, m/z (M + 1)⁺ C₂₆H₂₃Cl₂N₃O₄ requires 513 |
| 65 | 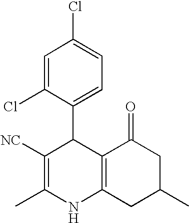 2-methyl-3-cyano-4-(2,4- dichlorophenyl)-5,6-cyclo-3-methyl- hexyl-1,4-dihydro-pyridine | ¹H NMR (MeOD, 400 MHz): 7.54 (broad s, 1H), 7.43 (s, 2H), 5.25 (s, 1H), 2.70 (dd, J = 15.6, 3.6 Hz, 1H), 2.51 (m, 3H), 2.25 (s, 3H), 2.18 (dd, J = 16.0, 96 Hz, 1H), 1.25 (t, J = 6.0 Hz, 3H). MS (ES⁺) 348, m/z (M + 1)⁺ C₁₈H₁₆Cl₂N₂O requires 348 |
| 66 | 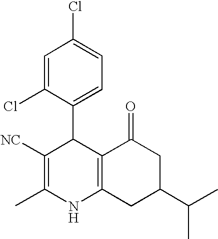 2-methyl-3-cyano-4-(2,4- dichlorophenyl)-5,6-cyclo-3-isopropyl- hexyl-1,4-dihydro-pyridine | ¹H NMR (MeOD, 400 MHz): 9.55 (broad s, 1H), 7.51 (d, J = 2.0 Hz, 1H), 7.36 (dd, J = 6.0, 2.4 Hz, 1H), 7.22 (d, J = 6.0 Hz, 1H), 4.93 (s, 1H), 2.45 (d, J = 7.2 Hz, 1H), 2.21 (dd, J = 16.0, 3.6 Hz, 1H), 2.02 (s, 3H), 2.00 (m, 1H), 1.84 (m, 1H), 1.52 (m, 1H), 0.89 (d, J = 6.8 Hz, 3H), 0.85 (d, J = 6.8 Hz, 3H). MS (ES⁺) 376 and 378, m/z (M + 1)⁺ C₁₈H₁₆Cl₂N₂O requires 376 |
| 67 | 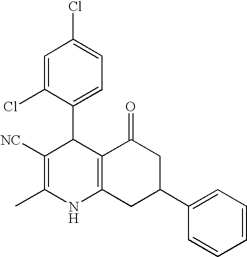 2-methyl-3-cyano-4-(2,4- dichlorophenyl)-5,6-cyclo-3-phenyl- hexyl-1,4-dihydro-pyridine | ¹H NMR (MeOD, 400 MHz): 9.61 (broad s, 1H), 7.51 (d, J = 2.4 Hz, 1H), 7.29 (m, 6H), 7.13 (d, J = 8.4 Hz, 1H), 4.99 (s, 1H), 4.11 (dd, J = 10.4, 5.2 Hz, 1H), 3.44 (m, 1H), 2.90 (dd, J = 16.4, 9.6 Hz, 1H), 2.70 (dd, J = 16.4, 4.4 Hz, 1H), 2.45 (dd, J = 16.4, 4.4 Hz, 1H), 2.03 (s, 3H). MS (ES⁺) 410 and 411, m/z (M + 1)⁺ C₁₈H₁₆Cl₂N₂O requires 410 |
| 68 | 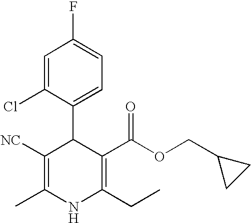 5-cyclopropylmethyl-2-methyl-3- cyano-4-(2-chloro-4-fluorophenyl)-6- ethyl-1,4-dihydro-pyridine-5- carboxylate | ¹H NMR CDCl₃, 400 MHz): 7.13 (dd, J = 8.8, 6.4 Hz, 1H), 6.96 (dd, J = 8.4, 2.4 Hz, 1H), 6.82 (td, J = 8.0, 2.4 Hz, 1H), 5.72 (broad s, 1H), 5.12 (s, 1H), 3.64 (m, 2H), 2.70 (m, 1H), 2.58 (m, 1H), 1.95 (s, 3H), 1.13 (t, J = 7.2 Hz, 3H), 0.83 (m, 1H), 0.32 (m, 2H), 0.01 (m, 1H), −0.07 (m, 1H). MS (ES⁺) 374, m/z (M + 1) 375 C₂₀H₁₉ClFN₂O₂requires 375. |
| 69 | 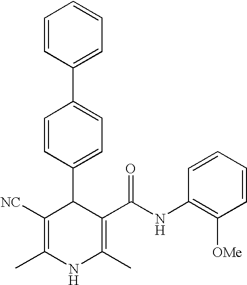 2,6-dimethyl-3-cyano-4-(4- phenylphenyl)-5-(2- methoxyphenyl)carbamoyl-1,4- dihydro-pyridine | ¹H NMR (CDCl₃, 400 MHz): 8.27 (dd, J = 8.0, 1.6 Hz, 1H), 7.63 (m, 5H), 7.46 (m, 4H), 7.36 (m, 1H), 6.94 (m, 1H), 6.89 (m, 1H), 6.71 (dd, J = 8.0, 1.6 Hz, 1H), 5.85 (s, 1H), 4.53 (s, 1H), 3.52 (s, 3H), 2.40 (s, 3H), 2.09 (s, 3H). MS (ES⁺) 435, m/z (M + 1) 436, C₂₈H₂₅N₃O₂ requires 436 |
| 70 | 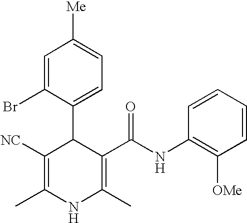 2,6-dimethyl-3-cyano-4-(2-bromo-4- methylphenyl)-5-(2- methoxyphenyl)carbamoyl-1,4- dihydro-pyridine | ¹H NMR (CDCl₃, 400 MHz): 8.02 (dd, J = 8.0, 1.2 Hz, 1H), 7.39 (s, 1H), 7.22 (s, 1H), 7.07 (m, 2H), 6.95 (m, 1H), 6.77 (m, 1H), 6.68 (m, 1H), 6.57 (d, J = 8.0 Hz, 1H), 5.52 (s, 1H), 4.96 (s, 1H), 3.49 (s, 3H), 2.12 (s, 6H), 1.87 (s, 3H). MS (ES⁺) 451, m/z (M + 1) 453, C₂₃H₂₂BrN₃O₂ requires 453 |
| 71 | 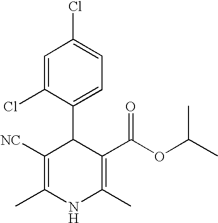 5-isopropyl-2,6-dimethyl-3-cyano-4- (2,4-dichlorophenyl)-1,4-dihydro- pyridine-5-carboxylate | ¹H NMR (CDCl₃, 400 MHz): 7.35 (d, J = 1.0 Hz, 1H), 7.20 (d, J = 1.0 Hz, 2H), 6.03 (s, 1H), 5.21 (s, 1H), 4.86 (m, 1H), 2.35 (s, 3H), 2.04 (s, 3H), 1.18 (d, J = 6.4 Hz, 3H), 0.86 (d, J = 6.4 Hz, 3H). MS (ES⁺) 366, m/z (M + 1) 366, C₁₈H₁₈Cl₂N₂O₂ requires 366 |
| 72 | 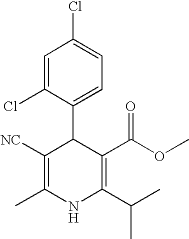 5-methyl-2-methyl-3-cyano-4-(2,4- dichlorophenyl)-6-isopropyl-1,4- dihydro-pyridin-5-carboxylate | ¹H NMR (CDCl₃, 400 MHz): 7.35 (d, J = 2.0 Hz, 1H), 7.18 (m, 2H), 5.90 (s, 1H), 5.19 (s, 1H), 4.20 (m, 1H), 3.55 (s, 3H), 2.09 (s, 3H), 1.21 (d, J = 6.8 Hz, 3H), 1.15 (d, J = 6.8 Hz, 3H). MS (ES⁺) 364, m/z (M + 1) 366, C₁₈H₁₈Cl₂N₂O₂ requires 366 |
| 73 | 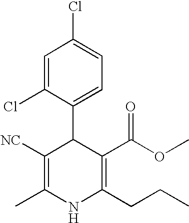 5-methyl-2-methyl-3-cyano-4-(2,4- dichlorophenyl)-6-propyl-1,4-dihydro- pyridine-5-carboxylate | ¹H NMR (CDCl₃, 400 MHz): 7.28 (d, J = 0.8 Hz, 1H), 7.13 (s, 2H), 6.03 (s, 1H), 5.14 (s, 1H), 3.48 (s, 3H), 2.70 (m, 1H), 2.53 (m, 1H), 1.99 (s, 3H), 1.59 (m, 2H), 0.95 (t, J = 7.2 Hz, 3H). MS (ES⁺) 364, m/z (M + 1) 366, C₁₈H₁₈Cl₂N₂O₂ requires 366 |
| 74 | 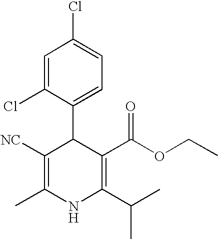 5-ethyl-2-methyl-3-cyano-4-(2,4- dichlorophenyl)-6-isopropyl-1,4- dihydro-pyridine-5-carboxylate | ¹H NMR (CDCl₃, 400 MHz): 7.35 (d, J = 2.0 Hz, 1H), 7.18 (m, 2H), 5.87 (s, 1H), 5.21 (s, 1H), 4.24 (m, 1), 3.95 (m, 2H), 2.08 (s, 3H), 1.23 (d, J = 7.2 Hz, 3H), 1.15 (d, J = 7.2 Hz, 3H), 1.09 (t, J = 4.0 Hz, 3H). MS (ES⁺) 378, m/z (M + 1) 380, C₁₉H₂₀Cl₂N₂O₂ requires 380 |
| 75 | 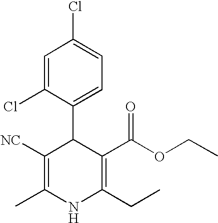 5-ethyl-2-methyl-3-cyano-4-(2,4- dichlorophenyl)-6-ethyl-1,4-dihydro- pyridine-6-carboxylate | ¹H NMR (CDCl₃, 400 MHz): 7.51 (s, 1H), 7.35 (m, 2H), 6.03 (s, 1H), 5.37 (s, 1H), 4.14 (m, 2H), 2.96 (m, 1H), 2.86 (m, 1H), 2.22 (s, 3H), 1.39 (t, J = 7.2 Hz, 3H), 1.24 (t, J = 7.2 Hz, 3H). MS (ES⁺) 364, m/z (M + 1) 366, C₁₈H₁₈Cl₂N₂O₂ requires 366 |
| 76 | 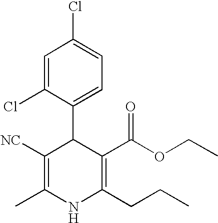 5-ethyl-2-methyl-3-cyano-4-(2,4- dichlorophenyl)-6-propyl-1,4-dihydro- pyridine-5-carboxylate | ¹H NMR (CDCl₃, 400 MHz): 7.35 (s, 1H), 7.19 (m, 2H), 5.92 (s, 1H), 5.22 (s, 1H), 3.98 (m, 2H), 2.78 (m,1H), 2.60 (m, 1H), 2.05 (s, 3H), 1.64 (m, 2H), 1.09 (t, J = 4.0 Hz, 3H), 1.00 (t, J = 5.6 Hz, 3H). MS (ES⁺) 378, m/z (M + 1) 380, C₁₉H₂₀Cl₂N₂O₂ requires 380 |
| 77 | 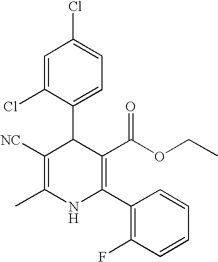 5-ethyl-2-methyl-3-cyano-4-(2,4- dichlorophenyl)-6-(2-fluorophenyl)- 1,4-dihydro-pyridine-5-carboxylate | ¹H NMR (CDCl₃, 400 MHz): 7.42 (m, 3H), 7.30 (m, 2H), 7.21 (m, 1H), 7.14 (m, 1H), 5.96 (s, 1H), 5.35 (s, 1H), 3.79 (q, J = 7.2 Hz, 2H), 2.08 (s, 3H), 0.85 (t, J = 7.2 Hz, 3H). MS (ES⁺) 430, m/z (M + 1) 432, C₂₂H₁₇Cl₂N₂O₂ requires 432 |
| 78 | 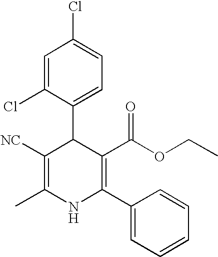 5-ethyl-2-methyl-3-cyano-4-(2,4- dichlorophenyl)-6-phenyl-1,4-dihydro- pyridine-5-carboxylate | ¹H NMR (CDCl₃, 400 MHz): 7.41 (m, 5H), 7.33 (m, 2H), 7.26 (m, 1H), 5.92 (s, 1H), 5.30 (s, 1H), 3.77 (q, J = 7.2 Hz, 2H), 2.09 (s, 3H), 0.82 (t, J = 7.2 Hz, 3H). MS (ES⁺) 412, m/z (M + 1) 414, C₂₂H₁₈Cl₂N₂O₂ requires 414. |
| 79 | 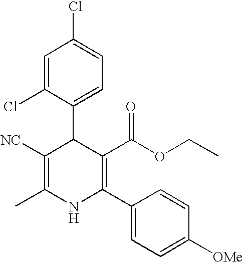 5-ethyl-2-methyl-3-cyano-4-(2,4- dichlorophenyl)-6-(4-methoxyphenyl)- 1,4-dihydro-pyridin-5-carboxylate | ¹H NMR (CDCl₃, 400 MHz): 7.60 (m, 2H), 7.47 (m, 3H), 7.15 (m, 2H), 6.07 (s, 1H), 5.51 (s, 1H), 4.06 (s, 3H), 4.02 (q, J = 7.2 Hz, 2H), 2.31 (s, 3H), 1.07 (t, J = 7.2 Hz, 3H). MS (ES⁺) 442, m/z (M + 1) 444, C₂₃H₂₀Cl₂N₂O₂ requires 444 |
| 80 | 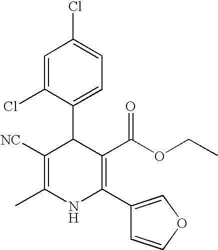 5-ethyl-2-methyl-3-cyano-4-(2,4- dichlorophenyl)-6-(3-furyl)-1,4- dihydro-pyridine-5-carboxylate | ¹H NMR (CDCl₃, 400 MHz): 7.49 (m, 1H), 7.29 (m, 1H), 7.22 (d, J = 2.0 Hz, 1H), 7.14 (d, J = 8.4 Hz, 1H), 7.06 (dd, J = 8.4, 2.0 Hz, 1H), 6.32 (m, 1H), 5.74 (s, 1H), 5.13 (s, 1H), 3.73 (m, 2H), 1.92 (s, 3H), 0.84 (m, 3H). MS (ES⁺) 402, m/z (M + 1) 404, C₂₀H₁₆Cl₂N₂O₂ requires 404 |
| 81 | 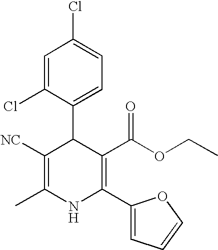 5-ethyl-2-methyl-3-cyano-4-(2,4- dichlorophenyl)-6-(2-furyl)-1,4- dihydro-pyridine-5-carboxylate | ¹H NMR (CDCl₃, 400 MHz): 7.50 (m, 1H), 7.38 (d, J = 2.0 Hz, 1H), 7.30 (d, J = 8.4 Hz, 1H), 7.22 (dd, J = 8.4, 2.0 Hz, 1H), 7.16 (d, J = 3.6 Hz, 1H), 6.58 (s, 1H), 6.53 (dd, J = 3.6, 2.0 Hz, 1H), 5.33 (s, 1H), 4.00 (m, 2H), 2.15 (s, 3H), 1.09 (t, J = 7.2 Hz, 3H). MS (ES⁺) 402, m/z (M + 1) 404,C₂₀H₁₆Cl₂N₂O₃ requires 404 |
| 82 | 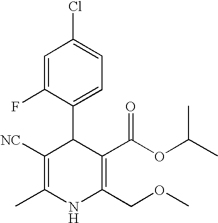 5-isopropyl-2-methyl-3-cyano-4-(2- fluoro-4-chlorophenyl)-6- (methoxymethyl)-1,4-dihydro-pyridine- 5-carboxylate | ¹H NMR (CDCl₃, 400 MHz): 7.04 (s, 1H), 7.01 (d, J = 8.0 Hz, 1H), 6.93 (m, 2H), 4.79 (s, 1H), 4.71 (m, 1H), 4.54 (m, 2H), 3.35 (s, 3H), 1.99 (s, 3H), 1.06 (d, J = 6.4 Hz, 3H), 0.78 (d, J = 6.4 Hz, 3H). MS (ES⁺) 378, m/z (M + 1) 379, C₁₉H₂₀ClFN₂O₃ requires 379 |
| 83 | 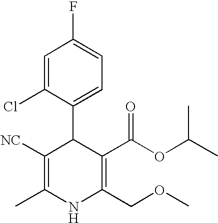 5-isopropyl-2-methyl-3-cyano-4-(2- chloro-4-fluorophenyl)-6- (methoxymethyl)-1,4-dihydro-pyridine- 5-carboxylate | ¹H NMR (CDCl₃, 400 MHz): 7.46 (dd, J = 8.4, 6.4 Hz, 1H), 7.35 (s, 1H), 7.30 (dd, J = 8.4, 2.4 Hz, 1H), 7.16 (m, 1H), 5.44 (s, 1H), 5.06 (m, 1H), 4.92 (s, 2H), 3.71 (s, 3H), 2.32 (s, 3H), 1.41 (d, J = 6.4 Hz, 3H), 1.06 (d, J = 6.4 Hz, 3H). MS (ES⁺) 378, m/z (M + 1) 379, C₁₉H₂₀ClFN₂O₃ requires 379 |
| 84 | 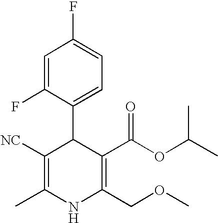 5-isopropyl-2-methyl-3-cyano-4-(2,4- difluorophenyl)-6-(methoxymethyl)- 1,4-dihydro-pyridine-5-carboxylate | ¹H NMR (CDCl₃, 400 MHz): 7.17 (m, 2H), 6.78 (m, 2H), 4.92 (s, 1H), 4.83 (m, 1H), 4.66 (m, 2H), 3.48 (s, 3H), 2.12 (s, 3H), 1.18 (d, J = 6.4 Hz, 3H), 0.91 (d, J = 6.4 Hz, 3H). MS (ES⁺) 363, m/z (M + 1) 363, C₁₉H₂₀F₂N₂O₃ requires 363 |
| 85 | 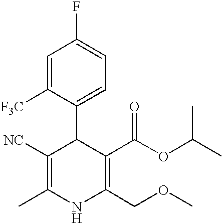 5-isopropyl-2-methyl-3-cyano-4-(2- trifluoromethyl-4-fluorophenyl)-6- (methoxymethyl)-1,4-dihydro-pyridine- 5-carboxylate | ¹H NMR (CDCl₃, 400 MHz): 7.23 (dd, J = 8.8, 5.2 Hz, 1H), 7.11 (dd, J = 9.6, 2.8 Hz, 1H), 6.99 (m, 2H), 4.86 (s, 1H), 4.66 (m, 1H), 4.48 (m, 2H), 3.29 (s, 3H), 1.91 (s, 3H), 0.91 (d, J = 6.4 Hz, 3H), 0.55 (d, J = 6.4 Hz, 3H). MS (ES⁺) 413, m/z (M + 1) 413,C₂₀H₂₀F₄N₂O₃ requires 413 |
| 86 | 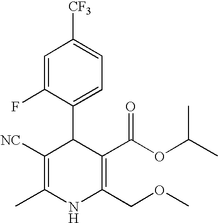 5-isopropyl-2-methyl-3-cyano-4-(2- fluoro-4-trifluoromethylphenyl)-6- (methoxymethyl)-1,4-dihydro-pyridine- 5-carboxylate | ¹H NMR (CDCl₃, 400 MHz): 7.36 (m, 2H), 7.28 (m, 1H), 7.22 (s, 1H), 5.02 (s, 1H), 4.84 (m, 1H), 4.68 (m, 2H), 3.49 (s, 3H), 2.13 (s, 3H), 1.18 (d, J = 6.4 Hz, 3H), 0.88 (d, J = 6.4 Hz, 3H), MS (ES⁺) 413, m/z (M + 1) 413, C₂₀H₂₀F₄N₂O₃ requires 413 |
| 87 | 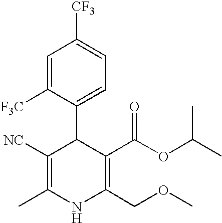 5-isopropyl-2-methyl-3-cyano-4-(2,4- bistrifluoromethylphenyl)-6- (methoxymethyl)-1,4-dihydro-pyridine- 5-carboxylate | ¹H NMR (CDCl₃, 400 MHz): 7.76 (s, 1H), 7.63 (d, J = 8.0 Hz, 1H), 7.51 (d, J = 8.0 Hz, 1H), m 7.14 (s, 1H), 5.05 (s, 1H), 4.74 (m, 1H), 4.58 (m, 2H), 3.39 (s, 3H), 2.00 (s, 3H), 0.99 (d, J = 6.4 Hz, 3H), 0.59 (d, J = 6.4 Hz, 3H). MS (ES⁺) 463, m/z (M + 1) 463,C₂₁H₂₀F₆N₂O₃ requires 463 |
| 88 | 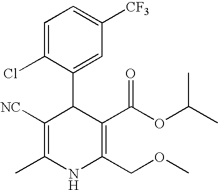 5-isopropyl-2-methyl-3-cyano-4-(2- chloro-5-trifluoromethylphenyl)-6- (methoxymethyl)-1,4-dihydro-pyridine- 5-carboxylate | ¹H NMR (CDCl₃, 400 MHz): 7.46 (m, 1H), 7.44 (s, 1H), 7.37 (dd, J = 8.4, 1.6 Hz, 1H), 7.18 (s, 1H), 5.28 (s, 1H), 4.80 (m, 1H), 4.68 (s, 2H), 3.47 (s, 3H), 2.11 (s, 3H), 1.14 (d, J = 6.4 Hz, 3H), 0.76 (d, J = 6.4 Hz, 3H). MS (ES⁺) 429, m/z (M + 1) 429,C₂₀H₂₀ClF₃N₂O₃ requires 429 |
| 89 | 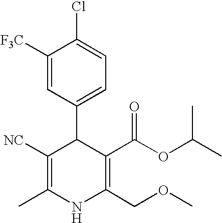 5-isopropyl-2-methyl-3-cyano-4-(3- trifluoromethyl-4-chlorophenyl)-6- (methoxymethyl)-1,4-dihydro-pyridine- 5-carboxylate | ¹H NMR (CDCl₃, 400 MHz): 7.40 (d, J = 2.0 Hz, 1H), 7.30 (d, J = 8.0 Hz, 1H), 7.23 (dd, J = 8.0, 2.0 Hz, 1H), 7.10 (s, 1H), 4.75 (m, 1H), 4.54 (m, 3H), 3.36 (s, 3H), 2.04 (s, 3H), 1.07 (d, J = 6.4 Hz, 3H), 0.83 (d, J = 6.4 Hz, 3H). MS (ES⁺) 429, m/z (M + 1) 429, C₂₀H₁₉ClF₃N₂O₃ requires 429 |
| 90 | 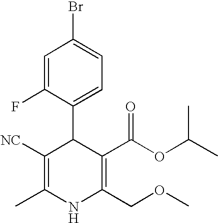 5-isopropyl-2-methyl-3-cyano-4-(2- fluoro-4-bromophenyl)-6- (methoxymethyl)-1,4-dihydro-pyridine- 5-carboxylate | ¹H NMR (CDCl₃, 400 MHz): 7.08 (m, 2H), 7.04 (s, 1H), 6.96 (m, 1H), 4.79 (s, 1H), 4.71 (m, 1H), 4.53 (m, 2H), 3.35 (s, 3H), 1.99 (s, 3H), 1.06 (d, J = 6.4 Hz, 3H), 0.79 (d, J = 6.4 Hz, 3H). MS (ES⁺) 424, m/z (M + 1) 424, C₁₉H₂₀BrFN₂O₃ requires 424 |
| 91 | 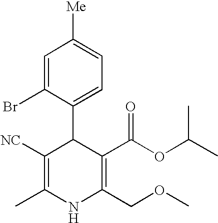 5-isopropyl-2-methyl-3-cyano-4-(2- bromo-4-fluorophenyl)-6- (methoxymethyl)-1,4-dihydro-pyridine- 5-carboxylate | ¹H NMR (CDCl₃, 400 MHz): 7.21 (d, J = 1.2 Hz, 1H), 6.99 (d, J = 8.0 Hz, 1H), 6.95 (s, 1H), 6.92 (dd, J = 8.0, 1.2 Hz, 1H), 5.07 (s, 1H), 4.71 (m, 1H), 4.57 (s, 2H), 3.36 (s, 3H), 2.15 (s, 3H), 1.95 (s, 3H), 1.05 (d, J = 6.4 Hz, 3H), 0.70 (d, J = 6.4 Hz, 3H). MS (ES⁺) 420, m/z (M + 1) 420, C₂₀H₂₃BrN₂O₃ requires 420 |
| 92 | 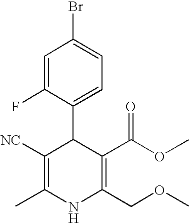 5-methyl-2-methyl-3-cyano-(2- fluoro-4-bromophenyl)-6- (methoxymethyl)-1,4-dihydro-pyridine- 5-carboxylate | ¹H NMR (CDCl₃, 400 MHz): 7.09 (m, 2H), 7.06 (d, J = 2.0 Hz,1H), 6.95 (m, 1H), 4.77 (s, 1H), 4.52 (m, 2H), 3.43 (s, 3H), 3.36 (s, 3H), 1.99 (s, 3H). MS (ES⁺) 396, m/z (M + 1) C₁₇H₁₆BrFN₂O₃ requires 396 |
| 93 | 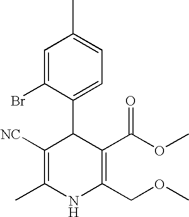 5-methyl-2-methyl-3-cyano-4-(2- bromo-4-methylphenyl)-6- (methoxymethyl)-1,4-dihydro-pyridine- 5-carboxylate | ¹H NMR (CDCl₃, 400 MHz): 7.22 (s,1H), 7.00 (m, 2H), 6.92 (m, 1H), 5.06 (s, 1H), 4.55 (m, 2H), 3.41 (s, 3H), 3.36 (s, 3H), 2.15 (s, 1H), 1.97 (s, 3H). MS (ES⁺) 392, m/z (M + 1) C₁₈H₁₉BrN₂O₃ requires 392 |
| 94 | 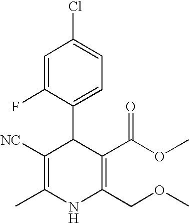 5-methyl-2-methyl-3-cyano-4-(2- fluoro-4-chlorophenyl)-6- (methoxymethyl)-1,4-dihydro-pyridine- 5-carboxylate | ¹H NMR (CDCl₃, 400 MHz): 7.10 (s, 1H), 7.01 (m, 1H), 6.94 (m, 2H), 4.78 (s, 1H), 4.52 (m, 2H), 3.43 (s, 3H), 3.36 (s, 3H), 1.99 (s, 3H). MS (ES⁺) 351, m/z (M + 1) C₁₉H₂₀ClFN₂O₃ requires 351 |
| 95 | 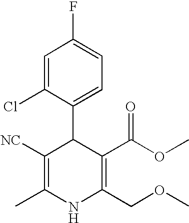 5-methyl-2-methyl-3-cyano-4-(2- chloro-4-fluorophenyl)-6- (methoxymethyl)-1,4-dihydro-pyridine- 5-carboxylate | ¹H NMR (CDCl₃, 400 MHz): 7.32 (dd, J = 8.4, 6.0 Hz, 1H), 7.27 (s, 1H), 7.17 (dd, J = 8.4, 2.8 Hz, 1H), 7.02 (m, 1H), 5.29 (s, 1H), 4.77 (m, 2H), 3.63 (s, 3H), 3.59 (s, 3H), 2.20 (s, 3H). MS( ES⁺) 351, m/z (M + 1) C₁₉H₂₀ClFN₂O₃ requires 351 |
| 96 | 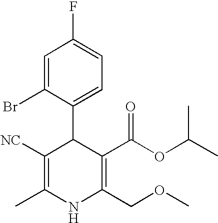 5-isopropyl-2-methyl-3-cyano-4-(2- bromo-4-fluorophenyl)-6- (methoxymethyl)-1,4-dihydro-pyridine- 5-carboxylate | ¹H NMR (CDCl₃, 400 MHz): 7.46 (m, 2H), 7.33 (s, 1H), 7.20 (m, 1H), 5.43 (s, 1H), 5.06 (m, 1H), 4.91 (s, 2H), 3.70 (s, 3H), 2.30 (s, 3H), 1.40 (d, J = 6.0 Hz, 3H), 1.05 (d, J = 6.0 Hz, 3H). MS (ES⁺) 423, m/z (M + 1) 3 C₁₉H₂₀BrFN₂O₃ requires 423 |
| 97 | 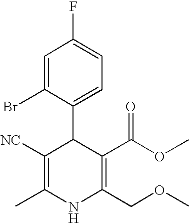 5-methyl-2-methyl-3-cyano-4-(2- bromo-4-fluorophenyl)-6- (methoxymethyl)-1,4-dihydro-pyridine- 5-carboxylate | ¹H NMR (CDCl₃, 400 MHz): 7.23 (m, 2H), 7.16 (s, 1H), 6.98 (m, 1H), 5.21 (s, 1H), 4.67 (m, 2H), 3.54 (s, 3H), 3.49 (s, 3H), 2.10 (s, 3H). MS (ES⁺) 396, m/z (M + 1) C₁₇H₁₆BrFN₂O₃ requires 396 |
| 98 | 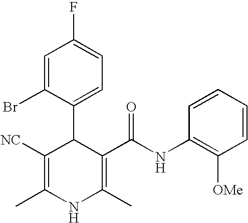 2,6-dimethyl-3-cyano-4-(2-bromo-4- fluorophenyl)-5-(2- methoxyphenyl)carbamoyl-1,4- dihydro-pyridine | ¹H NMR (CDCl₃, 400 MHz): 8.38 (dd, J = 8.0, 1.6 Hz, 1H), 7.75 (s, 1H), 7.60 (dd, J = 8.4, 6.4 Hz, 1H), 7.53 (dd, J = 8.4, 2.4 Hz, 1H), 7.27 (m, 1H), 7.20 (m, 1H), 7.08 (dd, J = 7.6, 1.2 Hz, 1H), 7.00 (dd, J = 8.4, 1.2 Hz, 1H), 6.32 (s, 1H), 5.39 (s, 1H), 3.92 (s, 3H), 2.48 (s, 3H), 2.27 (s, 3H). MS (ES⁺) 457,m/z (M + 1) C₂₂H₁₉BrFN₃O₂ requires 457 |
| 99 | 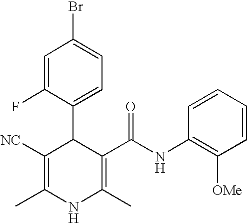 2,6-dimethyl-3-cyano-4-(2-fluoro-4- bromophenyl)-5-(2- methoxyphenyl)carbamoyl-1,4- dihydro-pyridine | ¹H NMR (CDCl₃, 400 MHz): 8.08 (dd, J = 8.0, 1.6 Hz, 1H), 7.52 (s, 1H), 7.18 (m, 2H), 7.08 (m, 1H), 6.87 (m, 1H), 6.79 (m, 1H), 6.66 (dd, J = 8.0, 1.2 Hz, 1H), 6.07 (s, 1H), 4.79 (s, 1H), 3.60 (s, 3H), 2.18 (s, 3H), 1.94 (s, 3H). MS (ES⁺) 457, m/z (M + 1) C₂₂H₁₉BrFN₃O₂ requires 457 |
| 100 | 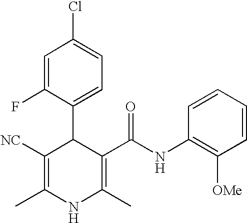 2,6-dimethyl-3-cyano-4-(2-fluoro-4- chlorophenyl)-5-(2- methoxyphenyl)carbamoyl-1,4- dihydro-pyridine | ¹H NMR (CDCl₃, 400 MHz): 8.42 (dd, J = 8.0, 1.2 Hz, 1H), 7.84 (s, 1H), 7.42 (m, 1H), 7.35 (m, 2H), 7.17 (m, 1H), 7.09 (m, 1H), 6.99 (dd, J = 8.0, 1.2 Hz, 1H), 6.34 (s, 1H), 5.12 (s, 1H), 3.92 (d, 3H), 2.51 (s, 3H), 2.66 (s, 3H), MS (ES⁺) 412, m/z (M + 1) C₂₂H₁₉ClFN₃O₂ requires 412 |
| 101 | 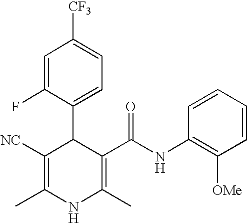 2,6-dimethyl-3-cyano-4-(2-fluoro-4- trifluoromethylphenyl)-5-(2- methoxyphenyl)carbamoyl)-1,4- dihydro-pyridine | ¹H NMR (CDCl₃, 400 MHz): 8.08 (dd, J = 8.0, 1.6 Hz, 1H), 7.50 (s, 1H), 7.34 (m, 2H), 7.17 (d, J = 10.0 Hz, 1H), 6.87 (m, 1H), 6.77 (m, 1H), 6.67 (dd, J = 8.0, 0.8 Hz, 1H), 6.15 (s, 1H), 4.89 (s, 1H), 3.59 (s, 3H), 2.19 (s, 3H), 1.95 (s, 3H). MS (ES⁺) 446, m/z (M + 1) C₂₃H₁₉F₄N₃O₂ requires 446 |
| 102 | 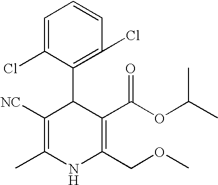 5-isopropyl-2-methyl-3-cyano-4-(2,6- dichlorophenyl)-6-(methoxymethyl)- 1,4-dihydro-pyridine-5-carboxylate | ¹H NMR (CDCl₃, 400 MHz): 7.29 (m, 2H), 7.23 (s, 1H), 7.09 (m, 1H), 5.83 (s, 1H), 4.83 (m, 1H), 4.60 (m, 2H), 3.46 (s, 3H), 2.09 (s, 3H), 1.16 (d, J = 6.4 Hz, 3H), 0.74 (d, J = 6.4 Hz, 3H). MS (ES⁺) 396, m/z (M + 1) C₁₉H₂₀Cl₂N₂O₃ requires 396 |
| 103 | 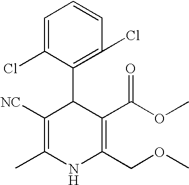 5-methyl-2-methyl-3-cyano-4-(2,6- dichlorophenyl)-6-(methoxymethyl)- 1,4-dihydro-pyridine-5-carboxylate | ¹H NMR (CDCl₃, 400 MHz): 7.29 (m, 2H), 7.24 (s, 1H), 7.09 (m, 1H), 5.84 (s, 1H), 4.58 (m, 2H), 3.48 (s, 3H), 3.46 (s, 3H), 2.08 (s, 3H). MS (ES⁺) 368, m/z (M + 1) C₁₇H₁₆Cl₂N₂O₃ requires 368 |
| 104 | 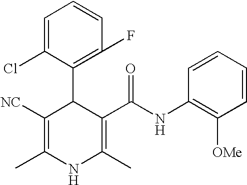 2,6-dimethyl-3-cyano-4-(2-fluoro-6- chlorophenyl)-5-(2- methoxyphenyl)carbamoyl-1,4- dihydro-pyridine | ¹H NMR (DMSO-d6, 400 MHz): 8.85 (s, 1H), 8.40 (s, 1H), 7.58 (dd, J = 8.0, 1.6 Hz, 1H), 7.22 (m, 2H), 7.14 (m, 1H), 6.91 (m, 2H), 6.77 (m, 1H), 5.41 (s, 1H), 3.68 (s, 3H), 1.96 (s, 3H), 1.94 (s, 3H). MS (ES⁺) 412, m/z (M + 1) C₂₂H₁₉ClFN₃O₂ requires 412 |
| 105 | 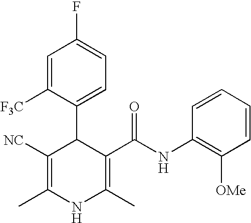 2,6-dimethyl-3-cyano-4-(2- trifluoromethyl-4-fluorophenyl)-5-(2- methoxyphenyl)carbamoyl-1,4- dihydro-pyridine | ¹H NMR (MeOD, 400 MHz): 7.83 (dd, J = 8.8, 5.2 Hz, 1H), 7.61 (dd, J = 8.0, 1.6 Hz, 1H), 7.44 (m, 1H), 7.36 (dd, J = 9.6, 2.8 Hz, 1H), 7.05 (m, 1H), 6.92 (dd, J = 8.4 1.2 Hz, 1H), 6.83 (m, 1H), 5.01 (s, 1H), 3.73 (s, 3H), 2.08 (s, 3H), 2.06 (s, 3H). MS (ES⁺) 446, m/z (M + 1)C₂₃H₁₉F₄N₃O₂ requires 446 |
| 106 | 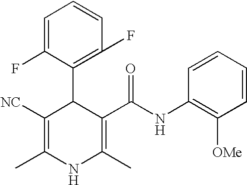 2,6-dimethyl-3-cyano-4-(2,6- difluorophenyl)-5-(2- methoxyphenyl)carbamoyl-1,4- dihydro-pyridine | ¹H NMR (CDCl₃, 400 MHz): 8.20 (d, J = 8.0 Hz, 1H), 7.67 (s, 1H), 7.29 (m, 1H), 6.99 (m, 1H), 6.87 (m, 3H), 6.80 (m, 1H), 6.34 (s, 1H), 4.91 (s, 1H), 3.72 (s, 3H), 2.29 (s, 3H), 2.05 (s, 3H). MS (ES⁺) 396, m/z (M + 1) C₂₂H₁₉F₂N₃O₂ requires 396 |
| 107 | 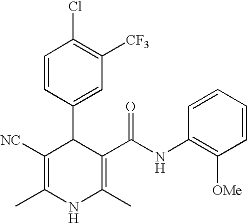 2,6-dimethyl-3-cyano-4-(4-fluoro-5- trifluoromethylphenyl)-5-(2- methoxyphenyl)-carbamoyl-1,4- dihydro-pyridine | ¹H NMR (CDCl₃, 400 MHz): 8.13 (dd, J = 8.0, 1.2 Hz, 1H), 7.65 (d, J = 2.0 Hz, 1H), 7.58 (m, 1H), 7.48 (m, 2H), 6.98 (m, 1H), 6.90 (m, 1H), 6.77 (dd, J = 8.0, 0.8 Hz, 1H), 5.98 (s, 1H), 4.61 (s, 1H), 3.67 (s, 3H), 2.33 (s, 3H), 2.09 (s, 3H). MS (ES⁺) 462, m/z (M + 1) C₂₃H₁₉ClF₃N₃O₂ requires 462 |
| 108 | 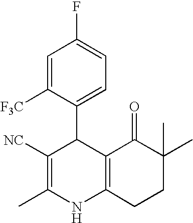 2-methyl-3-cyano-4-(2-trifluoromethyl- 4-fluorophenyl)-5,6-(3,3-dimethyl)- cyclohexan-2-one-1,4-dihydro-pyridine | ¹H NMR (DMSO-d6, 400 MHz): 9.57 (broad s, 1H), 7.75 (dd, J = 8.8, 1.8 Hz, 1H), 7.30 (dd, J = 8.8, 6.4 Hz, 1H), 7.18 (td, J = 8.4, 2.8 Hz, 1H), 4.97 (s, 1H), 2.55 (m, 2H), 2.04 (s, 3H), 1.82 (m, 2H), 1.01 (s, 3H), 0.90 (s, 3H). MS (ES⁺) 379, m/z (M + 1)⁺ C₂₀H₁₈F₄N₂O requires 379 |
| 109 | 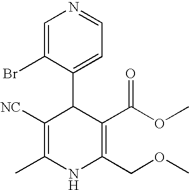 5-methyl-2-methyl-3-cyano-4-[4-(2- bromopyridine)]-6-(methoxymethyl)- 1,4-dihydro-pyridine-5-carboxylate | ¹H NMR (DMSO-d6, 400 MHz): 9.23 (broad s, 1H), 8.69 (s, 1H), 8.52 (d, J = 5.2 Hz, 1H), 7.31 (d, J = 5.2 Hz, 1H), 5.07 (s, 1H), 4.58 (s, 2H), 3.45 (s, 3H), 3.36 (s, 3H), 2.06 (s, 3H). MS (ES⁺) 379, m/z (M + 1)⁺ C₁₆H₁₆BrN₃O₃ requires 379 |
| 110 | 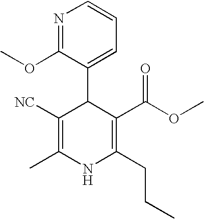 5-methyl-2-methyl-3-cyano-4-[3-(2- methoxypyridine)]-6-propyl-1,4- dihydro-pyridine-5-carboxylate | ¹H NMR (CD₃OD, 400 MHz): 7.97 (d, J = 4.8, 2.4 Hz, 1H), 7.42 (dd, J = 7.6, 5.2 Hz, 1H), 4.90 (s, 1H), 3.94 (s, 3H), 3.52 (s, 3H), 2.77 (m, 1H), 2.64 (m, 1H), 2.02 (s, 3H), 1.65 (m, 2H), 1.03 (t, J = 7.6 Hz, 3H). MS (ES⁺) 328, m/z (M + 1)⁺ C₁₈H₂₁N₃O₃ requires 328 |
| 111 | 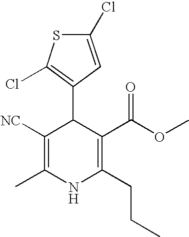 5-methyl-2-methyl-3-cyano-4-[3-(2,5- dichlorothiophene)]-6-propyl-1,4- dihydro-pyridine-5-carboxylate | ¹H NMR (DMSO-d6, 400 MHz): 9.13 (broad s, 1H), 6.62 (s, 1H), 4.53 (s, 1H), 3.35 (s, 3H), 2.48 (m, 1H), 2.39 (m, 1H), 1.84 (s, 3H), 1.36 (m, 2H), 0.75 (t, J = 7.2 Hz, 3H). MS (ES⁺) 372, m/z (M + 1)⁺ C₁₆H₁₆Cl₂N₂O₂S requires 372 |
| 112 | 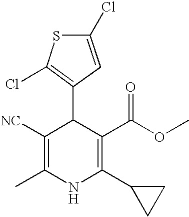 5-methyl-2-methyl-3-cyano-4-[3-(2,5- dichlorothiophene)]-6-cyclopropyl-1,4- dihydro-pyridine-5-carboxylate | ¹H NMR (DMSO-d6, 400 MHz): 8.18 (broad s, 1H), 6.78 (s, 1H), 4.71 (s, 1H), 3.55 (s, 3H), 2.80 (m, 1H), 2.02 (s, 3H), 0.98 (m, 1H), 0.85 (m, 3H). MS (ES⁺) 370, m/z (M + 1)⁺ C₁₆H₁₄Cl₂N₂O₂S requires 370 |
| 113 | 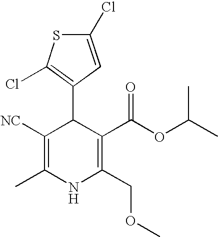 5-isopropyl-2-methyl-3-cyano-4-[3- (2,5-dichlorothiophene)]-6- (methoxymethyl)-1,4-dihydro-pyridine- 5-carboxylate | ¹H NMR (DMSO-d6, 400 MHz): 9.08 (broad s, 1H), 6.88 (s, 1H), 4.82 (m, 1H), 4.73 (s, 1H), 4.59 (d, J = 14.0 Hz, 1H), 4.51 (d, J = 14.0 Hz, 1H), 3.33 (s, 3H), 2.05 (s, 3H), 1.16 (d, J = 6.4 Hz, 3H), 0.95 (d, J = 6.4 Hz, 3H). MS (ES⁺) 402, m/z (M + 1)⁺ C₁₇H₁₈Cl₂O₂O₃S requires 402 |
| 114 | 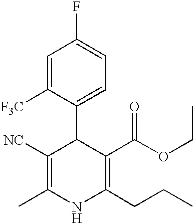 5-ethyl-2-methyl-3-cyano-4-(2- trifluoromethyl-4-fluorophenyl)-6- propyl-1,4-dihydro-pyridine-5- carboxylate | ¹H NMR (CDCl₃, 400 MHz): 7.45 (dd, J = 8.8, 5.6 Hz, 1H), 7.31 (dd, J = 9.2, 2.8 Hz, 1H), 7.20 (m, 1H), 5.86 (s, 1H), 5.07 (s, 1H), 3.97 (m, 2H), 2.67 (m, 2H), 2.09 (s, 3H), 1.66 (m, 2H), 1.03 (m, 6H). MS (ES⁺) 397, m/z (M + 1), C₂₀H₂₀F₄N₂O₂ requires 397 |
| 115 | 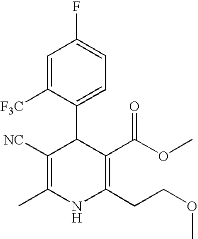 5-methyl-2-methyl-3-cyano-4-(2- trifluoromethyl-4-fluorophenyl)-6- (methoxyethyl)-1,4-dihydro-pyridine-5- carboxylate | ¹H NMR (CDCl₃, 400 MHz): 7.25 (dd, J = 8.8, 5.6 Hz, 1H), 7.09 (dd, J = 9.2, 2.8 Hz, 1H), 6.97 (m, 2H), 4.81 (s, 1H), 3.44 (m, 2H), 3.25 (s, 3H), 3.20 (s, 3H), 2.95 (m, 2H), 1.86 (m, 3H). MS (ES⁺) 399, m/z (M + 1), C₁₉H₁₈F₄N₂O₃ requires 399 |
| 116 | 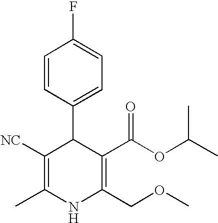 5-isopropyl-2-methyl-3-cyano-4-(4- fluorophenyl)-6-(methoxymethyl)-1,4- dihydro-pyridine-5-carboxylate | ¹H NMR (CDCl₃, 400 MHz): 7.20 (m, 2H), 7.13 (s, 1H), 6.97 (m, 2H), 4.85 (m, 1H), 4.68 (m, 2H), 4.58 (s, 1H), 3.48 (s, 3H), 2.13 (s, 3H), 1.20 (d, J = 6.4 Hz, 3H), 0.92 (d, J = 6.4 Hz, 3H). MS (ES⁺) 345, m/z (M + 1), C₁₉H₂₁FN₂O₃ requires 345 |
| 117 | 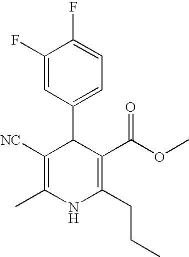 5-methyl-2-methyl-3-cyano-4-(3,4- difluorophenyl)-6-propyl-1,4-dihydro- pyridine-5-carboxylate | ¹H NMR (DMSO-d6, 400 MHz): 9.38 (broad s, 1H), 7.50 (dt, J = 10.8, 8.4 Hz, 1H), 7.24 (m, 1H), 7.12 (m, 1H), 4.61 (s, 1H), 3.60 (s, 3H), 2.77 (m, 1H), 2.62 (m, 1H), 2.14 (s, 3H), 1.67 (m, 2H), 1.04 (d, J = 7.2 Hz, 3H). MS (ES⁺) 333, m/z (M + 1)⁺ C₁₈H₁₈F₂N₂O₂ requires 333 |
| 118 | 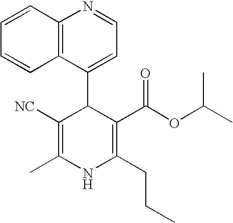 5-isopropyl-2-methyl-3-cyano-4-(4- quinoline)-6propyl-1,4-dihydro- pyridine-5-carboxylate | ¹H NMR (DMSO-d6, 400 MHz): 9.38 (broad s, 1H), 8.90 (d, J = 4.4 Hz, 1H), 8.45 (d, J 8.8 Hz 1H), 8.04 (d, J 8.0 Hz, 1H), 7.79 (t, J = 8.0 Hz,1H), 7.68 (t, J = 8.0 Hz, 1H), 7.32 (d, J = 4.4 Hz, 1H), 5.51 (s, 1H), 3.80 (m, 1H), 3.30 (s, 3H), 2.70 (m, 2H), 2.03 (s, 3H), 1.66 (m, 2H), 1.01 (d, J = 7.2 Hz, 3H). MS (ES⁺) 348, m/z (M + 1)⁺ C₂₁H₂₁N₃O₂ requires 348 |
| 119 | 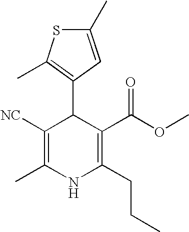 5-methyl-2-methyl-3-cyano-4-3-[2,5- dimethylthiophene)]-6-propyl-1,4- dihydro-pyridine-5-carboxylate | ¹H NMR (DMSO-d6, 400 MHz): 9.29 (broad s, 1H), 6.56 (s, 1H), 4.70 (s, 1H), 3.71 (s, 3H), 2.86 (m, 1H), 2.74 (m, 1H), 2.51 (s, 3H), 2.48 (s, 3H), 2.19 (s, 3H), 1.73 (m, 2H), 1.12 (d, J = 7.2 Hz, 3H). MS (ES⁺) 331, m/z (M + 1)⁺ C₁₈H₂₂N₂O₂S requires 331 |
| 120 | 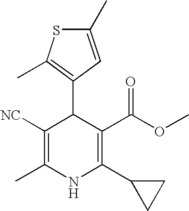 5-methyl-2-methyl-3-cyano-4-3-[2,5- dimethylthiophene)]-6-cyclopropyl-1,4- dihydro-pyridine-5-carboxylate | ¹H NMR (DMSO-d6, 400 MHz): 8.08 (broad s, 1H), 6.42 (s, 1H), 4.60 (s, 1H), 3.63 (s, 3H), 2.91 (m, 1H), 2.41 (s, 3H), 2.39 (s, 3H), 2.09 (s, 3H), 1.06 (m, 1H), 0.97 (m, 2H), 0.90 (m, 1H). MS (ES⁺) 329, m/z (M + 1)⁺ C₁₈H₂₀N₂O₂S requires 329 |

TABLE 3

| | | Physical Data |
|---|---|---|
| Compound | | ¹H NMR 400 MHz (DMSO-d6) |
| Number | Structure | and/or MS (m/z) |
| 121 | 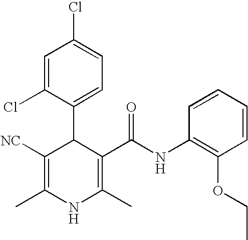 2,6-dimethyl-3-cyano-4-(2- ethoxyphenyl)-5-(2,4- dichlorophenyl)carbamoyl-1,4-dihydro- pyridine | ¹ H NMR (DMSO-d6, 400 MHz): 9.17 (broad s, 1H), 8.67 (broad s, 1H), 7.87 (dd, J = 8.0, 1.6 Hz, 1H), 7.85 (t, J = 1.2 Hz, 1H), 6.82 (d, J = 0.8 Hz, 2H), 7.23 (td, J = 8.0, 1.2 Hz, 1H), 7.18 (dd, J = 8.4, 1.2 Hz, 1H), 7.04 (td, J 8.4, 1.2 Hz, 1H), 5.38 (s, 1H), 4.23 (m, 2H), 2.36 (s, 3H), 2.25 (s, 3H), 1.48 (t, J = 6.8 Hz, 3H), MS(ES⁺) 443, m/z (M + 1) 428 C₂₃H₂₁Cl₂N₃O₂ requires 443. |
| 122 | 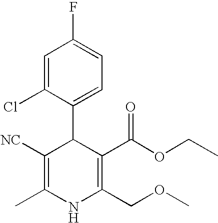 5-ethyl-2-methyl-3-cyano-4-(2-chloro- 4-fluorophenyl)-6-(2-methoxymethyl)- 1,4-dihydro-pyridine-5-carboxylate | ¹H NMR (DMSO-d6, 400 MHz):9.09 (broad s, 1H), 7.39 (dd, J = 9.2, 2.8 Hz, 1H), 7.33 (dd, J = 8.4, 6.0 Hz, 1H), 7.24 (td, J = 8.4, 2.4 Hz, 1H), 5.08 (s, 1H), 4.58 (s, 2H), 2.89 (m, 2H), 3.34 (s, 3H), 2.04 (s, 3H), 0.99 (t, J = 7.2 Hz, 3H). MS (ES⁺) 365, m/z (M + 1)⁺ C₁₈H₁₈ClFN₂O₃ requires 365 |
| 123 | 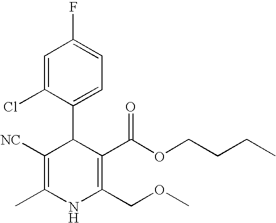 5-butyl-2-methyl-3-cyano-4-(2-chloro- 4-fluorophenyl)-6-(2-methoxymethyl)- 1,4-dihydro-pyridine-5-carboxylate | ¹H NMR (DMSO-d6, 400 MHz):9.05 (broad s, 1H), 7.35 (dd, J = 9.2, 2.8 Hz, 1H), 7.27 (dd, J = 8.8, 6.0 Hz, 1H), 7.18 (td, J = 8.4, 2.4 Hz, 1H), 5.03 (s, 1H), 4.54 (s, 2H), 3.30 (m, 1H), 1.98 (s, 3H), 1.29 (m, 2H), 0.90 (m, 2H), 0.69 (t, J = 7.6 Hz, 3H). MS (ES⁺) 393, m/z (M + 1)⁺ C₂₀H₂₂ClFN₂O₃ requires 393 |
| 124 | 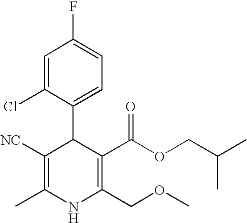 5-(3-methylpropyl)-2-methyl-3-cyano- 4-(2-chloro-4-fluorophenyl)-6-(2- methoxymethyl)-1,4-dihydro-pyridine- 5-carboxylate | ¹H NMR (DMSO-d6, 400 MHz):9.29 (broad s, 1H), 7.57 (dd, J = 8.8, 2.8 Hz, 1H), 7.49 (dd, J = 8.8, 6.4 Hz, 1H), 7.17 (td, J = 8.4, 2.8 Hz, 1H), 5.26 (s, 1H), 4.78 (s, 2H), 3.90 (dd, J = 10.4, 6.8 Hz, 1H), 3.52 (s, 3H), 2.22 (s, 3H), 1.89 (m, 1H), 0.80 (d, J = 6.8 Hz, 3H), 0.80 (d, J = 6.8 Hz, 3H). MS(ES⁺) 393, m/z (M + 1)⁺ C₂₀H₂₂ClFN₂O₃ requires 393 |
| 125 | 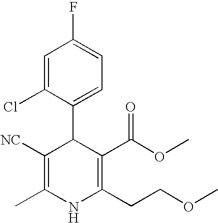 5-methyl-2-methyl-3-cyano-4-(2- chloro-4-fluorophenyl)-6-(2- methoxyethyl)-1,4-dihydro-pyridine-5- carboxylate | ¹H NMR (DMSO-d6, 400 MHz): 9.35 (broad s, 1H), 7.36 (dd, J = 9.2, 2.8 Hz, 1H), 7.31 (dd, J = 8.8, 6.4 Hz, 1H), 7.21 (td, J = 8.4, 2.8 Hz, 1H), 5.04 (s, 1H), 3.53 (m, 2H), 3.44 (s, 3H), 3.29 (s, 3H), 3.12 (m, 1H), 2.82 (m, 1H), 1.98 (s, 3H). MS (ES⁺) 365, m/z (M + 1)⁺ C₁₈H₁₈ClFN₂O₃ requires 365 |
| 126 | 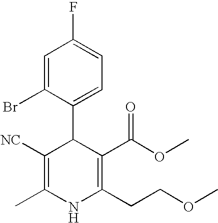 5-methyl-2-methyl-3-cyano-4-(2- bromo-4-fluorophenyl)-6-(2- methoxyethyl)-1,4-dihydro-pyridin-5- carboxylate | ¹H NMR (DMSO-d6, 400 MHz): 9.31 (broad s, 1H), 7.46 (dd, J = 8.8, 2.4 Hz, 1H), 7.27 (dd, J = 8.8, 6.4 Hz, 1H), 7.29 (td, J = 8.4, 2.8 Hz, 1H), 4.99 (s, 1H), 3.51 (m, 2H), 3.30 (s, 3H), 3.25 (s, 3H), 3.08 (m, 1H), 2.80 (m,1H), 1.95 (s, 3H). MS (ES⁺) 410, m/z (M + 1)⁺ C₁₈H₁₈BrFN₂O₃ requires 410 |
| 127 | 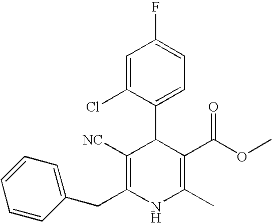 5-methyl-2-phenylmethyl-3-cyano-4- (2-chloro-4-fluorophenyl)-6-methyl- 1,4-dihydro-pyridine-5-carboxylate | ¹H NMR (DMSO-d6, 400 MHz): 9.31 (broad s, 1H), 7.29 (dd, J = 8.8, 2.4 Hz, 1H), 7.22 (m, 6H), 7.13 (td, J = 8.8, 2.4 Hz, 1H), 5.02 (s, 1H), 3.59 (d, J = 14.8 Hz, 1H), 3.52 (d, J = 14.8 Hz, 1H), 3.37 (s, 3H), 2.22 (s, 3H). MS (ES⁺) 397, m/z (M + 1)⁺ C₂₂H₁₈ClFN₂O₂ requires 397 |
| 128 | 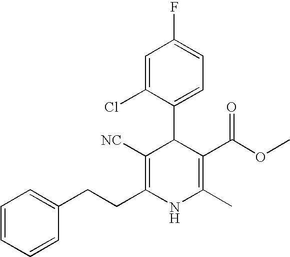 5-methyl-2-(2-phenyl)ethyl-3-cyano-4- (2-chloro-4-fluorophenyl)-6-methyl- 1,4-dihydro-pyridine-5-carboxylate | ¹H NMR (DMSO-d6, 400 MHz): 9.24 (broad s, 1H), 7.33 (dd, J = 8.8, 2.4 Hz, 1H), 7.25 (dd, J = 8.8, 6.4 Hz, 1H), 7.19 (td, J = 8.4, 2.8 Hz, 1H), 5.01 (s, 1H), 3.43 (s, 3H), 2.80 (m, 2H), 2.57 (m, 2H), 2.31 (s, 3H). MS (ES⁺) 411, m/z (M + 1)⁺ C₂₃H₂₀ClFN₂O₂ requires 411 |
| 129 | 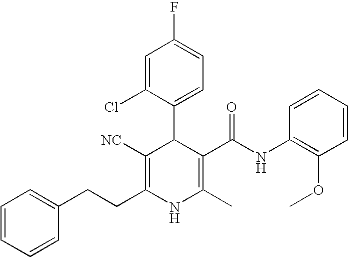 2-(2-phenylmethyl)-3-cyano-4-(2- chloro-4-fluorophenyl)-5-(2-chloro-4- fluorophenyl)carbamoyl-6-methyl-1,4- dihydro-pyridine | ¹H NMR (DMSO-d6, 400 MHz): 9.06 (broad s, 1H), 8.48 (broad s, 1H), 7.83 (dd, J = 8.0, 1.6 Hz, 1H), 7.50 (dd, J = 9.2, 2.8 Hz, 1H), 7.49 (s, 1H), 7.37 (m, 5H), 7.13 (td, J = 8.0, 1.2 Hz, 1H), 7.09 (dd, J 8.0, 1.2 Hz, 1H), 6.94 (td, J = 8.0, 1.2 Hz, 1H), 5.27 (s, 1H), 3.84 (s, 3H), 2.99 (m, 2H), 2.68 (m, 2H),2.26 (s, 3H). MS (ES⁺) 503, m/z (M + 1)⁺ C₂₉H₂₅ClFN₃O₂ requires 503 |
| 130 | 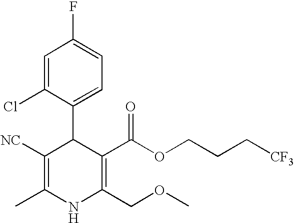 5-(3,3,3-trifluorobutyl)-2-methyl-3- cyano-4-(2-chloro-4-fluorophenyl)-1,4- dihydro-pyridine-5-carboxylate | MS (ES⁺) 446, m/z (M + 1) 447, C₂₀H₁₉ClF₄N₂O₃ requires 447 |
| 131 | 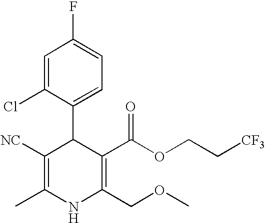 5-(3,3,3-trifluoropropyl)-2-methyl-3- cyano-4-(2-chloro-4-fluorophenyl)-6- methoxymethyl-1,4-dihydro-pyridine- 5-carboxylate | ¹H NMR (DMSO-d6): 9.36 (broad s, 1H), 7.65 (dd, J = 8.8, 2.4 Hz, 1H), 7.55 (dd, J = 8.8, 6.4 Hz, 1H), 7.46 (td, J = 8.4, 2.8 Hz, 1H), 5.31 (s, 1H), 4.81 (s, 2H), 4.33 (m, 2H), 3.61 (s, 3H), 2.30 (s, 3H), MS (ES⁺) 432, m/z (M + 1) 433, C₁₉H₁₇ClF₄N₂O₃ requires 433 |
| 132 | 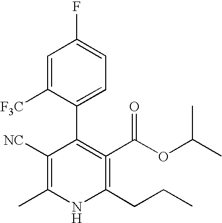 5-iso-propyl-2-methyl-3-cyano-4-(2- trifluoromethyl-4-fluorophenyl)-6- propyl-1,4-dihydro-pyridine-5- carboxylate | ¹H NMR (CDCl₃, 400 MHz): 7.53 (dd, J = 8.8, 5.6 Hz, 1H), 7.39 (dd, J = 9.2, 2.4 Hz, 1H), 7.29 (m, 1H), 5.88 (s, 1H), 5.15 (s, 1H), 4.96 (m, 1H), 2.71 (m, 2H), 2.16 (s, 3H), 1.75 (m, 2H), 1.19 (d, J = 6.4 Hz, 3H), 1.13 (t, J = 7.2 Hz, 3H), 0.89 (d, J = 6.4 Hz, 3H), MS (ES⁺) 410, m/z (M + 1) 411, C₂₁H₂₂F₄N₂O₂ requires 410. |
| 133 | 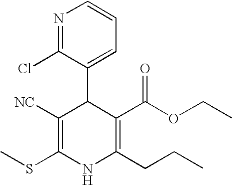 5-ethyl-2-thiomethyl-3-cyano-4-(2- chloro-3-pyridine)-6-propyl-1,4- dihydro-pyridine-5-carboxylate | ¹H NMR (CDCl₃, 400 MHz): 8.23 (dd, J = 4.8, 2.0 Hz, 1H), 7.55 (dd, J = 8.0, 2.0 Hz, 1H), 7.18 (dd, J = 4.8, 2.0 Hz, 1H), 6.39 (s, 1H), 5.17 (s, 1H), 3.93 (m, 2H), 2.65 (m, 2H), 2.40 (s, 3H), 1.58 (m, 2H), 1.05 (t, J = 7.2 Hz, 3H), 0.94 (t, J = 7.2 Hz, 3H). MS (ES⁺) 377, m/z (M + 1) 378,C₁₈H₂₀ClN₃O₂S requires 377. |
| 134 | 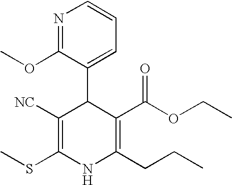 5-ethyl)-2-thiomethyl-3-cyano-4-(2- methoxy-3-pyridine)-6-propyl-1,4- dihydro-pyridine-5-carboxylate | ¹H NMR (CDCl₃, 400 MHz): 8.34 (dd, J = 5.6, 2.0 Hz, 1H), 7.70 (dd, J = 7.2, 2.0 Hz, 1H), 7.16 (dd, J = 7.2, 2.0 Hz, 1H), 6.47 (s, 1H), 5.20 (s, 1H), 4.29 (s, 3H), 4.19 (m, 2H), 2.91 (m, 2H), 2.62 (s, 3H), 1.82 (m, 2H), 1.30 (t, J = 7.2 Hz, 3H), 1.19 (t, J = 7.2 Hz, 3H). MS (ES⁺) 373, m/z (M + 1) 374, C₁₉H₂₃N₃O₃S requires 373. |
| 135 | 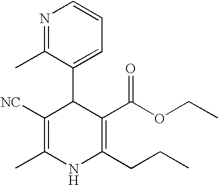 5-ethyl-2-methyl-3-cyano-4-(2-methyl- 3-pyridine)-6-propyl-1,4-dihydro- pyridine-5-carboxylate | ¹H NMR (CDCl₃, 400 MHz): 8.51 (d, J = 5.2 Hz, 1H), 8.31 (d, J = 8.0 Hz, 1H), 7.75 (m, 2H), 5.06 (s, 1H), 4.02 (q, J = 6.8, Hz, 2H), 3.03 (s, 3H), 2.73 (m, 2H), 2.10 (s, 3H), 1.65 (m, 2H), 1.15 (t, J = 6.8, Hz, 3H), 0.99 (t, J = 7.2 Hz, 3H). MS (ES⁺) 325, m/z (M + 1) 326, C₁₉H₂₃N₃O₂ requires 325. |
| 136 | 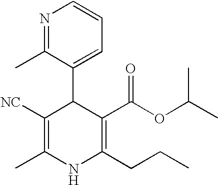 5-iso-propyl-2-methyl-3-cyano-4-(2- methyl-3-pyridine)-6-propyl-1,4- dihydro-pyridine-5-carboxylate | ¹H NMR (CDCl₃, 400 MHz): 8.66 (d, J = 2.8, Hz, 1H), 8.23 (d, J = 7.6 Hz, 1H), 7.72 (m, 1H), 6.66 (s, 1H), 5.22 (s, 1H), 4.90 (m, 1H), 3.03 (s, 3H), 2.72 (m, 2H), 2.12 (s, 3H), 1.67 (m, 2H), 1.17 (d, J = 6.4 Hz, 3H), 1.02 (m, 6H). MS (ES⁺) 339, m/z (M + 1) 340, C₂₀H₂₅N₃O₂ requires 339. |
| 137 | 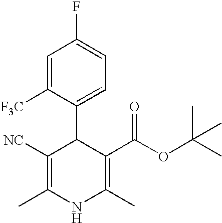 5-tert-butyl-2-methyl-3-cyano-4-(2- trifluoromethyl-4-fluorophenyl)-6- propyl-1,4-dihydro-pyridine-5- carboxylate | ¹H NMR (CDCl₃, 400 MHz): 7.35 (dd, J = 8.8, 5.6 Hz, 1H), 7.20 (dd, J = 9.6, 2.8 Hz, 1H), 7.10 (m, 1H), 5.61 (s, 1H), 4.92 (s, 1H), 2.14 (s, 3H), 1.95 (s, 3H), 1.12 (s, 9H). MS (ES⁺) 396, m/z (M + 1) 397, C₂₀H₂₀F₄N₂O₂ requires 396. |
| 138 | 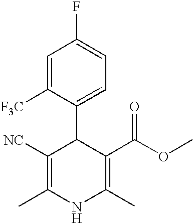 5-methyl-2,6-dimethyl-3-cyano-4-(2- trifluoromethyl-4-fluorophenyl)-1,4- dihydro-pyridine-5-carboxylate | ¹H NMR (CDCl₃, 400 MHz): 7.42 (dd, J = 8.8, 5.2 Hz, 1H), 7.35 (dd, J = 9.2, 2.8 Hz, 1H), 7.22 (m, 1H), 5.94 (s, 1H), 5.07 (s, 1H), 2.27 (s, 3H), 2.10 (s, 3H), 2.05 (s, 3H). MS (ES⁺) 354, m/z (M + 1) 355, C₁₇H₁₄F₄N₂O₂ requires 354. |
| 139 | 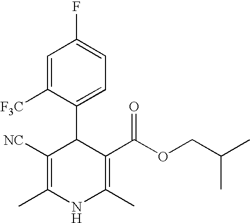 5-(2-methylpropyl)-2-methyl-3-cyano- 4-(2-trifluoromethyl-4-fluorophenyl)-6- methyl-1,4-dihydro-pyridine-5- carboxylate | ¹H NMR (CDCl₃, 400 MHz): 7.46 (dd, J = 8.8, 5.2 Hz, 1H), 7.32 (dd, J = 9.2, 2.8 Hz, 1H), 7.22 (m, 1H), 5.80 (s, 1H), 5.05 (s, 1H), 3.73 (m, 2H), 2.38 (s, 3H), 2.10 (s, 3H), 1.75 (m, 1H), 0.68 (t, J = 2.8 Hz, 6H). MS (ES⁺) 396, m/z (M + 1) 397, C₂₀H₂₀F₄N₂O₂ requires 396. |
| 140 | 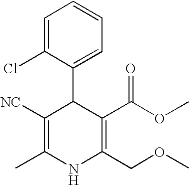 5-methyl-2-methyl-3-cyano-4-(2- chlorophenyl)-6-(2-methoxymethyl)- 1,4-dihydro-pyridine-5-carboxylate | ¹H NMR (DMSO-d6, 400 MHz): 9.03 (s, 1H), 7.32 (dd, J = 8.0, 1.2 Hz, 1H), 7.25 (dd, J = 8.0, 1.2 Hz, 1H), 7.22 (dd, J = 8.0, 1.6 Hz, 1H), 7.16 (m, 1H), 5.69 (s, 1H), 4.49 (s, 2H), 3.37 (s, 3H), 3.28 (s, 3H), 1.98 (s, 3H). MS (ES⁺) 332, m/z (M + 1) 333, C₁₇H₁₇ClN₂O₃ requires 332. |
| 141 | 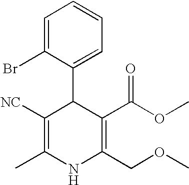 5-methyl-2-methyl-3-cyano-4-(2- cbromophenyl)-6-(2-methoxymethyl)- 1,4-dihydro-pyridine-5-carboxylate | ¹H NMR (CDCl₃, 400 MHz): 7.43 (d, J = 8.4 Hz, 1H), 7.17 (m, 2H), 7.09 (s, 1H), 6.97 (m, 1H), 5.17 (s, 1H), 4.60 (m, 2H), 3.45 (s, 3H), 3.41 (s, 3H), 2.03 (s, 3H). MS (ES⁺) 376, m/z (M + 1) 377, C₁₇H₁₇BrN₂O₃ requires 376. |
| 142 | 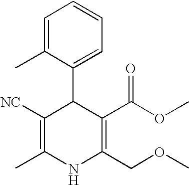 5-methyl-2-methyl-3-cyano-4-(2- methylphenyl)-6-(2-methoxymethyl)- 1,4-dihydro-pyridine-5-carboxylate | ¹H NMR (CDCl₃, 400 MHz): 7.10 (d, J = 7.2 Hz, 1H), 7.06 (dd, J = 6.4, 2.4 Hz, 1H), 7.00 (m, 3H), 4.84 (s, 1H), 4.60 (m, 2H), 3.45 (s, 3H), 3.41 (s, 3H), 2.44 (s, 3H), 2.02 (s, 3H). MS (ES⁺) 312, m/z (M + 1) 313, C₁₈H₂₀N₂O₃ requires 312. |
| 143 | 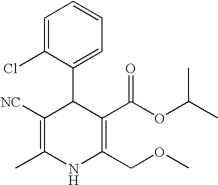 5-iso-propyl-2-methyl-3-cyano-4-(2- chlorophenyl)-6-(2-methoxymethyl)- 1,4-dihydro-pyridine-5-carboxylate | ¹H NMR (CDCl₃, 400 MHz): 7.30 (dd, J = 8.0, 1.2 Hz, 1H), 7.23 (m, 1H), 7.18 (m, 1H), 7.11 (dd, J = 7.6, 1.6 Hz, 1H), 7.08 (m, 1H), 5.23 (s, 1H), 4.79 (m, 1H), 4.68 (s, 2H), 3.47 (s, 3H), 2.07 (s, 3H), 1.15 (d, J = 6.0 Hz, 3H), 0.76 (d, J = 6.0 Hz, 3H), MS (ES⁺) 360, m/z (M + 1) 361, C₁₉H₂₁ClN₂O₃ requires 360. |
| 144 | 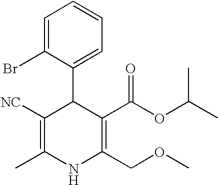 5-iso-propyl-2-methyl-3-cycno-4-(2- bromophenyl)-6-(2-methoxymethyl)- 1,4-dihydro-pyridine-5-carboxylate | ¹H NMR (CDCl₃, 400 MHz): 7.48 (d, J = 8.0 Hz, 1H), 7.22 (m, 2H), 7.08 (s, 1H), 7.01 (m, 1H), 5.23 (s, 1H), 4.79 (m, 1H), 4.68 (s, 2H), 3.47 (s, 3H), 2.07 (s, 3H), 1.16 (d, J = 6.0 Hz, 3H), 0.76 (d, J = 6.0 Hz, 3H), MS (ES⁺) 404, m/z (M + 1) 405, C₁₉H₂₁BrN₂O₃ requires 404. |
| 145 | 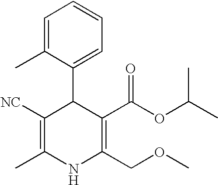 5-iso-propyl-2-methyl-3-cyano-4-(2- methylphenyl)-6-(2-methoxymethyl)- 1,4-dihydro-pyridine-5-carboxylate | ¹H NMR (CDCl₃, 400 MHz): 7.14 (m, 1H), 7.10 (dd, J = 6.0, 2.0 Hz, 1H), 7.03 (m, 3H), 4.89 (s, 1H), 4.79 (m, 1H), 4.68 (s, 2H), 3.45 (s, 3H), 2.50 (s, 3H), 2.05 (s, 3H), 1.14 (d, J = 6.4 Hz, 3H), 0.76 (d, J = 6.4 Hz, 3H). MS (ES⁺) 340, m/z (M + 1) 341, C₂₀H₂₄N₂O₃ requires 340. |
| 146 | 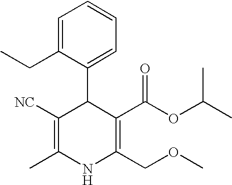 5-iso-propyl-2-methyl-3-cyano-4-(2- ethylphenyl)-6-(2-methoxymethyl)-1,4- dihydro-pyridine-5-carboxylate | ¹H NMR (CDCl₃, 400 MHz): 7.03 (m, 4H), 6.97 (s, 1H), 4.82 (s, 1H), 4.71 (m, 1H), 4.57 (s, 2H), 3.37 (s, 3H), 2.80 (m, 2H), 1.97 (s, 3H), 1.21 (t, J = 7.6 Hz, 3H), 1.04 (d, J = 6.4 Hz, 3H), 0.69 (d, J = 6.4 Hz, 3H). MS (ES⁺)354, m/z (M + 1) 355, C₂₁H₂₆N₂O₃ requires 354. |
| 147 | 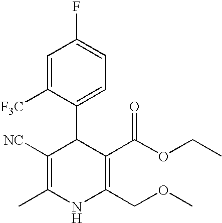 5-ethyl-2-methyl-3-cyano-4-(2- trifluoromethyl-4-fluorophenyl)-6-(2- methoxymethyl)-1,4-dihydro-pyridine- 5-carboxylate | ¹H NMR (CDCl₃, 400 MHz): 7.43 (dd, J = 8.8, 5.6 Hz, 1H), 7.29 (dd, J = 9.2, 2.8 Hz, 1H), 7.18 (m, 2H), 5.04 (s, 1H), 4.67 (m, 2H), 3.93 (m, 2H), 3.48 (s, 3H), 2.11 (s, 3H), 0.96 (t, J = 7.2 Hz, 3H). MS (ES⁺) 398, m/z (M + 1) 399, C₁₉H₁₈F₄N₂O₃ requires 398. |
| 148 | 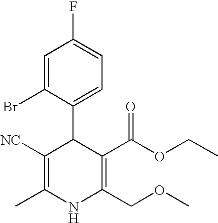 5-ethyl-2-methyl-3-cyano-4-(2-bromo- 4-fluorophenyl)-6-(2-methoxymethyl)- 1,4-dihydro-pyridine-5-carboxylate | ¹H NMR (CDCl₃, 400 MHz): 7.18 (dd, J = 8.0, 2.8 Hz, 1H), 7.15 (dd, J = 8.8, 6.0 Hz, 1H), 7.08 (s, 1H), 6.91 (m, 1H), 5.15 (s, 1H), 4.61 (m, 2H), 3.89 (m, 2H), 3.42 (s, 3H), 2.02 (s, 3H), 1.01 (t, J = 7.2 Hz, 3H). MS (ES⁺) 408, m/z (M + 1) 409, C₁₈H₁₈BrFN₂O₃ requires 408 |
| 149 | 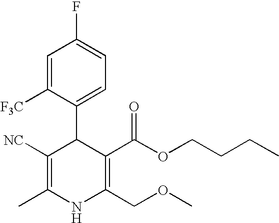 5-butyl-2-methyl-3-cyano-4-(2- trifluoromethyl-4-fluorophenyl)-6-(2- methoxymethyl)-1,4-dihydro-pyridine- 5-carboxylate | ¹H NMR (CDCl₃, 400 MHz): 7.33 (d, J = 8.8, 5.6 Hz, 1H), 7.20 (dd, J = 9.6, 2.8 Hz, 1H), 7.12 (s, 1H), 7.07 (m, 1H), 4.93 (s, 1H), 4.60 (m, 2H), 3.80 (m, 2H), 3.38 (s, 3H), 2.01 (s, 3H), 1.23 (m, 2H), 0.88 (m, 2H), 0.66 (t, J = 7.2 Hz, 3H). MS (ES⁺) 426, m/z (M + 1) 427,C₂₁H₂₂F₄N₂O₃ requires 426 |
| 150 | 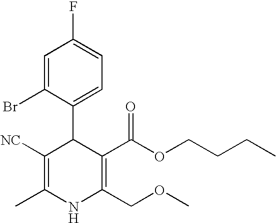 5-butyl-2-methyl-3-cyano-4-(2-bromo- 4-fluorophenyl)-6-(2-methoxymethyl)- 1,4-dihydro-pyridine-5-carboxylate | ¹H NMR (CDCl₃, 400 MHz): 7.15 (dd, J = 8.0, 2.8 Hz, 1H), 7.10 (dd, J = 8.8, 6.0 Hz, 1H), 7.04 (s, 1H), 6.87 (m, 1H), 5.09 (s, 1H), 4.57 (m, 2H), 3.82 (m, 2H), 3.37 (s, 3H), 1.97 (s, 3H), 1.31 (m, 2H), 0.95 (m, 2H), 0.69 (t, J = 7.2 Hz, 3H). MS (ES⁺) 436, m/z (M + 1) 437, C₂₀H₂₂BrFN₂O₃ requires 436 |
| 151 | 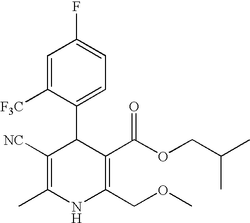 5-(2-methylpropyl)-2-methyl-3-cyano- 4-(2-trifluoromethyl-4-fluorophenyl)-6- (2-methoxymethyl)-1,4-dihydro- pyridine-5-carboxylate | ¹H NMR (CDCl₃, 400 MHz): 7.33 (dd, J = 8.8, 5.6 Hz, 1H), 7.20 (dd, J = 9.6, 2.8 Hz, 1H), 7.13 (s, 1H), 7.08 (m, 1H), 4.94 (s, 1H), 4.58 (s, 2H), 3.63 (m, 1H), 3.53 (m, 1H), 3.38 (s, 3H), 2.01 (s, 3H), 1.61 (m, 1H), 0.53 (d, J = 6.8 Hz, 3H), 0.50 (d, J = 6.8 Hz, 3H). MS (ES⁺) 426, m/z (M + 1) 427, C₂₁H₂₂F₄N₂O₃ requires 426 |
| 152 | 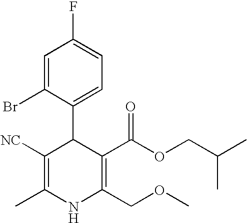 5-(2-methylpropyl)-2-methyl-3-cyano- 4-(2-bromo-4-fluorophenyl)-6-(2- methoxymethyl)-1,4-dihydro-pyridine- 5-carboxylate | ¹H NMR (CDCl₃, 400 MHz): 7.14 (dd, J = 8.0, 2.8 Hz, 1H), 7.10 (dd, J = 8.8, 6.0 Hz, 1H), 7.08 (s, 1H), 6.87 (m, 1H), 5.10 (s, 1H), 4.58 (s, 2H), 3.63 (m, 1H), 3.57 (m, 1H), 3.37 (s, 3H), 1.97 (s, 3H), 1.67 (m, 1H), 0.63 (d, J = 6.8 Hz, 3H), 0.55 (d, J = 6.8 Hz, 3H). MS (ES⁺) 436, m/z (M + 1) 437, C₂₀H₂₂BrFN₂O₃ requires 436 |
| 153 | 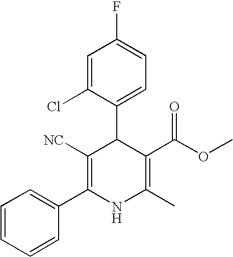 5-methyl-2-phenyl-3-cyano-4-(2- chloro-4-fluorophenyl)-6-methyl-1,4- dihydro-pyridine-5-carboxylate | ¹H NMR (CDCl₃, 400 MHz): 7.41 (m, 5H), 7.27 (dd, J = 8.8, 6.0 Hz, 1H), 7.04 (dd, J = 8.8, 2.8 Hz, 1H), 6.89 (m, 1H), 5.89 (s, 1H), 5.26 (s, 1H), 3.56 (s, 3H), 2.35 (s, 3H). MS (ES⁺) 382, m/z (M + 1) 383, C₂₁H₁₆ClFN₂O₂ requires 382 |
| 154 | 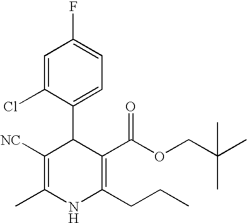 5-tert-amyl-2-methyl-3-cyano-4-(2- chloro-4-fluorophenyl)-6-propyl-1,4- dihydro-pyridine-5-carboxylate | ¹H NMR (CDCl₃, 400 MHz): 7.07 (dd, J = 8.4, 6.0 Hz, 1H), 6.97 (dd, J = 8.4, 2.4 Hz, 1H), 6.80 (m,1H), 5.76 (s, 1H), 5.11 (s, 1H), 3.55 (m, 2H), 2.68 (m, 1H), 2.54 (m, 1H), 1.94 (s, 3H), 1.56 (m, 2H), 0.92 (t, J = 7.2 Hz, 3H), 0.66 (s, 9H). MS (ES⁺) 404, m/z (M + 1) 405, C₂₂H₂₆ClFN₂O₂ requires 404 |
| 155 | 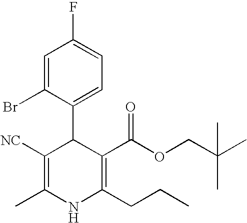 5-tert-amyl-2-methyl-3-cyano-4-(2- bromo-4-fluorophenyl)-6-propyl-1,4- dihydro-pyridine-5-carboxylate | ¹H NMR (CDCl₃, 400 MHz): 7.25 (dd, J = 8.0, 2.4 Hz, 1H), 7.17 (dd, J = 8.8, 6.0 Hz, 1H), 6.95 (m, 2H), 5.88 (s, 1H), 5.21 (s, 1H), 3.69 (m, 2H), 2.75 (m, 1H), 2.65 (m, 1H),2.04 (s, 3H), 1.66 (m, 2H), 1.02 (t, J = 7.2 Hz, 3H), 0.77 (s, 9H). MS (ES⁺) 448, m/z (M + 1) 449, C₂₂H₂₆BrFN₂O₂ requires 448 |
| 156 | 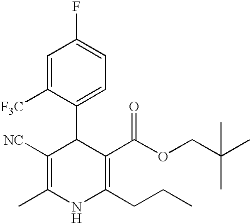 5-tert-amyl-2-methyl-3-cyano-4-(2- trifluoromethyl-4-fluorophenyl)-6- propyl-1,4-dihydro-pyridine-5- carboxylate | ¹H NMR (CDCl₃, 400 MHz): 7.34 (dd, J = 8.4,5.2 Hz, 1H), 7.19 (dd, J = 9.2, 2.8 Hz, 1H), 7.07 (m, 1H), 5.65 (s, 1H), 4.95 (s, 1H), 3.69 (m, 1H), 3.54 (m, 1H), 2.55 (m, 2H), 1.99 (s, 3H), 1.55 (m, 2H), 0.91 (t, J = 7.2 Hz, 3H), 0.63 (s, 9H). MS (ES⁺) 438, m/z (M + 1) 439,C₂₃H₂₆F₄N₂O₂ requires 438 |
| 157 | 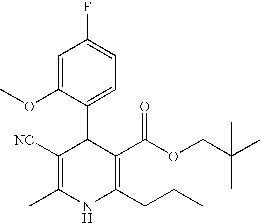 5-tert-amyl-2-methyl-3-cyano-4-(2- methoxy-4-fluorophenyl)-6-propyl-1,4- dihydro-pyridine-5-carboxylate | ¹H NMR (CDCl₃, 400 MHz): 6.87 (m, 1H), 6.44 (m, 2H), 5.68 (s, 1H), 5.01 (s, 1H), 3.69 (s, 3H), 3.53 (m, 1H), 3.43 (m, 1H), 2.73 (m, 1H), 2.55 (m, 1H), 1.91 (s, 3H), 1.57 (m, 2H), 0.93 (t, J = 7.2 Hz, 3H), 0.60 (s, 9H). MS (ES⁺) 400, m/z (M + 1) 401, C₂₃H₂₉FN₂O₃ requires 400 |
| 158 | 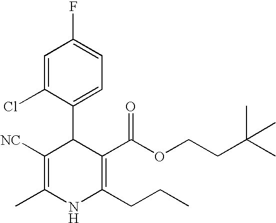 5-(3,3-dimethylbutyl)-2-methyl-3- cyano-4-(2-chloro-4-fluorophenyl)-6- propyl-1,4-dihydro-pyridine-5- carboxylate | ¹H NMR (CDCl₃, 400 MHz): 7.09 (dd, J = 8.4, 6.0 Hz, 1H),6.97 (dd, J = 8.4, 2.4 Hz, 1H), 6.80 (m, 1H), 5.64 (s, 1H), 5.08 (s, 1H), 3.85 (m, 2H), 2.65 (m, 1H), 2.50 (m, 1H), 1.94 (s, 3H), 1.56 (m, 2H), 1.30 (m, 1H), 1.24 (m, 1H), 0.91 (t, J = 7.2 Hz, 3H), 0.72 (s, 9H). MS (ES⁺) 418, m/z (M + 1) 419, C₂₃H₂₈ClFN₂O₂ requires 418 |
| 159 | 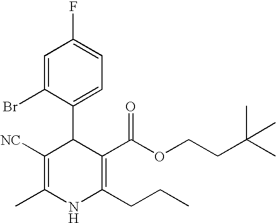 5-(3,3-dimethylbutyl)-2-methyl-3- cyano-4-(2-bromo-4-fluorophenyl)-6- propyl-1,4-dihydro-pyridine-5- carboxylate | ¹H NMR (CDCl₃, 400 MHz): 7.15 (dd, J = 8.4, 2.4 Hz, 1H), 7.09 (dd, J = 8.8, 6.0 Hz, 1H), 6.86 (m, 1H), 5.69 (s, 1H), 5.08 (s, 1H), 3.86 (m, 2H), 2.63 (m, 1), 2.52 (m, 1H), 1.94 (s, 3H), 1.56 (m, 2H), 1.31 (m, 1H), 1.26 (m, 1H), 0.91 (t, J = 7.2 Hz, 3H), 0.72 (s, 9H). MS (ES⁺) 462, m/z (M + 1) 463, C₂₃H₂₈BrFN₂O₂ requires 462 |
| 160 | 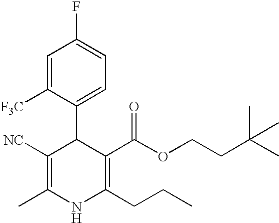 5-(3,3-dimethylbutyl)-2-methyl-3- cyano-4-(2-trifluoromethyl-4- fluorophenyl)-6-propyl-1,4-dihydro- pyridine-5-carboxylate | ¹H NMR (CDCl₃, 400 MHz): 7.32 (dd, J = 8.4, 5.2 Hz, 1H), 7.19 (dd, J = 9.2, 2.8 Hz, 1H), 7.07 (m, 1H), 5.78 (s, 1H), 4.93 (s, 1H), 3.88 (m, 2H), 2.59 (m, 1H), 2.51 (m, 1H), 1.97 (s, 3H), 1.55 (m, 2H), 1.17 (m, 2H), 0.90 (t, J = 7.2 Hz, 3H), 0.70 (s, 9H). MS (ES⁺) 452, m/z (M + 1) 453, C₂₄H₂₈F₄N₂O₂ requires 452 |
| 161 | 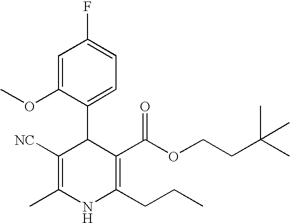 5-(3,3-dimethylbutyl)-2-methyl-3- cyano-4-(2-methoxy-4-fluorophenyl)-6- propyl-1,4-dihydro-pyridine-5- carboxylate | ¹H NMR (CDCl₃, 400 MHz): 6.89 (m, 1H), 6.46 (m, 2H), 5.65 (s, 1H), 4.93 (s, 1H), 3.84 (m, 2H), 3.71 (s, 3H), 2.68 (m, 1H), 2.46 (m, 1H), 1.91 (s, 3H), 1.55 (m, 2H), 1.23 (m, 2H), 0.91 (t, J = 7.2 Hz, 3H), 0.71 (s, 9H). MS (ES⁺) 414, m/z (M + 1) 415, C₂₄H₃₁FN₂O₃ requires 414 |
| 162 | 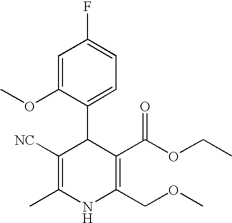 5-ethyl-2-methyl-3-cyano-4-(2- methoxy-4-fluorophenyl)-6-propyl-1,4- dihydro-pyridine-5-carboxylate | ¹H NMR (CDCl₃, 400 MHz): 7.00 (s, 1H), 6..95 (m, 1H), 6.50 (m, 2H), 4.98 (s, 1H), 4.61 (m, 2H), 3.87 (q, J = 7.2 Hz, 2H), 3.76 (s, 3H), 3.42 (s, 3H), 2.00 (s, 3H), 0.97 (d, J = 7.2 Hz, 3H). MS (ES⁺) 360, m/z (M + 1) 361, C₁₉H₂₁FN₂O₄ requires 360 |
| 163 | 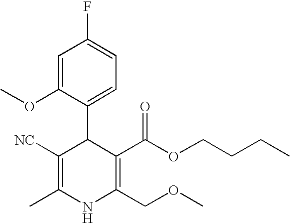 5-butyl-2-methyl-3-cyano-4-(2-bromo- 4-fluorophenyl)-6-(2-methoxymethyl)- 1,4-dihydro-pyridine-5-carboxylate | ¹H NMR (CDCl₃, 400 MHz): 7.02 (s, 1H), 6.94 (m, 1H), 6.50 (m, 2H), 4.99 (s, 1H), 4.63 (s, 2H), 3.86 (m, 2H), 3.76 (s, 3H), 3.41 (s, 3H), 1.99 (s, 3H), 1.32 (m, 2H), 1.04 (m, 2H), 0.72 (t, J = 7.2 Hz, 3H). MS (ES⁺) 388, m/z (M + 1) 389, C₂₁H₂₅FN₂O₄ requires 388 |
| 164 | 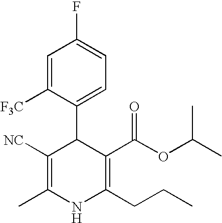 5-iso-propyl-2-methyl-3-cyano-4-(2- trifluoromethyl-4-fluorophenyl)-6- propyl-1,4-dihydro-pyridine-5- carboxylate | ¹H NMR (CDCl₃, 400 MHz): 7.33 (dd, J = 8.4, 5.2 Hz, 1H), 7.20 (dd, J = 9.2, 2.8 Hz, 1H), 7.07 (m, 1H), 5.59 (s, 1H), 4.96 (sm, 1H), 4.77 (m, 1H), 2.56 (m, 1H), 2.52 (m, 1H), 1.97 (s, 3), 1.54 (m, 2H), 1.00 (d, J = 6.0 Hz, 3H), 0.92 (t, J = 7.2 Hz, 3H), 0.69 (d, J = 6.0 Hz, 3H). MS (ES⁺) 410, m/z (M + 1) 411, C₂₁H₂₂F₄N₂O₂ requires 410 |
| 165 | 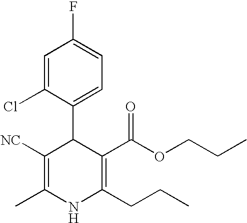 5-propyl-2-methyl-3-cyano-4-(2- chloro-4-fluorophenyl)-6-propyl-1,4- dihydro-pyridine-5-carboxylate | ¹H NMR (CDCl₃, 400 MHz): 7.09 (dd, J = 8.4, 6.0 Hz, 1H), 6.95 (dd, J = 8.4, 2.4 Hz, 1H), 6.80 (m, 1H), 5.67 (s, 1H), 5.09 (s, 1H), 3.77 (m, 2H), 2.65 (m, 1H), 2.51 (m, 1H), 1.94 (s, 3H), 1.55 (m, 2H), 1.37 (m, 2H), 0.91 (t, J = 7.2 Hz, 3H), 0.63 (t, J = 7.2 Hz, 3H). MS (ES⁺) 376, m/z (M + 1) 377, C₂₀H₂₂ClFN₂O₂ requires 376 |
| 166 | 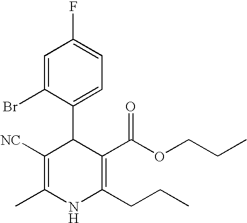 5-propyl-2-methyl-3-cyano-4-(2- bromo-4-fluorophenyl)-6-propyl-1,4- dihydro-pyridine-5-carboxylate | ¹H NMR (CDCl₃, 400 MHz): 7.25 (m, 1H), 7.19 (dd, J = 8.4, 6.0 Hz, 1H), 6.95 (m, 1H), 5.74 (s, 1H), 5.20 (s, 1H), 3.88 (m, 2H), 2.68 (m, 1H), 2.62 (m, 1H), 2.04 (s, 3H), 1.66 (m, 2H), 1.49 (m, 2H), 1.01 (t, J = 7.2 Hz, 3H), 0.72 (t, J = 7.2 Hz, 3H). MS (ES⁺) 420, m/z (M + 1) 421, C₂₀H₂₂BrFN₂O₂ requires 420 |
| 167 | 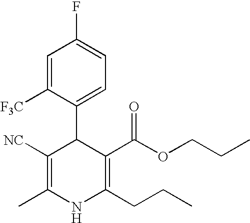 5-propyl-2-methyl-3-cyano-4-(2- trifluoromethyl-4-fluorophenyl)-6- propyl-1,4-dihydro-pyridine-5- carboxylate | ¹H NMR (CDCl₃, 400 MHz): 7.33 (dd, J = 8.4, 5.6 Hz, 1H), 7.19 (dd, J = 9.2, 2.8 Hz, 1H), 7.08 (m, 1H), 5.79 (s, 1), 4.94 (s, 1H), 3.77 (t, J = 6.8 Hz, 2H), 2.56 (m, 2H), 1.96 (s, 3H), 1.53 (m, 2H), 1.29 (m, 2H), 0.90 (t, J = 7.2 Hz, 3H), 0.57 (t, J = 7.2 Hz, 3H). MS (ES⁺) 410, m/z (M + 1) 411, C₂₁H₂₂F₄N₂O₂ requires 410 |
| 168 | 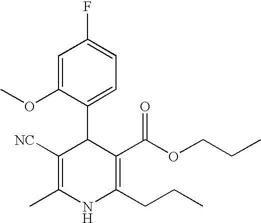 5-propyl-2-methyl-3-cyano-4-(2- methoxy-4-fluorophenyl)-6-propyl-1,4- dihydro-pyridine-5-carboxylate | ¹H NMR (CDCl₃, 400 MHz): 6.95 (m, 1H), 6.47 (m, 2H), 5.74 (s, 1H), 5.00 (s, 1H), 3.82 (m, 2), 3.75 (s, 3H), 2.73 (m, 1H), 2.73 (m, 1H), 2.52 (m, 1H), 1.95 (s, 3H), 1.60 (m, 2H), 1.38 (m, 2H), 0.95 (t, J = 7.2 Hz, 3H), 0.68 (t, J = 7.2 Hz, 3H). MS (ES⁺) 372, m/z (M + 1) 373, C₂₁H₂₅FN₂O₃ requires 372 |
| 169 | 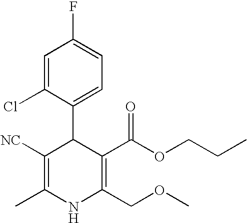 5-propyl-2-methyl-3-cyano-4-(2- chloro-4-fluorophenyl)-6-(2- methoxypropyl)-1,4-dihydro-pyridine- 5-carboxylate | ¹H NMR (CDCl₃, 400 MHz): 7.15 (dd, J = 8.4, 6.0 Hz, 1H), 7.09 (s, 1H), 7.00 (dd, J = 8.4, 2.4 Hz, 1H), 6.85 (m, 1H), 5.13 (s, 1H), 4.61 (s, 2H), 3.79 (m, 2H), 3.41 (s, 3H), 2.02 (s, 3H), 1.40 (m, 2H), 0.65 (t, J = 7.2 Hz, 3H). MS (ES⁺) 378, m/z (M + 1) 379, C₁₉H₂₀ClFN₂O₃ requires 378 |
| 170 | 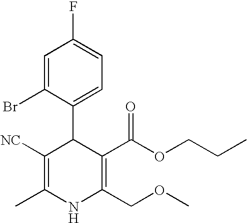 5-propyl-2-methyl-3-cyano-4-(2- bromo-4-fluorophenyl)-6-(2- methoxypropyl)-1,4-dihydro-pyridine- 5-carboxylate | ¹H NMR (CDCl₃, 400 MHz): 7.19 (dd, J = 8.4, 2.4 Hz, 1H), 7.15 (dd, J = 8.4, 6.0 Hz, 1H), 7.09 (s, 1H), 6.90 (m, 1H), 5.14 (s, 1H), 4.62 (s, 2), 3.80 (m, 2H), 3.41 (s, 3H), 2.01 (s, 3H), 1.40 (m, 2H), 0.64 (t, J = 7.2 Hz, 3H). MS (ES⁺) 422, m/z (M + 1) 423, C₁₉H₂₀BrFN₂O₃ requires 422 |
| 171 | 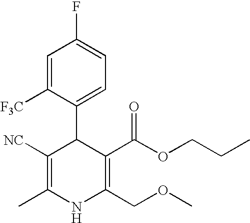 5-propyl-2-methyl-3-cyano-4-(2- trifluoromethyl-4-fluorophenyl)-6-(2- methoxyphenyl)-1,4-dihydro-pyridine- 5-carboxylate | ¹H NMR (CDCl₃, 400 MHz): 7.51 (dd, J = 8.4, 5.2 Hz, 1H), 7.38 (d, J = 9.2 Hz, 1H), 7.26 (m, 2H), 5.13 (s, 1H), 4.75 (m, 2H), 3.93 (m, 2H), 3.56 (s, 3H), 2.18 (s, 3H), 1.46 (m, 2H), 0.71 (t, J = 7.2 Hz, 3H). MS (ES⁺) 412, m/z (M + 1) 413, C₂₀H₂₀F₄N₂O₃ requires 412 |
| 172 | 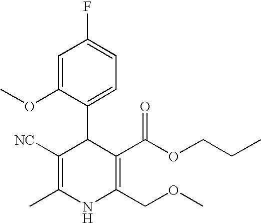 5-propyl-2-methyl-3-cyano-4-(2- methoxy-4-fluorophenyl)-6-(2- methoxypropyl)-1,4-dihydro-pyridine- 5-carboxylate | ¹H NMR (CDCl₃, 400 MHz): 7.00 (s, 1H), 6.93 (m, 1H), 6.47 (m, 2H), 4.99 (s, 1H), 4.63 (s, 2H), 3.81 (m, 2H), 3.75 (s, 3H), 3.41 (s, 3H), 1.99 (s, 3H), 1.35 (m, 2H), 0.65 (t, J = 7.2 Hz, 3H). MS (ES⁺) 374, m/z (M + 1) 375, C₂₀H₂₃FN₂O₄ requires 374 |
| 173 | 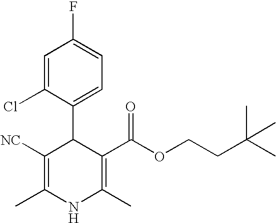 5-(3,3-dimethylbutyl)-2-methyl-3- cyano-4-(2-chloro-4-fluorophenyl)-6- methyl-1,4-dihydro-pyridine-5- carboxylate | ¹H NMR (CDCl₃, 400 MHz): 7.08 (dd, J = 8.4, 6.0 Hz, 1H), 6.95 (dd, J = 8.4, 2.4 Hz, 1H), 6.79 (m, 1H), 5.64 (s, 1H), 5.05 (s, 1H), 3.85 (m, 2H), 2.23 (s, 3H), 1.93 (s, 3H), 1.26 (m, 2H), 0.71 (s, 9H). MS (ES⁺) 390, m/z (M + 1) 391, C₂₁H₂₄ClFN₂O₂ requires 390 |
| 174 | 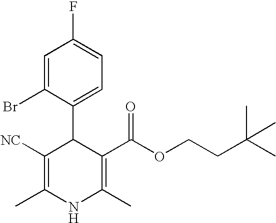 5-(3,3-dimethylbutyl)-2-methyl-3- cyano-4-(2-bromo-4-fluorophenyl)-6- methyl-1,4-dihydro-pyridine-5- carboxylate | ¹H NMR (CDCl₃, 400 MHz): 7.25 (m, 1H), 7.20 (dd, J = 8.4, 6.0 Hz, 1H), 6.95 (m, 1H), 5.81 (s, 1H), 5.17 (s, 1H), 3.96 (m, 2H), 2.34 (s, 3H), 2.03 (s, 3H), 1.38 (m, 2H), 0.82 (s,9H). MS (ES⁺) 434, m/z (M + 1) 435, C₂₁H₂₄BrFN₂O₂ requires 434 |
| 175 | 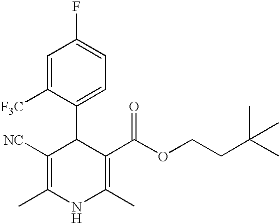 5-(3,3-dimethylbutyl)-2-methyl-3- cyano-4-(2-trifluoromethyl-4- fluorophenyl)-6-methyl-1,4-dihydro- pyridine-5-carboxylate | ¹H NMR (CDCl₃, 400 MHz): 7.32 (dd, J = 8.4, 5.2 Hz, 1H), 7.20 (dd, J = 9.2, 2.8 Hz, 1H), 7.08 (m, 1H), 5.68 (s, 1H), 4.93 (s, 1H), 3.85 (m, 2H), 2.24 (s, 3H), 1.97 (s, 3H), 1.15 (m, 2H), 0.70 (s, 9H). MS (ES⁺) 424, m/z (M + 1) 425, C₂₂H₂₄F₄N₂O₂ requires 424 |
| 176 | 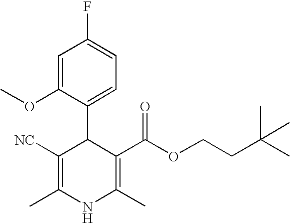 5-(3,3-dimethylbutyl)-2-methyl-3- cyano-4-(2-methoxy-4-fluorophenyl)-6- methyl-1,4-dihydro-pyridine-5- carboxylate | ¹H NMR (CDCl₃, 400 MHz): 7.10 (m,1H), 6.66 (m, 2H), 5.83 (s, 1H), 5.14 (s, 1H), 4.06 (m, 2H), 3.93 (s, 3H), 2.46 (s, 3H), 2.13 (s, 3H), 1.45 (m, 2H), 0.93 (s, 9H). MS (ES⁺) 386, m/z (M + 1) 387, C₂₂H₂₇FN₂O₃ requires 386 |
| 177 | 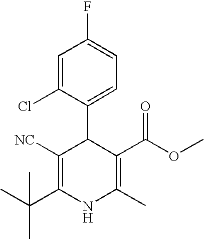 5-methyl-2-tert-butyl-3-cyano-4-(2- chloro-4-fluorophenyl)-6-methyl-1,4- dihydro-pyridine-5-carboxylate | ¹H NMR (CDCl₃, 400 MHz): 7.09 (dd, J = 8.8, 6.0 Hz, 1H), 6.95 (dd, J = 8.8, 2.4 Hz, 1H), 6.79 (m, 1H), 5.73 (s, 1H), 5.05 (s, 1H), 3.44 (s, 3H), 2.24 (s, 3H), 1.24 (s, 9H). MS (ES⁺) 362, m/z (M + 1) 363, C₁₉H₂₀ClFN₂O₂ requires 362 |
| 178 | 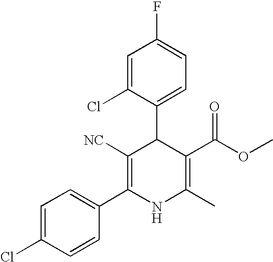 5-methyl-2-(4-chlorophenyl)-3-cyano- 4-(2-chloro-4-fluorophenyl)-6-methyl- 1,4-dihydro-pyridine-5-carboxylate | ¹H NMR (DMSO-d6, 400 MHz): 9.54 (s, 1H), 7.56 (m, 2H), 7.49 (m, 2H), 7.43 (dd, J = 8.8, 6.4 Hz, 1H), 7.37 (dd, J = 8.8, 2.4 Hz, 3H), 7.23 (m, 1H), 5.16 (s, 1H), 3.46 (s, 3H), 2.33 (s, 3H). MS (ES⁺) 416, m/z (M + 1) 417, C₂₁H₁₅Cl₂FN₂O₂ requires 416 |
| 179 | 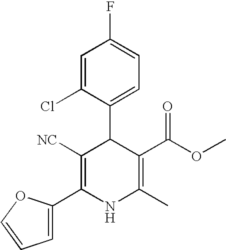 5-methyl-2-(2-furanyl)-3-cyano-4-(2- chloro-4-fluorophenyl)-6-methyl-1,4- dihydro-pyridine-5-carboxylate | ¹H NMR (CDCl₃, 400 MHz): 7.28 (d, J = 2.0 Hz, 1H), 7.11 (d, J = 3.6 Hz, 1H), 7.04 (dd, J = 8.8, 6.0 Hz, 1H), 6.87 (dd, J = 8.8, 2.8 Hz, 1H), 6.69 (m, 1H), 6.47 (s, 1H), 6.32 (dd, J = 3.6, 2.0 Hz, 1H), 5.10 (s, 1H), 3.35 (s, 3H), 2.25 (s, 3H). MS (ES⁺) 372, m/z (M + 1) 373,C₁₉H₁₄ClFN₂O₃ requires 372 |
| 180 | 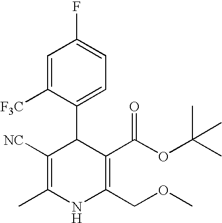 5-tert-butyl-2-methyl-3-cyano-4-(2- trifluoromethyl-4-fluorophenyl)-6-(2- methoxymethyl)-1,4-dihydro-pyridine- 5-carboxylate | ¹H NMR (CDCl₃, 400 MHz): 7.66 (d, J = 8.8, 5.6 Hz, 1H), 7.54 (dd, J = 9.2, 2.8 Hz, 1H), 7.43 (m, 1H), 7.34 (s, 1H), 5.29 (s, 1H), 4.84 (m, 2H), 3.72 (s, 3H), 2.32 (s, 3H), 1.44 (s, 9H). MS (ES⁺) 426, m/z (M + 1) 427, C₂₁H₂₂F₄N₂O₃ requires 426 |
| 181 | 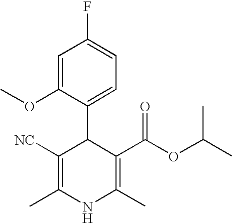 5-iso-propyl-2-methyl-3-cyano-4-(2- methoxy-4-fluorophenyl)-6-methyl-1,4- dihydro-pyridine-5-carboxylate | ¹H NMR (CDCl₃, 400 MHz): 7.13 (m, 1H), 6.65 (m, 2H), 5.65 (s, 1H), 5.17 (s, 1H), 4.93 (m, 1H), 3.94 (s, 3H), 2.46 (s, 3H) 2.14 (s, 3H), 1.24 (d, J = 6.0 Hz, 3H), 0.95 (d, J = 6.0 Hz, 3H). MS (ES⁺) 344, m/z (M + 1) 345, C₁₉H₂₁FN₂O₃ requires 344 |
| 182 | 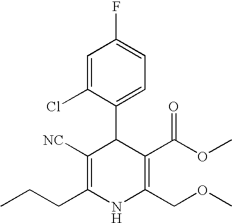 5-methyl-2-propyl-3-cyano-4-(2- chloro-4-fluorophenyl)-6- methoxymethyl-1,4-dihydro-pyridine- 5-carboxylate | ¹H NMR (DMSO-d6): 9.14 (broad s, 1H), 7.46 (dd, J = 9.2, 2.8 Hz, 1H), 7.41 (dd, J = 8.4, 6.0 Hz, 1H), 7.32 (td, J = 8.4, 2.8 Hz, 1H), 5.18 (s, 1H), 4.69 (d, J = 13.6 Hz, 1H), 4.64 (d, J = 13.6 Hz, 1H), 3.55 (s, 3H), 3.43 (s, 3H), 2.45 (m, 2H), 1.63 (m, 2H), 0.97 (t, J = 7.2 Hz, 3H), MS (ES⁺) 378, m/z (M+ 1) 379, C₁₉H₂₀ClFN₂O₃ requires 379 |
| 183 | 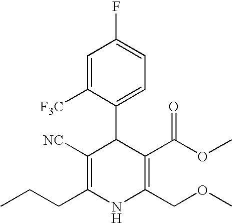 5-methyl-2-propyl-3-cyano-4-(2- trifluoromethyl-4-fluorophenyl)-6- methoxymethyl-1,4-dihydro-pyridine- 5-carboxylate | MS (ES⁺) 412, m/z (M + 1) 413, C₂₀H₂₀F₄N₂O₃ requires 413 |
| 184 | 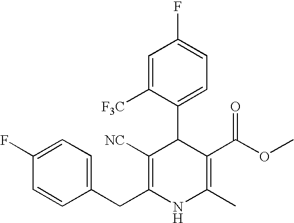 5-methyl-2-(4-fluorophenyl)methyl-3- cyano-4-(2-trifluoromethyl-4- fluorophenyl)-6-methyl-1,4-dihydro- pyridine-5-carboxylate | ¹H NMR (DMSO-d6): 9.46 (broad s, 1H), 7.52 (m, 3H), 7.28 (m, 2H), 7.16 (t, J = 6.3 Hz, 2H), 4.92 (s, 1H), 3.66 (d, J = 14.8 Hz, 1H), 3.59 (d, J = 14.8 Hz, 1H), 3.38 (s, 3H), 2.30 (s, 3H), MS (ES⁺) 448, m/z (M + 1) 449, C₂₃H₁₇F₅N₂O₂ requires 449 |
| 185 | 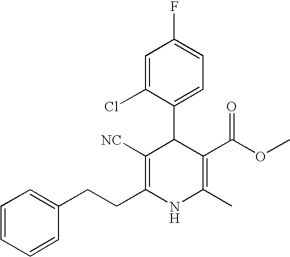 5-methyl-2-(4-fluorophenyl)methyl-3- cyano-4-(2-trifluoromethyl-4- fluorophenyl)-6-methyl-1,4-dihydro- pyridine-5-carboxylate | ¹H NMR (DMSO-d6): 9.63 (broad s, 1H), 7.45 (dd, J = 8.8, 2.4 Hz, 1H),7.30 (m, 2H), 7.24 (m, 5H), 4.92 (s, 1H), 3.31 (s, 3H), 2.88 (m, 2H), 2.61 (m, 2H), 2.06 (s, 3H), MS (ES⁺) 410, m/z (M + 1) 411, C₂₃H₂₀ClFN₂O₂ requires 411 |
| 186 | 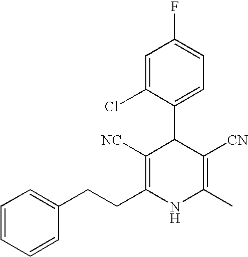 2-(2-phenyl)ethyl-3,5-dicyano-4-(2- chloro-4-fluorophenyl)-6-methyl-1,4- dihydro-pyridine | ¹H NMR (DMSO-d6): 9.80 (broad s, 1H), 7.62 (dd, J = 8.8, 2.4 Hz, 1H), 7.48 (m, 2H), 7.41 (m, 5H), 5.09 (s, 1H), 3.05 (m, 2H), 2.83 (m, 2H), 2.23 (s, 3H), MS (ES⁺) 378, m/z (M + 1) 379, C₂₂H₁₇ClFN₃ requires 379 |
| 187 | 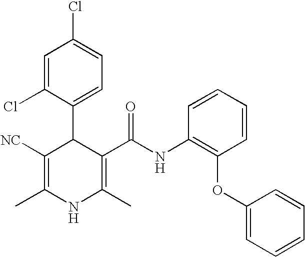 2,6-dimethyl-3-cyano-4-(2,4-dichloro)- 5-(2-methylphenyl)carbamoyl-1,4- dihydropyridine | ¹H NMR (DMSO-d6): 8.95 (broad s, 1H), 8.85 (broad s, 1H), 7.63 (m, 1H), 7.38 (m, 5H), 7.13 (t, J = 7.6 Hz, 1H), 7.06 (m, 2H), 6.86 (broad d, J = 7.6 Hz, 1H), 6.82 (m, 1H), 5.16 (s, 1H), 1.98 (s, 3H), 1.93 (s, 3H), MS (ES⁺) 490, m/z (M + 1) 491, C₂₇H₂₁Cl₂N₃O₂ requires 491 |
| 188 | 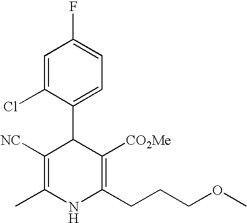 5-methyl-2-methyl-3-cyano-4-(2- chloro-4-fluorophenyl)-6-(3- methoxypropyl)-1,4-dihydro-pyridine- 5-carboxylate | ¹H NMR (CDCl₃): 7.45 (dd, J = 8.8, 6.0 Hz, 1H), 7.32 (dd, J = 8.8, 2.8 Hz, 1H), 7.22 (broad s, 1H), 7.16 (td, J = 8.0, 2.4 Hz, 1H), 5.43 (s, 1H), 3.77 (s, 3H), 3.73 (m, 2H), 3.65 (s, 3H), 3.22 (m, 2H), 3.08 (m, 1H), 2.14 (m, 2H), 1.88 (s, 3H), MS (ES⁺) 378, m/z (M + 1) 379, C₁₉H₂₀CLFN₂O₃ requires 379 |
| 189 | 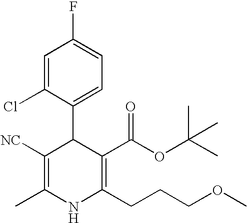 5-tert-butyl-2-methyl-3-cyano-4-(2- chloro-4-fluorophenyl)-6-(3- methoxypropyl)-1,4-dihydro-pyridine- 5-carboxylate | ¹H NMR (CDCl₃): 7.41 (dd, J = 8.4, 5.2 Hz, 1H), 7.24 (dd, J = 9.2, 2.8 Hz, 1H), 7.14 (td, J = 8.0, 2.8 Hz, 1H),6.86 (broad s, 1H), 4.97 (s, 1H), 3.42 (t, J = 6.0 Hz, 2H), 3.43 (s, 3H), 2.70 (m, 1H), 2.64 (m, 1H), 1.85 (m, 2H), 1.17 (s, 9H), MS (ES⁺) 421, m/z (M + 1) 422, C₂₃H₂₆ClFN₂O₃ requires 422 |
| 190 | 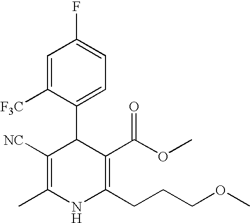 5-methyl-2-methyl-3-cyano-4-(2- trifluoromethyl-4-fluorophenyl)-6-(3- methoxypropyl)-1,4-dihydro-pyridine- 5-carboxylate | ¹H NMR (DMSO-d6): 9.31 (broad s, 1H), 7.53 (m, 3H), 4.86 (s, 1H), 3.40 (t, J = 6.0 Hz, 2H), 3.37 (s, 3H), 3.25 (s, 3H), 2.75 (m, 1H), 2.63 (m, 1H), 2.01 (s, 3H), 1.80 (m, 2H), MS (ES⁺) 412, m/z (M + 1) 413, C₂₀H₂₀F₄N₂O₃ requires 413 |
| 191 | 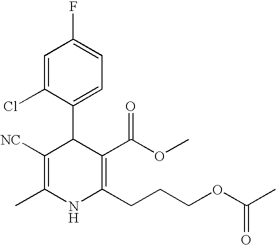 5-methyl-2-methyl-3-cyano-4-(2- chloro-4-fluorophenyl)-6-(3- acetoxypropyl)-1,4-dihydro-pyridine-5- carboxylate | ¹H NMR (DMSO-d6): 9.06 (broad s, 1H), 7.19 (dd, J = 8.8, 2.4 Hz, 1H), 7.14 (dd, J = 8.8, 6.4 Hz, 1H), 7.05 (td, J = 8.0, 2.8 Hz, 1H), 4.88 (s, 1H), 3.90 (t, J = 6.8 Hz, 2H), 3.28 (s, 3H), 2.63 (m, 1H), 2.52 (m, 1H), 1.86 (s, 3H), 1.83 (s, 3H), 1.70 (m, 2H). MS (ES⁺) 406, m/z (M + 1) 407,C₂₀H₂₀ClFN₂O₄ requires 407 |
| 192 | 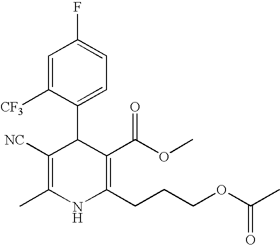 5-methyl-2-methyl-3-cyano-4-(2- trifluoromethyl-4-fluorophenyl)-6-(3- acetoxypropyl)-1,4-dihydro-pyridine-5- carboxylate | ¹H NMR (DMSO-d6): 9.10 (broad s, 1H), 7.19 (dd, J = 8.8, 2.4 Hz, 1H), 7.13 (dd, J = 8.8, 6.4 Hz, 1H), 7.04 (td, J = 8.4, 2.8 Hz, 1H), 4.88 (s, 1H), 3.90 (t, J = 6.8 Hz, 2H), 3.27 (s, 3H), 2.64 (m, 1H), 2.55 (m, 1H), 1.89 (s, 3H), 1.81 (s, 3H), 1.72 (m, 2H). MS (ES⁺) 440, m/z (M + 1)441, C₂₁H₂₀F₄N₂O₄ requires 441 |
| 193 | 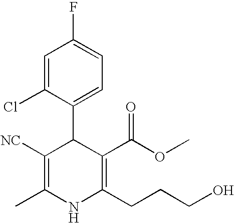 5-methyl-2-methyl-3-cyano-4-(2- chloro-4-fluorophenyl)-6-(3- hydroxypropyl)-1,4-dihydro-pyridine- 5-carboxylate | ¹H NMR (CDCl₃): 7.15 (dd, J = 8.4, 6.0 Hz, 1H), 7.01 (dd, J = 8.8, 2.8 Hz, 1H), 6.88 (td, J = 8.4, 2.8 Hz, 1H), 6.75 (broad s, 1H), 5.13 (s, 1H), 3.62 (m, 2H), 3.49 (s, 3H), 2.82 (m, 2H), 1.97 (s, 3H), 1.81 (m, 2H). MS (ES⁺) 364, m/z (M + 1) 365, C₁₈H₁₈ClFN₂O₃ requires 365 |
| 194 | 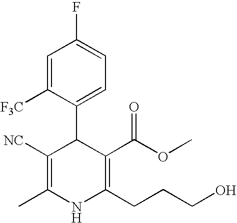 5-methyl-2-methyl-3-cyano-4-(2- trifluoromethyl-4-fluorophenyl)-6-(3- hydroxypropyl)-1,4-dihydro-pyridine- 5-carboxylate | ¹H NMR (CDCl₃): 7.17 (dd, J = 8.4, 6.0 Hz, 1H), 7.00 (dd, J = 8.8, 2.8 Hz, 1H), 6.86 (td, J = 8.4, 2.8 Hz, 1H), 6.74 (broad s, 1H), 5.14 (s, 1H), 3.65 (m, 2H), 3.43 (s, 3H), 2.80 (m, 2H), 1.99 (s, 3H), 1.88 (m, 2H). MS (ES⁺) 398, m/z (M + 1) 399, C₁₉H₁₈F₄N₂O₃ requires 399 |
| 195 | 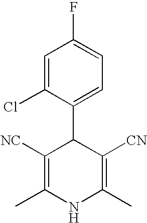 5-methyl-2-methyl-3-cyano-4-(2- trifluoromethyl-4-fluorophenyl)-6-(3- hydroxypropyl)-1,4-dihydro-pyridine- 5-carboxylate | ¹H NMR (DMSO-d6): 9.54 (broad s, 1H), 7.42 (dd, J = 8.4, 6.0 Hz, 1H), 7.37 (dd, J = 8.8, 2.8 Hz, 1H), 7.24 (td, J = 8.4, 2.4 Hz, 1H), 4.86 (s, 1H), 1.95 (s, 6H). MS (ES⁺) 287, m/z (M + 1) 288, C₁₅H₁₁ClFN₃ requires 288 |
| 196 | 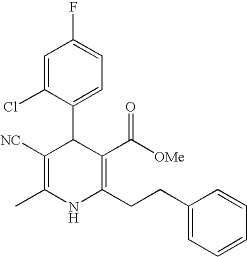 5-methyl-2-methyl-3-cyano-4-(2- chloro-4-fluorophenyl)-6-(2- phenylethyl)-1,4-dihydro-pyridine-5- carboxylate | ¹H NMR (DMSO-d6): 9.36 (broad s, 1H), 7.58 (dd, J = 9.2, 2.8 Hz, 1H), 7.52 (m, 2H), 7.44 (m, 5), 5.29 (s, 1H), 3.69 (s, 3H), 3.25 (m, 1H), 3.08 (m, 3H), 2.22 (s, 3H). MS (ES⁺) 410, m/z (M + 1) 411, C₁₅H₁₁ClFN₃ requires 411 |
| 197 | 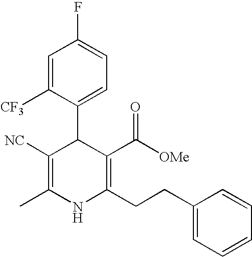 5-methyl-2-methyl-3-cyano-4-(2- trifluoromethyl-4-fluorophenyl)-6-(2- phenylethyl)-1,4-dihydro-pyridine-5- carboxylate | ¹H NMR (DMSO-d6): 9.41 (broad s, 1H), 7.52 (m, 3H), 7.33 (m, 4H), 7.25 (m, 1H), 4.91 (s, 1H), 3.42 (s, 3H), 3.03 (m, 1H), 2.91 (m, 3H), 2.02 (s, 3H). MS (ES⁺) 444, m/z (M + 1) 445, C₂₄H₂₀F₄N₂O₂ requires 445 |
| 198 | 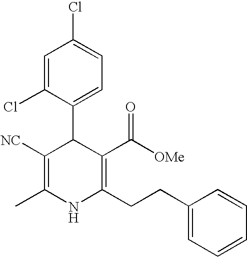 5-methyl-2-methyl-3-cyano-4-(2,4- dichlorophenyl)-6-(2-phenylethyl)-1,4- dihydro-pyridine-5-carboxylate | ¹H NMR (DMSO-d6): 9.20 (broad s, 1H), 7.39 (d, J = 2.0 Hz, 1H), 7.25 (dd, J = 8.4, 2.0 Hz, 1H), 7.18 (m, 4H), 7.06 (m, 3H), 4.92 (s, 1H), 3.32 (s, 3H), 2.87 (m, 1H), 2.71 (m, 3H), 1.86 (s, 3H). MS (ES⁺) 427, m/z (M + 1) 428, C₂₃H₂₀Cl₂N₂O₂ requires 428 |
| 199 | 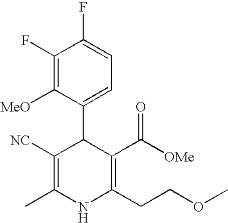 5-methyl-2-methyl-3-cyano-4-(2- methoxy-3,4-difluorophenyl)-6-(2- methoxyethyl)-1,4-ddihydro-pyridine-5- carboxylate | ¹H NMR (DMSO-d6): 9.19 (broad s, 1H), 7.03 (m, 1H), 6.87 (ddd, J = 8.4, 6.0, 2.0 Hz, 1H), 4.78 (s, 1H), 3.88 (d, J = 2.0 Hz, 3H), 3.47 (m, 2H), 3.40 (s, 3H), 3.22 (s, 3H), 3.06 (m, 1H), 2.81 (m, 1H), 1.91 (s, 3H). MS (ES⁺) 378, m/z (M + 1) 379, C₁₉H₂₀F₂N₂O₂ requires 379 |
| 200 | 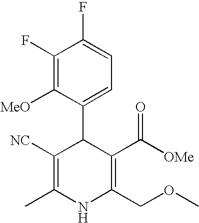 5-methyl-2-methyl-3-cyano-4-(2- methoxy-3,4-difluorophenyl)-6-(2- methoxymethyl)-1,4-dihydro-pyridine- 5-carboxylate | MS (ES⁺) 364, m/z (M + 1) 365, C₁₈H₁₈F₂N₂O₄ requires 365 |
| 201 | 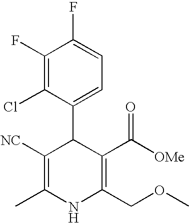 5-methyl-2-methyl-3-cyano-4-(2- chloro-3,4-difluorophenyl)-6-(2- methoxyethyl)-1,4-dihydro-pyridine-5- carboxylate | ¹H NMR (CDCl₃): 7.29 (broad s, 1H), 7.11 (m, 2H), 5.26 (s, 1H), 4.76 (d, J = 16.4 Hz, 1H), 4.69 (d, J = 16.4 Hz, 1H), 3.61 (s, 3H), 3.56 (s, 3H), 2.18 (s, 3H). MS (ES⁺) 368, m/z (M + 1) 369, C₁₇H₁₅ClF₂N₂O₃ requires 369 |
| 202 | 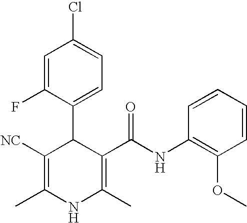 2,6-dimethyl-3-cyano-4-(2-fluoro-4- chloro)-5-(2- methoxyphenyl)carbamoyl-1,4- dihydropyridine | ¹H NMR (DMSO-d6): 8.95 (broad s, 1H), 8.47 (broad s, 1H), 7.70 (dd, J = 8.0, 1.6 Hz, 1H), 7.42 (dd, J = 10.0, 2.0 Hz, 1H), 7.38 (t, J = 8.0 Hz, 1H), 7.30 (dd, J = 8.4, 2.0 Hz, 1H), 7.03 (td, J = 7.6, 1.6 Hz, 1H), 6.97 (dd, J = 8.0, 1.6 Hz, 1H), 6.83 (td, J = 7.6, 1.6 Hz, 1H), 4.99 (s, 1H), 3.74 (s, 3H), 2.14 (s, 3H), 2.00 (s, 3H). MS (ES⁺) 411, m/z (M + 1) 412, C₂₂H₁₉ClFN₃O₂ requires 412 |
| 203 | 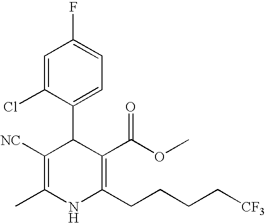 5-methyl-2-methyl-3-cyano-4-(2- chloro-4-fluorophenyl)-6-(5,5,5- trifluoropentyl)-1,4-dihydro-pyridine-5- carboxylate | ¹H NMR (DMSO-d6): 9.25 (broad s, 1H), 7.36 (dd, J = 8.8, 2.4 Hz, 1H), 7.27 (dd, J = 8.8, 2.4 Hz, 1), 7.19 (td, J = 8.4, 2.8 Hz, 1H), 5.04 (s, 1H), 3.44 (s, 3H), 2.61 (m, 1H), 2.52 (m, 1H), 2.29 (m, 2H), 2.01 (s, 3H), 1.64 (m, 2H), 1.57 (m, 2H). MS (ES⁺) 430, m/z (M + 1) 431,C₂₀H₁₉ClF₄N₂O₂ requires 431 |
| 204 | 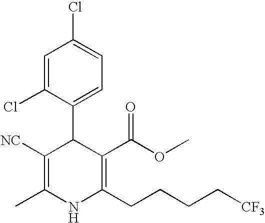 5-methyl-2-methyl-3-cyano-4-(2,4- dichlorophenyl)-6-(5,5,5- trifluoropentyl)-1,4-dihydro-pyridine-5- carboxylate | ¹H NMR (DMSO-d6): 9.49 (broad s, 1H), 7.47 (d, J = 2.2 Hz, 1H), 7.34 (dd, J = 8.4, 2.0 Hz, 1H), 7.19 (d, J = 8.4 Hz, 1H), 4.98 (s, 1H), 3.37 (s, 3H), 2.66 (m, 1H), 2.56 (m, 1H), 2.24 (m, 2H), 1.93 (s, 3H), 1.55 (m, 2H), 1.49 (m, 2H). MS (ES⁺) 447, m/z (M + 1) 448, C₂₀H₁₉Cl₂F₃N₂O₂ requires 448 |
| 205 | 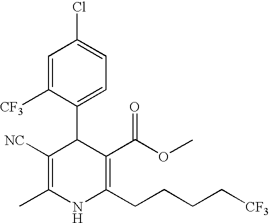 5-methyl-2-methyl-3-cyano-4-(2- trifluoromethyl-4-fluorophenyl)-6- (5,5,5-trifluoropentyl)-1,4-dihydro- pyridine-5-carboxylate | ¹H NMR (DMSO-d6): 9.32 (broad s, 1H), 7.53 (m, 3H), 4.88 (s, 1H), 3.39 (s, 3H), 2.74 (m, 1H), 2.55 (m, 1H), 2.27 (m, 2H), 2.02 (s, 3H), 1.62 (m, 2H), 1.48 (m, 2H). MS (ES⁺) 464, m/z (M + 1) 465, C₂₁H₁₉F₇N₂O₂ requires 465 |
| 206 | 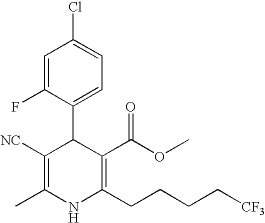 5-methyl-2-methyl-3-cyano-4-(2- fluoro-4-chlorophenyl)-6-(5,5,5- trifluoropentyl)-1,4-dihydro-pyridine-5- carboxylate | ¹H NMR (DMSO-d6): 9.49 (broad s, 1H), 7.58 (dd, J = 10.0, 2.0 Hz, 1H),7.47 (dd, J = 8.4, 2.0 Hz, 1H), 7.41 (t, J = 8.0 Hz, 1H), 5.02 (s, 1H), 3.68 (s, 3H), 2.95 (m, 1H), 2.83 (m, 1H), 2.51 (m, 2H), 2.22 (s, 3H), 1.79 (m, 2H), 1.48 (m, 2H). MS (ES⁺) 430, m/z (M + 1) 431, C₂₀H₁₉ClF₄N₂Ohd 2 requires 431 |
| 207 | 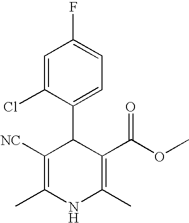 5-methyl-2,6-dimethyl-3-cyano-4-(2- chloro-4-fluorophenyl)-1,4-dihydro- pyridine-5-carboxylate | ¹H NMR (DMSO-d6): 9.31 (broad s, 1H), 7.41 (dd, J = 8.1, 2.4 Hz, 1H), 7.37 (dd, J = 8.8, 2.4 Hz, 1H), 7.26 (td, J = 8.4, 2.8 Hz, 1H), 5.09 (s, 1H), 3.49 (s, 3H), 2.35 (s, 3H), 2.05 (s, 3H). MS (ES⁺) 320, m/z (M + 1) 321, C₁₆H₁₄ClFN₂O₂ requires 321 |
| 208 | 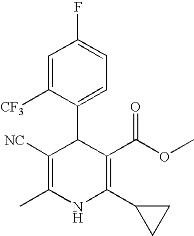 5-methyl-2-methyl-3-cyano-4-(2- trifluoromethyl-4-fluorophenyl)-6- cyclopropyl-1,4-dihydro-pyridine-5- carboxylate | ¹H NMR (DMSO-d6): 8.28 (broad s, 1H), 7.64 (td, J = 8.4, 2.8 Hz, 1H), 7.57 (m, 2H), 4.93 (s, 1H), 3.48 (s, 3H), 2.83 (m, 1H), 2.10 (s, 3H), 0.90 (m, 1H), 0.94 (m, 3H). MS (ES⁺) 380, m/z (M + 1) 381, C₁₉H₁₆F₄N₂O₂ requires 381 |
| 209 | 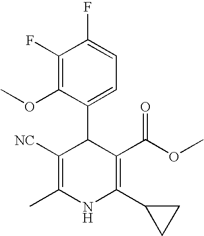 5-methyl-2-methyl-3-cyano-4-(2- methoxy-3,4-difluorophenyl)-6- cyclopropyl-1,4-dihydro-pyridine-5- carboxylate | ¹H NMR (DMSO-d6): 8.08 (broad s, 1H), 7.10 (m, 1H), 6.87 (ddd, J = 8.4, 6.0, 1.2 Hz, 1H), 4.83 (s, 1H), 3.47 (s, 3H), 3.12 (s, 3H), 2.83 (m, 1H), 1.99 (s, 3H), 1.00 (m, 1H), 0.86 (m, 3H). MS (ES⁺) 360, m/z (M + 1) 361, C₁₉H₁₈F₂N₂O₃ requires 361 |
| 210 | 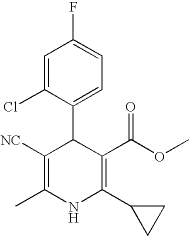 5-methyl-2-methyl-3-cyano-4-(2- chloro-4-fluorophenyl)-6-cyclopropyl- 1,4-dihydro-pyridine-5-carboxylate | ¹H NMR (DMSO-d6): 8.08 (broad s, 1H), 7.32 (dd, J = 8.8, 2.0 Hz, 1H), 7.21 (m, 2H), 5.01 (s, 1H), 3.43 (s, 3H), 2.81 (m, 1H), 1.97 (s, 3H), 0.97 (m, 1H), 0.86 (m, 3H). MS (ES⁺) 346, m/z (M + 1) 347, C₁₈H₁₆ClFN₂O₂ requires 347 |
| 211 | 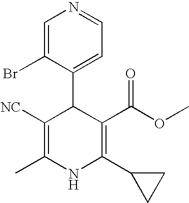 5-methyl-2-methyl-3-cyano-4-(2- bromo-4-pyridyl)-6-cyclopropyl-1,4- dihydro-pyridine-5-carboxylate | ¹H NMR (DMSO-d6): 8.68 (s, 1H), 8.52 (d, J = 4.8 Hz, 1H), 8.22 (broad s, 1H), 7.24 (d, J = 4.8 Hz, 1H), 5.03 (s, 1H), 3.46 (s, 3H), 2.88 (m, 1H), 2.02 (s, 3H), 1.18 (m, 1H), 0.90 (m, 3H). MS (ES⁺) 374, m/z (M + 1) 375, C₁₇H₁₆BrN₃O₂ requires 375 |
| 212 | 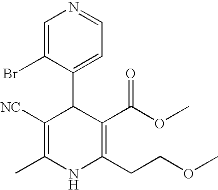 5-methyl-2-methyl-3-cyano-4-(2- bromo-4-pyridyl)-6-2-methoxyethyl- 1,4-dihydro-pyridine-5-carboxylate | ¹H NMR (DMSO-d6): 9.46 (broad s, 1H), 8.71 (s, 1H), 8.54 (d, J = 5.2 Hz, 1H), 7.33 (d, J = 5.2 Hz, 1H), 5.06 (s, 1H), 3.59 (m, 2H), 3.47 (s, 3H), 3.35 (s, 3H), 3.19 (m, 1H), 2.87 (m, 1H), 2.03 (s, 3H). MS (ES⁺) 392, m/z (M + 1) 393, C₁₇H₁₈BrN₃O₃ requires 393 |
| 213 | 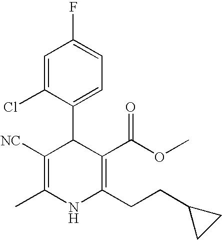 5-methyl-2-methyl-3-cyano-4-(2- chloro-4-fluorophenyl)-6-2- cyclopropylethyl-1,4-dihydro-pyridine- 5-carboxylate | ¹H NMR (DMSO-d6): 9.11 (broad s, 1H), 7.23 (dd, J = 9.2, 2.8 Hz, 1H), 7.17 (dd, J = 8.8, 6.4 Hz, 1H), 7.11 (td, J = 8.4, 2.4 Hz, 1H), 4.93 (s, 1H), 3.30 (s, 3H), 2.62 (m, 2H), 2.40 (s, 3H), 1.37 (m, 2H), 0.65 (m, 1H), 0.32 (m, 2H), 0.00 (m, 2H). MS (ES⁺) 374, m/z (M + 1) 375, C₂₀H₂₀ClFN₂O₂ requires 375 |
| 214 | 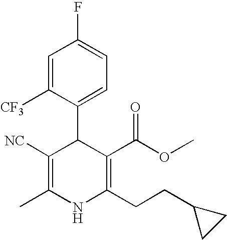 5-methyl-2-methyl-3-cyano-4-(2- trifluoromethyl-4-fluorophenyl)-6-2- cyclopropylethyl-1,4-dihydro-pyridine- 5-carboxylate | ¹H NMR (DMSO-d6): 9.18 (broad s, 1H), 7.43 (m, 3H), 4.76 (ms, 1H), 3.28 (s, 3H), 2.66 (m, 2H), 1.91 (s, 3H), 1.37 (m, 2H), 0.62 (m, 1H), 0.32 (m, 2H), 0.00 (m, 2H). MS (ES⁺) 408, m/z (M + 1) 409, C₂₁H₂₀F₄N₂O₂ requires 409 |
| 215 | 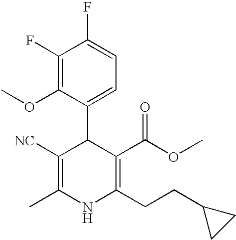 5-methyl-2-methyl-3-cyano-4-(2- methoxy-3,4-difluorophenyl)-6-2- cyclopropylethyl-1,4-dihydro-pyridine- 5-carboxylate | ¹H NMR (DMSO-d6): 9.06 (broad s, 1H), 6.99 (td, J = 9.6, 7.6 Hz, 1H), 6.78 (ddd, J = 8.0, 6.0, 2.0 Hz, 1H), 4.74 (s, 1H), 3.83 (s, 3H), 3.35 (s, 3H), 2.66 (m, 2H), 1.88 (s, 3H), 1.36 (m, 2H), 0.63 (m, 1H), 0.32 (m, 2H), 0.00 (m, 2H). MS (ES⁺) 408, m/z (M + 1) 409, C₂₁H₂₀F₄N₂O₂ requires 409 |
| 216 | 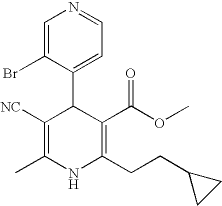 5-methyl-2-methyl-3-cyano-4-(2- bromo-4-pyridyl)-6-(2- cyclopropylethyl)-1,4-dihydro- pyridine-5-carboxylate | ¹H NMR (DMSO-d6): 9.21 (broad s, 1H), 8.55 (s, 1H), 8.40 (d, J = 4.8 Hz, 1H), 7.14 (d, J = 4.8 Hz, 1H), 4.91 (s, 1H), 3.32 (s, 3H), 2.67 (m, 2H), 1.89 (s, 3H), 1.34 (m, 2H), 0.63 (m, 1H), 0.32 (m, 2H), 0.00 (m, 2H). MS (ES⁺) 402, m/z (M + 1) 403, C₁₉H₂₀BrN₃O₂ requires 403 |
| 217 | 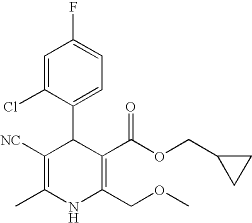 5-cyclopropylmethyl-2-methyl-3- cyano-4-(2-chloro-4-fluorophenyl)-6- (2-methoxyethyl)-1,4-dihydro-pyridine- 5-carboxylate | MS (ES⁺) 390, m/z (M + 1) 391, C₂₀H₂₀ClFN₂O₃ requires 391 |
| 218 | 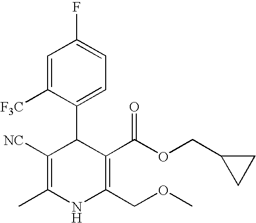 5-cyclopropylmethyl-2-methyl-3- cyano-4-(2-trifluoromethyl-4- fluorophenyl)-6-(2-methoxyethyl)-1,4- dihydro-pyridine-5-carboxylate | MS (ES⁺) 424, m/z (M + 1) 425, C₂₁H₂₀F₄N₂O₃ requires 425 |
| 219 | 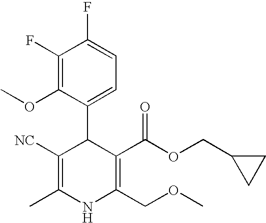 5-cyclopropylmethyl-2-methyl-3- cyano-4-(2-methoxy-3,4- difluorophenyl)-6-(2-methoxyethyl)- 1,4-dihydro-pyridine-5-carboxylate | MS (ES⁺) 404, m/z (M + 1) 405, C₂₁H₂₂F₂N₂O₄ requires 405 |
| 220 | 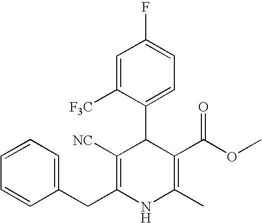 5-methyl-2-phenylmethyl-3-cyano-4- (2-trifluoromethyl-4-fluorophenyl)-6- methyl-1,4-dihydro-pyridine-5- carboxylate | ¹H NMR (CDCl₃, 400 MHz): 7.59 (dd, J = 8.8, 6.0 Hz, 1H), 7.52 (m, 4H), 7.39 (m, 3H), 5.73 (s, 1H), 5.28 (s, 1H), 3.92 (m, 2H), 3.66 (s, 3H), 2.42 (s, 3H). MS (ES⁺) 430, m/z (M + 1) 431, C₂₃H₁₈F₄N₂O₂ requires 430. |
| 221 | 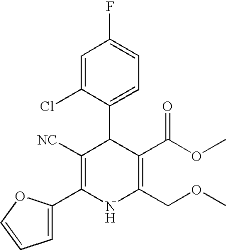 5-methyl-2-(2-furyl)-3-cyano-4-(2- chloro-4-fluorophenyl)-6-(2- methoxymethyl)-1,4-dihydro-pyridine- 5-carboxylate | ¹H NMR (CDCl₃, 400 MHz): 8.19 (s, 1H), 7.54 (d, J = 1.6 Hz, 1H), 7.34 (d, J = 4.0 Hz, 1H), 7.28 (dd, J = 8.8, 6.0 Hz, 1H), 7.10 (dd, J = 8.8, 2.4 Hz, 1H), 6.93 (dt, J = 8.8, 2.4 Hz, 1H), 6.56 (dd, J = 4.0, 1.6 Hz, 1H), 5.43 (s, 1H), 4.75 (m, 2H), 3.58 (s, 3H), 3.55 (s, 3H). MS (ES⁺) 402, m/z(M + 1) 403, C₂₀H₁₆ClFN₂O₄ requires 402 |
| 222 | 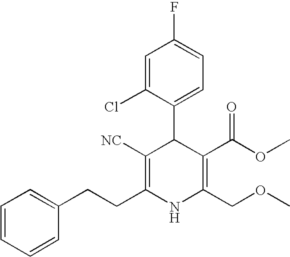 5-methyl-2-(2-phenylethyl)-3-cyano-4- (2-choloro-4-fluorophenyl)-6-(2- methoxymethyl)-1,4-dihydro-pyridine- 5-carboxylate | ¹H NMR (CDCl₃, 400 MHz): 7.23 (m, 2H), 7.18 (m, 1H), 7.13 (m, 2H), 7.04 (dd, J = 8.8, 6.0 Hz, 1H), 7.01 (dd, J = 8.8, 2.4 Hz, 1H), 6.91 (s, 1H), 6.85 (dt, J = 8.8, 2.4 Hz, 1H), 5.13 (s, 1H), 4.49 (m, 2H), 3.47 (s, 3H), 3.30 (s, 3H), 2.84 (m, 2H), 2.67 (m, 2H). MS (ES⁺) 440, m/z (M + 1) 441, C₂₄H₂₂ClFN₂O₃ requires 440 |
| 223 | 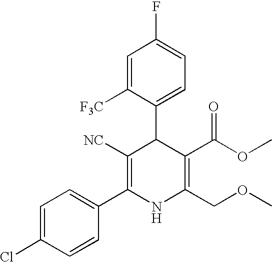 5-methyl-2-(4-chlorophenyl)-3-cyano- 4-(2-chloro-4-fluorophenyl)-6-(2- methoxymethyl)-1,4-dihydro-pyridine- 5-carboxylate | ¹H NMR (CDCl₃, 400 MHz): 7.56 (dd, J = 8.8, 5.6 hz, 1H), 7.48 (s, 1H), 7.45 (m, 4H), 7.36 (dd, J = 8.8, 2.8 Hz, 1H), 7.24 (dt, J = 8.8, 2.8 Hz, 1H), 5.21 (s, 1H), 4.76 (m, 2H), 3.53 (s, 3H), 3.51 (s, 3H). MS (ES⁺) 480, m/z (M + 1) 481, C₂₂H₁₇ClF₄N₂O₃ requires 480 |
| 224 | 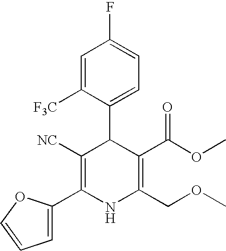 5-methyl-2-(2-furyl)-3-cyano-4-(2- trifluoromethyl-4-fluorophenyl)-6-(2- methoxymethyl)-1,4-dihydro-pyridine- 5-carboxylate | ¹H NMR (CDCl₃, 400 Mhz): 8.12 (s, 1H), 7.39 (d, J = 1.6 Hz, 1H), 7.36 (dd, J = 8.8, 5.6 Hz, 1H), 7.23 (d, J = 4.0 Hz, 1H), 7.18 (dd, J = 8.8, 2.8 Hz, 1H), 7.04 (dt, J = 8.8, 2.8 Hz, 1H), 6.41 (dd, J = 4.0, 1.6 Hz, 1H), 5.03 (s, 1H), 4.63 (s, 2H), 3.41 (s, 3H), 3.38 (s, 3H). MS (ES⁺)436, m/z (M + 1) 437, C₂₁H₁₆F₄N₂O₄ requires 436 |
| 225 | 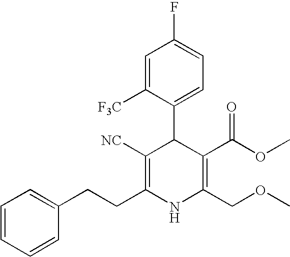 5-methyl-2-(2-phenylethyl))-3-cyano-4- (2-trifluoromethyl-4-fluorophenyl)-6- (2-methoxymethyl)-1,4-dihydro- pyridine-5-carboxylate | ¹H NMR (CDCl₃, 400 MHz): 7.22 (m, 4H), 7.08 (m, 4H), 6.89 (s, 1H), 4.93 (s, 1H), 4.48 (m, 2H), 3.37 (s, 3H), 3.26 (s, 3H), 2.82 (m, 2H), 2.66 (m, 2H). MS (ES⁺) 474, m/z (M + 1) 475, C₂₅H₂₂F₄N₂O₃ requires 474 |
| 226 | 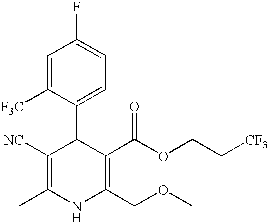 5-(3,3,3-trifluoropropyl)-2-methyl-3- cyano-4-(2-trifluoromethyl-4- fluorophenyl)-6-(2-methoxymethyl)- 1,4-dihydro-pyridine-5-carboxylate | ¹H NMR (CDCl₃, 400 MHz): 7.51 (dd, J = 8.4, 5.6 Hz, 1H), 7.41 (dd, J = 9.2, 2.8 Hz, 1H), 7.38 (s, 1H), 7.29 (dt, J = 8.4, 2.8 Hz, 1H), 5.12 (s, 1H), 4.76 (s, 2H), 4.23 (m, 2H), 3.59 (s, 3H), 2.35 (m, 2H), 2.21 (s, 3H). MS (ES⁺) 466, m/z (M + 1) 467, C₂₀H₁₇F₇N₂O₃ requires 466 |
| 227 | 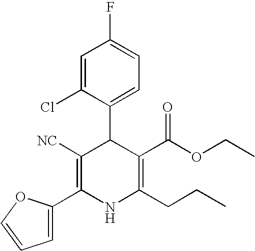 5-ethyl-2-(2-furyl)-3-cyano-4-(2- chloro-4-fluorophenyl)-6-propyl1,4- dihydro-pyridine-5-carboxylate | ¹H NMR (CDCl₃, 400 MHz): 7.44 (d, J = 1.6 Hz, 1H), 7.26 (d, J = 3.6 Hz, 1H), 7.20 (dd, J = 8.8, 6.0 Hz, 1H), 7.02 (dd, J = 8.8, 2.4 Hz, 1H), 6.85 (dt, J = 8.8, 2.4 Hz, 1H), 6.61 (s, 1H), 6.48 (dd, J = 3.6, 1.6 Hz, 1H), 5.29 (s, 1H), 3.94 (m, 2H), 2.76 (m, 2H), 1.65 (m, 2H), 1.05 (t, J = 6.8 Hz, 3H), 0.99 (t,J = 6.8 Hz, 3H). MS (ES⁺) 414, m/z (M + 1) 415, C₂₀H₂₀ClFN₂O₃ requires 414 |
| 228 | 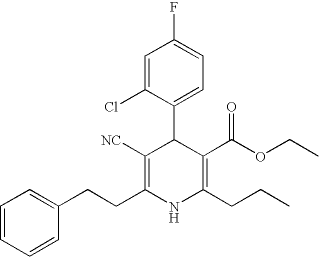 5-ethyl-2-(2-phenylethyl)-3-cyano-4- (2-chloro-4-fluorophenyl)-6-propyl1,4- dihydro-pyridine-5-carboxylate | ¹H NMR (CDCl₃, 400 MHz): 7.30 (m, 2H), 7.25 (m, 1H), 7.18 (m, 2H), 7.12 (dd, J = 8.8, 6.0 Hz, 1H), 7.06 (dd, J = 8.8, 2.4 Hz, 1H), 6.92 (dt, J = 8.8, 2.4 Hz, 1H), 5.62 (s, 1H), 5.21 (s, 1H), 3.96 (q, J = 7.2 Hz, 2H), 2.88 (m, 2H), 2.67 (m, 3H), 2.30 (m, 1H), 1.42 (m, 2H), 1.08 (t, J = 7.2 Hz, 3H), 0.91 (t, J = 6.8 Hz, 3H). MS (ES⁺) 452, m/z (M + 1) 453, C₂₆H₂₆ClFN₂O₂ requires 452 |
| 229 | 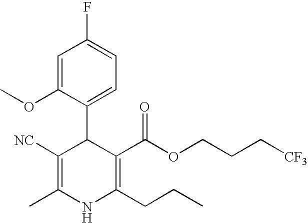 5-(4,4,4-trifluorobutyl)-2-methyl-3- cyano-4-(2-methoxy-4-fluorophenyl)-6- propyl1,4-dihydro-pyridine-5- carboxylate | ¹H NMR (CDCl₃, 400 MHz): 6.93 (dd, J = 8.4, 6.4 Hz, 1H), 6.52 (m, 2H), 5.72 (s, 1H), 5.01 (s, 1H), 3.92 (m, 1H), 3.82 (m, 1H), 3.76 (s, 3H), 2.74 (m, 1H), 2.56 (m, 1H), 1.95 (s, 3H), 1.74 (m, 2H), 1.61 (m, 4H), 0.96 (t, J = 7.2 Hz, 3H). MS (ES⁺) 440, m/z (M + 1) 441,C₂₂H₂₄F₄N₂O₃ requires 440 |
| 230 | 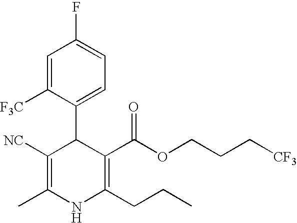 5-(4,4,4-trifluorobutyl)-2-methyl-3- cyano-4-(2-trifluoromethyl-4- fluorophenyl)-6-propyl-1,4-dihydro- pyridine-5-carboxylate | ¹H NMR (CDCl₃, 400 MHz): 7.38 (dd, J = 8.4, 5.6 Hz, 1H), 7.25 (dd, J = 9.2, 2.8 Hz, 1H), 7.14 (dt, J = 8.4, 2.8 Hz, 1H), 5.92 (s, 1H), 4.96 (s, 1H), 3.97 (m, 1H), 3.88 (m, 1H), 2.61 (m, 2H), 2.01 (s, 3H), 1.62 (m, 6H), 0.95 (t, J = 7.2 Hz, 3H). MS (ES⁺) 478, m/z (M + 1) 479, C₂₂H₂₁F₇N₂O₂ requires 478 |
| 231 | 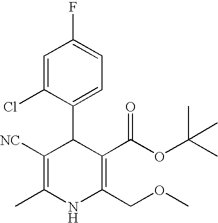 5-tert-butyl-2-methyl-3-cyano-4-(2- chloro-4-fluorophenyl)-6-(2- methoxymethyl,4-dihydro-pyridine-5- carboxylate | ¹H NMR (CDCl₃, 400 MHz): 7.19 (dd, J = 8.4, 6.0 Hz, 1H), 7.07 (m, 2H), 6.92 (dt, J = 8.4, 2.8 Hz, 1H), 5.14 (s, 1H), 4.65 (s, 2H), 3.46 (s, 3H), 2.05 (s, 3H), 1.22 (s, 9H). MS (ES⁺) 392, m/z (M + 1) 393, C₂₀H₂₂ClFN₂O₃ requires 392 |
| 232 | 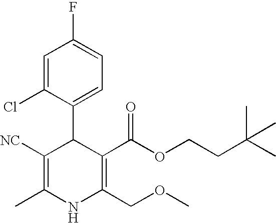 5-(3,3-dimethylbutyl)-2-methyl-3- cyano-4-(2-chloro-4-fluorophenyl)-6- (2-methoxymethyl,4-dihydro-pyridine- 5-carboxylate | ¹H NMR (CDCl₃, 400 MHz): 7.10 (dd, J = 8.4, 6.0 Hz, 1H), 7.04 (s, 1H), 6.96 (dd, J = 8.4, 2.8 Hz, 1H), 6.81 (dt, J = 8.4, 2.8 Hz, 1H), 5.07 (s,1H), 4.57 (m, 2H), 3.86 (m, 2H), 3.37 (s, 3H), 1.98 (s, 3H), 1.26 (m, 2H), 0.72 (s, 9H). MS (ES⁺) 420, m/z (M + 1) 421,C₂₂H₂₆ClFN₂O₃ requires 420 |
| 233 | 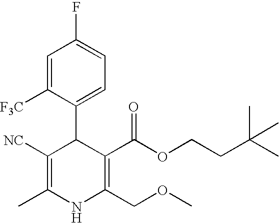 5-(3,3-dimethylbutyl)-2-methyl-3- cyano-4-(2-trifluoromethyl-4- fluorophenyl)-6-(2-methoxymethyl,4- dihydro-pyridine-5-carboxylate | ¹H NMR (CDCl₃, 400 MHz): 7.31 (dd, J = 8.4, 5.6 Hz, 1H), 7.20 (dd, J = 9.2, 2.8 Hz, 1H), 7.08 (m, 2H), 4.93 (s, 1H), 4.57 (s, 2H), 3.84 (m, 2H), 3.38 (s, 3H), 2.01 (s, 3H), 1.14 (m, 2H), 0.70 (s, 9H). MS (ES⁺) 454, m/z (M + 1) 455, C₂₃H₂₆F₄N₂O₃ requires 454 |
| 234 | 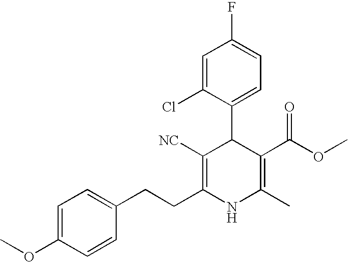 5-methyl-2-[2-(4- methoxyphenyl)ethyl]-3-cyano-4-(2- chloro-4-fluorophenyl)-6-methyl-1,4- dihydro-pyridine-5-carboxylate | ¹H NMR (CDCl₃, 400 MHz): 7.11 (m, 2H), 7.07 (m, 2H), 6.90 (dt, J = 8.0, 2.4 Hz, 1H), 6.85 (m, 2H), 5.36 (s, 1H), 5.18 (s, 1H), 3.79 (s, 3H), 3.53 (s, 3H), 2.87 (m, 2H), 2.65 (m, 2H), 2.14 (s, 3H). MS (ES⁺) 440, m/z (M + 1) 441, C₂₄H₂₂ClFN₂O₃ requires 440 |
| 235 | 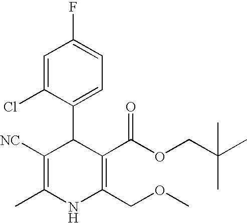 5-(2,2-dimethylpropyl)-2methyl-3- cyano-4-(2-chloro-4-fluorophenyl)-6- methy-1,4-dihydro-pyridine-5- carboxylate | ¹H NMR (CDCl₃, 400 MHz): 7.10 (s, 1H), 7.07 (dd, J = 8.4, 6.0 Hz, 1H), 6.97 (dd, J = 8.4, 2.8 Hz, 1H), 6.81 (dt, J = 8.4, 2.8 Hz, 1H), 5.11 (s, 1H), 4.60 (s, 2H), 3.53 (m, 2H), 3.81 (s, 3H), 1.97 (s, 3H), 0.64 (s, 9H). MS (ES⁺) 406, m/z (M + 1) 407, C₂₁H₂₄ClFN₂O₃ requires 406 |
| 236 | 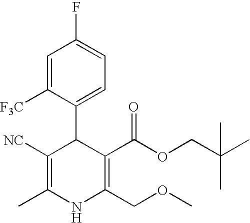 5-(2,2-dimethylpropyl)-2methyl-3- cyano-4-(2-trifluoromethyl-4- fluorophenyl)-6-(2-methoxymethyl)- 1,4-dihydro-pyridine-5-carboxylate | ¹H NMR (CDCl₃, 400 MHz): 7.32 (dd, J = 8.4, 5.6 Hz, 1H), 7.20 (m, 1H), 7.08 (m, 2H), 4.98 (s, 1H), 4.56 (s, 2H), 3.63 (m, 2H), 3.38 (s, 3H), 2.01 (s, 3H), 0.61 (s, 9H). MS (ES⁺) 440, m/z (M + 1) 441, C₂₂H₂₄F₄N₂O₃ requires 440 |
| 237 | 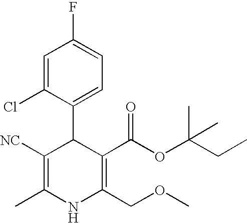 5-(1,1-dimethylpropyl)-2methyl-3- cyano-4-(2-chloro-4-fluorophenyl)-6- )2-methoxymethyl)-1,4-dihydro- pyridine-5-carboxylate | ¹H NMR (CDCl₃, 400 MHz): 7.09 (dd, J = 8.4, 6.0 Hz, 1H), 6.99 (s, 1H), 6.96 (dd, J = 8.4, 2.8 Hz, 1H), 6.81 (dt, J = 8.4, 2.8 Hz, 1H), 5.04 (s, 1H), 4.56 (s, 2H), 3.36 (s, 3H), 1.95 (s, 3H), 1.58 (m, 1H), 1.44 (m, 1H), 1.11 (s, 3H), 1.08 (s, 3H), 0.43 (t, J = 7.6 Hz, 3H). MS (ES⁺) 406, m/z (M + 1) 407, C₂₁H₂₄ClFN₂O₃ requires 406 |
| 238 | 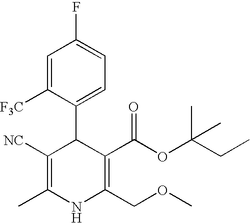 5-(1,1-dimethylpropyl)-2methyl-3- cyano-4-(2-trifluoromethyl-4- fluorophenyl)-6-(2-methoxymethyl)- 1,4-dihydro-pyridine-5-carboxylate | ¹H NMR (CDCl₃, 400 MHz): 7.42 (dd, J = 8.4, 5.6 Hz, 1H), 7.30 (dd, J = 9.2, 2.8 Hz, 1H), 7.18 (dt, J = 8.4, 2.8 Hz, 1H), 7.11 (s, 1H), 5.06 (s, 1H), 4.61 (ms, 2H), 3.47 (s, 3H), 2.08 (s, 3H), 1.67 (m, 1H), 1.53 (m, 1H), 1.19 (s, 3H), 1.14 (s, 3H), 0.52 (t, J = 7.6 Hz, 3H). MS (ES⁺) 440, m/z (M + 1) 441, C₂₂H₂₄F₄N₂O₃ requires 440 |
| 239 | 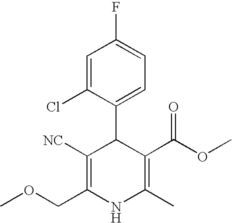 5-methyl-2-(2-methoxymethyl)-3- cyano-4-(2-trifluoromethyl-4- fluorophenyl)-6-(2-methoxymethyl)- 1,4-dihydro-pyridine-5-carboxylate | ¹H NMR (CDCl₃, 400 MHz): 7.15 (dd, J = 8.4, 6.0 Hz, 1H), 7.01 (dd, J = 8.4, 2.4 Hz, 1H), 6.86 (dt, J = 8.4, 2.4 Hz, 1H), 6.40 (s, 1H), 5.16 (s, 1H), 4.14 (m, 2H), 3.47 (s, 3H), 3.33 (s, 3H), 2.31 (s, 3H). MS (ES⁺) 350, m/z (M + 1) 351, C₁₇H₁₆ClFN₂O₃ requires 350 |
| 240 | 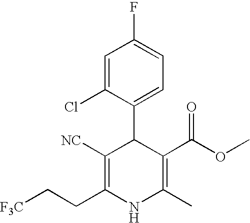 5-methyl-2-(3,3,3-trifluoropropyl)-3- cyano-4-(2-chloro-4-fluorophenyl)-6- methyl-1,4-dihydro-pyridine-5- carboxylate | ¹H NMR (CDCl₃, 400 MHz): 7.12 (dd, J = 8.4, 6.0 Hz, 1H), 7.01 (dd, J = 8.4, 2.4 Hz, 1H), 6.86 (dt, J = 8.4, 2.4 Hz, 1H), 6.44 (s, 1H), 5.12 (s, 1H), 3.47 (s, 3H), 2.52 (m, 2H), 2.29 (m, 2H), 2.25 (s, 3H). MS (ES⁺) 402, m/z (M + 1) 403, C₁₈H₁₅ClF₄N₂O₂ requires 402 |
| 241 | 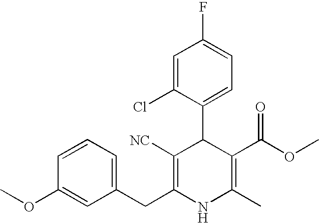 5-methyl-2-(3-methoxyphenylmethyl)- 3-cyano-4-(2-chloro-4-fluorophenyl)-6- methyl-1,4-dihydro-pyridine-5- carboxylate | ¹H NMR (CDCl₃, 400 MHz): 7.20 (m, 1H), 7.13 (dd, J = 8.8, 5.6 Hz, 1H), 7.02 (dd, J = 8.8, 2.8 Hz, 1H), 6.85 (dt, J = 8.0, 2.8 Hz, 1H), 6.78 (dd, J = 8.0, 2.8 Hz, 1H), 6.70 (d, J = 8.0 Hz, 1H), 6.65 (m, 1H), 5.51 (s, 1H), 5.18 (s, 1H), 3.71 (s, 3H), 3.62 (m, 2H), 3.46 (s, 3H), 2.16 (s, 3H). MS (ES⁺) 426, m/z (M + 1) 427, C₂₃H₂₀ClFN₂O₃ requires 426 |
| 242 | 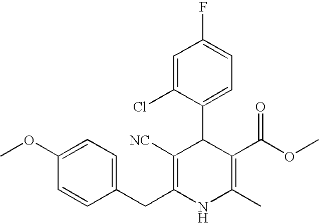 5-methyl-2-(4-methoxyphenylmethyl)- 3-cyano-4-(2-chloro-4-fluorophenyl)-6- methyl-1,4-dihydro-pyridine-5- carboxylate | ¹H NMR (CDCl₃, 400 MHz): 7.12 (dd, J = 8.4, 6.0 Hz, 1H), 7.04 (m, 2H), 7.01 (m, 1H), 6.85 (dt, J = 8.4, 2.8 Hz, 1H), 6.80 (m, 2H), 5.50 (s, 1H), 5.17 (s, 1H), 3.75 (s, 3H), 3.59 (s, 2H), 3.46 (s, 3H), 2.15 (s, 3H). MS (ES⁺) 426, m/z (M + 1) 427, C₂₃H₂₀ClFN₂O₃ requires 426 |
| 243 | 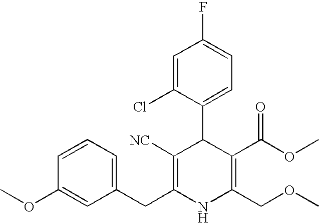 5-methyl-2-(3-methoxyphenylmethyl)- 3-cyano-4-(2-chloro-4-fluorophenyl)-6- (2-methoxymethyl)-1,4-dihydro- pyridine-5-carboxylate | ¹H NMR (CDCl₃, 400 MHz): 7.19 (m, 1H), 7.12 (dd, J = 8.8, 5.6 Hz, 1H), 7.08 (m, 1H), 7.02 (dd, J = 8.8, 2.8 Hz, 1H), 6.85 (m, 1H), 6.78 (dd, J = 8.0, 2.8 Hz, 1H), 6.72 (d, J = 8.0 Hz, 1H), 6.67 (m, 1H), 5.18 (s, 1H), 4.48 (m, 2H), 3.71 (s, 3H), 3.64 (m, 2H), 3.45 (s, 3H), 3.15 (s, 3H). MS (ES⁺) 456, m/z (M + 1) 457, C₂₄H₂₂ClFN₂O₄ requires 456 |
| 244 | 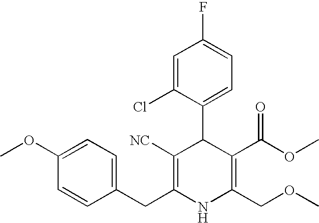 5-methyl-2-(4-methoxyphenylmethyl)- 3-cyano-4-(2-chloro-4-fluorophenyl)-6- (2-methoxymethyl)-1,4-dihydro- pyridine-5-carboxylate | ¹H NMR (CDCl₃, 400 MHz): 7.11 (dd, J = 8.8, 6.0 Hz, 1H), 7.07 (m, 2H), 7.01 (m, 2H), 6.85 (dt, J = 8.0, 2.8 Hz, 1H), 6.81 (m, 2H), 5.17 (s, 1H), 4.48 (m, 2H), 3.73 (s, 3H), 3.64 (m, 2H), 3.45 (s, 3H), 3.18 (s, 3H). MS (ES⁺) 456, m/z (M + 1) 457, C₂₄H₂₂ClFN₂O₄ requires 456 |
| 245 | 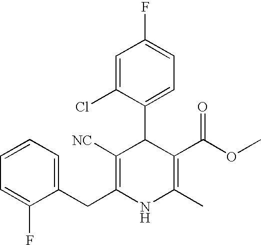 5-methyl-2-(2-fluorophenylmethyl)-3- cyano-4-(2-chloro-4-fluorophenyl)-6- methyl-1,4-dihydro-pyridine-5- carboxylate | ¹H NMR (CDCl₃, 400 MHz): 7.20 (m, 2H), 7.07 (dd, J = 8.8, 6.0 Hz, 1H), 7.03 (m, 1H), 6.96 (m, 2H), 6.79 (dt, J = 8.0, 2.8 Hz, 1H), 5.86 (s, 1H), 5.11 (s, 1H), 3.61 (s, 2H), 3.41 (s, 3H), 2.16 (s, 3H). MS (ES⁺) 414, m/z (M + 1) 415, C₂₂H₁₇ClF₂N₂O₂ requires 414 |
| 246 | 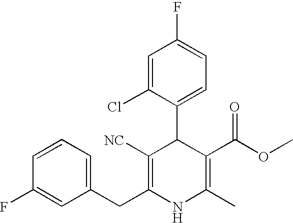 5-methyl-2-(3-fluorophenylmethyl)-3- cyano-4-(2-chloro-4-fluorophenyl)-6- methyl-1,4-dihydro-pyridine-5- carboxylate | ¹H NMR (CDCl₃, 400 MHz): 7.29 (m, 1H), 7.18 (dd, J = 8.8, 6.0 Hz, 1H), 7.07 (dd, J = 8.8, 2.8 Hz, 1H), 7.00 (dd, J = 8.8, 2.8 Hz, 1H), 6.96 (m, 1), 6.92 (dd, J = 8.0, 2.4 Hz, 1H), 6.89 (dt, J = 8.0, 2.4 Hz, 1H), 5.70 (s, 1H), 5.24 (s, 1H), 3.67 (m, 2H), 3.52 (s, 3H), 2.22 (s, 3H). MS (ES⁺) 414, m/z(M + 1) 415, C₂₂H₁₇ClF₂N₂O₂ requires 414 |
| 247 | 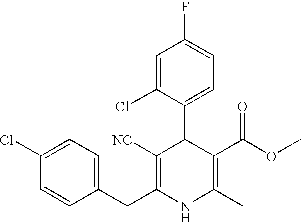 5-methyl-2-(4-chlorophenylmethyl)-3- cyano-4-(2-chloro-4-fluorophenyl)-6- methyl-1,4-dihydro-pyridine-5- carboxylate | ¹H NMR (CDCl₃, 400 MHz): 7.27 (m, 2H), 7.16 (dd, J = 8.8, 6.0 Hz, 1H), 7.10 (m, 2H), 7.07 (dd, J = 8.8, 2.8 Hz, 1H), 6.91 (dt, J = 8.0, 2.4 Hz, 1H), 5.89 (s,1H), 5.22 (s, 1H), 3.63 (m, 2H), 3.51 (s, 3H), 2.21 (s, 3H). MS (ES⁺) 430, m/z (M + 1) 431, C₂₂H₁₇Cl₂FN₂O₂ requires 430 |
| 248 | 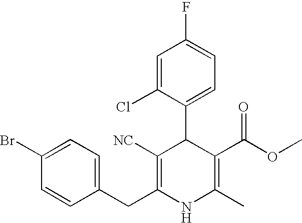 5-methyl-2-(4-bromophenylmethyl)-2- cyano-4-(2-chloro-4-fluorophenyl)-6- methyl-1,4-dihydro-pyridine-5- carboxylate | ¹H NMR (CDCl₃, 400 MHz): 7.42 (m, 2H), 7.16 (dd, J = 8.8, 6.0 Hz, 1H), 7.07 (dd, J = 8.8, 2.8 Hz, 1H), 7.04 (m, 2H), 6.91 (dt, J = 8.0, 2.8 Hz, 1H), 5.97 (s, 1H), 5.22 (s, 1H), 3.61 (m, 2H), 3.51 (s, 3H), 2.21 (s, 3H). MS (ES⁺) 474, m/z (M + 1) 475, C₂₂H₁₇BrClFN₂O₂ requires 474 |
| 249 | 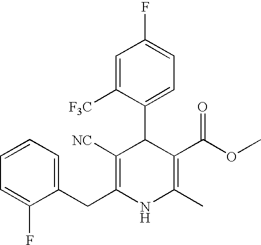 5-methyl-2-(2-fluorophenylmethyl)-3- cyano-4-(2-trifluoromethyl-4- fluorophenyl)-6-methyl-1,4-dihydro- pyridine-5-carboxylate | ¹H NMR (CDCl₃, 400 MHz): 7.37 (dd, J = 8.8, 5.6 Hz, 1H), 7.27 (m, 3H), 7.15 (dd, J = 8.8, 2.8 Hz, 1H), 7.11 (m, 1H), 7.07 (m, 1H), 6.03 (s, 1H), 5.04 (s, 1H), 3.72 (s, 2H), 3.45 (s, 3H), 2.27 (s, 3H). MS (ES⁺) 448, m/z (M + 1) 449, C₂₃H₁₇F₅N₂O₂ requires 448 |
| 250 | 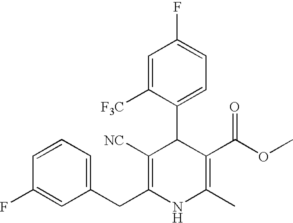 5-methyl-2-(3-fluorophenylmethyl)-3- cyano-4-(2-trifluoromethyl-4- fluorophenyl)-6-methyl-1,4-dihydro- pyridine-5-carboxylate | ¹H NMR (CDCl₃, 400 MHz): 7.60 (dd, J = 8.8, 5.6 Hz, 1H), 7.53 (dd, J = 9.2, 2.8 Hz, 1H), 7.49 (m, 1H), 7.39 (dt, J = 8.0, 2.8 Hz, 1H), 7.20 (dd, J = 8.8, 2.8 Hz, 1H), 7.15 (m, 1H), 7.07 (m, 1H), 6.10 (s, 1H), 5.29 (s, 1H), 3.87 (m, 2H), 3.68 (s, 3H), 2.44 (s, 3H). MS (ES⁺) 448, m/z (M + 1) 449, C₂₃H₁₇F₅N₂O₂ requires 448 |
| 251 | 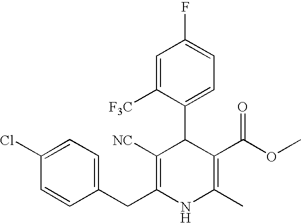 5-methyl-2-(4-chlorophenylmethyl)-3- cyano-4-(2-trifluoromethyl-4- fluorophenyl)-6-methyl-1,4-dihydro- pyridine-5-carboxylate | ¹H NMR (CDCl₃, 400 MHz): 7.37 (dd, J = 8.8, 6.0 Hz, 1H), 7.32 (dd, J = 9.2, 2.4 Hz, 1H), 7.29 (m, 2H), 7.17 (dt, J = 8.8, 2.4 Hz, 1H), 7.10 (m, 2H), 5.68 (s, 1H), 5.08 (s, 1H), 3.67 (m, 2H), 3.46 (s, 3H), 2.24 (s, 3H). MS (ES⁺) 464, m/z (M + 1) 465, C₂₃H₁₇ClF₄N₂O₂requires 464 |
| 252 | 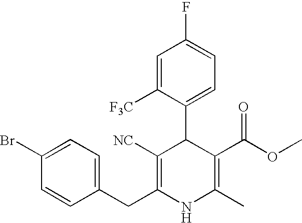 5-methyl-2-(4-bromophenylmethyl)-3- cyano-4-(2-trifluoromethyl-4- fluorophenyl)-6-methyl-1,4-dihydro- pyridine-5-carboxylate | ¹H NMR (CDCl₃, 400 MHz): 7.43 (m, 2H), 7.37 (dd, J = 8.8, 6.0 Hz, 1H), 7.32 (dd, J = 8.8, 2.8 Hz, 1H), 7.17 (dt, J = 8.0, 2.8 Hz, 1H), 7.03 (m, 2H), 5.78 (s, 1H), 5.07 (s, 1H), 3.65 (m, 2H), 3.46 (s, 3H), 2.24 (s, 3H). MS (ES⁺) 508, m/z (M + 1) 509, C₂₃H₁₇BrF₄N₂O₂requires 508 |
| 253 | 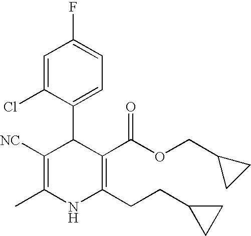 5-cyclopropylmethyl-2-methyl-3- cyano-4-(2-chloro-4-fluorophenyl)-6- (2-cyclopropyl)ethyl-1,4-dihydro- pyridine-5-carboxylate | MS (ES⁺) 415, m/z (M + 1) 416, C₂₃H₂₄ClFN₂O₂ requires 516. |
| 254 | 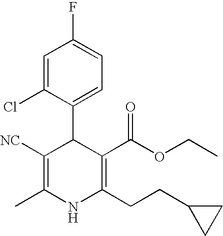 5-ethyl-2-methyl-3-cyano-4-(2-chloro- 4-fluorophenyl)-6-(2- cyclopropyl)ethyl-1,4-dihydro- pyridine-5-carboxylate | ¹H NMR (DMDO-d6, 400 Mhz): 9.08 (broad s, 1H), 7.24 (dd, J = 8.8, 2.0 Hz, 1H), 7.13 (m, 2H), 4.93 (s, 1H), 3.76 (m, 2H), 2.64 (m, 2H), 1.88 (s, 3H), 1.34 (m, 2H), 0.88 (t, J = 7.1 Hz, 3H), 0.63 (m, 1H), 0.31 (broad s, J = 8.0 Hz, 2H), 0.01 (broad d, J = 4.0 Hz, 2H). MS (ES⁺) 389, m/z (M + 1) 390,C₂₁H₂₂ClFN₂O₂ requires 390 |
| 255 | 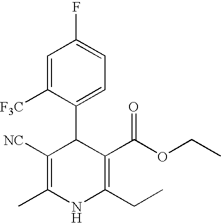 5-ethyl-2-methyl-3-cyano-4-(2- trifluoromethyl-4-fluorophenyl)-6- ethyl-1,4-dihydro-pyridine-5- carboxylate | ¹ H NMR (DMDO-d6, 400 Mhz): 9.28 (broad s, 1H), 7.57 (dd, J = 8.8, 2.0 Hz, 1H), 7.18 (m, 2H), 4.89 (s, 1H), 3.88 (m, 2H), 2.67 (m, 2H), 2.02 (s, 3H), 1.17 (t, J = 7.2 Hz, 3H), 0.91 (t, J = 7.2 Hz, 3H). MS (ES⁺) 382, m/z (M + 1) 383, C₁₉H₁₈F₄N₂O₂ requires 383 |
| 256 | 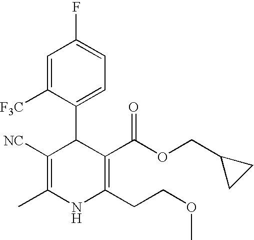 5-cyclopropylmethyl-2-methyl-3- cyano-4-(2-trifluoromethyl-4- fluorophenyl)-6-(2-methoxyethyl)-1,4- dihydro-pyridine-5-carboxylate | ¹H NMR (CDCl₃, 400 Mhz): 7.45 (dd, J = 8.8, 5.6 Hz, 1H), 7.27 (dd, J = 9.6, 2.8 Hz, 1H), 7.16 (td, J = 8.0, 2.4 Hz, 1H), 7.11 (broad s, 1H), 5.05 (s, 1H), 3.65 (m, 4H), 3.39 (s, 3H), 3.13 (m, 1H), 2.03 (s, 3H), 0.86 (m, 1H), 0.32 (m, 2H), 0.00 (m, 2H). MS (ES⁺) 438, m/z (M + 1) 439, C₂₂H₂₂F₄N₂O₃ requires 439. |
| 257 | 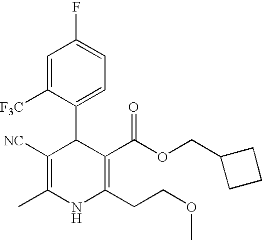 5-cyclobutylmethyl-2-methyl-3-cyano- 4-(2-trifluoromethyl-4-fluorophenyl)-6- (2-methoxyethyl)-1,4-dihydro-pyridine- 5-carboxylate | ¹H NMR (CDCl₃, 400 MHz): 7.52 (dd, J = 8.8, 5.2 hz, 1H), 7.35 (dd, J = 9.2, 2.4 Hz, 1H), 7.23 (td, J = 8.4, 2.8 Hz, 1H), 7.18 (broad s, 1H), 5.08 (s, 1H), 3.94 (d, J = 7.2 Hz, 2H), 3.71 (m, 2H), 3.68 (s, 3H), 3.20 (m, 2H), 2.43 (m, 1H), 2.11 (s, 3H), 1.84 (m, 4H), 1.56 (m, 2H). MS (ES⁺) 452, m/z (M + 1) 453, C₂₃H₂₄F₄N₂O₃ requires 453. |
| 258 | 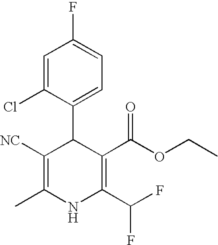 5-ethyl-2-methyl-3-cyano-4-(2-chloro- 4-fluorophenyl)-6-difluoromethyl-1,4- dihydro-pyridine-5-carboxylate | ¹H NMR DMSO-d6, 400 MHz): 9.86 (broad s, 1H), 7.58 (t, J = 53.2 Hz, 1H), 7.53 (dd, J = 8.8, 2.4 Hz, 1H), 7.43 (m, 1H), 7.38 (td, J = 8.4, 2.4 Hz, 1H), 5.26 (s, 1H), 4.07 (dd, J = 14.0, 6.8 Hz, 2H), 2.17 (s, 3H), 1.13 (t, J = 7.2 Hz, 3H). MS (ES⁺) 370, m/z (M + 1) 371, C₁₇H₁₄ClF₃N₂O₂ requires 371. |
| 259 | 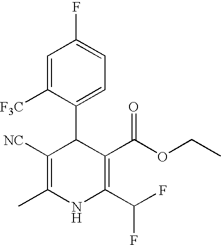 5-ethyl-2-methyl-3-cyano-4-(2- trifluoromethyl-4-fluorophenyl)-6- difluoromethyl-1,4-dihydro-pyridine-5- carboxylate | ¹H NMR DMSO-d6, 400 MHz): 9.82 (broad s, 1H), 7.65 (dd, J = 8.8, 2.4 Hz, 1H), 7.62 (m, 1H), 7.53 (td, J = 8.4, 2.4 Hz, 1H), 7.41 (t, J = 53.2 Hz, 1H), 4.97 (s, 1H), 3.93 (m, 2H), 2.09 (s, 3H), 0.92 (t, J = 7.2 Hz, 3H). MS (ES⁺) 404, m/z (M + 1) 405, C₁₈H₁₄F₆N₂O₂ requires 405. |
| 260 | 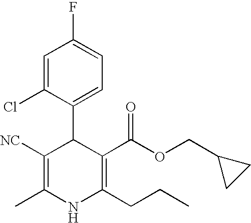 5-cyclopropylmethyl-2-methyl-3- cyano-4-(2-trifluoromethyl-4- fluorophenyl)-6-propyl-1,4-dihydro- pyridine-5-carboxylate | ¹H NMe CDCl₃, 400 Mhz): 7.19 (dd, J = 8.8, 6.4 Hz, 1H), 6.96 (dd, J = 8.4, 2.4 Hz, 1H), 6.81 (td, J = 8.0, 2.4 Hz, 1H), 5.88 (broad s, 1H), 5.13 (s, 1H), 3.63 (d, J = 8.0 Hz, 2H), 2.66 (m, 1H), 2.51 (m, 1H), 1.93 (s, 3H), 1.55 (m, 2H), 0.90 (t, J = 7.3 Hz, 3H), 0.88 (m, 1H), 0.30 (m, 2H), 0.00 (m, 1H), −0.07 (m, 1H). MS (ES⁺) 389, m/z (M + 1) 390, C₂₁H₂₂ClFN₂O₂ requires 390. |
| 261 | 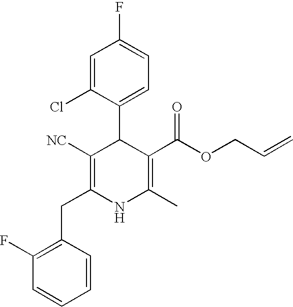 5-allyl-2-(2-fluorophenyl)methyl-3- cyano-4-(2-trifluoromethyl-4- fluorophenyl)-6-methyl-1,4-dihydro- pyridine-5-carboxylate | ¹H NMR CDCl₃, 400 MHz): 7.17 (m, 2H), 7.06 (dd, J = 8.8, 6.0 Hz, 1H), 7.00 (t, J = 7.6 Hz, 1H), 6.95 (m, 1H), 6.92 (dd, J = 8.4, 2.8 Hz, 1H), 6.77 (td, J = 8.4, 2.8 Hz, 1H), 5.87 (broad s, 1H), 5.59 (m, 1H), 5.12 (s, 1H), 4.97 (d, J = 7.1 Hz, 1H), 4.95 (d, J = 17.2 Hz, 1H), 4.29 (d, J = 5.6Hz, 2H), 3.59 (s, 2H), 2.15 (s, 3H). MS (ES⁺) 440, m/z (M + 1) 441, C₂₄H₁₉ClF₂N₂O₂ requires 441. |
| 262 | 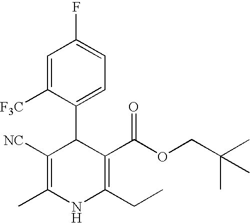 5-(2,2-dimethylpropyl)-2-methyl-3- cyano-4-(2-trifluoromethyl-4- fluorophenyl)-6-ethyl-1,4-dihydro- pyridine-5-carboxylate | ¹H NMR CDCl₃, 400 MHz): 7.43 (dd, J = 8.4, 5.2 Hz, 1H), 7.28 (dd, J = 9.6, 2.8 Hz, 1H), 7.18 (td, J = 8.0, 2.4 Hz, 1H), 5.93 (broad s, 1H), 5.04 (s, 1H), 3.79 (d, J = 10.8 Hz, 1H), 3.66 (d, J = 10.8 Hz, 1H), 2.67 (m, 2H), 2.07 (s, 3H), 1.27 (t, J = 7.6 Hz, 3H), 0.74 (s, 9H). MS (ES⁺) 424, m/z (M + 1) 425 C₂₂H₂₄F₄N₂O₂ requires 425. |
| 263 | 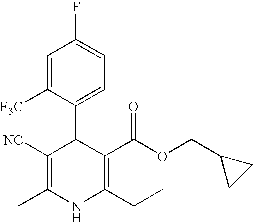 5-cyclopropylmethyl-2-methyl-3- cyano-4-(2-trifluoromethyl-4- fluorophenyl)-6-ethyl-1,4-dihydro- pyridine-5-carboxylate | ¹H NMR CDCl₃, 400 MHz): 7.41 (dd, J = 8.4, 5.2 Hz, 1H), 7.27 (dd, J = 9.2, 2.8 Hz, 1H), 7.15 (td, J = 8.0, 2.4 Hz, 1H), 5.83 (broad s, 1H), 5.04 (s, 1H), 3.69 (m, 2H), 2.73 (m, 2H), 2.05 (s, 3H), 1.21 (t, J = 7.2 Hz, 3H), 0.86 (m, 1H), 0.33 (m, 2H), −0.01 (m, 2H). MS (ES⁺) 408, m/z (M + 1) 409 C₂₁H₂₀F₄N₂O₂ requires 409. |
| 264 | 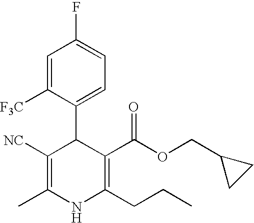 5-cyclopropylmethyl-2-methyl-3- cyano-4-(2-trifluoromethyl-4- fluorophenyl)-6-propyl-1,4-dihydro- pyridine-5-carboxylate | ¹H NMR CDCl₃, 400 MHz): 7.41 (dd, J = 8.8, 5.6 Hz, 1H), 7.26 (dd, J = 9.2, 2.8 Hz, 1H), 7.15 (td, J = 8.4, 2.8 Hz, 1H), 5.80 (broad s, 1H), 5.05 (s, 1H), 3.68 (m, 2H), 2.65 (m, 2H), 2.04 (s, 3H), 1.61 (m, 2H), 0.98 (t, J = 7.1 Hz, 3H), 0.85 (m, 1H), 0.33 (m, 2H), −0.01 (m, 2H). MS (ES⁺) 422, m/z (M + 1) 423 C₂₂H₂₂F₄N₂O₂ requires 423. |
| 265 | 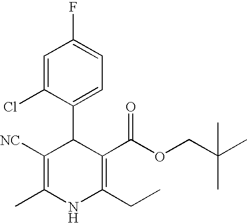 5-(2-methyl)propyl-2-methyl-3-cyano- 4-(2-trifluoromethyl-4-fluorophenyl)-6- ethyl-1,4-dihydro-pyridine-5- carboxylate | ¹H NMR CDCl₃, 400 MHz): 7.09 (dd, J = 8.4, 6.0 Hz, 1H), 6.97 (dd, J = 8.4, 2.8 Hz, 1H), 6.81 (td, mJ = 8.4, 2.4 Hz, 1H), 5.78 (broad s, 1H), 5.08 (s, 1H), 3.62 (m, 2H), 2.71 (m, 2H), 2.62 (m, 2H), 1.93 (s, 3H), 1.67 (m, 1H), 1.11 (t, J = 7.0 Hz, 3H), 0.66 (d, J = 6.4 Hz, 3H), 0.58 (d, J = 6.4 Hz, 3H). MS (ES⁺) 376, m/z (M + 1) 377 C₂₀H₂₂ClFN₂O₂ requires 377. |
| 266 | 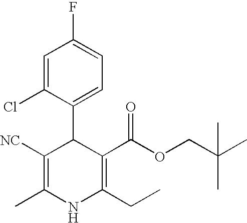 5-(2,2-dimethyl)propyl-2-methyl-3- cyano-4-(2-chloro-4-fluorophenyl)-6- ethyl-1,4-dihydro-pyridine-5- carboxylate | ¹H NMR CDCl₃, 400 MHz): 7.16 (dd, J = 8.8, 6.0 Hz, 1H), 7.07 (dd, J = 8.4, 2.4 Hz, 1H), 6.90 (td, J = 8.4, 2.8 Hz, 1H), 5.96 (broad s, 1H), 5.19 (s, 1H), 3.66 (dd, J = 10.4 Hz, 2H), 2.77 (m, 2H), 2.04 (s, 3H), 1.29 (t, J = 7.2 Hz, 3H), 0.77 (s, 9H). MS (ES⁺) 390, m/z (M + 1) 391 C₂₁H₂₄ClFN₂O₂ requires 391. |

### Example 4

### Functional Assay of Mineralocorticoid Receptor Antagonism

The MR antagonist activity of the compounds is determined in a mammalian two hybrid reporter system. The N-terminus of MR (MR-NT, sequence coding amino acid 1-597) is fused to the activation domain of the VP16 gene. The ligand binding domain of MR (MR-LBD, sequence encoding amino acid 672-984) is fused to the DNA binding domain of the yeast Gal4 gene. The MR gene is cloned from a human kidney cDNA library with PCR.

The assay is performed in 384 well plates. Briefly, 293T cells (ATCC) are transfected with expression vectors for Gal4-MR-LBD and VP16-MR NT, and a luciferase reporter vector containing Gal4 binding sequence (pG5-Luc). Cells are plated in 384 well plates immediately after transfection (approximately 3×10⁴ cells/well in 50 μl medium). The medium is supplemented with 3% charcoal-dextran treated fetal bovine serum (Hyclone). Twenty four hours after transfection, compounds prepared in DMSO are transferred to the cells. The cells are then stimulated with 0.4 nM final concentration of aldosterone (Acros) and incubated at 37° C. for another 24 hours before the luciferase activity is assayed with 20 μl of Bright-Glo (Promega) using a luminometer (CLIPR). The expression of luciferase is used as an indicator of aldosterone-induced MR trans-activation. Each compound is tested in duplicate with 12-concentration titration. IC50 values (defined as the concentration of test compound required to antagonize 50% of aldosterone-induced MR activity) are determined from the dose-response curve.

### Example 5

### Functional Assay of Glucocorticoid Receptor Antagonism

The GR antagonist activity of the compounds is determined in a mammalian two hybrid reporter system. The ligand binding domain of GR (GR-LBD, sequence encoding amino acid 541-778) is fused to the DNA binding domain of the yeast Gal4 gene. The GR gene is cloned from a human lung cDNA library with PCR.

The assay is performed in 384 well plates: COS-7 cells (ATCC) are transfected with expression vectors for Gal-4-GR-LBD and a luciferase reporter vector containing Gal4 binding sequence (pG5-Luc). Cells are plated in 384 well plates immediately after transfection (approximately 8000 cells/well in 50 μl medium). The medium is supplemented with 3% charcoal-dextran treated fetal bovine serum (Hyclone). Twenty four hours after transfection, compounds prepared in DMSO are transferred to the cells. The cells are then stimulated with 10 nM final concentration of dexamethasone (Sigma) and incubated at 37° C. for another 24 hours before the luciferase activity is assayed with 20 μl of Bright-Glo (Promega) using a luminometer (CLIPR). The expression of luciferase is used as an indicator of dexamethasone-induced GR trans-activation. Each compound is tested in duplicate with a 12-concentration titration. IC50 values (defined as the concentration of test compound required to antagonize 50% of dexamethasone-induced GR activity) are determined from the dose-response curve.

### Example 6

### Functional Assay of Progesterone Receptor Antagonism

The PR antagonist activity of the compounds is determined by progesterone-induced alkaline phosphatase activity in the T-47D cell line (ATCC). In the T-47D breast cancer cells, progesterone specifically induces de novo synthesis of a membrane-associated alkaline phosphatase enzyme in a time and dose-dependent manner (Di Lorenzo et al., Cancer Research, 51: 4470-4475 (1991)). The alkaline phosphatase enzymatic activity can be measured with a chemiluminescent substrate, such as CSPD® (Applied Biosystems).

The assay is performed in 384 well plates. Briefly, T-47D cells are plated in 384 well plates at a density of approximately 2.5×10⁴ cells/well in 50 μl medium supplemented with 10% fetal bovine serum. Twenty four hours later, the medium is aspirated. New medium that is free of phenol red and serum is added to the cells. Compounds prepared in DMSO are transferred to the cells. The cells are then stimulated with 3 nM final concentration of progesterone (Sigma) and incubated at 37° C. for another 24 hours before the alkaline phosphatase is assayed with 25 μl of CSPD® (Applied Biosystems) using a luminometer (CLIPR). The expression of alkaline phosphatase is used as an indicator of progesterone-induced PR trans-activation. Each compound is tested in duplicate with a 12-concentration titration. IC50 values (defined as the concentration of test compound required to antagonize 50% of progesterone-induced PR activity) are determined from the dose-response curve.

### Example 7

### Functional Assay of Androgen Receptor Antagonism

The AR antagonist activity of the compounds is determined with the MDA-Kb2 cell line (ATCC), which stably expresses the MMTV luciferase reporter. The MMTV promoter is a mouse mammary tumor virus promoter that contains androgen receptor response elements. The MDA-kb2 cells was derived from the MDA-MB-453 cells, which has been shown to express high levels of functional, endogenous androgen receptor (Wilson et al., Toxicological Sciences, 66: 69-81 (2002)). Upon stimulation with AR ligands, such as dihydrotestosterone, the MMTV luciferase reporter can be activated.

The assay is performed in 384 well plates. Briefly, MDA-kb2 cells are plated in 384 well plates at a density of approximately 2.4×10⁴ cells/well in 50 μl medium. The medium is supplemented with 5% charcoal-dextran treated fetal bovine serum (Hyclone). Twenty four hours later, compounds prepared in DMSO are transferred to the cells. The cells are then stimulated with 0.3 nM final concentration of dihydrotestosterone (Sigma) and incubated at 37° C. for another 24 hours before the luciferase activity is assayed with 20 μl of Bright-Glo (Promega) using a luminometer (CLIPR). The expression of luciferase is used as an indicator of dihydrotestosterone-induced AR trans-activation. Each compound is tested in duplicate with a 12-concentration titration. IC50 values (defined as the concentration of test compound required to antagonize 50% of dihydrotestosterone-induced AR activity) are determined from the dose-response curve.

### Example 8

### Calcium Channel Binding Assay

Competitive binding of the compounds of the invention (test compounds) with a known L-type calcium channel blocker is measured with membrane prepared from rat cortex. ³H-PN-200-100 (150 pM) is incubated together with membrane (50 μg) and test compounds at room temperature for 90 minutes. At the end of the incubation, the reaction mixture is transferred to 96 well filter plates and washed 3 times by flash filtration with ice cold buffer. The plate is dried. The radioactivity is counted in Topcount by liquid scintilation. Non-specific binding is determined in the presence of 1 μM nitrendipine and subtracted from the total binding to obtain the specific binding of test compounds.

### Example 9

### Potassium-Induced Rat Aorta Ring Contractility Assay

The calcium antagonist function of the compounds of the invention (test compounds) is evaluated in the potassium-induced aorta contractility assay at concentrations ranging from 0.1 μM to 10 μM. Briefly, an endothelial denuded aortic ring obtained from Wister-derived rats is placed under 2 g of tension in a 10 ml bath containing Kreb solution (pH 7.4) and 1 μM meclofenamate at 37° C. Any contraction induced by a test compound is recorded isometrically within 5 minutes of the compound addition. If no significant agonist activity is observed, the ability of the test compound to reduce 60 mM KCl-induced contractile response is measured. An inhibition of KCl-induced response by ≧50% indicates antagonist activity.

Compounds of Formula I, in free form or in pharmaceutically acceptable salt form, exhibit valuable pharmacological properties, for example, as indicated by the in vitro tests described in this application (Examples 4-7). The compounds of the invention preferably exhibit inhibitory activity for steroid hormone nuclear receptors and L-type calcium channel with an IC₅₀ in the range of 1×10⁻⁹ to 1×10⁻⁵M, preferably less than 1 μM, more preferably less than 500 nM. For example:

(i). 2-methylthio-3-cyano-4-(2-bromophenyl)-5-(2-methoxyphenyl)carbamoyl-6-methyl-1,4-dihydro-pyridine (Compound 14) has an IC₅₀ of 8 nM and 2.6 μM for MR and AR, respectively;

(ii). 5-isopropyl-2-methyl-3-cyano-4-(2-bromo-4-fluorophenyl)-6-(methoxymethyl) 1,4-dihydro-pyridine-5-carboxylate (Compound 96) has an IC₅₀ of 9 nM, 39.8 μM, 2.3 μM and 3.1 μM for MR, AR, PR and GR, respectively;

(iii). 5-methyl-2,6-dimethyl-3-cyano-4-(2-chloro-4-fluorophenyl)-1,4-dihydro-pyridine-5-carboxylate (Example 207) has an IC₅₀ of 41 nM for MR 0.478 μM for PR, 2.86 μM for AR, 6.85 μM for GR and 0.24 μM for L-type calcium channel; and

The compounds of the present invention are, therefore, useful for the treatment and/or prevention of diseases in which steroidal nuclear hormone receptor activity and/or L-type calcium channel activity contributes to the pathology and/or symptomatology of the disease.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A compound of Formula I: 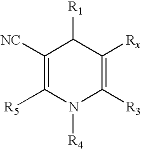
in which:
R₁ is selected from C₆₋₁₀aryl and C₅₋₁₀heteroaryl; wherein any aryl or heteroaryl of R₁ is optionally substituted by 1 to 3 radicals independently selected from halo, C₁₋₆alkyl, C₁₋₆alkoxy, phenyl, halo-substituted-C₁₋₆alkyl and halo-substituted-C₁₋₆alkoxy;
Rₓ is selected from cyano and —C(O)R₂; wherein R₂ is selected from —NR₆R₇ and —OR₇; wherein R₆ is selected from hydrogen, C₁₋₆alkyl and 1-hydroxy-vinyl; and R₇ is selected from C₁₋₆alkyl, halo-substituted-C₁₋₆alkyl, C₃₋₁₂cycloalkyl, C₆₋₁₀aryl and C₅₋₁₀heteroaryl; wherein any cycloalkyl, aryl or heteroaryl of R₇ is optionally substituted by 1 to 3 radicals independently selected from halo, nitro, C₁₋₆alkyl, C₁₋₆alkoxy, phenyl, phenoxy, halo-substituted-C₁₋₆alkyl and halo-substituted-C₁₋₆alkoxy; or R₆ and R₇ together with the nitrogen to which they are both attached form C₅₋₁₀heteroaryl or C₃₋₈heterocycloalkyl;
R₃ is selected from C₁₋₆alkyl, C₄₋₁₂cycloalkyl-C₀₋₄alkyl, C₆₋₁₀aryl-C₀₋₄alkyl and C₅₋₁₀heteroaryl-C₀₋₄alkyl; wherein any alkyl of R₃ can optionally have a methylene replaced with a divalent radical independently selected from —O—, —OC(O)—, —NR₆— and —S(O)₀₋₂—; wherein any alkyl of R₃ can optionally be substituted by 1 to 3 radicals independently selected from halo-substituted-C₁₋₆alkyl; wherein any cycloalkyl, aryl or heteroaryl of R₃ can optionally be substituted with 1 to 2 radicals independently selected from halo, C₁₋₆alkyl and C₁₋₆alkoxy; or R₂ and R₃ together with the atoms to which R₂ and R₃ are attached form C₃₋₁₂cycloalkyl optionally substituted with 1 to 2 radicals independently selected from halo, nitro, C₁₋₆alkyl, C₁₋₆alkoxy, phenyl, halo-substituted-C₁₋₆alkyl and halo-substituted-C₁₋₆alkoxy;
R₄ is selected from hydrogen, C₁₋₆alkyl, halo-substituted-C₁₋₆alkyl and —C(O)R₈; wherein R₈ is selected from hydrogen and C₁₋₆alkyl;
R₅ is selected from C₁₋₆alkyl, —SXC(O)OR₉, —SXOC(O)R₉, —SXR₉, —SXC(O)R₉, —SXNR₉R₉ and —XR₉; wherein X is a bond or C₁₋₆alkylene; R₉ is independently selected from hydroxy, C₁₋₆alkyl, halo-substituted-C₁₋₆alkyl, C₆₋₁₀aryl and C₅₋₁₀heteroaryl; wherein any aryl or heteroaryl of R₉ is optionally substituted with 1 to 3 radicals independently selected from halo, hydroxy, nitro, amino, cyano, C₁₋₆alkyl, C₁₋₆alkoxy, halo-substituted-C₁₋₆alkyl, halo-substituted-C₁₋₆alkoxy, —C(O)OR₁₀, —OR₁₀ and —C(O)R₁₀; wherein R₁₀ is selected from methyl and phenyl; and the pharmaceutically acceptable salts, hydrates, solvates and isomers thereof.

2. The compound of claim 1 in which:
R₁ is selected from phenyl, pyridinyl, thienyl and quinolinyl; wherein any aryl or heteroaryl of R₁ is optionally substituted with 1 to 3 radicals independently selected from chloro, bromo, fluoro, trifluoromethyl, methyl, ethyl, methoxy, allyloxy and phenyl;
Rₓ is selected from cyano and —C(O)R₂; wherein R₂ is selected from —NR₆R₇ and —OR₇; wherein R₆ is selected from hydrogen and C₁₋₆alkyl; and R₇ is selected from methyl, ethyl, isopropyl, trifluoro-butyl, 2,2-dimethyl-propyl, 3,3-dimethyl-butyl, phenyl and pyridinyl; wherein any aryl or heteroaryl of R₇ is optionally substituted by 1 to 3 radicals independently selected from halo, methoxy, ethoxy and phenoxy;
R₃ is selected from methyl, propyl, cyclopropyl, butyl, isobutyl, phenyl, furanyl, optionally substituted with halo; wherein any alkyl of R₃ can optionally have a methylene replaced with —O—; wherein any cycloalkyl, aryl or heteroaryl of R₃ can optionally be substituted with 1 to 2 radicals independently selected from halo and methoxy; or R₂ and R₃ together with the atoms to which R₂ and R₃ are attached form cyclohexanone optionally substituted with 1 to 2 radicals independently selected from methyl, ethyl, propyl, isopropyl and phenyl;
R₄ is hydrogen; and
R₅ is selected from C₁₋₆alkyl, —SXC(O)OR₉, —SXOC(O)R₉, —SXR₉, —SXC(O)R₉, —SXNR₉R₉ and —XR₉; wherein X is a bond or C₁₋₆alkylene; R₉ is independently selected from hydroxy, C₁₋₆alkyl, halo-substituted-C₁₋₆alkyl, C₆₋₁₀aryl and C₅₋₁₀heteroaryl; wherein any aryl or heteroaryl of R₉ is optionally substituted with 1 to 3 radicals independently selected from halo, hydroxy, nitro, amino, cyano, C₁₋₆alkyl, C₁₋₆alkoxy, halo-substituted-C₁₋₆alkyl, halo-substituted-C₁₋₆alkoxy, —C(O)OR₁₀, —OR₁₀ and —C(O)R₁₀; wherein R₁₀ is selected from methyl and phenyl.

3. The compound of claim 2 in which R₅ is C₁₋₆alkyl or —XR₉; wherein X is a bond or C₁₋₆alkylene; R₉ is independently selected from hydroxy, C₁₋₆alkyl, halo-substituted-C₁₋₆alkyl, C₆₋₁₀aryl and C₅₋₁₀heteroaryl; wherein any aryl or heteroaryl of R₉ is optionally substituted with 1 to 3 radicals independently selected from halo, hydroxy, nitro, amino, cyano, C₁₋₆alkyl, C₁₋₆alkoxy, halo-substituted-C₁₋₆alkyl, halo-substituted-C₁₋₆alkoxy, —C(O)OR₁₀, —OR₁₀ and —C(O)R₁₀; wherein R₁₀ is selected from methyl and phenyl

4. The compound of claim 3 of Formula Ia: 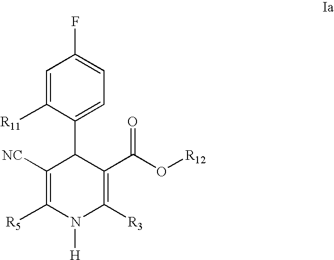
in which:
R₃ is selected from methyl, ethyl, propyl, methoxy-methyl, methoxy-ethyl, methoxy-propyl, methyl-carbonyl-oxy-propyl, hydroxy-propyl, phenethyl, trifluoromethyl-butyl, cyclopropyl, cyclopropyl-methyl, cyclopropyl-ethyl and difluoromethyl;
R₅ is selected from methyl, propyl, benzyl optionally substituted with fluoro, bromo, chloro or methoxy, phenethyl optionally substituted with methoxy, phenyl optionally substituted with chloro, isobutyl, furanyl, methoxy-methyl and trifluoromethyl-ethyl;
R₁₁ is selected from chloro, bromo, fluoro, trifluoromethyl and methoxy; and
R₁₂ is selected from cyclopropyl-methyl, isopropyl, methyl, ethyl, propyl, butyl, isobutyl, trifluoromethyl-propyl, trifluoromethyl-ethyl, t-butyl, t-butyl-methyl, t-butyl-ethyl, isopropyl-ethyl, 1,1-dimethyl-propyl, cyclobutyl-methyl and allyl.

5. The compound of claim 4 selected from: 5-cyclopropylmethyl-2-methyl-3-cyano-4-(2-chloro-4-fluorophenyl)-6-ethyl-1,4-dihydro-pyridine-5-carboxylate; 5-isopropyl-2-methyl-3-cyano-4-(2-chloro-4-fluorophenyl)-6-(methoxymethyl)-1,4-dihydro-pyridine-5-carboxylate; 5-isopropyl-2-methyl-3-cyano-4-(2,4-difluorophenyl)-6-(methoxymethyl)-1,4-dihydro-pyridine-5-carboxylate; 5-isopropyl-2-methyl-3-cyano-4-(2-trifluoromethyl-4-fluorophenyl)-6-(methoxymethyl)-1,4-dihydro-pyridine-5-carboxylate; 5-methyl-2-methyl-3-cyano-4-(2-chloro-4-fluorophenyl)-6-(methoxymethyl)-1,4-dihydro-pyridine-5-carboxylate; 5-isopropyl-2-methyl-3-cyano-4-(2-bromo-4-fluorophenyl)-6-(methoxymethyl)-1,4-dihydro-pyridine-5-carboxylate; 5-methyl-2-methyl-3-cyano-4-(2-bromo-4-fluorophenyl)-6-(methoxymethyl)-1,4-dihydro-pyridine-5-carboxylate; 5-ethyl-2-methyl-3-cyano-4-(2-trifluoromethyl-4-fluorophenyl)-6-propyl-1,4-dihydro-pyridine-5-carboxylate; 5-methyl-2-methyl-3-cyano-4-(2-trifluoromethyl-4-fluorophenyl)-6-(methoxyethyl)-1,4-dihydro-pyridine-5-carboxylate; 5-isopropyl-2-methyl-3-cyano-4-(4-fluorophenyl)-6-(methoxymethyl)-1,4-dihydro-pyridine-5-carboxylate; 5-ethyl-2-methyl-3-cyano-4-(2-chloro-4-fluorophenyl)-6-(2-methoxymethyl)-1,4-dihydro-pyridine-5-carboxylate; 5-butyl-2-methyl-3-cyano-4-(2-chloro-4-fluorophenyl)-6-(2-methoxymethyl)-1,4-dihydro-pyridine-5-carboxylate; 5-(3-methylpropyl)-2-methyl-3-cyano-4-(2-chloro-4-fluorophenyl)-6-(2-methoxymethyl)-1,4-dihydro-pyridine-5-carboxylate; 5-methyl-2-methyl-3-cyano-4-(2-chloro-4-fluorophenyl)-6-(2-methoxyethyl)-1,4-dihydro-pyridine-5-carboxylate; 5-methyl-2-methyl-3-cyano-4-(2-bromo-4-fluorophenyl)-6-(2-methoxyethyl)-1,4-dihydro-pyridine-5-carboxylate; 5-methyl-2-phenylmethyl-3-cyano-4-(2-chloro-4-fluorophenyl)-6-methyl-1,4-dihydro-pyridine-5-carboxylate; 5-methyl-2-(2-phenyl)ethyl-3-cyano-4-(2-chloro-4-fluorophenyl)-6-methyl-1,4-dihydro-pyridine-5-carboxylate; 5-(3,3,3-trifluorobutyl)-2-methyl-3-cyano-4-(2-chloro-4-fluorophenyl)-1,4-dihydro-pyridine-5-carboxylate; 5-(3,3,3-trifluoroproryl)-2-methyl-3-cyano-4-(2-chloro-4-fluorophenyl)-6-methoxymethyl-1,4-dihydro-pyridine-5-carboxylate; 5-iso-propyl-2-methyl-3-cyano-4-(2-trifluoromethyl-4-fluorophenyl)-6-propyl-1,4-dihydro-pyridine-5-carboxylate; 5-tert-butyl-2-methyl-3-cyano-4-(2-trifluoromethyl-4-fluorophenyl)-6-propyl-1,4-dihydro-pyridine-5-carboxylate; 5-methyl-2,6-dimethyl-3-cyano-4-(2-trifluoromethyl-4-fluorophenyl)-1,4-dihydro-pyridine-5-carboxylate; 5-(2-methylpropyl)-2-methyl-3-cyano-4-(2-trifluoromethyl-4-fluorophenyl)-6-methyl-1,4-dihydro-pyridine-5-carboxylate; 5-ethyl-2-methyl-3-cyano-4-(2-trifluoromethyl-4-fluorophenyl)-6-(2-methoxymethyl)-1,4-dihydro-pyridine-5-carboxylate; 5-ethyl-2-methyl-3-cyano-4-(2-bromo-4-fluorophenyl)-6-(2-methoxymethyl)-1,4-dihydro-pyridine-5-carboxylate; 5-butyl-2-methyl-3-cyano-4-(2-trifluoromethyl-4-fluorophenyl)-6-(2-methoxymethyl)-1,4-dihydro-pyridine-5-carboxylate; 5-butyl-2-methyl-3-cyano-4-(2-bromo-4-fluorophenyl)-6-(2-methoxymethyl)-1,4-dihydro-pyridine-5-carboxylate; 5-(2-methylpropyl)-2-methyl-3-cyano-4-(2-trifluoromethyl-4-fluorophenyl)-6-(2-methoxymethyl)-1,4-dihydro-pyridine-5-carboxylate; 5-(2-methylpropyl)-2-methyl-3-cyano-4-(2-bromo-4-fluorophenyl)-6-(2-methoxymethyl)-1,4-dihydro-pyridine-5-carboxylate; 5-methyl-2-phenyl-3-cyano-4-(2-chloro-4-fluorophenyl)-6-methyl-1,4-dihydro-pyridine-5-carboxylate; 5-tert-amyl-2-methyl-3-cyano-4-(2-chloro-4-fluorophenyl)-6-propyl-1,4-dihydro-pyridine-5-carboxylate; 5-tert-amyl-2-methyl-3-cyano-4-(2-bromo-4-fluorophenyl)-6-propyl-1,4-dihydro-pyridine-5-carboxylate; 5-tert-amyl-2-methyl-3-cyano-4-(2-trifluoromethyl-4-fluorophenyl)-6-propyl-1,4-dihydro-pyridine-5-carboxylate; 5-tert-amyl-2-methyl-3-cyano-4-(2-methoxy-4-fluorophenyl)-6-propyl-1,4-dihydro-pyridine-5-carboxylate; 5-(3,3-dimethylbutyl)-2-methyl-3-cyano-4-(2-chloro-4-fluorophenyl)-6-propyl-1,4-dihydro-pyridine-5-carboxylate; 5-(3,3-dimethylbutyl)-2-methyl-3-cyano-4-(2-bromo-4-fluorophenyl)-6-propyl-1,4-dihydro-pyridine-5-carboxylate; 5-(3,3-dimethylbutyl)-2-methyl-3-cyano-4-(2-trifluoromethyl-4-fluorophenyl)-6-propyl-1,4-dihydro-pyridine-5-carboxylate; 5-(3,3-dimethylbutyl)-2-methyl-3-cyano-4-(2-methoxy-4-fluorophenyl)-6-propyl-1,4-dihydro-pyridine-5-carboxylate; 5-ethyl-2-methyl-3-cyano-4-(2-methoxy-4-fluorophenyl)-6-propyl-1,4-dihydro-pyridine-5-carboxylate; 5-butyl-2-methyl-3-cyano-4-(2-bromo-4-fluorophenyl)-6-(2-methoxymethyl)-1,4-dihydro-pyridine-5-carboxylate; 5-iso-propyl-2-methyl-3-cyano-4-(2-trifluoromethyl-4-fluorophenyl)-6-propyl-1,4-dihydro-pyridine-5-carboxylate; 5-propyl-2-methyl-3-cyano-4-(2-chloro-4-fluorophenyl)-6-propyl-1,4-dihydro-pyridine-5-carboxylate; 5-propyl-2-methyl-3-cyano-4-(2-bromo-4-fluorophenyl)-6-propyl-1,4-dihydro-pyridine-5-carboxylate; 5-propyl-2-methyl-3-cyano-4-(2-trifluoromethyl-4-fluorophenyl)-6-propyl-1,4-dihydro-pyridine-5-carboxylate; 5-propyl-2-methyl-3-cyano-4-(2-methoxy-4-fluorophenyl)-6-propyl-1,4-dihydro-pyridine-5-carboxylate; 5-propyl-2-methyl-3-cyano-4-(2-chloro-4-fluorophenyl)-6-(2-methoxypropyl)-1,4-dihydro-pyridine-5-carboxylate; 5-propyl-2-methyl-3-cyano-4-(2-bromo-4-fluorophenyl)-6-(2-methoxypropyl)-1,4-dihydro-pyridine-5-carboxylate; 5-propyl-2-methyl-3-cyano-4-(2-trifluoromethyl-4-fluorophenyl)-6-(2-methoxypropyl)-1,4-dihydro-pyridine-5-carboxylate; 5-propyl-2-methyl-3-cyano-4-(2-methoxy-4-fluorophenyl)-6-(2-methoxypropyl)-1,4-dihydro-pyridine-5-carboxylate; 5-(3,3-dimethylbutyl)-2-methyl-3-cyano-4-(2-chloro-4-fluorophenyl)-6-methyl-1,4-dihydro-pyridine-5-carboxylate; 5-(3,3-dimethylbutyl)-2-methyl-3-cyano-4-(2-bromo-4-fluorophenyl)-6-methyl-1,4-dihydro-pyridine-5-carboxylate; 5-(3,3-dimethylbutyl)-2-methyl-3-cyano-4-(2-trifluoromethyl-4-fluorophenyl)-6-methyl-1,4-dihydro-pyridine-5-carboxylate; 5-(3,3-dimethylbutyl)-2-methyl-3-cyano-4-(2-methoxy-4-fluorophenyl)-6-methyl-1,4-dihydro-pyridine-5-carboxylate; 5-methyl-2-tert-butyl-3-cyano-4-(2-chloro-4-fluorophenyl)-6-methyl-1,4-dihydro-pyridine-5-carboxylate; 5-methyl-2-(4-chlorophenyl)-3-cyano-4-(2-chloro-4-fluorophenyl)-6-methyl-1,4-dihydro-pyridine-5-carboxylate; 5-methyl-2-(2-furanyl)-3-cyano-4-(2-chloro-4-fluorophenyl)-6-methyl-1,4-dihydro-pyridine-5-carboxylate; 5-tert-butyl-2-methyl-3-cyano-4-(2-trifluoromethyl-4-fluorophenyl)-6-(2-methoxymethyl)-1,4-dihydro-pyridine-5-carboxylate; 5-iso-propyl-2-methyl-3-cyano-4-(2-methoxy-4-fluorophenyl)-6-methyl-1,4-dihydro-pyridine-5-carboxylate; 5-methyl-2-propyl-3-cyano-4-(2-chloro-4-fluorophenyl)-6-methoxymethyl-1,4-dihydro-pyridine-5-carboxylate; 5-methyl-2-propyl-3-cyano-4-(2-trifluoromethyl-4-fluorophenyl)-6-methoxymethyl-1,4-dihydro-pyridine-5-carboxylate; 5-methyl-2-(4-fluorophenyl)methyl-3-cyano-4-(2-trifluoromethyl-4-fluorophenyl)-6-methyl-1,4-dihydro-pyridine-5-carboxylate; 5-methyl-2-(4-fluorophenyl)methyl-3-cyano-4-(2-trifluoromethyl-4-fluorophenyl)-6-methyl-1,4-dihydro-pyridine-5-carboxylate; 2-(2-phenyl)ethyl-3,5-dicyano-4-(2-chloro-4-fluorophenyl)-6-methyl-1,4-dihydro-pyridine; 2,6-dimethyl-3-cyano-4-(2,4-dichloro)-5-(2-methoxyphenyl)carbamoyl-1,4-dihydropyridine; 5-methyl-2-methyl-3-cyano-4-(2-chloro-4-fluorophenyl)-6-(3-methoxypropyl)-1,4-dihydro-pyridine-5-carboxylate; 5-tert-butyl-2-methyl-3-cyano-4-(2-chloro-4-fluorophenyl)-6-(3-methoxypropyl)-1,4-dihydro-pyridine-5-carboxylate; 5-methyl-2-methyl-3-cyano-4-(2-trifluoromethyl-4-fluorophenyl)-6-(3-methoxypropyl)-1,4-dihydro-pyridine-5-carboxylate; 5-methyl-2-methyl-3-cyano-4-(2-chloro-4-fluorophenyl)-6-(3-acetoxypropyl)-1,4-dihydro-pyridine-5-carboxylate; 5-methyl-2-methyl-3-cyano-4-(2-trifluoromethyl-4-fluorophenyl)-6-(3-acetoxypropyl)-1,4-dihydro-pyridine-5-carboxylate; 5-methyl-2-methyl-3-cyano-4-(2-chloro-4-fluorophenyl)-6-(3-hydroxypropyl)-1,4-dihydro-pyridine-5-carboxylate; 5-methyl-2-methyl-3-cyano-4-(2-trifluoromethyl-4-fluorophenyl)-6-(3-hydroxypropyl)-1,4-dihydro-pyridine-5-carboxylate; 5-methyl-2-methyl-3-cyano-4-(2-trifluoromethyl-4-fluorophenyl)-6-(3-hydroxypropyl)-1,4-dihydro-pyridine-5-carboxylate; 5-methyl-2-methyl-3-cyano-4-(2-chloro-4-fluorophenyl)-6-(2-phenylethyl)-1,4-dihydro-pyridine-5-carboxylate; 5-methyl-2-methyl-3-cyano-4-(2-trifluoromethyl-4-fluorophenyl)-6-(2-phenylethyl)-1,4-dihydro-pyridine-5-carboxylate; 5-methyl-2-methyl-3-cyano-4-(2-chloro-4-fluorophenyl)-6-(5,5,5-trifluoropentyl)-1,4-dihydro-pyridine-5-carboxylate; 5-methyl-2-methyl-3-cyano-4-(2-trifluoromethyl-4-fluorophenyl)-6-(5,5,5-trifluoropentyl)-1,4-dihydro-pyridine-5-carboxylate; 5-methyl-2,6-dimethyl-3-cyano-4-(2-chloro-4-fluorophenyl)-1,4-dihydro-pyridine-5-carboxylate; 5-methyl-2-methyl-3-cyano-4-(2-trifluoromethyl-4-fluorophenyl)-6-cyclopropyl-1,4-dihydro-pyridine-5-carboxylate; 5-methyl-2-methyl-3-cyano-4-(2-chloro-4-fluorophenyl)-6-cyclopropyl-1,4-dihydro-pyridine-5-carboxylate; 5-methyl-2-methyl-3-cyano-4-(2-chloro-4-fluorophenyl)-6-2-cyclopropylethyl-1,4-dihydro-pyridine-5-carboxylate; 5-cyclopropylmethyl-2-methyl-3-cyano-4-(2-chloro-4-fluorophenyl)-6-(2-methoxyethyl)-1,4-dihydro-pyridine-5-carboxylate; 5-methyl-2-phenylmethyl-3-cyano-4-(2-trifluoromethyl-4-fluorophenyl)-6-methyl-1,4-dihydro-pyridine-5-carboxylate; 5-methyl-2-(2-furyl)-3-cyano-4-(2-chloro-4-fluorophenyl)-6-(2-methoxymethyl)-1,4-dihydro-pyridine-5-carboxylate; 5-methyl-2-(2-phenylethyl)-3-cyano-4-(2-chloro-4-fluorophenyl)-6-(2-methoxymethyl)-1,4-dihydro-pyridine-5-carboxylate; 5-methyl-2-(4-chlorophenyl)-3-cyano-4-(2-chloro-4-fluorophenyl)-6-(2-methoxymethyl)-1,4-dihydro-pyridine-5-carboxylate; 5-methyl-2-(2-furyl)-3-cyano-4-(2-trifluoromethyl-4-fluorophenyl)-6-(2-methoxymethyl)-1,4-dihydro-pyridine-5-carboxylate; 5-methyl-2-(2-phenylethyl)-3-cyano-4-(2-trifluoromethyl-4-fluorophenyl)-6-(2-methoxymethyl)-1,4-dihydro-pyridine-5-carboxylate; 5-(3,3,3-trifluoropropyl)-2-methyl-3-cyano-4-(2-trifluoromethyl-4-fluorophenyl)-6-(2-methoxymethyl)-1,4-dihydro-pyridine-5-carboxylate; 5-ethyl-2-(2-furyl)-3-cyano-4-(2-chloro-4-fluorophenyl)-6-propyl1,4-dihydro-pyridine-5-carboxylate; 5-ethyl-2-(2-phenylethyl)-3-cyano-4-(2-chloro-4-fluorophenyl)-6-propyl1,4-dihydro-pyridine-5-carboxylate; 5-(4,4,4-trifluorobutyl)-2-methyl-3-cyano-4-(2-methoxy-4-fluorophenyl)-6-propyl1,4-dihydro-pyridine-5-carboxylate; 5-(4,4,4-trifluorobutyl)-2-methyl-3-cyano-4-(2-trifluoromethyl-4-fluorophenyl)-6-propyl1,4-dihydro-pyridine-5-carboxylate; 5-tert-butyl-2-methyl-3-cyano-4-(2-chloro-4-fluorophenyl)-6-(2-methoxymethyl1,4-dihydro-pyridine-5-carboxylate; 5-(3,3-dimethylbutyl)-2-methyl-3-cyano-4-(2-chloro-4-fluorophenyl)-6-(2-methoxymethyl1,4-dihydro-pyridine-5-carboxylate; 5-(3,3-dimethylbutyl)-2-methyl-3-cyano-4-(2-trifluoromethyl-4-fluorophenyl)-6-(2-methoxymethyl1,4-dihydro-pyridine-5-carboxylate; 5-methyl-2-[2-(4-methoxyphenyl)ethyl]-3-cyano-4-(2-chloro-4-fluorophenyl)-6-methyl-1,4-dihydro-pyridine-5-carboxylate; 5-(2,2-dimethylpropyl)-2methyl-3-cyano-4-(2-chloro-4-fluorophenyl)-6-methyl-1,4-dihydro-pyridine-5-carboxylate; 5-(2,2-dimethylpropyl)-2methyl-3-cyano-4-(2-trifluoromethyl-4-fluorophenyl)-6-(2-methoxymethyl)-1,4-dihydro-pyridine-5-carboxylate; 5-(1,1-dimethylpropyl)-2methyl-3-cyano-4-(2-chloro-4-fluorophenyl)-6-)2-methoxymethyl)-1,4-dihydro-pyridine-5-carboxylate; 5-(1,1-dimethylpropyl)-2methyl-3-cyano-4-(2-trifluoromethyl-4-fluorophenyl)-6-(2-methoxymethyl)-1,4-dihydro-pyridine-5-carboxylate; 5-methyl-2-(2-methoxymethyl)-3-cyano-4-(2-trifluoromethyl-4-fluorophenyl)-6-(2-methoxymethyl)-1,4-dihydro-pyridine-5-carboxylate; 5-methyl-2-(3,3,3-trifluoropropyl)-3-cyano-4-(2-chloro-4-fluorophenyl)-6-methyl-1,4-dihydro-pyridine-5-carboxylate; 5-methyl-2-(3-methoxyphenylmethyl)-3-cyano-4-(2-chloro-4-fluorophenyl)-6-methyl-1,4-dihydro-pyridine-5-carboxylate; 5-methyl-2-(4-methoxyphenylmethyl)-3-cyano-4-(2-chloro-4-fluorophenyl)-6-methyl-1,4-dihydro-pyridine-5-carboxylate; 5-methyl-2-(3-methoxyphenylmethyl)-3-cyano-4-(2-chloro-4-fluorophenyl)-6-(2-methoxymethyl)-1,4-dihydro-pyridine-5-carboxylate; 5-methyl-2-(4-methoxyphenylmethyl)-3-cyano-4-(2-chloro-4-fluorophenyl)-6-(2-methoxymethyl)-1,4-dihydro-pyridine-5-carboxylate; 5-methyl-2-(2-fluorophenylmethyl)-3-cyano-4-(2-chloro-4-fluorophenyl)-6-methyl-1,4-dihydro-pyridine-5-carboxylate; 5-methyl-2-(3-fluorophenylmethyl)-3-cyano-4-(2-chloro-4-fluorophenyl)-6-methyl-1,4-dihydro-pyridine-5-carboxylate; 5-methyl-2-(4-chlorophenylmethyl)-3-cyano-4-(2-chloro-4-fluorophenyl)-6-methyl-1,4-dihydro-pyridine-5-carboxylate; 5-methyl-2-(4-bromophenylmethyl)-3-cyano-4-(2-chloro-4-fluorophenyl)-6-methyl-1,4-dihydro-pyridine-5-carboxylate; 5-methyl-2-(2-fluorophenylmethyl)-3-cyano-4-(2-trifluoromethyl-4-fluorophenyl)-6-methyl-1,4-dihydro-pyridine-5-carboxylate; 5-methyl-2-(3-fluorophenylmethyl)-3-cyano-4-(2-trifluoromethyl-4-fluorophenyl)-6-methyl-1,4-dihydro-pyridine-5-carboxylate; 5-methyl-2-(4-chlorophenylmethyl)-3-cyano-4-(2-trifluoromethyl-4-fluorophenyl)-6-methyl-1,4-dihydro-pyridine-5-carboxylate; 5-methyl-2-(4-bromophenylmethyl)-3-cyano-4-(2-trifluoromethyl-4-fluorophenyl)-6-methyl-1,4-dihydro-pyridine-5-carboxylate; 5-cyclopropylmethyl-2-methyl-3-cyano-4-(2-chloro-4-fluorophenyl)-6-(2-cyclopropyl)ethyl-1,4-dihydro-pyridine-5-carboxylate; 5-ethyl-2-methyl-3-cyano-4-(2-chloro-4-fluorophenyl)-6-(2-cyclopropyl)ethyl-1,4-dihydro-pyridine-5-carboxylate; 5-ethyl-2-methyl-3-cyano-4-(2-trifluoromethyl-4-fluorophenyl)-6-ethyl-1,4-dihydro-pyridine-5-carboxylate; 5-cyclopropylmethyl-2-methyl-3-cyano-4-(2-trifluoromethyl-4-fluorophenyl)-6-(2-methoxyethyl)-1,4-dihydro-pyridine-5-carboxylate; 5-cyclobutylmethyl-2-methyl-3-cyano-4-(2-trifluoromethyl-4-fluorophenyl)-6-(2-methoxyethyl)-1,4-dihydro-pyridine-5-carboxylate; 5-ethyl-2-methyl-3-cyano-4-(2-chloro-4-fluorophenyl)-6-difluoromethyl-1,4-dihydro-pyridine-5-carboxylate; 5-ethyl-2-methyl-3-cyano-4-(2-trifluoromethyl-4-fluorophenyl)-6-difluoromethyl-1,4-dihydro-pyridine-5-carboxylate; 5-cyclopropylmethyl-2-methyl-3-cyano-4-(2-trifluoromethyl-4-fluorophenyl)-6-propyl-1,4-dihydro-pyridine-5-carboxylate; 5-allyl-2-(2-fluorophenyl)methyl-3-cyano-4-(2-trifluoromethyl-4-fluorophenyl)-6-methyl-1,4-dihydro-pyridine-5-carboxylate; 5-(2,2-dimethylpropyl)-2-methyl-3-cyano-4-(2-trifluoromethyl-4-fluorophenyl)-6-ethyl-1,4-dihydro-pyridine-5-carboxylate; 5-cyclopropylmethyl-2-methyl-3-cyano-4-(2-trifluoromethyl-4-fluorophenyl)-6-ethyl-1,4-dihydro-pyridine-5-carboxylate; 5-cyclopropylmethyl-2-methyl-3-cyano-4-(2-trifluoromethyl-4-fluorophenyl)-6-propyl 1,4-dihydro-pyridine-5-carboxylate; 5-(2-methyl)propyl-2-methyl-3-cyano-4-(2-trifluoromethyl-4-fluorophenyl)-6-ethyl-1,4-dihydro-pyridine-5-carboxylate; and 5-(2,2-dimethyl)propyl-2-methyl-3-cyano-4-(2-chloro-4-fluorophenyl)-6-ethyl-1,4-dihydro-pyridine-5-carboxylate.

6. The compound of claim 3 of Formula Ib: 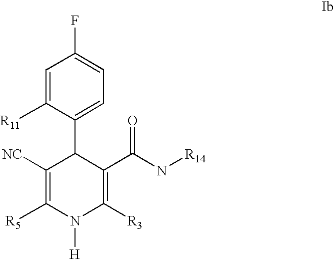
in which:
R₃ is selected from methyl, ethyl, propyl, methoxy-methyl, methoxy-ethyl, methoxy-propyl, methyl-carbonyl-oxy-propyl, hydroxy-propyl, phenethyl, trifluoromethyl-butyl, cyclopropyl, cyclopropyl-methyl, cyclopropyl-ethyl and difluoromethyl;
R₅ is selected from methyl, propyl, benzyl optionally substituted with fluoro, bromo, chloro or methoxy, methyl-thio, ethyl-thio, propyl-thio, butyl-thio, trifluoromethyl-propyl-thio, phenethyl optionally substituted with methoxy, phenyl optionally substituted with chloro, isobutyl, furanyl, methoxy-methyl and trifluoromethyl-ethyl;
R₁₁ is selected from chloro, bromo, fluoro, trifluoromethyl and methoxy; and
R₁₄ is selected from cyclopropyl-methyl, isopropyl, methyl, ethyl, propyl, butyl, isobutyl, trifluoromethyl-propyl, trifluoromethyl-ethyl, t-butyl, t-butyl-methyl, t-butyl-ethyl, isopropyl-ethyl, 1,1-dimethyl-propyl, cyclobutyl-methyl and allyl.

7. The compound of claim 6 selected from: 2-ethylthio-3-cyano-4-(2,4-difluorophenyl)-5-(2-methoxyphenyl)carbamoyl-6-methyl-1,4-dihydro-pyridine; 2-butylthio-3-cyano-4-(2,4-difluorophenyl)-5-(2-methoxyphenyl)carbamoyl-6-methyl-1,4-dihydro-pyridine; 2-(4,4,4-trifluorobutyl)thio-3-cyano-4-(2,4-difluorophenyl)-5-(2-methoxyphenyl)carbamoyl-6-methyl-1,4-dihydro-pyridine; and 2-(2-phenylmethyl)-3-cyano-4-(2-chloro-4-fluorophenyl)-5-(2-chloro-4-fluorophenyl)carbamoyl-6-methyl-1,4-dihydro-pyridine.

8. The compounds of claim 3 selected from: N-methyl-4-morpholinium-6-methyl-4-(2-fluoro-4-bromophenyl)-5-(2-methoxyphenyl)carbamoyl-3-cyano-1,4-dihydro-pyridine-2-thiolate 1; 2-(4-methylbenzyl)thio-3-cyano-4-(2-chlorophenyl)-5-(2-methoxyphenyl)carbamoyl-6-methyl-1,4-dihydro-pyridine; 2,6-dimethyl-3-cyano-4-(2-chlorophenyl)-5-(2-methoxyphenyl)carbamoyl-1,4-dihydro-pyridine; 2-(4,4,4-trifluorobutyl)thio-3-cyano-4-(2-chlorophenyl)-5-(2-methoxyphenyl)carbamoyl-6-methyl-1,4-dihydro-pyridine; 2-(2-methylbenzyl)thio-3-cyano-4-(2-chlorophenyl)-5-(2-methoxyphenyl)carbamoyl-6-methyl-1,4-dihydro-pyridine; 2-(3,5-dimethylbenzylbenzyl)thio-3-cyano-4-(2-chlorophenyl)-5-(2-methoxyphenyl)carbamoyl-6-methyl-1,4-dihydro-pyridine; 2-(3-nitrobenzylbenzyl)thio-3-cyano-4-(2-chlorophenyl)-5-(2-methoxyphenyl)carbamoyl-6-methyl-1,4-dihydro-pyridine; 2-methylthio-3-cyano-4-(2,4-dichlorophenyl)-5-(2-methoxyphenyl)carbamoyl-6-methyl-1,4-dihydro-pyridine; 2-ethylthio-3-cyano-4-(2,4-dichlorophenyl)-5-(2-methoxyphenyl)carbamoyl-6-methyl-1,4-dihydro-pyridine; 2-butylthio-3-cyano-4-(2,4-dichlorophenyl)-5-(2-methoxyphenyl)carbamoyl-6-methyl-1,4-dihydro-pyridine; 2-(3-fluoropropyl)thio-3-cyano-4-(2,4-dichlorophenyl)-5-(2-methoxyphenyl)carbamoyl-6-methyl-1,4-dihydro-pyridine; 2-(4,4,4-trifluorofluorobutyl)thio-3-cyano-4-(2,4-dichlorophenyl)-5-(2-methoxyphenyl)carbamoyl-6-methyl-1,4-dihydro-pyridine; 2-benzylthio-3-cyano-4-(2,4-dichlorophenyl)-5-(2-methoxyphenyl)carbamoyl-6-methyl-1,4-dihydro-pyridine; 2-methylthio-3-cyano-4-(2-bromophenyl)-5-(2-methoxyphenyl)carbamoyl-6-methyl-1,4-dihydro-pyridine; 2-butylthio-3-cyano-4-(2-bromophenyl)-5-(2-methoxyphenyl)carbamoyl-6-methyl-1,4-dihydro-pyridine; 2-(4,4,4-trifluorobutylthio-3-cyano-4-(2-bromophenyl)-5-(2-methoxyphenyl)carbamoyl-6-methyl-1,4-dihydro-pyridine; 2-(3-nitrobenzyl)thio-3-cyano-4-(2,4-dichlorophenyl)-5-(2-methoxyphenyl)carbamoyl-6-methyl-1,4-dihydro-pyridine; 2-(3-nitrobenzylthio-3-cyano-4-(2-bromophenyl)-5-(2-methoxyphenyl)carbamoyl-6-methyl-1,4-dihydro-pyridine; 2-(4-carboxymethylbenzylthio-3-cyano-4-(2-bromophenyl)-5-(2-methoxyphenyl)carbamoyl-6-methyl-1,4-dihydro-pyridine; 2-(2-cyanobenzylbenzyl)thio-3-cyano-4-(2-chlorophenyl)-5-(2-methoxyphenyl)carbamoyl-6-methyl-1,4-dihydro-pyridine; 2-(3-cyanobenzylbenzyl)thio-3-cyano-4-(2-chlorophenyl)-5-(2-methoxyphenyl)carbamoyl-6-methyl-1,4-dihydro-pyridine; 2-(3-hydroxymethyl)thio-3-cyano-4-(2-chlorophenyl)-5-(2-methoxyphenyl)carbamoyl-6-methyl-1,4-dihydro-pyridine; 2-(2-cyanobenzylthio-3-cyano-4-(2-bromophenyl)-5-(2-methoxyphenyl)carbamoyl-6-methyl-1,4-dihydro-pyridine; 2-(4-cyanobenzylbenzyl)thio-3-cyano-4-(2-bromophenyl)-5-(2-methoxyphenyl)carbamoyl-6-methyl-1,4-dihydro-pyridine; 2-butylthio-3-cyano-4-(2-trifluoromethylphenyl)-5-(2-methoxyphenyl)carbamoyl-6-methyl-1,4-dihydro-pyridine; 2-(2-hydroxyethyl)thio-3-cyano-4-(2-chlorophenyl)-5-(2-methoxyphenyl)carbamoyl-6-methyl-1,4-dihydro-pyridine; 2-(acetoxyethyl)thio-3-cyano-4-(2-chlorophenyl)-5-(2-methoxyphenyl)carbamoyl-6-methyl-1,4-dihydro-pyridine; 2-(hydroxyethyl)thio-3-cyano-4-(2-bromophenyl)-5-(2-methoxyphenyl)carbamoyl-6-methyl-1,4-dihydro-pyridine; 2-(N,N-diethylaminoethyl)thio-3-cyano-4-(2-chlorophenyl)-5-(2-methoxyphenyl)carbamoyl-6-methyl-1,4-dihydro-pyridine; 2-benzylthio-3-cyano-4-(2-trifluoromethylphenyl)-5-(2-methoxyphenyl)carbamoyl-6-methyl-1,4-dihydro-pyridine; 5-ethyl-2-(hydroxyethyl)thio-3-cyano-4-(2,4-dichlorophenyl)-6-propyl-1,4-dihydro-pyridine-5-carboxylate; 5-ethyl-2-(hydroxypropyl)thio-3-cyano-4-(2,4-dichlorophenyl)-6-propyl-1,4-dihydro-pyridine-5-carboxylate; 2-(4-methylbenzyl)thio-3-cyano-4-(2-bromophenyl)-5-(2-methoxyphenyl)carbamoyl-6-methyl-1,4-dihydro-pyridine; 2-methylthio-3-cyano-4-(2-fluoro-4-bromophenyl)-5-(2-methoxyphenyl)carbamoyl-6-methyl-1,4-dihydro-pyridine; 2-methylthio-3-cyano-4-[3-(2-chloropyridine)]-5-(2-methoxyphenyl)carbamoyl-6-methyl-1,4-dihydro-pyridine; 2-methylthio-3-cyano-4-(2-methoxyphenyl)-5-(2-methoxyphenyl)carbamoyl-6-methyl-1,4-dihydro-pyridine; 2-ethylthio-3-cyano-4-(2-methoxyphenyl)-5-(2-methoxyphenyl)carbamoyl-6-methyl-1,4-dihydro-pyridine; 2-butylthio-3-cyano-4-(2-methoxyphenyl)-5-(2-methoxyphenyl)carbamoyl-6-methyl-1,4-dihydro-pyridine; 2-ethylthio-3-cyano-4-[3-(2-chloropyridine)]-5-(2-methoxyphenyl)carbamoyl-6-methyl-1,4-dihydro-pyridine; 2-(4,4,4-trifluorobutyl)thio-3-cyano-4-[3-(2-chloropyridine)]-5-(2-methoxyphenyl)carbamoyl-6-methyl-1,4-dihydro-pyridine; 2-butylthio-3-cyano-4-[3-(2-chloropyridine)]-5-(2-methoxyphenyl)carbamoyl-6-methyl-1,4-dihydro-pyridine; 2-benzylthio-3-cyano-4-[2-(5-bromothiophene)]-carbamoyl-6-methyl-1,4-dihydro-pyridine; 2-methylthio-3-cyano-4-(2-fluoro-4-chlorophenyl)-carbamoyl-6-methyl-1,4-dihydro-pyridine; 2-ethylthio-3-cyano-4-(2-fluoro-4-chlorophenyl)-carbamoyl-6-methyl-1,4-dihydro-pyridine; 2-propylthio-3-cyano-4-(2-fluoro-4-chlorophenyl)-carbamoyl-6-methyl-1,4-dihydro-pyridine; 2-butylthio-3-cyano-4-(2-fluoro-4-chlorophenyl)-carbamoyl-6-methyl-1,4-dihydro-pyridine; 2-(3-nitro-4-methylbenzyl)thio-3-cyano-4-(2-trifluoromethylphenyl)-carbamoyl-6-methyl-1,4-dihydro-pyridine; 2,6-dimethyl-3-cyano-4-(2,4-dichlorophenyl)-5-(2-methoxyphenyl)carbamoyl-1,4-dihydro-pyridine; 2,6-dimethyl-3-cyano-4-(4-bromophenyl)-5-(2-methoxyphenyl)carbamoyl-1,4-dihydro-pyridine; 2,6-dimethyl-3-cyano-4-(2-allyloxyphenyl)-5-(2-methoxyphenyl)carbamoyl-1,4-dihydro-pyridine; 2,6-dimethyl-3-cyano-4-(2-methoxyphenyl)-5-(2-methoxyphenyl)carbamoyl-1,4-dihydro-pyridine; 2,6-dimethyl-3-cyano-4-[3-(2-methoxypyridine)]-5-(2-methoxyphenyl)carbamoyl-1,4-dihydro-pyridine; 2,6-dimethyl-3-cyano-4-(2,4-dichlorophenyl)-5-phenylcarbamoyl-1,4-dihydro-pyridine; 5-methyl-2-methyl-3-cyano-4-(2,4-dichlorophenyl)-6-cyclopropyl-1,4-dihydro-pyridine-5-carboxylate; 5-methyl-2-methyl-3-cyano-4-(2,4-dichlorophenyl)-6-(methoxymethyl)-1,4-dihydro-pyridine-5-carboxylate; 2,6-dimethyl-3-cyano-4-(2,4-dichlorophenyl)-5-N-(2-methoxyphenyl)-N-(1-hydroxyvynyl)carbamoyl-1,4-dihydro-pyridine; 2-methyl-3-cyano-4-(2,4-dichlorophenyl)-5,6-cyclo-3-methyl-hexyl-1,4-dihydro-pyridine; 2-methyl-3-cyano-4-(2,4-dichlorophenyl)-5,6-cyclo-3-isopropyl-hexyl-1,4-dihydro-pyridine; 2-methyl-3-cyano-4-(2,4-dichlorophenyl)-5,6-cyclo-3-phenyl-hexyl-1,4-dihydro-pyridine; 2,6-dimethyl-3-cyano-4-(4-phenylphenyl)-5-(2-methoxyphenyl)-carbamoyl-1,4-dihydro-pyridine; 2,6-dimethyl-3-cyano-4-(2-bromo-4-methylphenyl)-5-(2-methoxyphenyl)carbamoyl-1,4-dihydro-pyridine; 5-isopropyl-2,6-dimethyl-3-cyano-4-(2,4-dichlorophenyl)-1,4-dihydro-pyridine-5-carboxylate; 5-methyl-2-methyl-3-cyano-4-(2,4-dichlorophenyl)-6-isopropyl-1,4-dihydro-pyridine-5-carboxylate; 5-methyl-2-methyl-3-cyano-4-(2,4-dichlorophenyl)-6-propyl-1,4-dihydro-pyridine-5-carboxylate; 5-ethyl-2-methyl-3-cyano-4-(2,4-dichlorophenyl)-6-isopropyl-1,4-dihydro-pyridine-5-carboxylate; 5-ethyl-2-methyl-3-cyano-4-(2,4-dichlorophenyl)-6-ethyl-1,4-dihydro-pyridine-5-carboxylate; 5-ethyl-2-methyl-3-cyano-4-(2,4-dichlorophenyl)-6-propyl-1,4-dihydro-pyridine-5-carboxylate; 5-ethyl-2-methyl-3-cyano-4-(2,4-dichlorophenyl)-6-(2-fluorophenyl)-1,4-dihydro-pyridine-5-carboxylate; 5-ethyl-2-methyl-3-cyano-4-(2,4-dichlorophenyl)-6-phenyl-1,4-dihydro-pyridine-5-carboxylate; 5-ethyl-2-methyl-3-cyano-4-(2,4-dichlorophenyl)-6-(4-methoxyphenyl)-1,4-dihydro-pyridine-5-carboxylate; 5-ethyl-2-methyl-3-cyano-4-(2,4-dichlorophenyl)-6-(3-furyl)-1,4-dihydro-pyridine-5-carboxylate; 5-ethyl-2-methyl-3-cyano-4-(2,4-dichlorophenyl)-6-(2-furyl)-1,4-dihydro-pyridine-5-carboxylate; 5-isopropyl-2-methyl-3-cyano-4-(2-fluoro-4-chlorophenyl)-6-(methoxymethyl)-1,4-dihydro-pyridine-5-carboxylate; 5-isopropyl-2-methyl-3-cyano-4-(2-fluoro-4-trifluoromethylphenyl)-6-(methoxymethyl)-1,4-dihydro-pyridine-5-carboxylate; 5-isopropyl-2-methyl-3-cyano-4-(2,4-bistrifluoromethylphenyl)-6-(methoxymethyl)-1,4-dihydro-pyridine-5-carboxylate; 5-isopropyl-2-methyl-3-cyano-4-(2-chloro-5-trifluoromethylphenyl)-6-(methoxymethyl)-1,4-dihydro-pyridine-5-carboxylate; 5-isopropyl-2-methyl-3-cyano-4-(3-trifluoromethyl-4-chlorophenyl)-6-(methoxymethyl)-1,4-dihydro-pyridine-5-carboxylate; 5-isopropyl-2-methyl-3-cyano-4-(2-fluoro-4-bromophenyl)-6-(methoxymethyl)-1,4-dihydro-pyridine-5-carboxylate; 5-isopropyl-2-methyl-3-cyano-4-(2-bromo-4-fluorophenyl)-6-(methoxymethyl)-1,4-dihydro-pyridine-5-carboxylate; 5-methyl-2-methyl-3-cyano-4-(2-fluoro-4-bromophenyl)-6-(methoxymethyl)-1,4-dihydro-pyridine-5-carboxylate; 5-methyl-2-methyl-3-cyano-4-(2-bromo-4-methylphenyl)-6-(methoxymethyl)-1,4-dihydro-pyridine-5-carboxylate; 5-methyl-2-methyl-3-cyano-4-(2-fluoro-4-chlorophenyl)-6-(methoxymethyl)-1,4-dihydro-pyridine-5-carboxylate; 2,6-dimethyl-3-cyano-4-(2-fluoro-4-bromophenyl)-5-(2-methoxyphenyl)carbamoyl-1,4-dihydro-pyridine; 2,6-dimethyl-3-cyano-4-(2-fluoro-4-chlorophenyl)-5-(2-methoxyphenyl)carbamoyl-1,4-dihydro-pyridine; 2,6-dimethyl-3-cyano-4-(2-fluoro-4-trifluoromethylphenyl)-5-(2-methoxyphenyl)carbamoyl-1,4-dihydro-pyridine; 5-isopropyl-2-methyl-3-cyano-4-(2,6-dichlorophenyl)-6-(methoxymethyl)-1,4-dihydro-pyridine-5-carboxylate; 5-methyl-2-methyl-3-cyano-4-(2,6-dichlorophenyl)-6-(methoxymethyl)-1,4-dihydro-pyridine-5-carboxylate; 2,6-dimethyl-3-cyano-4-(2-fluoro-6-chlorophenyl)-5-(2-methoxyphenyl)carbamoyl-1,4-dihydro-pyridine; 2,6-dimethyl-3-cyano-4-(2,6-difluorophenyl)-5-(2-methoxyphenyl)carbamoyl-1,4-dihydro-pyridine; 2,6-dimethyl-3-cyano-4-(4-fluoro-5-trifluoromethylphenyl)-5-(2-methoxyphenyl)carbamoyl-1,4-dihydro-pyridine; 2-methyl-3-cyano-4-(2-trifluoromethyl-4-fluorophenyl)-5,6-(3,3-dimethyl)-cyclohexan-2-one-1,4-dihydro-pyridine; 5-methyl-2-methyl-3-cyano-4-[4-(2-bromopyridine)]-6-(methoxymethyl)-1,4-dihydro-pyridine-5-carboxylate; 5-methyl-2-methyl-3-cyano-4-[3-(2-methoxypyridine)]-6-propyl-1,4-dihydro-pyridine-5-carboxylate; 5-methyl-2-methyl-3-cyano-4-[3-(2,5-dichlorothiophene)]-6-propyl-1,4-dihydro-pyridine-5-carboxylate; 5-methyl-2-methyl-3-cyano-4-[3-(2,5-dichlorothiophene)]-6-cyclopropyl-1,4-dihydro-pyridine-5-carboxylate; 5-isopropyl-2-methyl-3-cyano-4-[3-(2,5-dichlorothiophene)]-6-(methoxymethyl)-1,4-dihydro-pyridine-5-carboxylate; 5-methyl-2-methyl-3-cyano-4-(3,4-difluorophenyl)-6-propyl-1,4-dihydro-pyridine-5-carboxylate; 5-isopropyl-2-methyl-3-cyano-4-(4-quinoline)-6propyl-1,4-dihydro-pyridine-5-carboxylate;
5-methyl-2-methyl-3-cyano-4-3-[2,5-dimethylthiophene)]-6-propyl-1,4-dihydro-pyridine-5-carboxylate; 5-methyl-2-methyl-3-cyano-4-3-[2,5-dimethylthiophene)]-6-cyclopropyl-1,4-dihydro-pyridine-5-carboxylate; 2,6-dimethyl-3-cyano-4-(2-ethoxyphenyl)-5-(2,4-dichlorophenyl)carbamoyl-1,4-dihydro-pyridine; 5-ethyl-2-thiomethyl-3-cyano-4-(2-chloro-3-pyridine)-6-propyl-1,4-dihydro-pyridine-5-carboxylate; 5-ethyl)-2-thiomethyl-3-cyano-4-(2-methoxy-3-pyridine)-6-propyl-1,4-dihydro-pyridine-5-carboxylate; 5-ethyl-2-methyl-3-cyano-4-(2-methyl-3-pyridine)-6-propyl-1,4-dihydro-pyridine-5-carboxylate; 5-iso-propyl-2-methyl-3-cyano-4-(2-methyl-3-pyridine)-6-propyl-1,4-dihydro-pyridine-5-carboxylate; 5-methyl-2-methyl-3-cyano-4-(2-chlorophenyl)-6-(2-methoxymethyl)-1,4-dihydro-pyridine-5-carboxylate; 5-methyl-2-methyl-3-cyano-4-(2-cbromophenyl)-6-(2-methoxymethyl)-1,4-dihydro-pyridine-5-carboxylate; 5-methyl-2-methyl-3-cyano-4-(2-methylphenyl)-6-(2-methoxymethyl)-1,4-dihydro-pyridine-5-carboxylate; 5-iso-propyl-2-methyl-3-cyano-4-(2-chlorophenyl)-6-(2-methoxymethyl)-1,4-dihydro-pyridine-5-carboxylate; 5-iso-propyl-2-methyl-3-cyano-4-(2-bromophenyl)-6-(2-methoxymethyl)-1,4-dihydro-pyridine-5-carboxylate; 5-iso-propyl-2-methyl-3-cyano-4-(2-methylphenyl)-6-(2-methoxymethyl)-1,4-dihydro-pyridine-5-carboxylate; 5-iso-propyl-2-methyl-3-cyano-4-(2-ethylphenyl)-6-(2-methoxymethyl)-1,4-dihydro-pyridine-5-carboxylate; 5-methyl-2-methyl-3-cyano-4-(2,4-dichlorophenyl)-6-(2-phenylethyl)-1,4-dihydro-pyridine-5-carboxylate; 5-methyl-2-methyl-3-cyano-4-(2-methoxy-3,4-difluorophenyl)-6-(2-methoxyethyl)-1,4-dihydro-pyridine-5-carboxylate; 5-methyl-2-methyl-3-cyano-4-(2-methoxy-3,4-difluorophenyl)-6-(2-methoxymethyl)-1,4-dihydro-pyridine-5-carboxylate; 5-methyl-2-methyl-3-cyano-4-(2-chloro-3,4-difluorophenyl)-6-(2-methoxyethyl)-1,4-dihydro-pyridine-5-carboxylate; 2,6-dimethyl-3-cyano-4-(2-fluoro-4-chloro)-5-(2-methoxyphenyl)carbamoyl-1,4-dihydropyridine; 5-methyl-2-methyl-3-cyano-4-(2,4-dichlorophenyl)-6-(5,5,5-trifluoropentyl)-1,4-dihydro-pyridine-5-carboxylate; 5-methyl-2-methyl-3-cyano-4-(2-fluoro-4-chlorophenyl)-6-(5,5,5-trifluoropentyl)-1,4-dihydro-pyridine-5-carboxylate; 5-methyl-2-methyl-3-cyano-4-(2-methoxy-3,4-difluorophenyl)-6-cyclopropyl-1,4-dihydro-pyridine-5-carboxylate; 5-methyl-2-methyl-3-cyano-4-(2-bromo-4-pyridyl)-6-cyclopropyl-1,4-dihydro-pyridine-5-carboxylate; 5-methyl-2-methyl-3-cyano-4-(2-bromo-4-pyridyl)-6-2-methoxyethyl-1,4-dihydro-pyridine-5-carboxylate; 5-methyl-2-methyl-3-cyano-4-(2-trifluoromethyl-4-fluorophenyl)-6-2-cyclopropylethyl-1,4-dihydro-pyridine-5-carboxylate; 5-methyl-2-methyl-3-cyano-4-(2-methoxy-3,4-difluorophenyl)-6-2-cyclopropylethyl-1,4-dihydro-pyridine-5-carboxylate; 5-methyl-2-methyl-3-cyano-4-(2-bromo-4-pyridyl)-6-(2-cyclopropylethyl)-1,4-dihydro-pyridine-5-carboxylate; 5-cyclopropylmethyl-2-methyl-3-cyano-4-(2-trifluoromethyl-4-fluorophenyl)-6-(2-methoxyethyl)-1,4-dihydro-pyridine-5-carboxylate; and 5-cyclopropylmethyl-2-methyl-3-cyano-4-(2-methoxy-3,4-difluorophenyl)-6-(2-methoxyethyl)-1,4-dihydro-pyridine-5-carboxylate.

9. A pharmaceutical composition comprising a therapeutically effective amount of a compound of claim 1 in combination with a pharmaceutically acceptable excipient.

10. A method for treating a disease in an animal in which modulation of steroid nuclear hormone receptor activity can prevent, inhibit or ameliorate the pathology and/or symptomatology of the disease, which method comprises administering to the animal a therapeutically effective amount of a compound of claim 1.

11. A method for treating a disease in an animal in which modulation of steroid nuclear hormone receptor activity and L-type calcium channel activity can prevent, inhibit or ameliorate the pathology and/or symptomatology of the disease, which method comprises administering to the animal a therapeutically effective amount of a compound of Formula I in which: R₁ is selected from phenyl and pyridinyl; wherein any phenyl or pyridinyl of R₁ is optionally substituted with 1 to 3 radicals independently selected from chloro, bromo, fluoro, trifluoromethyl, methyl, ethyl and C₁₋₆alkoxy; Rₓ is selected from C(O)OC₁₋₁₀alkyl and halo-substituted-C(O)OC₁₋₁₀alkyl; R₃ is selected from C₁₋₆alkyl optionally substituted with 1-5 halo radicals; wherein any alkyl of R₃ can optionally have a methylene replaced with —O—; R₄ is hydrogen; and R₅ is selected from C₁₋₆alkyl and —XR₉; wherein X is a bond or C₁₋₆alkylene; R₉ is independently selected from hydroxy, C₁₋₆alkyl, halo-substituted-C₁₋₆alkyl, C₆₋₁₀aryl and C₅₋₁₀heteroaryl; wherein any aryl or heteroaryl of R₉ is optionally substituted with 1 to 3 radicals independently selected from halo, hydroxy, nitro, amino, cyano, C₁₋₆alkyl, C₁₋₆alkoxy, halo-substituted-C₁₋₆alkyl, halo-substituted-C₁₋₆alkoxy, —C(O)OR₁₀, —OR₁₀ and C(O)R₁₀; and wherein R₁₀ is selected from methyl and phenyl.

12. The method of claim 11 in which: R₅ is selected from C₁₋₆alkyl, halo-C₁₋₆alkyl and —XR₉; wherein X is a bond or C₁₋₆alkylene; R₉ is independently selected from hydroxy, C₁₋₆alkyl, halo-substituted-C₁₋₆alkyl, C₆₋₁₀aryl and C₅₋₁₀heteroaryl; wherein any aryl or heteroaryl of R₉ is optionally substituted with 1 to 3 radicals independently selected from halo, hydroxy, nitro, amino, cyano, C₁₋₆alkyl, C₁₋₆alkoxy, halo-substituted-C₁₋₆alkyl, halo-substituted-C₁₋₆alkoxy, —C(O)OR₁₀, —OR₁₀ and —C(O)R₁₀; wherein R₁₀ is selected from methyl and phenyl.

13. The use of a compound of claim 1 in the manufacture of a medicament for treating a disease in an animal in which aberrant steroid nuclear hormone receptor activity and/or L-type calcium channel activity contributes to the pathology and/or symptomatology of the disease.
